(19) United States
(12) Patent Application Publication
(10) Pub. No.: US 2026/0337629 A1
(43) Pub. Date: Oct. 29, 2026

(54) NOVEL RESISTANCE GENES ASSOCIATED WITH DISEASE RESISTANCE IN SOYBEANS

(71) Applicants: Qingli Liu, Durham, NC (US); Andrew David Farmer, Santa Fe, NM (US); Yinping Lucy Qin, Durham, NC (US); Xiaoping Tan, Durham, NC (US)

(72) Inventors: Qingli Liu, Durham, NC (US); Andrew David Farmer, Santa Fe, NM (US); Yinping Lucy Qin, Durham, NC (US); Xiaoping Tan, Durham, NC (US)

(73) Assignee: Syngenta Crop Protection AG, Basel (CH)

(21) Appl. No.: 19/148,773

(22) PCT Filed: Jan. 25, 2024
(86) PCT No.: PCT/US2024/012883

§ 371 Date: Jul. 17, 2025

Related U.S. Application Data
(60) Provisional application No. 63/481,627, filed on Jan. 26, 2023.

(51) Int. Cl. C12N 15/82; C07K 14/415

(52) U.S. Cl. CPC C12N 15/8282; C07K 14/415; C12N 15/8201

(57) ABSTRACT

Methods and compositions for identifying, selecting and/or producing a pathogen resistant plant, plant cell or seed (e.g., a legume or soybean plant, plant cell or seed) are provided. Polynucleotides and polypeptides are provided, which when expressed in a plant increase disease and/or pathogen resistance (e.g., to Asian Soy Rust) of the plant. Plants, plant cells and seed are provided comprising such polypeptides and polynucleotides, as are methods for their use.

### RELATED APPLICATIONS

This application is a 371 of International Application No. PCT/US2024/012883, filed 25 Jan. 2024, which claims priority to U.S. Provisional Patent Application No. 63/481,627, filed Jan. 26, 2023, the contents of which are incorporated by reference herein in their entirety.

### FIELD OF THE INVENTION

The present invention relates to compositions and methods for identifying, selecting and producing enhanced disease and/or pathogen resistant plants using novel resistance genes.

### STATEMENT REGARDING ELECTRONIC SUBMISSION OF A SEQUENCE LISTING

This application is accompanied by a sequence listing entitled 82653_PCT.xml, created Jan. 18, 2024, which is approximately 100 kb in size. This sequence listing filed herewith via EFS-Web in compliance with 37 C.F.R. § 1.831-1.835, is incorporated by reference into the specification in its entirety.

### BACKGROUND

Plant pathogens are known to cause considerable damage to important crops, resulting in significant agricultural losses with widespread consequences for both the food supply and other industries that rely on plant materials. As such, applicant desires to reduce the incidence and/or impact of agricultural pathogens on crop production.

Several pathogens have been associated with damage to soybeans, which individually and collectively have the potential to cause significant yield losses in the United States and throughout the world. Exemplary pathogens include, but are not limited to fungi (e.g., genus Phytophthora and Asian Soybean rust Phakopsora pachyrhizi), nematodes (e.g., genus Meloidogyne, particularly, Meloidogyne javanica), soybean stem canker, and bacteria (e.g., Pseudomonas syringae). Given the significant threat to global food supplies that these pathogens present as well as the time and expense associated with treating soybean crops to prevent yield loss, new methods for producing pathogen resistant soybean cultivars are needed. What is needed is novel resistance genes (herein, “R-Genes”) that can be introduced into plants to control pathogens.

### SUMMARY OF THE INVENTION

Compositions and methods are provided to increase disease resistance and/or pathogen resistance of a plant, particularly legume plants, and more particularly soybean plants. The disclosure provides RG31 and RG35 polypeptides, variants and active fragments of RG31 and RG35 polypeptides, as well as fusion proteins of RG31 and RG35 polypeptides, capable of enhancing disease resistance. The disclosure further provides nucleic acids encoding the disease resistance enhancing polypeptides and fusion proteins, as well as plants expressing the same. Methods are also provided for enhancing disease resistance by providing plants with nucleic acids encoding the disclosed polypeptides.

The foregoing and other objects and aspects of the present invention are explained in detail in the drawings and specification set forth below.

### BRIEF DESCRIPTION OF THE DRAWINGS

FIG. 1A depicts the results of rust bioassay experiments conducted on leaves collected from primary soybean events comprising the novel R-genes RG35 (constructs 25227, 26228, and 26229) or RG31 (constructs 25905, 25951, and 25900). Events comprising the novel R-gene show increased ASR resistance relative to the control against rust population 21BR08.

FIG. 1B shows a graph comparing fungal biomass accumulation, as measured by mRNA of β-tubulin, in events comprising the R-gene RG35 (constructs 25227, 26228, 26229) or the R-gene RG31 (constructs 25900, 25905, 25951), when infected with the rust population 21BR08. The quantitative results match the phenotypic results of FIG. 1A.

FIG. 2A depicts the results of rust bioassay experiments conducted on leaves collected from primary soybean events generated from binary vectors comprising the R-gene RG35 (constructs 25227, 26228, and 26229) or the R-gene RG31 (constructs 25905, 25951, and 25900). Events comprising the R-gene show increased ASR resistance relative to the control against rust population 21BRM.

FIG. 2B shows a graph comparing fungal biomass accumulation, as measured by mRNA of β-tubulin, in events comprising the R-gene RG35 (constructs 25227, 26228, 26229) or the R-gene RG31 (constructs 25900, 25905, 25951), when infected with the rust population 21BRM. The quantitative results match the phenotypic results of FIG. 2A.

FIG. 3 depicts the results of rust bioassay experiments conducted on single-copy T0 events and single-copy T1 events comprising either the RG35 gene (construct 26228) or the RG31 gene (construct 25905), with expression of the R-gene in the construct driven by a Medicago promoter (prMt51186, SEQ ID NO: 12). The average and standard deviation of disease rate for each construct was calculated. T-test analysis of the data reveal that events comprising the RG35 gene exhibit significantly higher resistance to soy rust as compared to events comprising the RG31 gene. The same trend was observed for T0 and T1 events. The results indicate that Medicago promoter driven RG35 has higher disease resistance than Medicago promoter driven RG31.

FIG. 4 depicts the results of rust bioassay experiments conducted on single-copy T0 events and single-copy T1 events comprising either the RG35 gene (construct 25227) or the RG31 gene (construct 25951), with expression of the R-gene in the construct driven by the corresponding native promoter (that is, RG31 expression was driven by the native RG31 promoter of SEQ ID NO: 7 and RG35 expression was driven by the native RG35 promoter of SEQ ID NO: 10). The average and standard deviation of disease rate for each construct was calculated. T-test analysis of the data reveal that events comprising the RG35 gene exhibit significantly higher resistance to soy rust as compared to events comprising the RG31 gene. The same trend was observed for TO and T1 events. The results indicate that native promoter driven RG35 has higher disease resistance than native promoter driven RG31.

FIG. 5 shows the results of an effector protein and R-protein interaction assay in a heterologous assay system. Presence of interaction between an effector protein and an R-protein is indicated by the presence of an infiltrated area showing a Hypersensitive Response (HR) in Plant cells. Soy rust effectors (in this example, SPE-35 and SPE-01) were expressed in different halves of N. tabacum leaves via Agrobacterium mediated transient assay with RG31, RG35, or RG1. SPE-35 was found to specifically trigger a hypersensitive localized cell death response at the site of inoculation when co-infiltrated with RG31 (construct 25905; infiltration position 3) or RG35 (construct 26228; infiltration position 2). Co-infiltration of the effector with a different R-protein, RG01 (construct 23968, infiltration position 1; or construct 24732, infiltration position 4) does not trigger a similar response, indicating the specificity of SPE-35 for RG31 and RG35. This result also indicates that the mode of action for resistance proteins RG31 and RG35 is different from the mode of action for resistance protein RG1, despite the sequence similarity. Co-infiltration of the effector with the control construct expressing GUS (R-gene control, infiltration position 5) also does not trigger a hypersensitive localized cell death response at the site of inoculation, as expected.

FIG. 6 shows the results of an effector protein and R-protein interaction assay in a heterologous assay system. Presence of interaction between an effector protein and an R-protein is indicated by the presence of an infiltrated area showing a Hypersensitive Response (HR) in Plant cells. Soy rust effectors (in this example, SPE-35 and SPE-01) were expressed in different halves of N. tabacum leaves via Agrobacterium mediated transient assay with RG31, RG35, or RG1. SPE-35 specifically triggers a hypersensitive localized cell death response at the site of inoculation when co-infiltrated with RG31 (construct 25905; infiltration position 4) or RG35 (construct 26228; infiltration position 5). Co-infiltration of the effector with a different R-protein, RG01 (construct 23968, infiltration position 3; or construct 24732, infiltration position 2) does not trigger a similar response, indicating the specificity of SPE-35 for RG31 and RG35. This results also indicates that the mode of action for resistance proteins RG31 and RG35 is different from the mode of action for resistance protein RG1, despite the sequence homology. Co-infiltration of the effector with the control construct expressing GUS (R-gene control, infiltration position 1) also does not trigger a hypersensitive localized cell death response at the site of inoculation, as expected.

### BRIEF DESCRIPTION OF THE SEQUENCE LISTING

SEQ ID NO: 1 is the amino acid sequence for a RG31 resistance protein encoded by the RG31 resistance gene of Glycine tomentella.

SEQ ID NO: 2 is the amino acid sequence for a RG35 resistance protein encoded by the RG35 resistance gene of Glycine tomentella.

SEQ ID NO: 3 is the genomic sequence for the RG31 gene. SEQ ID NO: 4 is the intron-less cDNA sequence for the RG31 gene. Each of SEQ ID NOS: 3-4 encodes the protein of SEQ ID NO: 1.

SEQ ID NO: 5 is the genomic sequence for the RG35 gene. SEQ ID NO: 6 is the intron-less cDNA sequence for the RG35 gene. Each of SEQ ID NOS: 5-6 encodes the protein of SEQ ID NO: 2.

SEQ ID NO: 7 is the native promoter, prGtoRG31-01, of the RG31 gene from G. tomentella. SEQ ID NO: 8 is a modified native promoter, prGtoRG31-02, of the RG31 gene from G. tomentella. SEQ ID NO: 9 is the native terminator, tGtoRG31, of the RG31 gene from G. tomentella.

SEQ ID NO: 10 is the native promoter, prGtoRG35, of the RG35 gene from G. tomentella. SEQ ID NO: 11 is the native terminator, tGtoRG35, of the RG35 gene from G. tomentella.

SEQ ID NO: 12 is a plant active constitutive promoter, prMt51186, derived from Medicago truncatula. SEQ ID NO: 13 is a plant active terminator, tMt51186, derived from Medicago truncatula.

SEQ ID NO: 14 is a rust reactive/inducible promoter, prGmACO3, derived from the first intron of the rust reactive promoter of an ACO3 gene encoding an oxidoreductase (1-Aminocyclopropane-1-Carboxylate Oxidase 3-Related) protein on chromosome 2 in soybean (Glyma.02G268200).

SEQ ID NO: 15 is a rust reactive/inducible promoter, prGmMYB, of the gene encoding a Myb-Like DNA-Binding protein on chromosome 2 in soybean (Glyma. 19G164600).

SEQ ID NO: 16 is the amino acid sequence for effector protein SPE-35 from Phakopsora pachyrhizi. SEQ ID NO: 17 is a polynucleotide sequence encoding the protein of SEQ ID NO: 16.

SEQ ID NO: 18 is the amino acid sequence for effector protein SPE-01 from Phakopsora pachyrhizi. SEQ ID NO: 19 is a polynucleotide sequence encoding the protein of SEQ ID NO: 18.

SEQ ID NO: 20 is the amino acid sequence for a longer splice variant of the RG35 polypeptide derived from the Rg35 gene of Glycine canescens. SEQ ID NOS: 21 and 22 are nucleotide sequences that encode the splice variant protein of SEQ ID NO: 20.

SEQ ID NO: 23 is the amino acid sequence for effector protein SPE-38 from Phakopsora pachyrhizi. SEQ ID NO: 24 is a polynucleotide sequence encoding the protein of SEQ ID NO: 22.

### DETAILED DESCRIPTION OF THE INVENTION

Compositions and methods are provided to increase disease resistance and/or pathogen resistance of a plant, particularly soybean plants, via the expression of novel resistance proteins RG31 and/or RG35. The RG31 and RG35 proteins are surprisingly beneficial in that, when expressed in a plant, the proteins are able of enhancing disease resistance through a novel mode of action. This novel mode of action distinguishes the protein from other R-proteins to which RG31 and RG35 have high sequence homology, such as the RG1 resistance protein disclosed in WIPO Publication No. WO2019103918A1 (as SEQ ID NO: 47).

### 1. DEFINITIONS

Unless defined otherwise, all technical and scientific terms used herein have the same meaning as commonly understood to one of ordinary skill in the art to which the presently disclosed subject matter belongs.

Although the following terms are believed to be well understood by one of ordinary skill in the art, the following definitions are set forth to facilitate understanding of the presently disclosed subject matter.

All references listed below, as well as all references cited in the instant disclosure, including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.

Nucleotide sequences provided herein are presented in the 5′ to 3′ direction, from left to right and are presented using the standard code for representing nucleotide bases as set forth in 37 CFR §§ 1.821-1.825 and §§ 1.831-1.835 and the World Intellectual Property Organization (WIPO) Standards ST.25 and ST.26, for example: adenine (A), cytosine (C), thymine (T), and guanine (G).

Amino acids are likewise indicated using the WIPO Standard ST.25, for example: alanine (Ala; A), arginine (Arg; R), asparagine (Asn; N), aspartic acid (Asp; D), cysteine (Cys; C), glutamine (Gln; Q), glutamic acid (Glu; E), glycine (Gly; G), histidine (His; H), isoleucine (Ile; 1), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

The singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The term “about,” as used herein when referring to a measurable value such as a dosage, application rate, or time period and the like, is meant to encompass variations of ±20%, ±10%, +5%, ±1%, ±0.5%, or even ±0.1% of the specified amount. As used herein, phrases such as “between about X and Y” mean “between about X and about Y” and phrases such as “from about X to Y” mean “from about X to about Y.”

As used herein, phrases such as “between about X and Y”, “between about X and about Y”, “from X to Y” and “from about X to about Y” (and similar phrases) should be interpreted to include X and Y, unless the context indicates otherwise.

As used herein, a “coding sequence” or “CDS” is a nucleic acid sequence that is transcribed into RNA such as mRNA, rRNA, tRNA, snRNA, sense RNA or antisense RNA. In embodiments, the RNA is then translated to produce a protein. In example embodiments, the CDS is derived from a cDNA sequence and includes the sequence of spliced exons of a transcript in DNA notation and does not include any intron or 5′ or 3′-untranslated regions (UTRs). In other example embodiments, the CDS is derived from a genomic DNA sequence and includes the sequence of spliced exons of a transcript in DNA notation as well as one or more introns, and 5′ and/or 3′-untranslated regions (UTRs).

As used herein, a “codon optimized” nucleotide sequence means a nucleotide sequence of a recombinant, transgenic, or synthetic polynucleotide wherein the codons are chosen to reflect the particular codon bias that a host cell or organism may have. This is typically done in such a way as to preserve the amino acid sequence of the polypeptide encoded by the codon optimized nucleotide sequence. In certain embodiments, a nucleotide sequence is codon optimized for the cell (e.g., an animal, plant, fungal or bacterial cell) in which the construct is to be expressed. For example, a construct to be expressed in a plant cell can have all or parts of its sequence codon optimized for expression in a plant. See, for example, U.S. Pat. No. 6,121,014. In embodiments, the polynucleotides provided herein are codon-optimized for expression in a plant cell (e.g., a dicot cell, a monocot cell, a soybean cell) or bacterial cell.

The term “comprise”, “comprises” or “comprising,” when used in this specification, indicates the presence of the stated features, integers, steps, operations, elements, or components, but does not preclude the presence or addition of one or more other features, integers, steps, operations, elements, components, and/or groups thereof.

As used herein, the transitional phrase “consisting essentially of” (and grammatical variants) means that the scope of a claim is to be interpreted to encompass the specified materials or steps recited in the claim “and those that do not materially alter the basic and novel characteristic(s)” of the claimed invention. Thus, the term “consisting essentially of” when used in a claim of this invention is not intended to be interpreted to be equivalent to “comprising.”

“Expression cassette” as used herein means a nucleic acid molecule capable of directing expression of at least one polynucleotide of interest, such as a polynucleotide encoding an RG31 polypeptide or active variant or fragment thereof, or a polypeptide encoding an RG35 polypeptide or active variant or fragment thereof in an appropriate host cell, and comprises a promoter operably linked to the polynucleotide of interest which is operably linked to a termination signal. An “expression cassette” can comprise additional polynucleotides to facilitate proper translation of the polynucleotide of interest. The expression cassette may comprise other polynucleotides not related to the expression of a polynucleotide of interest, but which are present due to convenient restriction sites for removal of the cassette from an expression vector. In embodiments, at least one of the components in the expression cassette may be heterologous (i.e., foreign) with respect to at least one of the other components (e.g., a heterologous promoter, a terminator, an intron, and/or any regulatory element operatively associated with a polynucleotide of interest). In other embodiments, the expression cassette may be naturally occurring and comprises the native regulatory elements, one or more or all of the native introns, and native genomic DNA to allow for the expression of the RG31 or RG35 polypeptide, or active variants or fragments or fusions thereof. The expression cassette can be heterologous with respect to the host, i.e., the expression cassette (or even the polynucleotide of interest) does not occur naturally in the host cell and has been introduced into the host cell by a transformation process or a breeding process.

In embodiments of the present invention, a cell is provided that comprises an RG31 polypeptide (or an active variant or fragment thereof) or an RG35 polypeptide (or an active variant or fragment thereof). In an example embodiment, a cell is provided comprising a heterologous polynucleotide encoding an RG31 polypeptide, such as a polynucleotide encoding a polypeptide having at least 90% identity or at least 95% identity to SEQ ID NO: 1 or 2, wherein increased expression of the polypeptide in a plant increases disease resistance in the plant; or a polypeptide comprising SEQ ID NO: 1 or 2. In other example embodiments, the cell comprises a heterologous polynucleotide comprising a nucleotide sequence having at least 90%, or at least 95% sequence identity to SEQ ID NO: 3, 4 5, or 6; or a nucleotide sequence comprising SEQ ID NO: 3 or 4 or 5 or 6. In particular embodiments, the cell is a plant cell, and the polynucleotide is stably integrated into the genome of the cell. In specific embodiments, the plant cell has an increased level of expression of the polypeptide and the plant cell has increased disease resistance relative to a control plant cell.

The term “introduced” or “introducing” defines a process of altering the content of a cell or a plant through the use of traditional breeding or recombinant transformation techniques. Any means can be used to introduce polynucleotides into a cell or a plant cell, including methods that result in stable transformation, transient transformation, or a gene edit. These include the use of calcium phosphate transfection, polybrene, protoplast fusion, PEG, electroporation, ultrasonic methods (e.g., sonoporation), liposomes, microinjection, naked DNA, plasmid vectors, viral vectors, both episomal and integrative, introgression, transgenic, Clustered Regularly Interspaced Short Palindromic Repeats modification (CRISPR), Transcription activator-like effector nucleases (TALENs) (Feng et al. 2013, Joung & Sander 2013), meganucleases, or zinc finger nucleases (ZFNs) and any of the other well-known methods for introducing cloned genomic DNA, cDNA, synthetic DNA or other foreign genetic material into a host cell.

As used herein, the term “wild glycine” refers to a perennial Glycine plant, for example any one of G. canescens, G. argyrea, G. clandestine, G. latrobeana, G. albicans, G. aphyonota, G. arenaria, G. curvata, G. cyrtoloba, G. dolichocarpa, G. falcate, G. gracei, G. hirticaulis, G. lactovirens, G. latifolia, G. microphylla, G. montis-douglas, G. peratosa, G. pescadrensis, G. pindanica, G. pullenii, G. rubiginosa, G. stenophita, G. syndetika, or G. tomentella.

As used herein, the term “allele” refers to one of two or more different nucleotides or nucleotide sequences that occur at a specific locus.

A marker is “associated with” a trait when it is linked to it and when the presence of the marker is an indicator of whether and/or to what extent the desired trait or trait form will occur in a plant/germplasm comprising the marker. Similarly, a marker is “associated with” an allele when it is linked to it and when the presence of the marker is an indicator of whether the allele is present in a plant/germplasm comprising the marker. For example, “a marker associated with enhanced pathogen resistance” or “a marker associated with enhanced disease resistance” refers to a marker whose presence or absence can be used to predict whether and/or to what extent a plant will display a pathogen resistant phenotype.

A marker may be, but is not limited to, an allele, a gene, a haplotype, a restriction fragment length polymorphism (RFLP), a simple sequence repeat (SSR), random amplified polymorphic DNA (RAPD), cleaved amplified polymorphic sequences (CAPS) (Rafalski and Tingey, Trends in Genetics 9:275 (1993)), an amplified fragment length polymorphism (AFLP) (Vos et al., Nucleic Acids Res. 23:4407 (1995)), a single nucleotide polymorphism (SNP) (Brookes, Gene 234:177 (1993)), a sequence-characterized amplified region (SCAR) (Paran and Michelmore, Theor. Appl. Genet. 85:985 (1993)), a sequence-tagged site (STS) (Onozaki et al., Euphytica 138:255 (2004)), a single-stranded conformation polymorphism (SSCP) (Orita et al., Proc. Natl. Acad. Sci. USA 86:2766 (1989)), an inter-simple sequence repeat (ISSR) (Blair et al., Theor. Appl. Genet. 98:780 (1999)), an inter-retrotransposon amplified polymorphism (IRAP), a retrotransposon-microsatellite amplified polymorphism (REMAP) (Kalendar et al., Theor. Appl. Genet. 98:704 (1999)), a chromosome interval, or an RNA cleavage product (such as a Lynx tag). A marker may be present in genomic or expressed nucleic acids (e.g., ESTs). The term marker may also refer to nucleic acids used as probes or primers (e.g., primer pairs) for use in amplifying, hybridizing to and/or detecting nucleic acid molecules according to methods well known in the art (e.g., using PCR).

As used herein, the terms “backcross” and “backcrossing” refer to the process whereby a progeny plant is repeatedly crossed back to one of its parents. In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. For example, see Ragot, M. et al. Marker-assisted Backcrossing: A Practical Example, in TECHNIQUES ET UTILISATIONS DES MARQUEURS MOLECULAIRES LES COLLOQUES, Vol. 72, pp. 45-56 (1995); and Openshaw et al., Marker-assisted Selection in Backcross Breeding, in PROCEEDINGS OF THE SYMPOSIUM “ANALYSIS OF MOLECULAR MARKER DATA,” pp. 41-53 (1994). The initial cross gives rise to the F1 generation. The term “BC1” refers to the second use of the recurrent parent, “BC2” refers to the third use of the recurrent parent, and so on.

A centimorgan (“cM”) is a unit of measure of recombination frequency. One cM is equal to a 1% chance that a marker at one genetic locus will be separated from a marker at a second locus due to crossing over in a single generation.

As used herein, the terms “cross” or “crossed” refer to the fusion of gametes via pollination to produce progeny (e.g., cells, seeds or plants). The term encompasses both sexual crosses (the pollination of one plant by another) and selfing (self-pollination, e.g., when the pollen and ovule are from the same plant). The term “crossing” refers to the act of fusing gametes via pollination to produce progeny.

As used herein, the terms “cultivar” and “variety” refer to a group of similar plants that by structural or genetic features and/or performance can be distinguished from other varieties within the same species.

As used herein, the terms “desired allele”, “favorable allele” and “allele of interest” are used interchangeably to refer to an allele associated with a desired trait (e.g., ASR resistance).

As used herein, “disease resistance gene” or “resistance gene” or “R-gene” refers to a nucleic acid having a nucleotide sequence (e.g., DNA sequence) encoding a polypeptide, R-protein, or Resistance protein, that when expressed in a plant cell, is capable of enhancing or improving or increasing a defense or immune response in the plant cell, thereby conferring the plant with increased resistance to one or more plant pathogens. In specific embodiments, the Rg31 and Rg35 genes of the present invention are disease resistance genes, or R-genes, encoding polypeptides that confer enhanced pathogen resistance when expressed in a plant cell. The encoded RG31 and RG35 polypeptides, or their active variants or fragments, can be expressed in plants to enhance pathogen resistance to a plant pathogen, such as a fungal pathogen. As non-limiting examples, the R-genes of the present invention, and their encoded polypeptides, can be used to enhance resistance to the funal pathogen Phytopthora, and to the disease Asian Soybean Rust. In other examples, the R-genes of the present invention and their encoded polypeptides can be used to enhance resistance to other fungal pathogens, such as those responsible for Powdery mildew, as well as bacterial pathogens and nematodes, such as soybean cyst nematode (SCN) and root knot nematode.

R-gene embodiments may comprise one or more motifs that correlate with one or more domains of the corresponding R-protein. Embodiments of an R-gene, such as the RG31 and RG35 genes of the present disclosure, encode polypeptides having a TNLWK motif comprising a Toll/Interleukin-1 receptor (TIR) domain, a nucleotide-binding site (NBS), and a leucine rich-repeat (LRR) domain, a protein kinase domain, and a WRKY domain. In other embodiments, an R-gene may encode polypeptides having a CNL motif comprising a coiled coil (CC) domain, a nucleotide-binding site (NBS), and a leucine rich-repeat (LRR) domain. In still other embodiments, an R-gene may encode polypeptides having a TNL motif comprising a Toll/Interleukin-1 receptor (TIR) domain, a nucleotide-binding site (NBS), and a leucine rich-repeat (LRR) domain. In some embodiments, R-genes may additionally or alternatively encode polypeptides having domains of unknown function, wherein the function of the domains is unknown at the time of gene discovery. In still further embodiments, R-genes may additionally or alternatively encode polypeptides having putative effector binding domains and/or one or more putative transmembrane helices. In further embodiments, R-genes may encode polypeptides having fewer or additional domains and motifs.

In embodiments, the nucleic acid sequence of the R-gene is derived from a wild plant exhibiting increased resistance to the pathogen and includes, at least, a coding sequence encoding the resistance conferring polypeptide. The nucleic acid sequence of the R-gene may further comprise nucleic acid sequences corresponding to one or more native regulatory elements (such as native introns, native promoters, native UTRs), one or more heterologous regulatory elements (such as a heterologous promoter and one or more introns), and combinations thereof. Insertion of the R-gene into a plant that has decreased resistance to the pathogen (e.g., no resistance or partially or fully susceptible), at a chromosomal location (e.g., stably integrated into the plant genome) or extra-chromosomal location (e.g., on a vector or plasmid) results in conferring of the wild plant-derived pathogen resistance to the recipient plant. For example, in representative embodiments, an R-gene of the present invention is derived from Glycine tomentella, Glycine canescens, or Glycine clandestina and can be inserted into Glycine max plants to confer or enhance resistance of G. max plants to pathogens such as those responsible for causing Asian Soy Rust.

As used herein, “variants” of a polynucleotide sequence encoding the disease resistance R-gene and/or “variants” of a polypeptide sequence encoding a corresponding R-protein conferring disease resistance include, as non-limiting examples, annotation and splice variants. As used herein, annotation variants refer to sequences that differ from one another due to differences in annotation of regulatory sequences, including but not limited to transcription start site, position of starting ATG codon, position of splice sites, position of introns and/or exons, etc. In embodiments, a first annotation variant of a polypeptide may be longer than a second annotation variant due to the selection of an upstream start codon (e.g., an upstream Methionine annotated as the start codon in the first annotation variant while a downstream Methionine is annotated as the start codon in the second annotation variant). In further embodiments, the first annotation variant comprises the second annotation variant. RG31 and RG35 annotation variants have RG31 and RG35 activity, respectively, including the ability to confer increased disease resistance when expressed in a plant.

As used herein, “splice variants” refer to sequences that differ from a reference sequence due to alterations in the DNA sequence that occur during splicing at a splice site. As used herein, “splicing” refers to the process by which a pre-mRNA transcript is transformed into a mRNA molecule that can be translated into protein. Typically, this occurs by the removal of introns and the splicing back together of exons of the transcript. However, genetic alterations of the sequence can occur at the boundary of an exon and an intron, that is, at a splice site. The inclusion of one or more introns, or removal of one or more exons, during splicing can result in the creation of different mRNA molecules from the same gene, which in turn results in correspondingly different protein sequences being expressed from a single gene. These differing sequences, at the DNA, RNA and/or protein level, are referred to herein as “alternate splice variants”. Splice variant polypeptides encoded by alternative splicing of a gene sequence can include variants having a larger or smaller number of amino acids in the sequence and/or variants having alternate amino acids at particular positions within the sequence. In embodiments, alternate splice variants of a gene may be predicted based on the differing annotation of regions of a gene sequence as an exon or an intron (e.g., via analysis of a sequence using a sequence predicting software). In embodiments, RG31 and RG35 splice variants have RG31 and RG35 activity, respectively, including the ability to confer increased disease resistance when expressed in a plant.

As used herein, the terms “disease tolerance”, “disease tolerant”, “disease resistant” or “disease resistance” means a statistically significant increase in disease tolerance and/or a statistically significant decrease, or the absence, in one or more disease symptoms of a plant caused by a plant pathogen when compared to an appropriate control plant. Thus, the terms refer to a plant's ability to endure and/or thrive despite being infected with the respective disease. In some embodiments, an increase in disease tolerance or resistance can be (1) measured by a plant's ability to endure and/or thrive despite being infected with a respective disease; (2) measured by infected disease resistant legume or soybean plants yielding as well as (or nearly as well) as uninfected legume or soybean plants; or (3) measured by a delay or the prevention of proliferation of a pathogen (e.g., fungi), including a delay or prevention in disease related symptoms. In still other embodiments, a plant or germplasm can be labeled as “disease resistant” if it displays “enhanced or increased pathogen resistance” when compared to a control plant.

As used herein, the terms “enhanced pathogen resistance”, “enhanced disease resistance”, “increased resistance to a pathogen,” or “confers pathogen resistance” refers to an improvement, enhancement, or increase in a plant's ability to endure and/or thrive despite being infected with a pathogen or disease (e.g., Asian soybean rust) as compared to one or more control plants. Enhanced disease resistance includes a reduction in symptoms indicative of infection for a disease such as Asian soybean rust (“ASR”). An enhanced plant pathogen resistance may comprise any statistically significant increase in resistance to the plant pathogen, including, for example, an increase of at least 1%, 5%, 10%, 20%, 30%, 40%, 50%, 60%, 70%, 80%, 90%, 100% or higher. Conferring or enhancing or increasing resistance may include a reduction (partial reduction or complete reduction) in symptoms or phenotypic characteristics associated with susceptibility to the pathogen and/or an increase in phenotypic characteristics associated with resistance to the pathogen. In example embodiments, conferring or increasing of resistance to Asian Soy Rust can include a statistically significant reduction in the number, size, and/or density of lesions, change in the color of lesions (such as from a tan coloration to a reddish-brown coloration), reduction in number and density of pustule formation, reduction in sporulation, reduction in defoliation, a reduction in yield loss, or any combination thereof. Further, enhanced pathogen resistance can include the prevention or delay of proliferation of a pathogen (e.g., fungus) in the plant.

A “control” or “control plant” or “control plant cell” provides a reference point for measuring changes in phenotype of the subject plant or plant cell. A control plant or plant cell may comprise, for example: (a) a wild-type plant or cell, i.e., of the same genotype as the starting material for the genetic alteration which resulted in the subject plant or cell; (b) a plant or plant cell of the same genotype as the starting material but which has been transformed with a null construct (i.e., with a construct which has no known effect on the trait of interest, such as a construct comprising a marker gene); (c) a plant or plant cell which is a non-transformed segregant among progeny of a subject plant or plant cell; (d) a plant or plant cell genetically identical to the subject plant or plant cell but which is not exposed to conditions or stimuli that would induce expression of the gene of interest; or (e) the subject plant or plant cell itself, under conditions in which the gene of interest is not expressed.

As used herein, “cisgenic” or “cisgenesis” involves the insertion, optionally into a genome (e.g., a plant genome), of one or more genes of the same or a related species, or from a crossable donor. As used herein a “cisgenic construct” is a recombinant nucleic acid sequence present in a cell, and optionally integrated into the cell's genome, wherein the recombinant nucleic acid sequence comprises a regulatory element operably linked to a nucleic acid sequence for a gene of interest, wherein the regulatory element and gene of interest are both native to the plant, or from a related species, or from a crossable donor, and are operably linked in the native cell at a genomic location different from the genomic location where they are integrated as the cisgenic construct. The introduction of specific alleles/genes present in the gene pool, without any DNA sequence change, via cisgenesis accelerates the breeding of species with long reproduction cycles with no linkage drag.

An “elite line” or “elite strain” is an agronomically superior line that has resulted from many cycles of breeding and selection for superior agronomic performance. Numerous elite lines are available and known to those of skill in the art of soybean breeding. An “elite population” is an assortment of elite individuals or lines that can be used to represent the state of the art in terms of agronomically superior genotypes of a given crop species, such as soybean. Similarly, an “elite germplasm” or elite strain of germplasm is an agronomically superior germplasm, typically derived from and/or capable of giving rise to a plant with superior agronomic performance, such as an existing or newly developed elite line of soybean.

An “elite” plant is any plant from an elite line, such that an elite plant is a representative plant from an elite variety. Non-limiting examples of elite soybean varieties that are commercially available to farmers or soybean breeders include: AG00802, A0868, AG0902, A1923, AG2403, A2824, A3704, A4324, A5404, AG5903, AG6202 AG0934; AG1435; AG2031; AG2035; AG2433; AG2733; AG2933; AG3334; AG3832; AG4135; AG4632; AG4934; AG5831; AG6534; and AG7231 (Asgrow Seeds, Des Moines, Iowa, USA); BPR0144RR, BPR 4077NRR and BPR 4390NRR (Bio Plant Research, Camp Point, Ill., USA); DKB17-51 and DKB37-51 (DeKalb Genetics, DeKalb, Ill., USA); DP 4546 RR, and DP 7870 RR (Delta & Pine Land Company, Lubbock, Tex., USA); JG 03R501, JG 32R606C ADD and JG 55R503C (JGL Inc., Greencastle, Ind., USA); NKS 13-K2 (NK Division of Syngenta Seeds, Golden Valley, Minnesota, USA); 90M01, 91M30, 92M33, 93M11, 94M30, 95M30, 97B52, P008T22R2; P16T17R2; P22T69R; P25T51R; P34T07R2; P35T58R; P39T67R; P47T36R; P46T21R; and P56T03R2 (Pioneer Hi-Bred International, Johnston, Iowa, USA); SG4771NRR and SG5161NRR/STS (Soygenetics, LLC, Lafayette, Ind., USA); S00-K5, S11-L2, S28-Y2, S43-B1, S53-A1, S76-L9, S78-G6, S0009-M2; S007-Y4; S04-D3; S14-A6; S20-T6; S21-M7; S26-P3; S28-N6; S30-V6; S35-C3; S36-Y6; S39-C4; S47-K5; S48-D9; S52-Y2; S58-Z4; S67-R6; S73-S8; and S78-G6 (Syngenta Seeds, Henderson, Ky., USA); Richer (Northstar Seed Ltd. Alberta, CA); 14RD62 (Stine Seed Co. Ia., USA); or Armor 4744 (Armor Seed, LLC, Ar., USA).

The terms “agronomically elite” as used herein, means a genotype that has a culmination of many distinguishable traits such as emergence, vigor, vegetative vigor, disease resistance, seed set, standability, yield and threshability which allows a producer to harvest a product of commercial significance.

A “native” or “wild type” nucleic acid, nucleotide sequence, polypeptide or amino acid sequence refers to a naturally occurring or endogenous nucleic acid, nucleotide sequence, polypeptide or amino acid sequence. Thus, for example, a “wild type mRNA” is an mRNA that is naturally occurring in, or endogenous to, the organism.

The terms “nucleic acid,” “nucleic acid molecule,” “nucleotide sequence,” “oligonucleotide”, “polynucleic acids” and “polynucleotide” are used interchangeably herein, unless the context indicates otherwise, and refer to a heteropolymer of nucleotides. These terms include without limitation DNA and RNA molecules, including cDNA, genomic DNA, synthetic (e.g., chemically synthesized) DNA and RNA, plasmid DNA, mRNA, anti-sense RNA, and RNA/DNA hybrids, any of which can be linear or branched, single stranded or double stranded, or a combination thereof. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

By “operably linked” or “operably associated” as used herein, it is meant that the indicated elements are functionally related to each other and are also generally physically related. Thus, the term “operably linked” or “operably associated” as used herein, refers to nucleotide sequences on a single nucleic acid molecule that are functionally associated. Thus, a first nucleotide sequence that is operably linked to a second nucleotide sequence, means a situation when the first nucleotide sequence is placed in a functional relationship with the second nucleotide sequence. For instance, a promoter is operably associated with a nucleotide sequence if the promoter effects the transcription or expression of said nucleotide sequence. Those skilled in the art will appreciate that the control sequences (e.g., promoter, intron, terminator, enhancer) need not be contiguous with the nucleotide sequence to which it is operably associated, as long as the control sequences function to direct the expression thereof. Thus, for example, intervening untranslated, yet transcribed, sequences can be present between a promoter and a nucleotide sequence, and the promoter can still be considered “operably linked” to or “operatively associated” with the nucleotide sequence.

As used herein, the term “endogenous” refers to materials originating from within an organism or cell. “Exogenous” refers to materials originating from outside of an organism or cell. This typically applies to nucleic acid molecules used in producing transformed or transgenic host cells and plants. For example, a nucleic acid molecule comprising the RG31 polypeptide or active variant or fragment thereof is an exogenous nucleic acid used to confer or enhance pathogen resistance in a plant cell transformed with the nucleic acid molecule. As another example, a nucleic acid molecule comprising the RG35 polypeptide or active variant or fragment thereof is an exogenous nucleic acid used to confer or enhance pathogen resistance in a plant cell transformed with the nucleic acid molecule.

As used herein, the terms “exotic,” “exotic line” and “exotic germplasm” refer to any plant, line or germplasm that is not elite. In general, exotic plants/germplasms are not derived from any known elite plant or germplasm, but rather are selected to introduce one or more desired genetic elements into a breeding program (e.g., to introduce novel alleles into a breeding program).

As used herein, the term “genome” as it applies to plant cells encompasses not only chromosomal DNA found within the nucleus, but organelle DNA found within subcellular components of the cell.

The term “gene” means a nucleic acid that comprises chromosomal DNA, genomic DNA, plasmid DNA, cDNA, an artificial DNA polynucleotide, or other DNA encoding a polypeptide of interest. In particular embodiments, the nucleic acid sequence of the gene encodes a protein that, when expressed, is responsible, at least in part, for a particular characteristic or trait. In embodiments, the gene may be native, modified (e.g., by directed recombination or site-specific mutation), or synthetic. In example embodiments, the gene is transcribed into an RNA molecule (e.g., an mRNA) in a cell wherein the RNA may encode a peptide, polypeptide, or protein of interest, and in some examples may also encode genetic elements flanking the coding sequence that are involved in the regulation of expression of the mRNA or polypeptide of the present invention. A gene may thus comprise several operably linked sequences, such as a promoter sequence, a 5′ leader sequence comprising, for example, sequences involved in translation initiation, a (protein) coding region (comprising cDNA or genomic DNA), a 3′ non-translated sequence comprising, for example, transcription termination sequence sites, introns (e.g., one or more native, foreign, or modified introns). In example embodiments, the nucleic acid sequence of the isolated gene may include introns, exons, 5′ or 3′-untranslated regions (UTRs), and native regulatory elements (such as native promoters). In other example embodiments, the gene comprises a coding sequence for a polypeptide of interest without including any regulatory elements. As such the nucleic acids encoding the RG31 protein or active variants or fragment thereof, or the RG35 protein or active variants or fragments thereof, can comprise all the native regulatory elements and/or all of the native introns. Alternatively, the nucleic acids encoding the RG31 protein or active variants or fragment thereof, or the RG35 protein or active variants or fragments thereof, lack all native or foreign/heterologous introns, can have only a subset of all the native introns (e.g., only one or two or three of them, or all but one or two or three of them), can have one, two, three or more or all of the native introns replaced with foreign or modified introns, or have one or more of the native regulatory elements (promoters, 5′ UTRs, 3′ UTR and/or terminators) replaced with foreign/heterologous or modified regulatory elements (promoters, 5′ UTRs, 3′ UTRs or and/or terminators), or any combination thereof.

As used herein, “heterologous” in reference to a polypeptide or polynucleotide sequence is a sequence that originates from a foreign species; or, if from the same species, is substantially modified from its native form in composition and/or genomic locus by deliberate human intervention. As such, heterologous sequences are in a configuration not found in nature and introduced via deliberate human intervention.

As used herein, the term “hybrid” refers to a seed and/or plant produced when at least two genetically dissimilar parents are crossed.

As used herein, the term “inbred” refers to a substantially homozygous plant or variety. The term may refer to a plant or variety that is substantially homozygous throughout the entire genome or that is substantially homozygous with respect to a portion of the genome that is of particular interest.

As used herein, the terms “introgression,” “introgressing” and “introgressed” refer to both the natural and artificial transmission of a desired allele or combination of desired alleles of a genetic locus or genetic loci from one genetic background to another. For example, a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele may be a selected allele of a marker, a QTL, a transgene, or the like. Offspring comprising the desired allele can be repeatedly backcrossed to a line having a desired genetic background and selected for the desired allele, with the result being that the desired allele becomes fixed in the desired genetic background. For example, an RG31 polypeptide or active variant or fragment thereof or marker associated with enhanced ASR tolerance or resistance may be introgressed from a donor into a recurrent parent that is not disease resistant. As another example, an RG35 polypeptide or active variant or fragment thereof or marker associated with enhanced ASR tolerance or resistance may be introgressed from a donor into a recurrent parent that is not disease resistant. The resulting offsprings could then be repeatedly backcrossed and selected until the progeny possess the ASR tolerance allele(s) in the recurrent parent background.

As used herein, an “isolated” nucleic acid molecule or gene is substantially separated away from other nucleic acid or gene sequences with which the nucleic acid is normally associated, such as, from the chromosomal or extrachromosomal DNA of a cell in which the nucleic acid or gene naturally occurs. A nucleic acid molecule is an isolated nucleic acid molecule when it comprises a transgene or part of a transgene present in the genome of another organism. The term also embraces nucleic acids that are biochemically purified to substantially remove contaminating nucleic acids and other cellular components.

A polypeptide is “isolated” if it has been separated from the cellular components (nucleic acids, lipids, carbohydrates, and other polypeptides) that naturally accompany it or that is chemically synthesized or recombinant. A polypeptide molecule is an isolated polypeptide molecule when it is expressed from a transgene in another organism. A monomeric polypeptide is isolated when at least 60% by weight of a sample is composed of the polypeptide, preferably 90% or more, more preferably 95% or more, and most preferably more than 99%. Protein purity or homogeneity is indicated, for example, by polyacrylamide gel electrophoresis of a protein sample, followed by visualization of a single polypeptide band upon staining the polyacrylamide gel; high pressure liquid chromatography; or other conventional methods. Proteins can be purified by any of the means known in the art, for example as described in Guide to Protein Purification, ed. Deutscher, Meth. Enzymol. 185, Academic Press, San Diego, 1990; and Scopes, Protein Purification: Principles and Practice, Springer Verlag, New York, 1982.

A “locus” is a position on a chromosome where a gene or marker or allele is located. In some embodiments, a locus may encompass one or more nucleotides.

A “non-naturally occurring variety of legume or soybean” is any variety of legume or soybean that does not naturally exist in nature. A “non-naturally occurring variety of legume or soybean” may be produced by any method known in the art, including, but not limited to, transforming a legume or soybean plant or germplasm, transfecting a legume or soybean plant or germplasm and crossing a naturally occurring variety of legume or soybean with a non-naturally occurring variety of soybean. In some embodiments, a “non-naturally occurring variety of legume or soybean” may comprise one or more heterologous nucleotide sequences. In some embodiments, a “non-naturally occurring variety of soybean” may comprise a non-natural combination of two or more naturally occurring nucleotide sequences (i.e., two or more naturally occurring genes that do not naturally occur in the same soybean, for instance genes not found in Glycine max lines such as polynucleotides from wild glycine species).

As used herein, the terms “phenotype,” “phenotypic trait” or “trait” refer to one or more traits and/or manifestations of an organism. The phenotype can be a manifestation that is observable to the naked eye, or by any other means of evaluation known in the art, e.g., microscopy, biochemical analysis, or an electromechanical assay. In some cases, a phenotype or trait is directly controlled by a single gene or genetic locus, i.e., a “single gene trait.” In other cases, a phenotype or trait is the result of several genes.

As used herein, the term “plant” may refer to a whole plant, any part thereof, or a cell or tissue culture derived from a plant. Thus, the term “plant” can refer to any of: whole plants, plant components or organs (e.g., roots, stems, leaves, buds, flowers, pods, etc.), plant tissues, seeds and/or plant cells. A plant cell is a cell of a plant, taken from a plant, or derived through culture from a cell taken from a plant. Thus, the term “soybean plant” may refer to a whole soybean plant, one or more parts of a soybean plant (e.g., roots, root tips, stems, leaves, buds, flowers, pods, seeds, cotyledons, etc.), soybean plant cells, soybean plant protoplasts and/or soybean plant calli.

A “plant cell” is a structural and physiological unit of a plant, comprising a protoplast and a cell wall. The plant cell may be in the form of an isolated single cell or a cultured cell, or as a part of a higher organized unit such as, for example, plant tissue, a plant organ, or a whole plant. In embodiments, the plant cell is non-propagating and/or cannot regenerate a whole plant.

A “plant cell culture” means a culture of plant units such as, for example, protoplasts, cell culture cells, cells in plant tissues, pollen, pollen tubes, ovules, embryo sacs, zygotes and embryos at various stages of development.

“Plant material” or “plant part” refers to leaves, stems, roots, flowers or flower parts, fruits, pollen, egg cells, zygotes, seeds, cuttings, cell or tissue cultures, or any other part or product of a plant.

A “plant organ” is a distinct and visibly structured and differentiated part of a plant such as a root, stem, leaf, flower bud, or embryo.

As used herein, the term “plant part” includes but is not limited to single cells and tissues from embryos, pollen, ovules, egg cells, seeds, leaves, flowers, flower parts, branches, fruit, stems, stalks, roots, root tips, anthers, cuttings and seeds, zygotes, anthers, shoots, scions, rootstocks, and/or plant cells including plant cells that are intact in plants and/or parts of plants, plant protoplasts, plant tissues, plant cell tissue cultures, plant calli, plant clumps, and the like. In some embodiments, the plant part or plant cell can be regenerated into a plant, while in other embodiments, the plant part or plant cell cannot be regenerated into a plant.

“Plant tissue” as used herein means a group of plant cells organized into a structural and functional unit. Any tissue of a plant in planta or in culture is included. This term includes, but is not limited to, whole plants, plant organs, plant seeds, tissue culture and any groups of plant cells organized into structural or functional units. The use of this term in conjunction with, or in the absence of, any specific type of plant tissue as listed above or otherwise embraced by this definition is not intended to be exclusive of any other type of plant tissue.

“Plant pathogen” or “fungal pathogen” is used herein to mean fungal pathogen. In specific embodiments, the fungal pathogen is from the genus Phakopsora, including the species Phakopsora pachyrhizi and Phakopsora meibomiae. These species are known to cause ASR in plants.

“Polyadenylation signal” or “polyA signal” refers to a nucleic acid sequence located 3′ to a coding region that causes the addition of adenylate nucleotides to the 3′ end of the mRNA transcribed from the coding region.

“Polymerase chain reaction (PCR)” refers to a DNA amplification method that uses an enzymatic technique to create multiple copies of one sequence of nucleic acid (amplicon). Copies of a DNA molecule are prepared by shuttling a DNA polymerase between two amplimers. The basis of this amplification method is multiple cycles of temperature changes to denature, then re-anneal amplimers (DNA primer molecules), followed by extension to synthesize new DNA strands in the region located between the flanking amplimers. Nucleic-acid amplification can be accomplished by any of the various nucleic-acid amplification methods known in the art, including the polymerase chain reaction (PCR). A variety of amplification methods are known in the art and are described, inter alia, in U.S. Pat. Nos. 4,683,195 and 4,683,202 and in PCR Protocols: A Guide to Methods and Applications, ed. Innis et al., Academic Press, San Diego, 1990. PCR amplification methods have been developed to amplify up to 22 kb of genomic DNA and up to 42 kb of bacteriophage DNA (Cheng et al., Proc. Natl. Acad. Sci. USA 91:5695-5699, 1994). These methods as well as other methods known in the art of DNA amplification may be used in the practice of the present invention.

As used herein, the term “primer” refers to an oligonucleotide which is capable of annealing to a nucleic acid target and serving as a point of initiation of DNA synthesis when placed under conditions in which synthesis of a primer extension product is induced (e.g., in the presence of nucleotides and an agent for polymerization such as DNA polymerase and at a suitable temperature and pH). A primer (in some embodiments an extension primer and in some embodiments an amplification primer) is in some embodiments single stranded for maximum efficiency in extension and/or amplification. In some embodiments, the primer is an oligodeoxyribonucleotide. A primer is typically sufficiently long to prime the synthesis of extension and/or amplification products in the presence of the agent for polymerization. The minimum length of the primer can depend on many factors, including, but not limited to temperature and composition (A/T vs. G/C content) of the primer. In the context of amplification primers, these are typically provided as a pair of bi-directional primers consisting of one forward and one reverse primer or provided as a pair of forward primers as commonly used in the art of DNA amplification such as in PCR amplification. As such, it will be understood that the term “primer,” as used herein, can refer to more than one primer, particularly in the case where there is some ambiguity in the information regarding the terminal sequence(s) of the target region to be amplified. Hence, a “primer” can include a collection of primer oligonucleotides containing sequences representing the possible variations in the sequence or includes nucleotides which allow a typical base pairing. Primers can be prepared by any suitable method known in the art. Methods for preparing oligonucleotides of specific sequence include, for example, cloning and restriction of appropriate sequences and direct chemical synthesis. Chemical synthesis methods can include, for example, the phospho di- or tri-ester method, the diethylphosphoramidate method and the solid support method disclosed in U.S. Pat. No. 4,458,066. Primers can be labeled, if desired, by incorporating detectable moieties by for instance spectroscopic, fluorescence, photochemical, biochemical, immunochemical, or chemical moieties. Primers diagnostic (i.e. able to identify or select based on presence of ASR resistant alleles) for ASR resistance can be created to any favorable SNP. The PCR method is well described in handbooks and known to the skilled person. After amplification by PCR, target polynucleotides can be detected by hybridization with a probe polynucleotide, which forms a stable hybrid with the target sequence under stringent to moderately stringent hybridization and wash conditions. If it is expected that the probes are essentially completely complementary (i.e., about 99% or greater) to the target sequence, stringent conditions can be used. If some mismatching is expected, for example if variant strains are expected with the result that the probe will not be completely complementary, the stringency of hybridization can be reduced. In some embodiments, conditions are chosen to rule out non-specific/adventitious binding. Conditions that affect hybridization, and that select against non-specific binding are known in the art, and are described in, for example, Sambrook & Russell (2001). Molecular Cloning: A Laboratory Manual, Third Edition, Cold Spring Harbor Laboratory Press, Cold Spring Harbor, New York, United States of America. Generally, lower salt concentration and higher temperature hybridization and/or washes increase the stringency of hybridization conditions.

As used herein, the terms “progeny” and “progeny plant” refer to a plant generated from a vegetative or sexual reproduction from one or more parent plants. A progeny plant may be obtained by cloning or selfing a single parent plant (i.e., the same plant acts as the donor of both male and female gametes), or by crossing two parental plants. The descendant(s) can be, for example, of the F1, the F2, or any subsequent generation.

The term “promoter” or “promoter region” refers to a polynucleic acid molecule that functions as a regulatory element, usually found upstream (5′) to a coding sequence, that controls expression of the coding sequence by controlling production of messenger RNA (mRNA) by providing the recognition site for RNA polymerase and/or other factors necessary for start of transcription at the correct site. As contemplated herein, a promoter or promoter region includes variations of promoters derived by means of ligation to various regulatory sequences, random or controlled mutagenesis, and addition or duplication of enhancer sequences. The promoter region disclosed herein, and biologically functional equivalents thereof, are responsible for driving the transcription of coding sequences under their control when introduced into a host as part of a suitable recombinant DNA construct, as demonstrated by its ability to produce mRNA. In some embodiments, the vector constructs or expression constructs or nucleic acid sequences disclosed herein comprise a promoter that is heterologous to the nucleic acid sequence encoding the RG31 and/or RG35 polypeptide or an active variant or fragment thereof. In other examples, the vector constructs, expression constructs or nucleic acid sequences comprise a promoter that is native or endogenous to the nucleic acid sequence encoding the RG31 and/or RG35 polypeptide or an active variant or fragment thereof. In particular embodiments, the vector constructs, expression constructs or nucleic acid sequences comprise a promoter that is native to the nucleic acid sequence of the genomic locus comprising the Rg31 or Rg35 gene.

As used herein, the term “recombinant” refers to a non-naturally occurring DNA, protein, cell, seed, or organism that is the result of genetic engineering and as such would not normally be found in nature. A “recombinant DNA molecule” is a DNA molecule comprising a DNA sequence that is not naturally found in nature and as such is the result of human intervention, such as a DNA molecule comprised of at least two DNA molecules heterologous to each other. An example of a recombinant DNA molecule is a DNA molecule provided herein encoding the RG31 and/or RG35 polypeptide or active variant or fragment thereof, operably linked to a heterologous regulatory element, such as a heterologous promoter, heterologous terminator or comprising one or more heterologous intron or the deletion of one or more native introns. A “recombinant protein” is a protein comprising an amino acid sequence that does not naturally occur and as such is the result of human intervention, such as an engineered protein or a chimeric protein. A recombinant cell, seed, or organism is a cell, seed, or organism comprising transgenic DNA, for example a transgenic cell, seed, plant, or plant part comprising a recombinant DNA molecule and therefore produced as a result of plant transformation.

The phrase “substantially identical,” in the context of two nucleic acids or two amino acid sequences, refers to two or more sequences or subsequences that have at least about 50% nucleotide or amino acid residue identity when compared and aligned for maximum correspondence as measured using a sequence comparison algorithm or by visual inspection. In certain embodiments, substantially identical sequences have at least about 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity at the nucleotide or amino acid level. In certain embodiments, substantial identity exists over a region of the sequences that is at least about 50 amino acid residues, 100 amino acid residues, 150 amino acid residues, 200 amino acid residues, 250 amino acid residues, 300 amino acid residues, 350 amino acid residues, 400 amino acid residues, 450 amino acid residues, 500 amino acid residues, 525 amino acid residues, 526, amino acid residues 527 amino acid residues, 528 amino acid residues, 529 amino acid residues, 530 amino acid residues, 531 amino acid residues, 532 amino acid residues, 533 amino acid residues, 534 amino acid residues, 535 amino acid residues, 536 amino acid residues or more with respect to the protein sequence or the nucleotide sequence encoding the same.

The term “identity” or “identical” in the context of two nucleic acid or amino acid sequences, refers to the percentage of identical nucleotides or amino acids in a linear polynucleotide or amino acid sequence of a reference (“query”) sequence (or its complementary strand) as compared to a test (“subject”) sequence when the two sequences are globally aligned. Unless otherwise stated, sequence identity as used herein refers to the value obtained using the Needleman and Wunsch algorithm ((1970) J. Mol. Biol. 48:443-453) implemented in the EMBOSS Needle alignment tool using default matrix files EBLOSUM62 for protein with default parameters (Gap Open=10, Gap Extend=0.5, End Gap Penalty=False, End Gap Open=10, End Gap Extend=0.5) or DNAfull for nucleic acids with default parameters (Gap Open=10, Gap Extend=0.5, End Gap Penalty=False, End Gap Open=10, End Gap Extend=0.5); or any equivalent program thereof. EMBOSS Needle is available, e.g., from EMBL-EBI such as at the following website: ebi.ac.uk/Tools/psa/emboss_needle/and as described in the following publication: “The EMBL-EBI search and sequence analysis tools APIs in 2019.” Madeira et al. Nucleic Acids Research, June 2019, 47 (W1): W636-W641. The term “equivalent program” as used herein refers to any sequence comparison program that, for any two sequences in question, generates an alignment having identical nucleotide or amino acid residue matches and an identical percent sequence identity when compared to the corresponding alignment generated by EMBOSS Needle. In some embodiment, substantially identical nucleic acid or amino acid sequences may perform substantially the same function.

Two nucleotide sequences can also be considered to be substantially identical when the two sequences hybridize to each other under stringent conditions. In representative embodiments, two nucleotide sequences considered to be substantially identical hybridize to each other under highly stringent conditions.

The terms “stringent conditions” or “stringent hybridization conditions” include reference to conditions under which a nucleic acid will selectively hybridize to a target sequence to a detectably greater degree than other sequences (e.g., at least 2-fold over a non-target sequence), and optionally may substantially exclude binding to non-target sequences. Stringent conditions are sequence-dependent and will vary under different circumstances. By controlling the stringency of the hybridization and/or washing conditions, target sequences can be identified that can be up to 100% complementary to the reference nucleotide sequence. Alternatively, conditions of moderate or even low stringency can be used to allow some mismatching in sequences so that lower degrees of sequence similarity are detected. For example, those skilled in the art will appreciate that to function as a primer or probe, a nucleic acid sequence only needs to be sufficiently complementary to the target sequence to substantially bind thereto so as to form a stable double-stranded structure under the conditions employed. Thus, primers or probes can be used under conditions of high, moderate or even low stringency. Likewise, conditions of low or moderate stringency can be advantageous to detect homolog, ortholog and/or paralog sequences having lower degrees of sequence identity than would be identified under highly stringent conditions.

The terms “complementary” or “complementarity” (and similar terms), as used herein, refer to the natural binding of polynucleotides under permissive salt and temperature conditions by base-pairing. For example, the sequence “A-G-T” binds to the complementary sequence “T-C-A.” Complementarity between two single-stranded molecules may be partial, in which only some of the nucleotides bind, or it may be complete when total complementarity exists between the single stranded molecules. The degree of complementarity between nucleic acid strands has significant effects on the efficiency and strength of hybridization between the molecules. As used herein, the term “substantially complementary” (and similar terms) means that two nucleic acid sequences are at least about 50%, 60%, 70%, 75%, 80%, 85%, 90%, 95%, 96%, 97%, 98%, 99% or more complementary. Alternatively, the term “substantially complementary” (and similar terms) can mean that two nucleic acid sequences can hybridize together under high stringency conditions (as described herein).

As used herein, “specifically” or “selectively” hybridizing (and similar terms) refers to the binding, duplexing, or hybridizing of a molecule to a particular nucleic acid target sequence under stringent conditions when that sequence is present in a complex mixture (e.g., total cellular DNA or RNA) to the substantial exclusion of non-target nucleic acids, or even with no detectable binding, duplexing or hybridizing to non-target sequences. Specifically or selectively hybridizing sequences typically are at least about 40% complementary and are optionally substantially complementary or even completely complementary (i.e., 100% identical).

For DNA-DNA hybrids, the Tₘ can be approximated from the equation of Meinkoth and Wahl, Anal. Biochem., 138:267-84 (1984): Tₘ=81.5 °C±16.6 (log M)±0.41 (% GC)−0.61 (% formamide)−500/L; where M is the molarity of monovalent cations, % GC is the percentage of guanosine and cytosine nucleotides in the DNA, % formamide is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1 °C for each 1% of mismatching; thus, Tₘ, hybridization and/or wash conditions can be adjusted to hybridize to sequences of the desired degree of identity. For example, if sequences with >90% identity are sought, the Tₘ can be decreased 10 °C. Generally, stringent conditions are selected to be about 5 °C lower than the thermal melting point (Tₘ) for the specific sequence and its complement at a defined ionic strength and pH. However, highly stringent conditions can utilize a hybridization and/or wash at the thermal melting point (Tₘ) or 1, 2, 3 or 4 °C lower than the thermal melting point (Tₘ); moderately stringent conditions can utilize a hybridization and/or wash at 6, 7, 8, 9 or 10 °C lower than the thermal melting point (Tₘ); low stringency conditions can utilize a hybridization and/or wash at 11, 12, 13, 14, 15 or 20 °C lower than the thermal melting point (Tₘ). If the desired degree of mismatching results in a Tₘ of less than 45 °C (aqueous solution) or 32 °C (formamide solution), optionally the SSC concentration can be increased so that a higher temperature can be used. An extensive guide to the hybridization of nucleic acids is found in Tijssen, Laboratory Techniques in Biochemistry and Molecular Biology-Hybridization with Nucleic Acid Probes, part I, chapter 2, “Overview of principles of hybridization and the strategy of nucleic acid probe assays,” Elsevier, New York (1993); Current Protocols in Molecular Biology, chapter 2, Ausubel, et al., eds, Greene Publishing and Wiley-Interscience, New York (1995); and Green & Sambrook, In: Molecular Cloning, A Laboratory Manual, 4th Edition, Cold Spring Harbor Press, Cold Spring Harbor, N.Y. (2012).

Typically, stringent conditions are those in which the salt concentration is less than about 1.5 M Na ion, typically about 0.01 to 1.0 M Na ion concentration (or other salts) at about pH 7.0 to pH 8.3 and the temperature is at least about 30 °C for short probes (e.g., 10 to 50 nucleotides) and at least about 60 °C for longer probes (e.g., greater than 50 nucleotides). Stringent conditions may also be achieved with the addition of destabilizing agents such as formamide or Denhardt's (5 g Ficoll, 5 g polyvinylpyrrolidone, 5 g bovine serum albumin in 500 ml of water). Exemplary low stringency conditions include hybridization with a buffer solution of 30% to 35% formamide, 1 M NaCl, 1% SDS (sodium dodecyl sulfate) at 37 °C and a wash in 1× to 2×SSC (20×SSC=3.0 M NaCl/0.3 M trisodium citrate) at 50 °C to 55 °C. Exemplary moderate stringency conditions include hybridization in 40% to 45% formamide, 1 M NaCl, 1% SDS at 37 °C and a wash in 0.5× to 1×SSC at 55 °C to 60 °C. Exemplary high stringency conditions include hybridization in 50% formamide, 1 M NaCl, 1% SDS at 37 °C and a wash in 0.1×SSC at 60 °C to 65 °C. A further non-limiting example of high stringency conditions include hybridization in 4×SSC, 5×Denhardt's, 0.1 mg/ml boiled salmon sperm DNA, and 25 mM Na phosphate at 65 °C and a wash in 0.1×SSC, 0.1% SDS at 65 °C Another illustration of high stringency hybridization conditions includes hybridization in 7% SDS, 0.5 M NaPO₄, 1 mM EDTA at 50 °C with washing in 2×SSC, 0.1% SDS at 50 °C, alternatively with washing in 1×SSC, 0.1% SDS at 50 °C, alternatively with washing in 0.5×SSC, 0.1% SDS at 50 °C, or alternatively with washing in 0.1×SSC, 0.1% SDS at 50 °C, or even with washing in 0.1×SSC, 0.1% SDS at 65 °C. Those skilled in the art will appreciate that specificity is typically a function of post-hybridization washes, the relevant factors being the ionic strength and temperature of the final wash solution.

Nucleic acids that do not hybridize to each other under stringent conditions are still substantially identical if the proteins that they encode are substantially identical (e.g., due to the degeneracy of the genetic code).

A further indication that two nucleic acids or proteins are substantially identical is that the protein encoded by the first nucleic acid is immunologically cross reactive with the protein encoded by the second nucleic acid. Thus, a protein is typically substantially identical to a second protein, for example, where the two proteins differ only by conservative substitutions.

As used herein, the term “transgene” refers to a DNA molecule artificially incorporated into an organism's genome because of human intervention, such as a plant transformation method. As used herein, the term “transgenic” means comprising a transgene, for example a “transgenic plant” refers to a plant comprising a transgene in its genome and a “transgenic trait” refers to a characteristic or phenotype conveyed or conferred by the presence of a transgene incorporated into the plant genome. Because of such genomic alteration, the transgenic plant is something distinctly different from the related wild-type plant and the transgenic trait is a trait not naturally found in the wild-type plant. Transgenic plants can comprise the recombinant DNA molecules and engineered proteins provided herein.

As used herein, the term “transgenic” and grammatical variations thereof refer to a plant, including any part derived from the plant, such as a cell, tissue or organ, in which a heterologous nucleic acid is integrated into the genome. In specific embodiments, the heterologous nucleic acid is a recombinant construct, vector or expression cassette comprising one or more nucleic acids.

The term “vector” refers to a composition for transferring, delivering or introducing a nucleic acid (or nucleic acids) into a cell. A vector comprises a nucleic acid molecule comprising the nucleotide sequence(s) to be transferred, delivered or introduced.

### 2. POLYNUCLEOTIDES AND POLYPEPTIDES THAT CONFER INCREASED DISEASE RESISTANCE

Compositions, polypeptides, polynucleotides and active fragments and variants thereof that confer increased disease resistance are provided.

### I. RG31 Polypeptides and Polynucleotides Encoding RG31 Polypeptides

An RG31 polypeptide comprising SEQ ID NO: 1 or an active fragment or variant of SEQ ID NO: 1 is provided. Further provided are polynucleotides comprising a nucleotide sequence encoding the polypeptide of SEQ ID NO: 1 or an active variant or fragment thereof; and polynucleotides comprising any one of SEQ ID NOS: 3 and 4, or an active fragment or variant thereof.

The RG31 polypeptide (SEQ ID NO: 1) encodes a resistance protein (herein also referred to as an R-protein) that is 1735 aa in length. The RG31 polypeptide (SEQ ID NO: 1) comprises several conserved domains including a TIR domain (Toll/Interleukin-1 Receptor domain; pfam Clan No. CL0173), an NB-ARC domain (Nucleotide-Binding adaptor shared by APAF-1, certain R gene products and CED-4; pfam Clan No. CL0023), an LRR domain (Leucine Rich Repeat domain; pfam Clan No. CL0022); a WRKY domain (WRKY DNA-binding domain; pfam Clan No. CL0274); a PK domain (Protein Kinase domain; pfam Clan No. CL0016) and a Kinase-like domain (pfam Clan No. CL0016). The positions of the conserved domains within the RG31 polypeptide are listed at Table A.

Polynucleotides are provided comprising a coding sequence encoding the RG31 polypeptide (SEQ ID NO: 1) or an active variant or fragment thereof. In particular embodiments, the polynucleotide encoding the RG31 polypeptide (SEQ ID NO: 1) or an active variant or fragment thereof, has a coding sequence comprising, or derived from, a genomic sequence of the Rg31 gene, such as polynucleotides comprising SEQ ID NO: 3, or an active fragment or variant thereof.

In example embodiments, a polynucleotide encoding the RG31 polypeptide, or an active variant thereof, that is derived from the genomic sequence of the Rg31 gene comprises all the native exons of the Rg31 gene and one or more of the native introns of the gene, such as only 1 native intron, only 2 native introns, only 3 native introns, all but 1 native intron, all but 2 native introns, all but 3 native introns, and so on. In example embodiments, the polynucleotide encoding the RG31 polypeptide, or an active variant thereof, that is derived from the genomic sequence of the Rg31 gene comprises at least 1, at least 2, at least 3, at least 4, at least 5, at least 6, at least 7, at least 8, at least 9, at least 10, at least 11, at least 12, at least 13, or all 14 native introns of the Rg31 genomic sequence (SEQ ID NO: 3).

In particular embodiments, a polynucleotide encoding the RG31 polypeptide of SEQ ID NO: 1, or an active variant or fragment thereof, comprises one or more of (i) a first native intron of the Rg31 gene (RG31_intron1, position 996 to 1,171 of SEQ ID NO: 3), (ii) a second native intron of the Rg31 gene (RG31_intron2, position 4,173 to 4,357 of SEQ ID NO: 3), (iii) a third native intron of the Rg31 gene (RG31_intron3, position 4,532 to 8,649 of SEQ ID NO: 3), (iv) a fourth native intron of the Rg31 gene (RG31_intron4, position 8,805 to 9,496 of SEQ ID NO: 3), (v) a fifth native intron of the Rg31 gene (RG31_intron5, position 9,654 to 10,351 of SEQ ID NO: 3); (vi) a sixth native intron of the Rg31 gene (RG31_intron6, position 10,515 to 12,562 of SEQ ID NO: 3); (vii) a seventh native intron of the Rg31 gene (RG31_intron7, position 12,654 to 12,723 of SEQ ID NO: 3); (viii) an eighth native intron of the Rg31 gene (RG31_intron8, position 12,764 to 13,781 of SEQ ID NO: 3); (ix) a ninth native intron of the Rg31 gene (RG31_intron9, position 13,917 to 14,981 of SEQ ID NO: 3); (x) a tenth native intron of the Rg31 gene (RG31_intron10, position 15,207 to 15,308 of SEQ ID NO: 3); (xi) an eleventh native intron of the Rg31 gene (RG31_intron11, position 15,492 to 15,587 of SEQ ID NO: 3); (xii) a twelfth native intron of the Rg31 gene (RG31_intron12, position 15,818 to 16,249 of SEQ ID NO: 3); (xiii) a thirteenth native intron of the Rg31 gene (RG31_intron13, position 16,296 to 16,373 of SEQ ID NO: 3); and (xiv) a fourteenth native intron of the Rg31 gene (RG31_intron14, position 16,441 to 16,803 of SEQ ID NO: 3). In still further embodiments, one or more of the native introns (RG31_intron1 through RG31_intron14) may be replaced with other introns. Further still, the polynucleotide derived from the genomic sequence comprising at least 1, at least 2, at least 3, at least 4, at least 5, at least 6, at least 7, at least 8, at least 9, at least 10, at least 11, at least 12, at least 13, or all 14 native introns of the genomic sequence encoding the RG31 polypeptide may comprise one or more additional introns derived from other sources that enhance expression of the RG31 polypeptide.

In other specific embodiments, the polynucleotide encoding the RG31 polypeptide (SEQ ID NO: 1) or an active variant or fragment thereof, has a coding sequence comprising or derived from an intronless cDNA sequence of the Rg31 gene, such as polynucleotides comprising SEQ ID NO: 4, or an active fragment or variant thereof. In still further embodiments, the polynucleotide encoding the RG31 polypeptide, or an active variant thereof, that is derived from the cDNA sequence of the Rg31 gene comprises at least 1, at least 2, at least 3, at least 4, at least 5, at least 6, at least 7, at least 8, at least 9, at least 10, at least 11, at least 12, at least 13, or all 14 native introns of the Rg31 genomic sequence, as listed herein, in addition to the cDNA sequence (SEQ ID NO: 4) to enhance expression of the RG31 polypeptide. In other embodiments, the polynucleotide encoding the RG31 polypeptide, or an active variant thereof, that is derived from the cDNA sequence of the Rg31 gene comprises one or more heterologous introns in addition to the cDNA sequence (SEQ ID NO: 4) to enhance expression of the RG31 polypeptide. In still other embodiments, the coding sequence comprising or derived from the intronless cDNA sequence (SEQ ID NO: 4) of the Rg31 gene may comprise one or more of the native 3′UTR (RG31_3′UTR, position 16,854 to 17,357 of SEQ ID NO: 3) and the native 5′UTR (RG31_5′UTR, position 1 to 504 of SEQ ID NO: 3) to enhance expression of the RG31 polypeptide.

The RG31 polypeptide of SEQ ID NO: 1, or an active fragment or variant of SEQ ID NO: 1, confers disease resistance to a plant, plant part or seed, such as to a legume plant, legume plant part, or legume seed, when expressed in the plant, plant part or seed. In particular embodiments, expression of the RG31 polypeptide of SEQ ID NO: 1, or an active fragment or variant of SEQ ID NO: 1, confers ASR resistance to a soybean plant, plant part or seed when expressed in the soybean plant, plant part or seed.

Likewise, polynucleotides encoding the RG31 polypeptide of SEQ ID NO: 1, or an active fragment or variant of SEQ ID NO: 1, such as polynucleotides comprising any of SEQ ID NOS: 3-4, or an active fragment or variant thereof, confer disease resistance to a plant, plant part or seed, such as to a legume plant, legume plant part, or legume seed, when expressed in the plant, plant part, or seed. In particular embodiments, transcription and expression of the polynucleotide of any one of SEQ ID NOS: 3-4, or an active fragment or variant of any one of SEQ ID NOS: 3-4, confers ASR resistance to a soybean plant, plant part or seed when expressed in the soybean plant, plant part, or seed.

### II. RG35 Polypeptides and Polynucleotides Encoding RG35 Polypeptides

An RG35 polypeptide comprising SEQ ID NO: 2 or an active fragment or variant of SEQ ID NO: 2 is provided. Further provided are polynucleotides comprising a nucleotide sequence encoding the polypeptide of SEQ ID NO: 2 or an active variant or fragment thereof; and polynucleotides comprising any one of SEQ ID NOS: 5 and 6, or an active fragment or variant thereof.

The RG35 polypeptide (SEQ ID NO: 2) encodes a resistance protein (herein also referred to as an R-protein) that is 1728 aa in length. The RG35 polypeptide (SEQ ID NO: 2) comprises several conserved domains including a TIR domain (Toll/Interleukin-1 Receptor domain; pfam Clan No. CL0173), an NB-ARC domain (Nucleotide-Binding adaptor shared by APAF-1, certain R gene products and CED-4; pfam Clan No. CL0023), an LRR domain (Leucine Rich Repeat domain; pfam Clan No. CL0022); a WRKY domain (WRKY DNA-binding domain; pfam Clan No. CL0274); and a PK domain (Protein Kinase domain; pfam Clan No. CL0016). The position of the conserved domains within the RG35 polypeptide is listed at Table A.

Polynucleotides are provided comprising a coding sequence encoding the RG35 polypeptide (SEQ ID NO: 2) or an active variant or fragment thereof. In particular embodiments, the polynucleotide encoding the RG35 polypeptide (SEQ ID NO: 2) or an active variant or fragment thereof, has a coding sequence comprising, or derived from, a genomic sequence of the Rg35 gene, such as polynucleotides comprising SEQ ID NO: 5, or an active fragment or variant thereof.

In example embodiments, a polynucleotide encoding the RG35 polypeptide, or an active variant thereof, that is derived from the genomic sequence of the Rg35 gene comprises all the native exons of the Rg35 gene and one or more of the native introns of the gene, such as only 1 native intron, only 2 native introns, only 3 native introns, all but 1 native intron, all but 2 native introns, all but 3 native introns, and so on. In example embodiments, the polynucleotide encoding the RG35 polypeptide, or an active variant thereof, that is derived from the genomic sequence of the Rg35 gene comprises at least 1, at least 2, at least 3, at least 4, at least 5, at least 6, at least 7, at least 8, at least 9, at least 10, at least 11, at least 12, at least 13, at least 14 or all 15 native introns of the Rg35 genomic sequence (SEQ ID NO: 5).

In particular embodiments, a polynucleotide encoding the RG35 polypeptide of SEQ ID NO: 2, or an active variant or fragment thereof, comprises one or more of (i) a first native intron of the Rg35 gene (RG35_intron1, position 141 to 1,068 of SEQ ID NO: 5), (ii) a second native intron of the Rg35 gene (RG35_intron2, position 1,246 to 1,487 of SEQ ID NO: 5), (iii) a third native intron of the Rg35 gene (RG35_intron3, position 2,121 to 2,296 of SEQ ID NO: 5), (iv) a fourth native intron of the Rg35 gene (RG35_intron4, position 5,289 to 6,007 of SEQ ID NO: 5), (v) a fifth native intron of the Rg35 gene (RG35_intron5, position 6,173 to 8,273 of SEQ ID NO: 5); (vi) a sixth native intron of the Rg35 gene (RG35_intron6, position 8,426 to 9,122 of SEQ ID NO: 5); (vii) a seventh native intron of the Rg35 gene (RG35_intron7, position 9,280 to 9,973 of SEQ ID NO: 5); (viii) an eighth native intron of the Rg35 gene (RG35_intron8, position 10,139 to 12,192 of SEQ ID NO: 5); (ix) a ninth native intron of the Rg35 gene (RG35_intron9, position 12,322 to 13,324 of SEQ ID NO: 5); (x) a tenth native intron of the Rg35 gene (RG35_intron10, position 13,460 to 14,524 of SEQ ID NO: 5); (xi) an eleventh native intron of the Rg35 gene (RG35_intron11, position 14,750 to 14,851 of SEQ ID NO: 5); (xii) a twelfth native intron of the Rg35 gene (RG35_intron12, position 15,035 to 15,130 of SEQ ID NO: 5); (xiii) a thirteenth native intron of the Rg35 gene (RG35_intron13, position 15,361 to 15,780 of SEQ ID NO: 5); (xiv) a fourteenth native intron of the Rg35 gene (RG35_intron14, position 15,827 to 15,904 of SEQ ID NO: 5); and (xv) a fifteenth native intron of the Rg35 gene (RG35_intron15, position 15,972 to 16,334 of SEQ ID NO: 5). In still further embodiments, one or more of the native introns (RG35_intron1 through RG35_intron15) may be replaced with other introns. Further still, the polynucleotide derived from the genomic sequence comprising at least 1, at least 2, at least 3, at least 4, at least 5, at least 6, at least 7, at least 8, at least 9, at least 10, at least 11, at least 12, at least 13, at least 14 or all 15 native introns of the genomic sequence encoding the RG35 polypeptide may comprise one or more additional introns derived from other sources that enhance expression of the RG35 polypeptide.

In other specific embodiments, the polynucleotide encoding the RG35 polypeptide (SEQ ID NO: 2) or an active variant or fragment thereof, has a coding sequence comprising or derived from an intronless cDNA sequence of the Rg35 gene, such as polynucleotides comprising SEQ ID NO: 6, or an active fragment or variant thereof. In still further embodiments, the polynucleotide encoding the RG31 polypeptide, or an active variant thereof, that is derived from the cDNA sequence of the Rg31 gene comprises at least 1, at least 2, at least 3, at least 4, at least 5, at least 6, at least 7, at least 8, at least 9, at least 10, at least 11, at least 12, at least 13, at least 14 or all 15 native introns of the Rg35 genomic sequence, as listed herein, in addition to the cDNA sequence (SEQ ID NO: 6) to enhance expression of the RG35 polypeptide. In other embodiments, the polynucleotide encoding the RG35 polypeptide, or an active variant thereof, that is derived from the cDNA sequence of the Rg35 gene comprises one or more heterologous introns in addition to the cDNA sequence (SEQ ID NO: 6) to enhance expression of the RG35 polypeptide. In still other embodiments, the coding sequence comprising or derived from the intronless cDNA sequence (SEQ ID NO: 6) of the Rg35 gene may comprise one or more of the native 3′UTR (RG35_3′UTR, position 16,381 to 16,798 of SEQ ID NO: 5) and the native 5′UTR (RG35_5′UTR, position 1 to 1629 of SEQ ID NO: 5) to enhance expression of the RG35 polypeptide.

The RG35 polypeptide of SEQ ID NO: 2, or an active fragment or variant of SEQ ID NO: 2, confers disease resistance to a plant, plant part or seed, such as to a legume plant, legume plant part, or legume seed, when expressed in the plant, plant part or seed. In particular embodiments, expression of the RG35 polypeptide of SEQ ID NO: 2, or an active fragment or variant of SEQ ID NO: 2, confers ASR resistance to a soybean plant, plant part or seed when expressed in the soybean plant, plant part or seed.

Likewise, polynucleotides encoding the RG35 polypeptide of SEQ ID NO: 2, or an active fragment or variant of SEQ ID NO: 2, such as polynucleotides comprising any of SEQ ID NOS: 5-6, or an active fragment or variant thereof, confer disease resistance to a plant, plant part or seed, such as to a legume plant, legume plant part, or legume seed, when expressed in the plant, plant part, or seed. In particular embodiments, transcription and expression of the polynucleotide of any one of SEQ ID NOS: 5-6, or an active fragment or variant of any one of SEQ ID NOS: 5-6, confers ASR resistance to a soybean plant, plant part or seed when expressed in the soybean plant, plant part, or seed.

TABLE A
FUNCTIONAL ANNOTATION OF RG31 AND RG35 POLYPEPTIDES

| | SEQ ID | PFAM Clan | PFAM Domain | | | |
|---|---|---|---|---|---|---|
| Polypeptide | NO: | No. | Name | Start | End | E-value |
| RG31 | 1 | CL0173 | TIR domain | 13 | 203 | 1.2e−27 |
| | 1 | CL0023 | NB-ARC | 360 | 547 | 0.0001 |
| | 1 | CL0022 | Leucine Rich Repeat (LRR8) | 688 | 748 | 2.6e−08 |
| | 1 | CL0022 | Leucine Rich Repeat (LRR8) | 753 | 794 | 0.045 |
| | 1 | CL0022 | Leucine Rich Repeat (LRR8) | 796 | 850 | 350 |
| | 1 | CL0274 | WRKY | 1254 | 1314 | 2.8e−23 |
| | 1 | CL0016 | Protein Kinase domain | 1440 | 1702 | 5.9e−69 |
| | 1 | CL0016 | Kinase-like domain | 1195 | 1296 | 650 |
| RG35 | 2 | CL0173 | TIR domain | 13 | 204 | 1.3e−28 |
| | 2 | CL0023 | NB-ARC | 362 | 547 | 0.005 |
| | 2 | CL0022 | Leucine Rich Repeat (LRR8) | 688 | 748 | 5.7e−09 |
| | 2 | CL0022 | Leucine Rich Repeat (LRR8) | 753 | 794 | 0.044 |
| | 2 | CL0022 | Leucine Rich Repeat (LRR8) | 795 | 850 | 340 |
| | 2 | CL0022 | Leucine Rich Repeat (LRR8) | 957 | 1054 | 1100 |
| | 2 | CL0274 | WRKY | 1247 | 1307 | 2.9e−24 |
| | 2 | CL0016 | Protein Kinase domain | 1433 | 1695 | 5.7e−09 |

### III. Fragments and Variants of RG31 and RG35 Polypeptides and Polynucleotides

Active fragments and variants of a RG31 polypeptide (SEQ ID NO: 1) and/or active fragments or variants of a RG35 polypeptide (SEQ ID NO: 2) are also provided. Further provided are polynucleotides comprising a nucleotide sequence encoding an active fragment or variant of the polypeptide of any one of SEQ ID NOS: 1 and 2; and polynucleotides comprising any active variant or fragment of any one of SEQ ID NOS: 3-6.

In specific embodiments, the polynucleotide sequence (SEQ ID NOS: 3-4) and polypeptide sequence (SEQ ID NO: 1) of RG31, and active variants and fragments thereof increase disease resistance in a plant when expressed in a plant, plant part or seed. In other specific embodiments, the polynucleotide sequence (SEQ ID NOS: 5-6) and polypeptide sequence (SEQ ID NO: 2) of RG35, and active variants and fragments thereof increase disease resistance in a plant when expressed in a plant, plant part or seed. In particular embodiments, when expressed in a plant, plant part or seed, the polynucleotide sequence (SEQ ID NOS: 3-4) and polypeptide sequence (SEQ ID NO: 1) of the RG31 polypeptide and active variants or fragments thereof, or the polynucleotide sequence (SEQ ID NO: 5-6) and polypeptide sequence (SEQ ID NO: 2) of the RG35 polypeptide and active variants or fragments thereof, increase the disease resistance of the plant when compared to an appropriate control plant. Various methods by which such an increase in disease resistance can be measured are provided in the Examples and are discussed elsewhere herein.

Fragments of the RG31 polypeptide that increase disease resistance when expressed in a plant, plant part, or seed include RG31 polypeptide fragments that are shorter than the full-length sequence and can comprise a truncation at either the N or C terminus or an internal deletion. An active fragment of a RG31 polypeptide when expressed in a plant can be a polypeptide that is, for example, 10, 25, 50, 100, 150, 200, 250 or more amino acids in length of SEQ ID NO: 1. Such biologically active portions can be prepared by recombinant techniques and evaluated for activity of being able to confer increased resistance when expressed in a plant, plant part or seed. As used herein, a fragment comprises at least 8 contiguous amino acids of SEQ ID NO: 1. An active fragment of the RG31 polypeptide include fragments that retain the ability to (i) mount an immune response when expressed in a plant and/or (ii) increase disease resistance in a plant when expressed in a plant, plant part or seed.

Fragments of the RG35 polypeptide that increase disease resistance when expressed in a plant, plant part, or seed include RG35 polypeptide fragments that are shorter than the full-length sequence and can comprise a truncation at either the N or C terminus or an internal deletion. An active fragment of a RG35 polypeptide when expressed in a plant can be a polypeptide that is, for example, 10, 25, 50, 100, 150, 200, 250 or more amino acids in length of SEQ ID NO: 2. Such biologically active portions can be prepared by recombinant techniques and evaluated for activity of being able to confer increased resistance when expressed in a plant, plant part, or seed. As used herein, a fragment comprises at least 8 contiguous amino acids of SEQ ID NO: 2. Embodiments of an active fragment of the RG35 polypeptide include fragments retaining the ability to (i) mount an immune response when expressed in a plant and/or (ii) increase disease resistance in a plant when expressed in a plant, plant part or seed.

Variant RG31 polypeptides comprise an amino acid sequence that has at least 60%, about 65%, about 70%, about 75%, about 80%, about 85%, about 86%, about 87%, about 88%, about 89%, about 90%, about 91%, about 92%, about 93%, about 94%, about 95%, about 96%, about 97%, about 98% or about 99% identity to the amino acid sequence of SEQ ID NO: 1. Such active variants will increase disease resistance in a plant when expressed in a plant, plant part or seed.

In some embodiments, a variant RG31 polypeptide comprises a deletion and/or addition of one or more amino acids at one or more internal sites within the native polypeptide of SEQ ID NO: 1 and/or a substitution (e.g., conservative substitution) of one or more amino acids at one or more sites in the native polypeptide of SEQ ID NO: 1.

Variant RG35 polypeptides comprise an amino acid sequence that has at least 60%, about 65%, about 70%, about 75%, about 80%, about 85%, about 86%, about 87%, about 88%, about 89%, about 90%, about 91%, about 92%, about 93%, about 94%, about 95%, about 96%, about 97%, about 98% or about 99% identity to the amino acid sequence of SEQ ID NO: 2. Such active variants will increase disease resistance in a plant when expressed in a plant, plant part or seed.

In some embodiments, a variant RG35 polypeptide comprises a deletion and/or addition of one or more amino acids at one or more internal sites within the native polypeptide of SEQ ID NO: 2 and/or a substitution (e.g., conservative substitution) of one or more amino acids at one or more sites in the native polypeptide of SEQ ID NO: 2.

In some embodiments, a variant RG31 polypeptide or a variant RG35 polypeptide includes an annotation variant of the polypeptide comprising a different (e.g., larger or fewer) number of amino acids relative to the native polypeptide of SEQ ID NO: 1 or SEQ ID NO: 2, respectively. These annotation variants may occur due to annotation of the Rg31 or Rg35 gene sequence or annotation of the translated gene sequence with an alternate start codon. In embodiments, annotation variants of SEQ ID NO: 1 or 2 may comprise an amino acid sequence that has at least 60%, about 65%, about 70%, about 75%, about 80%, about 85%, about 86%, about 87%, about 88%, about 89%, about 90%, about 91%, about 92%, about 93%, about 94%, about 95%, about 96%, about 97%, about 98% or about 99% identity to the amino acid sequence of SEQ ID NO: 1 or 2, respectively.

In still other embodiments, a variant RG31 polypeptide or a variant RG35 polypeptide includes an alternative splice variant (or simple “splice variant”) of the native polypeptide comprising a different (e.g., larger or fewer) number of amino acids relative to the polypeptide of SEQ ID NO: 1 or 2, respectively, and/or comprising one or more substitutions relative to the polypeptide of SEQ ID NO: 1 or 2, respectively. These splice variants may occur due to alternative splicing of exons and introns of the Rg31 or Rg35 gene sequence resulting in the creation of variant mRNA transcripts that are expressed to form variant protein sequences. Splice variants may also occur due to alternate annotation of the position of introns and exons within the gene sequence. In embodiments, splice variants of SEQ ID NO: 1 or 2 may comprise an amino acid sequence that has at least 60%, about 65%, about 70%, about 75%, about 80%, about 85%, about 86%, about 87%, about 88%, about 89%, about 90%, about 91%, about 92%, about 93%, about 94%, about 95%, about 96%, about 97%, about 98% or about 99% identity to the amino acid sequence of SEQ ID NO: 1 or 2, respectively.

In one particular embodiment, a splice variant of the RG35 polypeptide comprises the amino acid sequence of SEQ ID NO: 20 and encodes a resistance protein that is 1570 aa in length (that is, shorter than the RG35 protein of SEQ ID NO: 2). The splice variant RG35 polypeptide of SEQ ID NO: 20 is encoded by the nucleotide sequence of SEQ ID NO: 21 (nucleotide sequence including UTRs) and/or SEQ ID NO: 22 (coding sequence without UTRs). In embodiments, a DNA construct comprising a polynucleotide encoding the RG35 polypeptide of SEQ ID NO: 20, or an active variant or fragment thereof, comprises the nucleotide sequence of SEQ ID NO: 5, 6, 21, or 22 and includes one or more alternatively spliced native introns and exons. As non-limiting examples, the DNA construct may comprise one or more of (i) a first native intron at position 141 to 1,068 of SEQ ID NO: 5, (ii) a second native intron at position 1,246 to 1,490 of SEQ ID NO: 5, (iii) a third native intron at position 2,121 to 2,296 of SEQ ID NO: 5, (iv) a fourth native intron at position 5,289 to 6,007 of SEQ ID NO: 5, (v) a fifth native intron at position 6,173 to 12192 of SEQ ID NO: 5; (vi) a sixth native intron at position 12,322 to 13,324 of SEQ ID NO: 5; (vii) a seventh native at position 13,460 to 14,524 of SEQ ID NO: 5; (xi) an eighth native intron at position 14,750 to 14,851 of SEQ ID NO: 5; (xii) a ninth native intron at position 15,035 to 15,130 of SEQ ID NO: 5; (xiii) a tenth native intron at position 15,361 to 15,780 of SEQ ID NO: 5; (xiv) an eleventh native intron at position 15,827 to 15,904 of SEQ ID NO: 5; and (xv) a twelfth native intron at position 15,972 to 16,334 of SEQ ID NO: 5. In still further embodiments, one or more of the alternatively spliced native introns listed above may be replaced with other introns. Further still, the polynucleotide derived from the genomic sequence comprising at least 1, at least 2, at least 3, at least 4, at least 5, at least 6, at least 7, at least 8, at least 9, at least 10, at least 11, or all 12 of the alternatively spliced native introns of the genomic sequence encoding the RG35 splice variant polypeptide may comprise one or more additional introns derived from other sources that enhance expression of the RG35 splice variant.

In other instances, the RG31 polypeptide variant and/or RG35 polypeptide variant comprises a tag, such as a His tag. In still other instances, the polypeptide variant comprises a detectable marker, such as a detectable peptide marker.

Fragments and variants of a nucleotide sequence can encode protein fragments that retain the biological activity of natural proteins and have the ability to increase disease resistance. Alternatively, nucleotide sequence fragments or variants that can be used as hybridization probes or in recombinant DNA constructs designed for gene editing do not necessarily code protein fragments that maintain biological activity. Thus, the fragment of the nucleotide sequence may be in the range of at least about 15, 50, 100, 150, 200, 250, 300, 350, 400, 450, 500, 550, 600, 650, 700, 750, 800, 750, 900, 950, 1000 nucleotides or less than the full-length nucleotide sequence coding the proteins disclosed herein (i.e, any one of SEQ ID NOS: 3-6 or 21-22).

A variant of the nucleotide sequence has as at least 60%, about 65%, about 70%, about 75%, about 80%, about 85%, about 86%, about 87%, about 88%, about 89%, about 90%, about 91%, about 92%, about 93%, about 94%, about 95%, about 96%, about 97%, about 98% or about 99% identity to the nucleotide sequence of any one of SEQ ID NOS: 3-6 or 21-22. In specific embodiments, the variant nucleotide sequence encodes an active polypeptide of the invention. In other embodiments, the variant polynucleotide need not encode an active variant polypeptide and can be used as components of a gene editing construct or as probes or primers or other tools useful in generating the plants and seeds provided herein.

The term “corresponding to” in the context of nucleic acid sequences means that when the nucleic acid sequences of certain sequences are aligned with each other, the nucleic acids that “correspond to” certain enumerated positions in the present invention are those that align with these positions in a reference sequence, but that are not necessarily in those exact numerical positions relative to a particular nucleic acid sequence of the invention. Optimal alignment of sequences for comparison can be conducted by computerized implementations of known algorithms or by visual inspection. Readily available sequence comparison and multiple sequence alignment algorithms are, respectively, the Basic Local Alignment Search Tool (BLAST) and ClustalW/ClustalW2/Clustal Omega programs available on the Internet (e.g., the website of the EMBL-EBI). Other suitable programs include, but are not limited to, GAP, BestFit, Plot Similarity, and FASTA, which are part of the Accelrys GCG Package available from Accelrys, Inc. of San Diego, Calif., United States of America. See also Smith & Waterman, 1981; Needleman & Wunsch, 1970; Pearson & Lipman, 1988; Ausubel et al., 1988; and Sambrook & Russell, 2001, by computerized implementations of these algorithms (GAP, BESTFIT, FASTA, and TFASTA in the Wisconsin Genetics Software Package, Genetics Computer Group, 575 Science Dr., Madison, Wis.), or by visual inspection.

Unless otherwise stated, a “corresponding” amino acid position to a given SEQ ID NO is determined using Geneious as a global alignment tool/program with free end gaps having the following parameters: cost matrix Blossum 62, gap open penalty 12, gap extension penalty 3, refinement iterations 2; or an equivalent program thereof. The term “equivalent program” as used herein refers to any sequence comparison program that, for any two sequences in question, generates an alignment having identical corresponding nucleotide or amino acid residue matches when compared to the corresponding alignment generated by the program provided above.

One example of an algorithm that is suitable for determining percent sequence identity and sequence similarity as well as for aligning sequences is the BLAST algorithm, which is described in Altschul et al, J. Mol. Biol. 215:403-410 (1990). Software for performing BLAST analyses is publicly available through the National Center for Biotechnology Information (National Center for Biotechnology Information, U.S. National Library of Medicine, 8600 Rockville Pike, Bethesda, MD 20894 USA). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990).

These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

The variants and fragments disclosed herein can be altered, for example, by including amino acid substitutions, deletions, truncations, and insertions. Methods for such manipulations are known in the art. For example, amino acid sequence variants and fragments of the RG31 and/or RG35 polypeptides can be prepared by mutations in the corresponding polynucleotide sequence. Methods for mutagenesis and polynucleotide alterations are known in the art.

Variant polynucleotides and polypeptides also encompass sequences and polypeptides derived from mutagenic or recombinogenic procedures, including and not limited to procedures such as DNA shuffling. Strategies for such DNA shuffling are known in the art.

Variants may be made by making random mutations in the RG31 and/or RG35 polypeptide sequences. In other embodiments, the variants may be specifically designed. In the case of designed mutants, it is possible to generate variants with similar biological activity to the original polypeptide when amino acid identity is maintained in regions of the polypeptide which account for biological activity or are involved in determining a three-dimensional configuration of the polypeptide which is responsible for the biological activity. It is also possible to retain the biological activity if conservative substitutions are made wherein amino acids of a given class are replaced with another amino acid of the same class. As such, it is known that amino acids can be placed in one of the following classes: aliphatic or cyclic (Glycine, Alanine, Valine, Leucine, Isoleucine, Proline), Aromatic (Phenylalanine, Tyrosine, Tryptophan), Acidic (Aspartic acid, Glutamic acid, Asparagine, Glutamine), Basic (Histidine, Lysine, Arginine), and Sulfur or hydroxyl containing groups (Serine, Cysteine, Methionine, Threonine). Conservative substitutions whereby an amino acid of one class is replaced with another amino acid of the same type are least likely to materially alter the biological activity of the variant.

Variants of polypeptide and polynucleotides also comprise sequences from other organisms, particularly other plants, isolated based on their sequence identity to the polypeptide and polynucleotide sequences disclosed herein. Such sequences include sequences that are orthologs of the disclosed sequences. The term “orthologs” refers to genes derived from a common ancestral gene and which are found in different species as a result of speciation. Genes found in different species are considered orthologs when their nucleotide sequences and/or their encoded polypeptide sequences share at least about 60%, about 70%, about 75%, about 80%, about 85%, about 90%, about 91%, about 92%, about 93%, about 94%, about 95%, about 96%, about 97%, about 98%, about 99%, or greater sequence identity. Functions of orthologs are often highly conserved among species. Thus, isolated polynucleotides that encode for a RG31 or RG35 polypeptide that confers or enhances disease resistance and that hybridize to the sequences disclosed herein, or to variants or fragments thereof, are encompassed by the present disclosure.

Variants of polypeptide and polynucleotides also comprise sequences from the same organism present at a different location of the genome, such as on a different chromosome, isolated based on their sequence identity to the polypeptide and polynucleotide sequences disclosed herein. Such sequences include sequences that are paralogs of the disclosed sequences. The term “paralogs” refers to gene copies created by a duplication event on the same genome. Genes found on the same genome are considered paralogs when their nucleotide sequences and/or their encoded polypeptide sequences share at least about 60%, about 70%, about 75%, about 80%, about 85%, about 90%, about 91%, about 92%, about 93%, about 94%, about 95%, about 96%, about 97%, about 98%, about 99%, or greater sequence identity. Functions of paralogs can be conserved, though they may be expressed only under different selective pressures. Paralogs may also develop different functions due to missing selective pressure on the duplicated copy of a gene.

Variants of polypeptide and polynucleotides also comprise sequences that are allelic variants of the disclosed sequences. The term “allelic variants” refers to different variants of the same gene at a single gene locus that can cause the same or similar phenotypic expression (e.g., of increasing disease resistance). These include genes from the same locus of the same organism, as well as genes from the same locus of an organism belonging to the same genus. Such allelic variants are isolated based on their sequence identity to the polypeptide and polynucleotide sequences disclosed herein. Genes found at the same locus are considered allelic variants when their nucleotide sequences and/or their encoded polypeptide sequences share at least about 60%, about 70%, about 75%, about 80%, about 85%, about 90%, about 91%, about 92%, about 93%, about 94%, about 95%, about 96%, about 97%, about 98%, about 99%, or greater sequence identity. Functions of allelic variants can be conserved.

Variants of the polypeptides and polynucleotides also comprise sequences from other organisms, particularly other plants, identified based on their sequence identity to the polypeptide and polynucleotide sequences disclosed herein, and having functional identity with the polypeptide and polynucleotide sequences disclosed herein. Such variants having “functional identity” are referred to herein as “functional identity variants”. As used herein, “functional identity” refers to the presence of a common functional activity, such as a common enzymatic activity or common mode of action. Polypeptides having functional identity may have low sequence identity, for example, their nucleotide sequences and/or their encoded polypeptide sequences may share about 40%, about 50%, about 60%, about 70%, about 80%, about 90%, about 95%, about 96%, about 97%, about 98%, about 99%, or greater sequence identity. However, they may share specificity or preference to the same substrate, share kinetic parameters, etc. In one example, variant polypeptides and polynucleotides of the RG31 and/or RG35 polypeptides, and polynucleotide sequences encoding such polypeptides, comprise proteins that interact with the same set of effector proteins to generate a hypersensitive response in a plant cell.

As used herein, “effectors” or “effector proteins” or “plant pathogen effectors” refer to polypeptide molecules secreted by pathogens to counteract the endogenous defense system of a plant. Effector proteins interact (e.g., physically interact) with a plant-based resistance protein, or a polypeptide encoded by a plant-based resistance gene, to elicit a localized immune response comprising a hypersensitive programmed cell death response in infected plant cells/tissues. Typically, each R-protein interacts with a distinct set of effector proteins, thereby defining a distinct mode of action or site of action for conferring disease resistance. In one example embodiment, the RG31 polypeptide and/or RG35 polypeptide are R-proteins that each interact with a distinct set of effector proteins to elicit a hypersensitive response in a plant cell. In particular embodiments, despite the presence of a high sequence identity to the R-protein RG1 (as described in WO2019103918A1 as SEQ ID NO: 47 (protein) and SEQ ID NO: 6 (gene)), RG31 and RG35 have a novel mode of action that is different from the mode of action of RG1, as indicated by the difference in specificity for effector proteins in assays detecting localized hypersensitive responses (as disclosed at Example 3). As used herein, the term “site of action” or “mode of action” refers to specific interactions of an effector polypeptide derived from a pathogen with a disease resistance protein (R-protein) derived from a plant.

In particular embodiments, variants of the polypeptides and polynucleotides comprise sequences from other organisms, particularly other plants, that when expressed in a plant and assayed, interact with most or all of the effector proteins recognized by the RG31 and/or RG35 polypeptides to generate a localized hypersensitive response. Assays for identifying the interaction of an expressed R-protein, or a variant or fragment thereof, with an effector protein, and identifying the presence of a localized hypersensitive response are disclosed herein at Example 3.

### 3. EXPRESSION CASSETTES AND REGULATORY ELEMENTS

Polynucleotides provided herein can be provided in expression cassettes for expression (herein also referred to as “DNA constructs”) in an organism of interest. The expression cassette will include 5′ and 3′ regulatory sequences operably linked to a polynucleotide encoding a RG31 polypeptide or a RG35 polypeptide, or active variants or fragments of an RG31 or RG35 polypeptide, that allows for expression of the polynucleotide. The cassette may additionally contain at least one additional gene or genetic element to be co-transformed into the organism. Where additional genes or elements are included, the components are operably linked. Alternatively, the additional gene(s) or element(s) can be provided on multiple expression cassettes. Such an expression cassette is provided with a plurality of restriction sites and/or recombination sites for insertion of the polynucleotides to be under the transcriptional regulation of the regulatory elements or regions. The expression cassette may additionally contain a selectable marker gene.

“DNA construct” refers to the genetic elements operably linked to each other making up a recombinant DNA molecule and may comprise elements that provide expression of a DNA polynucleotide molecule in a host cell and elements that provide maintenance of the construct in the host cell. The various genetic elements within the DNA construct can be native to polynucleotide encoding the polypeptide or heterologous to the native polynucleotide encoding the polypeptide.

DNA constructs, vectors, and expression cassettes can be prepared that incorporate the nucleotide sequence encoding the RG31 polypeptide or RG35 polypeptide, or an active variant or fragment of the RG31 polypeptide or RG35 polypeptide, for use in directing the expression of the sequences directly from the host plant cell. Examples of such constructs suitable for this purpose and methods are generally described, for example, in Svab et al., Proc. Natl. Acad. Sci. USA 87:8526-8530, (1990) and Svab et al., Proc. Natl. Acad. Sci. USA 90:913-917 (1993) and in U.S. Pat. No. 5,693,507.

A plant expression cassette comprises the operable linkage of genetic elements that when transferred into a plant cell provides expression of a desirable gene product. “Plant expression cassette” refers to a DNA construct comprising the regulatory elements that are operably linked to provide the expression of a desired nucleic acid in a plant. Promoters, leaders, introns, transit peptide encoding polynucleic acids, 3′ transcriptional termination regions are all genetic elements that may be operably linked by those skilled in the art of plant molecular biology to provide a desirable level of expression or functionality to a RG31 polypeptide or an RG35 polypeptide, or an active variant or fragment of any of the aforementioned.

In embodiments, a DNA construct comprising a polynucleotide encoding the RG31 polypeptide of SEQ ID NO: 1, or an active variant or fragment thereof, comprises one or more of the native introns of the genomic sequence (SEQ ID NO: 3) of an Rg31 gene encoding the RG31 polypeptide. As non-limiting examples, the DNA construct may comprise one or more of (i) a first native intron of the Rg31 gene (RG31_intron1, position 996 to 1,171 of SEQ ID NO: 3), (ii) a second native intron of the Rg31 gene (RG31_intron2, position 4,173 to 4,357 of SEQ ID NO: 3), (iii) a third native intron of the Rg31 gene (RG31_intron3, position 4,532 to 8,649 of SEQ ID NO: 3), (iv) a fourth native intron of the Rg31 gene (RG31_intron4, position 8,805 to 9,496 of SEQ ID NO: 3), (v) a fifth native intron of the Rg31 gene (RG31_intron5, position 9,654 to 10,351 of SEQ ID NO: 3); (vi) a sixth native intron of the Rg31 gene (RG31_intron6, position 10,515 to 12,562 of SEQ ID NO: 3); (vii) a seventh native intron of the Rg31 gene (RG31_intron7, position 12,654 to 12,723 of SEQ ID NO: 3); (viii) an eighth native intron of the Rg31 gene (RG31_intron8, position 12,764 to 13,781 of SEQ ID NO: 3); (ix) a ninth native intron of the Rg31 gene (RG31_intron9, position 13,917 to 14,981 of SEQ ID NO: 3); (x) a tenth native intron of the Rg31 gene (RG31_intron10, position 15,207 to 15,308 of SEQ ID NO: 3); (xi) an eleventh native intron of the Rg31 gene (RG31_intron11, position 15,492 to 15,587 of SEQ ID NO: 3); (xii) a twelfth native intron of the Rg31 gene (RG31_intron12, position 15,818 to 16,249 of SEQ ID NO: 3); (xiii) a thirteenth native intron of the Rg31 gene (RG31_intron13, position 16,296 to 16,373 of SEQ ID NO: 3); and (xiv) a fourteenth native intron of the Rg31 gene (RG31_intron14, position 16,441 to 16,803 of SEQ ID NO: 3). In still further embodiments, one or more of the native introns (RG31_intron1 through RG31_intron14) may be replaced with other introns. Further still, the polynucleotide derived from the genomic sequence comprising at least 1, at least 2, at least 3, at least 4, at least 5, at least 6, at least 7, at least 8, at least 9, at least 10, at least 11, at least 12, at least 13, or all 14 native introns of the genomic sequence encoding the RG31 polypeptide may comprise one or more additional introns derived from other sources that enhance expression of the RG31 polypeptide.

In embodiments, a DNA construct comprising a polynucleotide encoding the RG35 polypeptide of SEQ ID NO: 2, or an active variant or fragment thereof, comprises one or more of the native introns of a genomic sequence (SEQ ID NO: 5) of an Rg35 gene encoding the RG35 polypeptide. As non-limiting examples, the DNA construct may comprise one or more of (i) a first native intron of the Rg35 gene (RG35_intron1, position 141 to 1,068 of SEQ ID NO: 5), (ii) a second native intron of the Rg35 gene (RG35_intron2, position 1,246 to 1,487 of SEQ ID NO: 5), (iii) a third native intron of the Rg35 gene (RG35_intron3, position 2,121 to 2,296 of SEQ ID NO: 5), (iv) a fourth native intron of the Rg35 gene (RG35_intron4, position 5,289 to 6,007 of SEQ ID NO: 5), (v) a fifth native intron of the Rg35 gene (RG35_intron5, position 6,173 to 8,273 of SEQ ID NO: 5); (vi) a sixth native intron of the Rg35 gene (RG35_intron6, position 8,426 to 9,122 of SEQ ID NO: 5); (vii) a seventh native intron of the Rg35 gene (RG35_intron7, position 9,280 to 9,973 of SEQ ID NO: 5); (viii) an eighth native intron of the Rg35 gene (RG35_intron8, position 10,139 to 12,192 of SEQ ID NO: 5); (ix) a ninth native intron of the Rg35 gene (RG35_intron9, position 12,322 to 13,324 of SEQ ID NO: 5); (x) a tenth native intron of the Rg35 gene (RG35_intron10, position 13,460 to 14,524 of SEQ ID NO: 5); (xi) an eleventh native intron of the Rg35 gene (RG35_intron11, position 14,750 to 14,851 of SEQ ID NO: 5); (xii) a twelfth native intron of the Rg35 gene (RG35_intron12, position 15,035 to 15,130 of SEQ ID NO: 5); (xiii) a thirteenth native intron of the Rg35 gene (RG35_intron13, position 15,361 to 15,780 of SEQ ID NO: 5); (xiv) a fourteenth native intron of the Rg35 gene (RG35_intron14, position 15,827 to 15,904 of SEQ ID NO: 5); and (xv) a fifteenth native intron of the Rg35 gene (RG35_intron15, position 15,972 to 16,334 of SEQ ID NO: 5). In still further embodiments, one or more of the native introns (RG35_intron1 through RG35_intron15) may be replaced with other introns. Further still, the polynucleotide derived from the genomic sequence comprising at least 1, at least 2, at least 3, at least 4, at least 5, at least 6, at least 7, at least 8, at least 9, at least 10, at least 11, at least 12, at least 13, at least 14 or all 15 native introns of the genomic sequence encoding the RG35 polypeptide may comprise one or more additional introns derived from other sources that enhance expression of the RG35 polypeptide.

In embodiments, a DNA construct comprising a polynucleotide encoding the RG35 polypeptide of SEQ ID NO: 20, or an active variant or fragment thereof, comprises the nucleotide sequence of SEQ ID NO: 5, 6, 21, or 22 and includes one or more alternatively spliced native introns and exons of a genomic sequence (SEQ ID NO: 5) of an Rg35 gene encoding the RG35 polypeptide. As non-limiting examples, the DNA construct may comprise one or more of the following alternatively spliced introns (i) a first native intron at position 141 to 1,068 of SEQ ID NO: 5, (ii) a second native intron at position 1,246 to 1,490 of SEQ ID NO: 5, (iii) a third native intron at position 2,121 to 2,296 of SEQ ID NO: 5, (iv) a fourth native intron at position 5,289 to 6,007 of SEQ ID NO: 5, (v) a fifth native intron at position 6,173 to 12192 of SEQ ID NO: 5; (vi) a sixth native intron at position 12,322 to 13,324 of SEQ ID NO: 5; (vii) a seventh native at position 13,460 to 14,524 of SEQ ID NO: 5; (xi) an eighth native intron at position 14,750 to 14,851 of SEQ ID NO: 5; (xii) a ninth native intron at position 15,035 to 15,130 of SEQ ID NO: 5; (xiii) a tenth native intron at position 15,361 to 15,780 of SEQ ID NO: 5; (xiv) an eleventh native intron at position 15,827 to 15,904 of SEQ ID NO: 5; and (xv) a twelfth native intron at position 15,972 to 16,334 of SEQ ID NO: 5. In still further embodiments, one or more of the alternatively spliced native introns listed above may be replaced with other introns. Further still, the polynucleotide derived from the genomic sequence comprising at least 1, at least 2, at least 3, at least 4, at least 5, at least 6, at least 7, at least 8, at least 9, at least 10, at least 11, or all 12 of the alternatively spliced native introns of the genomic sequence encoding the RG35 splice variant polypeptide may comprise one or more additional introns derived from other sources that enhance expression of the RG35 splice variant.

A DNA construct can contain one or more plant expression cassettes expressing the DNA molecules of the present invention or other DNA molecules useful in the genetic engineering of crop plants. One example of a DNA construct that may be used for expressing the RG31 polypeptide or active variant or fragment thereof is a vector with a nucleic acid sequence encoding the RG31 polypeptide or active fragment or variant thereof. One example of a DNA construct that may be used for expressing the RG35 polypeptide or active variant or fragment thereof is a vector with a nucleic acid sequence encoding the RG35 polypeptide or active fragment or variant thereof.

In embodiments, the nucleic acid sequence is operably linked to a heterologous regulatory element. In one particular example, the vector comprises an expression cassette comprising a polynucleotide encoding the RG31 polypeptide or RG35 polypeptide, or active fragment or variant thereof, operably coupled to a heterologous regulatory element, such as a plant active promoter driving expression of the RG31 polypeptide, RG35 polypeptide or active fragment or variant thereof in a plant, plant part or seed.

Expression of the DNA constructs, expression cassettes and vectors of the present disclosure in a plant, plant part or seed confers the plant with disease resistance. In particular embodiments, expression of a RG31 polypeptide (or active fragments and variants thereof) or an RG35 polypeptide (or active variants and fragments thereof) via expression of a nucleic acid molecule comprising a nucleotide sequence comprising a polynucleotide encoding the RG31 polypeptide or a polynucleotide encoding the RG35 polypeptide confers a legume plant, plant part or seed (e.g., soybean plant, plant part or seed) with disease resistance (e.g., fungal pathogen resistance and/or ASR resistance).

The translation leader sequence means a DNA molecule located between the promoter of a gene and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences include maize and petunia heat shock protein leaders, plant virus coat protein leaders, plant rubisco gene leaders among others (Turner and Foster, Molecular Biotechnology 3:225, 1995).

The “3′ non-translated sequences”, or “3′ untranslated sequences” or “3′-UTRs” mean DNA sequences located downstream of a structural polynucleotide sequence and include sequences encoding polyadenylation and other regulatory signals capable of affecting mRNA processing or gene expression. The polyadenylation signal functions in plants to cause the addition of multiple adenylate nucleotides to the 3′ end of the mRNA precursor. The polyadenylation sequence can be derived from the natural gene, from a variety of plant genes, or from T-DNA. In particular embodiments, the 3′-UTR of the RG31 gene may be included, wherein the 3′-UTR of the RG31 gene is derived from the genomic sequence of SEQ ID NO: 3 and may include at least a 500 bp, 1000 bp or 2000 bp region lying immediately downstream of the stop codon. In one particular embodiment, the 3′-UTR of the RG31 gene (RG31_3′UTR) comprises position 16,854 to 17,357 of SEQ ID NO: 3). In other particular embodiments, the 3′-UTR of the RG35 gene may be included wherein the 3′-UTR of the RG35 gene is derived from the genomic sequence of SEQ ID NO: 5 and may include at least a 500 bp, 100 bp or 2000 bp region lying immediately downstream of the stop codon. In one particular embodiment, the 3′-UTR of the RG35 gene comprises position 16,381 to position 16,798 of SEQ ID NO: 5.

An example of the polyadenylation sequence is the nopaline synthase 3′ sequence (nos 3′; Fraley et al., Proc. Natl. Acad. Sci. USA 80:4803-4807, 1983). The use of different 3′ non-translated sequences is exemplified by Ingelbrecht et al., Plant Cell 1:671-680, 1989.

In specific embodiments, the various 3′-UTRs disclosed in WO2019103918, WO2021000878, WO2021022022, WO2022173659, WO2021260673, WO2021263249, or U.S. Provisional Applications 63/481,627, 63/426,524, 63/509586, or 63/383609 may be used; each of which is herein incorporated by reference in its entirety, including for example, those disclosed in WO2022173659 as SEQ ID NO: 20.

A variety of transcriptional terminators are available for use in expression cassettes. These are responsible for the termination of transcription beyond the transgene and correct mRNA polyadenylation. The termination region may be native with the transcriptional initiation region, may be native with the operably linked DNA sequence of interest, may be native with the plant host, or may be derived from another source (i.e., foreign or heterologous to the promoter, the DNA sequence of interest, the plant host, or any combination thereof). Appropriate transcriptional terminators are those that are known to function in plants and include the CAMV 35S terminator, the tm1 terminator, the nopaline synthase terminator and the pea rbcs E9 terminator. These can be used in both monocotyledons and dicotyledons. Still other known plant terminators can be used such as terminators derived from Medicago truncatula (e.g., tMt51186; SEQ ID NO: 13). In addition, a gene's native transcription terminator may be used. In particular embodiments, the native terminator of the RG31 gene (tGtoRG31; SEQ ID NO: 9) and/or the native terminator of the RG35 gene (tGtoRG35; SEQ ID NO: 11) may be used. Termination regions used in the expression cassettes can be obtained from, e.g., the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

In specific embodiments, the various terminators disclosed in WO2019103918, WO2021000878, WO2021022022, WO2022173659, WO2021260673, WO202163249, or U.S. Provisional Applications 63/481,627, 63/426,524, 63/509,586, or 63/383,609 may be used including for example, those disclosed in WO2019103918 including SEQ ID NO: 8 (RG1), SEQ ID NO: 11, SEQ ID NO: 14, SEQ ID NO: 17, SEQ ID NO: 20, SEQ ID NO: 23, SEQ ID NO: 26, SEQ ID NO: 29, or SEQ ID NO: 32; those disclosed in WO2022173659 including SEQ ID NO: 18 (RG30); or those disclosed in WO2021022022 as SEQ ID NO:10 (TirA terminator) or SEQ ID NO: 15 (TirB terminator); or those disclosed in U.S. Provisional Application 63/481,627 as SEQ ID NO: 9 (RG31 terminator) or SEQ ID NO: 11 (RG35 terminator); or those disclosed in U.S. Provisional application 63/383,609 as SEQ ID NO: 28 (RG3a terminator) or SEQ ID NO: 29 (RG3b terminator); each of which is herein incorporated by reference in its entirety.

The “5′ non-translated sequences”, or “5′ untranslated sequences” or “5′-UTRs” mean DNA sequences located upstream of an initiation codon of structural polynucleotide sequence and include sequences capable of affecting translation of an mRNA sequence. The 5′-UTR sequence is also referred to as a leader sequence. In different organisms, the 5′-UTR may remain untranslated, and form complex secondary structures to regulate translation of the downstream sequence. The leader sequence can be derived from the natural gene or from a variety of plant genes. In particular embodiments, the 5′-UTR of the RG31 gene may be included, wherein the 5′-UTR of the RG31 gene is derived from the genomic sequence of SEQ ID NO: 3 and may include at least a 500 bp, 1000 bp, or 2000 bp region lying immediately upstream of the start codon or a 500 bp region lying immediately downstream from a transcription start site. In one particular embodiment, the 5′-UTR of the RG31 gene (RG31_5′UTR) comprises position 1 to 504 of SEQ ID NO: 3. In other particular embodiments, the 5′-UTR of the RG35 gene may be included wherein the 5′-UTR of the RG35 gene is derived from the genomic sequence of SEQ ID NO: 5 and may include at least a 500 bp, 1000 bp, or 2000 bp region lying immediately upstream of the start codon or a 500 bp region lying immediately downstream from a transcription start site. In one particular embodiment, the 5′-UTR of the RG35 gene comprises position 1 to position 1,629 of SEQ ID NO: 5. In another embodiment, the 5′-UTR of the RG35 gene comprises the sequence from position 1 to position 1,629 of SEQ ID NO: 5, excluding the first native intron at position 141 to 1068 and the second native intron from position 1246 to 1487 of SEQ ID NO: 5. In one particular embodiment, the 5′-UTR of the RG35 gene comprises the sequence from position 1 to 140 and 1488 to 1629 of SEQ ID NO: 5.

In specific embodiments, the various 5′-UTRs disclosed in WO2019103918, WO2021000878, WO2021022022, WO2022173659, WO2021260673, WO2021263249, or U.S. Provisional Applications 63/481,627, 63/426,524, 63/509586, or 63/383,609 may be used; each of which is herein incorporated by reference in its entirety, including for example, those disclosed in WO2022173659 as SEQ ID NO: 19.

A number of non-translated leader sequences derived from viruses are also known to enhance expression, and these are particularly effective in dicotyledonous cells. The expression cassette may comprise one or more of such leader sequences. Specifically, leader sequences from tobacco mosaic virus (TMV, the “W-sequence”), maize chlorotic mottle virus (MCMV), and alfalfa mosaic virus (AMV) have been shown to be effective in enhancing expression (e.g., Gallie et al. Nucl. Acids Res. 15:8693-8711 (1987); Skuzeski et al. Plant Molec. Biol. 15:65-79 (1990)). Other leader sequences known in the art include but are not limited to: picomavirus leaders, for example, EMCV leader (encephalomyocarditis 5′ noncoding region) (Elroy-Stein, O., Fuerst, T. R., and Moss, B. PNAS USA 86:6126-6130 (1989)); potyvirus leaders, for example, tobacco etch virus (TEV) leader (Allison et al., 1986); maize dwarf mosaic virus (MDMV) leader; Virology 154:9-20); human immunoglobulin heavy-chain binding protein (BiP) leader, (Macejak, D. G., and Samow, P., Nature 353:90-94 (1991); untranslated leader from the coat protein mRNA of alfalfa mosaic virus (AMV RNA 4), (Jobling, S. A., and Gehrke, L., Nature 325:622-625 (1987); tobacco mosaic virus leader (TMV), (Gallie, D. R. et al., Molecular Biology of RNA, 237-256 (1989); and maize chlorotic mottle virus leader (MCMV) (Lommel, S. A. et al., Virology 81:382-385 (1991). See also, Della-Cioppa et al., Plant Physiology 84:965-968 (1987).

In specific embodiments, the various 3′-UTRs disclosed in WO2019103918, WO2021000878, WO2021022022, WO2022173659, WO2021260673, WO2021263249, and WO2022173659 are each incorporated by reference in their entirety. In further specific embodiments, the various 5′-UTRs disclosed in U.S. Provisional Apps. Nos. 63/426,524, 63/509,586, and 63/383,609 are each incorporated by reference in their entirety.

Additional regulatory signals include, but are not limited to, transcriptional initiation start sites, operators, activators, enhancers, other regulatory elements, ribosomal binding sites, an initiation codon, termination signals, and the like. See, for example, U.S. Pat. Nos. 5,039,523 and 4,853,331; EPO 0480762A2; Sambrook et al. (1992) Molecular Cloning: A Laboratory Manual, ed. Maniatis et al. (Cold Spring Harbor Laboratory Press, Cold Spring Harbor, N.Y.), hereinafter “Sambrook 11”; Davis et al, eds. (1980).

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin, 5-enolpyruvylshikimate-3-phosphate synthase (EPSPS) or acetolactate synthase (ALS). Selection markers used routinely in transformation include the nptll gene, which confers resistance to kanamycin and related antibiotics (Messing & Vierra Gene 19:259-268 (1982); Bevan et al., Nature 304:184-187 (1983)), the pat and bar genes, which confer resistance to the herbicide glufosinate (also called phosphinothricin; see White et al., Nucl. Acids Res 18:1062 (1990), Spencer et al. Theor. Appl. Genet 79:625-631 (1990) and U.S. Pat. Nos. 5,561,236 and 5,276,268), the hph gene, which confers resistance to the antibiotic hygromycin (Blochinger & Diggelmann, Mol. Cell Biol. 4:2929-2931), and the dhfr gene, which confers resistance to methatrexate (Bourouis et al., EMBO J. 2 (7): 1099-1104 (1983)), the EPSPS gene, which confers resistance to glyphosate (U.S. Pat. Nos. 4,940,935 and 5,188,642), the glyphosate N-acetyltransferase (GAT) gene, which also confers resistance to glyphosate (Castle et al. (2004) Science, 304:1151-1154; U.S. Patent App. Pub. Nos. 20070004912, 20050246798, and 20050060767); and the mannose-6-phosphate isomerase gene, which provides the ability to metabolize mannose (U.S. Pat. Nos. 5,767,378 and 5,994,629).

### A. Promoters

A number of promoters can be used in the various methods and compositions disclosed herein. The promoters can be selected based on the desired outcome. The nucleic acids can be combined with constitutive, inducible, tissue-preferred, or other promoters for expression in the organism of interest. See, for example, promoters set forth in WO 99/43838 and in U.S. Pat. Nos. 8,575,425; 7,790,846; 8,147,856; 8,586,832; 7,772,369; 7,534,939; 6,072,050; 5,659,026; 5,608,149; 5,608,144; 5,604,121; 5,569,597; 5,466,785; 5,399,680; 5,268,463; 5,608,142; and 6,177,611; herein incorporated by reference.

For expression in plants, constitutive promoters can be used. Non-limiting examples of constitutive promoters include CaMV 35S promoter (Odell et al. (985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten e/a/. (1984) EMBO J. 3:2723-2730). Inducible promoters include those that drive expression of pathogenesis-related proteins (PR proteins), which are induced following infection by a pathogen. See, for example, Redolfi et al. (1983) Neth. J. Plant Pathol. 89:245-254; Uknes et al. (1992) Plant Cell 4:645-656; and Van Loon (1985) Plant Mol. Virol. 4:111-116; and WO 99/43819, herein incorporated by reference. Promoters that are expressed locally at or near the site of pathogen infection may also be used (Marineau et al. (1987) Plant Mol. Biol. 9:335-342; Matton et al. (1989) Molecular Plant-Microbe Interactions 2:325-331; Somsisch et al. (1986) Proc. Natl. Acad. Sci. USA 83:2427-2430; Somsisch et al. (1988) Mol. Gen. Genet. 2:93-98; and Yang (1996) Proc. Natl. Acad. Sci. USA 93:14972-14977; Chen et al. (1996) Plant J. 10:955-966; Zhang et al. (1994) Proc. Natl. Acad. Sci. USA 91:2507-2511; Warner et al. (1993) Plant J. 3:191-201; Siebertz et al. (1989) Plant Cell 1:961-968; Cordero et al. (1992) Physiol. Mol. Plant Path. 41:189-200; U.S. Pat. No. 5,750,386 (nematode-inducible); and the references cited therein).

In specific embodiments, the various constitutive promoters disclosed in WO2019103918, WO2021000878, WO2021022022, WO2022173659, WO2021260673, WO2021263249, or U.S. Provisional Applications 63/481,627, 63/426,524, 63/509586, or 63/383,609 may be used; each of which is herein incorporated by reference in its entirety, including for example, those disclosed in WO2019103918 as SEQ ID NO: 16 (prGmUbi1), and SEQ ID NO: 19 (prMt51186); those disclosed in WO2021022022 as SEQ ID NO: 19 (prAtEF1aA1), SEQ ID NO: 20 (prUBQ3), or SEQ ID NO: 21 (prGmUbm); those disclosed in WO2021260673 and WO2021263249 as SEQ ID NOS: 6 and 8; those disclosed in WO2022173659 as SEQ ID NO: 13 (prMt12344) or SEQ ID NO: 14 (prMt51186); those disclosed in U.S. Provisional App. No. 63/426,524 as SEQ ID NO: 13 (prUBQ3), and SEQ ID NO: 14 (prGmUbi1), each of which are included herein by reference in their entirety. An example embodiment of a constitutive promoter (prMt51186) is also disclosed herein at SEQ ID NO: 12.

Wound-inducible promoters may be used in the constructions of the invention. Such wound-inducible promoters include pin II promoter (Ryan (1990) Ann. Rev. Phytopath. 28:425-449; Ouan et al. (1996) Nature Biotechnology 14:494-498); wun1 and wun2 (U.S. Pat. No. 5,428,148); win1 and win2 (Stanford et al. (1989) Mol. Gen. Genet. 215:200-208); systemin (McGurl et al. (1992) Science 225:1570-1573); WIP1 (Rohmeier et al. (1993) Plant Mol. Biol. 22:783-792; Eckelkamp et al. (1993) FEBS Letters 323:73-76); MPI gene (Corderok et al. (1994) Plant J. 6 (2): 141-150); and the like, herein incorporated by reference).

Still other inducible promoters may be used for the expression of a polypeptide of the invention in the constructs of the invention. In embodiments, the inducible promoter is a rust-reactive or rust-inducible promoter. As used herein, a “rust-inducible promoter” is a plant promoter that is induced or activated in response to rust exposure or rust infection of the plant. In particular embodiments, a rust-reactive or rust-inducible promoter (e.g., prLuFIS1) of the Fis1 gene from flax (Linum usitatissimum) may be used, such as disclosed in WO2021022022 as SEQ ID NO: 22 and in U.S. Provisional App. No. 63/383,609 as SEQ ID NO: 25, the contents of which are incorporated by reference herein in their entirety. In other specific embodiments, a rust reactive promoter (e.g., prGmACO3; SEQ ID NO: 14) derived from the ACO3 gene (Glyma.02G268200) from soybean (Glycine max) or a rust reactive promoter (e.g., prGmMYB; SEQ ID NO: 15) of a MYB gene (Glyma. 19G164600) from soybean (Glycine max) may be used.

Tissue-preferred promoters for use in the invention include those set forth in Yamamoto et al. (1997) Plant J. 12 (2): 255-265; Kawamata et al. (1997) Plant Cell Physiol. 38 (7): 792-803; Hansen et al. (1997) Mol. Gen Genet. 254 (3): 337-343; Russell et al. (1997) Transgenic Res. 6 (2): 157-168; Rinehart et al. (1996) Plant Physiol. 112 (3): 1331-1341; Van Camp et al. (1996) Plant Physiol. 112 (2): 525-535; Canevascim et al. (1996) Plant Physiol. 112 (2): 513-524; Yamamoto et al. (1994) Plant Cell Physiol. 35 (5): 773-778; Lam (1994) Results Probl. Cell Differ. 20:181-196; Orozco et al. (1993) PlantMolBiol. 23 (6): 1129-1138; Matsuoka et al. (1993) Proc Natl. Acad. Sci. USA 90 (20): 9586-9590; and Guevara-Garcia et al. (1993) Plant J. 4 (3): 495-505.

Leaf-preferred promoters include those set forth in Yamamoto et al. (1997) Plant J. 12 (2): 255-265; Kwon et al. (1994) Plant Physiol. 105:357-67; Yamamoto et al. (1994) Plant Cell Physiol. 35 (5): 773-778; Gotor et al. (1993) Plant J. 3:509-18; Orozco et al. (1993) Plant Mol. Biol. 23 (6): 1129-1138; and Matsuoka et al. (1993) Proc. Natl. Acad. Sci. USA 90 (20): 9586-9590.

Root-preferred promoters are known and include those set forth in Hire et al. (1992) Plant Mol. Biol. 20 (2): 207-218 (soybean root-specific glutamine synthetase gene); Keller and Baumgartner (1991) Plant Cell 3 (10): 1051-1061 (root-specific control element); Sanger et al. (1990) Plant Mol. Biol. 14 (3): 433-443 (mannopine synthase (MAS) gene of Agrobacterium tumefaciens); and Miao et al. (1991) Plant Cell 3 (1): 11-22 (cytosolic glutamine synthetase (GS)); Bogusz et al. (1990) Plant Cell 2 (7): 633-641; Leach and Aoyagi (1991) Plant Science (Limerick) 79 (1): 69-76 (rolC and rolD); Teeri et al. (1989) EMBO J. 8 (2): 343-350; Kuster et al. (1995) Plant Mol. Biol. 29 (4): 759-772 (the VfENOD-GRP3 gene promoter); and, Capana et al. (1994) Plant Mol. Biol. 25 (4): 681-691 (rolB promoter). See also U.S. Pat. Nos. 5,837,876; 5,750,386; 5,633,363; 5,459,252; 5,401,836; 5,110,732; and 5,023,179.

“Seed-preferred” promoters include both “seed-specific” promoters (promoters active during seed development such as promoters of seed storage proteins) as well as “seed-germinating” promoters (promoters active during seed germination). See Thompson et al. (1989) BioEssays 10:108. Seed-preferred promoters include, but are not limited to, Ciml (cytokinin-induced message); cZ19Bl (maize 19 kDa zein); milps (myo-inositol-1-phosphate synthase) (see WO 00/11177 and U.S. Pat. No. 6,225,529). Gamma-zein is an endosperm-specific promoter. Globulin 1 (Gib-1) is a representative embryo-specific promoter. For dicots, seed-specific promoters include, but are not limited to, bean β-phaseolin, napin, beta-conglycinin, soybean lectin, cruciferin, and the like. For Monocots, seed-specific promoters include, but are not limited to, maize 15 kDa zein, 22 kDa zein, 27 kDa zein, gamma-zein, waxy, shrunken 1, shrunken 2, Globulin 1, etc. See also WO 00/12733, where seed-preferred promoters from end1 and end2 genes are disclosed.

In some embodiments, promoters that control expression of resistance genes can be used to express the polynucleotide of interest. Such promoters include, but are not limited to the various native R-gene promoters set forth in WO2019103918, WO2021000878, WO2021022022, WO2022173659, WO2021260673, WO2021263249, or U.S. Provisional Applications 63/481,627, 63/426,524, 63/509586, or 63/383,609; each of which is herein incorporated by reference in its entirety, including, for example, those disclosed in WO2019103918 as SEQ ID NO: 7 (RG1 promoter), SEQ ID NO: 10, SEQ ID NO: 13, SEQ ID NO: 16, SEQ ID NO: 19, SEQ ID NO: 22, SEQ ID NO: 25, SEQ ID NO: 28, or SEQ ID NO: 31; in WO2021000878 as SEQ ID NO: 7 (Rpp6907 promoter); in WO2021022022 as SEQ ID NO: 9 (TirA promoter) and SEQ ID NO: 14 (TirB promoter); in WO2022173659 as SEQ ID NO: 15 (RG30 promoter); in WO2021260673 and WO202263249 as SEQ ID NO: 7 (RG21 promoter); and those disclosed in U.S. Provisional Application 63/383,609 as SEQ ID NO: 26 (RG3a promoter) and SEQ ID NO: 27 (RG3b promoter), and in U.S. Provisional Applications 63/426,524 and 63/509,586 as SEQ ID NO: 8 (RG32 promoter), SEQ ID NOS: 10-11 (RG34 promoters), and SEQ ID NO: 18 (bidirectional promoter); each of which is herein incorporated by reference in their entirety. Still other native promoters include the native Rg31 promoter (prGcaRG31) as set forth herein at SEQ ID NO: 7 and a variant thereof as set forth herein at SEQ ID NO: 8, and the native Rg35 promoter (prGcaRG35) as set forth herein at SEQ ID NO: 10.

In particular embodiments, the native promoter for an R-gene is a promoter sequence derived from the genomic sequence or genomic locus of the corresponding R-gene. In other specific embodiments, the native promoter sequence derived from the genomic sequence of a given R-gene may have one or more ORFs within the 5′-UTR sequence or within the native promoter sequence modified, replaced, or removed. In a particular example embodiment, a native promoter derived from the genomic sequence of the Rg31 gene with one or more ORFs of the 5′-UTR sequence modified or removed comprises the sequence as set forth herein at SEQ ID NO: 8. Another specific embodiment of a modified native promoter that can be used for controlling expression of a gene of interest is provided at U.S. Provisional App. No. 63/426,524 as SEQ ID NO: 11, which is herein incorporated by reference in its entirety.

For expression in a bacterial host, promoters that function in bacteria are known in the art. Such promoters include any of the known crystal protein gene promoters, including the promoters of any of the proteins of the invention, and promoters specific for B. thuringiensis sigma factors. Alternatively, mutagenized or recombinant crystal protein-encoding gene promoters may be recombinantly engineered and used to promote expression of the novel gene segments disclosed herein.

### B. Native Regulatory Elements

Compositions comprising novel regulatory elements are provided. In one embodiment, a polynucleotide comprising a regulatory element operably linked to a polynucleotide of interest is provided. Such regulatory elements include promoters and comprise the nucleotide sequence set forth in SEQ ID NO: 7, 8 and 10 or an active variant or fragment thereof. An active variant or fragment of the promoter will retain the ability to direct expression of the operably linked polynucleotide sequence. As such, active variants of the promoter sequence comprise at least 80%, 85%, 90%, 91%, 92%, 93%, 94%, 95%, 96%, 97%, 98% or 99% identity to SEQ ID NO: 7, 8, or 10 and retain the ability to direct expression of an operably linked nucleotide sequence. Fragments of such promoter sequences are also provided and can comprise at least 100, 200, 250, 300, 350, 400 or more nucleotides of the sequence set forth in SEQ ID NO: 7, 8, or 10. Fragments of such promoters can be active fragment and retain the ability to direct expression of an operably linked nucleotide sequence.

Regulatory elements comprising terminator sequences set forth in SEQ ID NO: 9 or 11 or an active variant or fragment thereof are provided. An active variant or fragment of the terminator sequence will retain the ability to regulate the expression of an operably linked polynucleotide sequence. As such, active variants of the terminator sequence comprise at least 80%, 85%, 90%, 91%, 92%, 93%, 94%, 95%, 96%, 97%, 98% or 99% identity to SEQ ID NO: 9 or 11 and retain the ability to direct expression of an operably linked nucleotide sequence. Fragments of such terminator sequences are also provided and can comprise at least 100, 200, 250, 300, 350, 400, 450, 500, 550, 600, 650, 700, 750, 800, 850, 900, 950, 1000 or more nucleotides of the sequence set forth in SEQ ID NO: 9 or 11. Fragments of such terminators can be active fragment and retain the ability to regulate expression of an operably linked nucleotide sequence.

Regulatory elements comprising intron sequences as set forth at as (i) a first native intron of the Rg31 gene (RG31_intron1, position 996 to 1,171 of SEQ ID NO: 3), (ii) a second native intron of the Rg31 gene (RG31_intron2, position 4,173 to 4,357 of SEQ ID NO: 3), (iii) a third native intron of the Rg31 gene (RG31_intron3, position 4,532 to 8,649 of SEQ ID NO: 3), (iv) a fourth native intron of the Rg31 gene (RG31_intron4, position 8,805 to 9,496 of SEQ ID NO: 3), (v) a fifth native intron of the Rg31 gene (RG31_intron5, position 9,654 to 10,351 of SEQ ID NO: 3); (vi) a sixth native intron of the Rg31 gene (RG31_intron6, position 10,515 to 12,562 of SEQ ID NO: 3); (vii) a seventh native intron of the Rg31 gene (RG31_intron7, position 12,654 to 12,723 of SEQ ID NO: 3); (viii) an eighth native intron of the Rg31 gene (RG31_intron8, position 12,764 to 13,781 of SEQ ID NO: 3); (ix) a ninth native intron of the Rg31 gene (RG31_intron9, position 13,917 to 14,981 of SEQ ID NO: 3); (x) a tenth native intron of the Rg31 gene (RG31_intron10, position 15,207 to 15,308 of SEQ ID NO: 3); (xi) an eleventh native intron of the Rg31 gene (RG31_intron11, position 15,492 to 15,587 of SEQ ID NO: 3); (xii) a twelfth native intron of the Rg31 gene (RG31_intron12, position 15,818 to 16,249 of SEQ ID NO: 3); (xiii) a thirteenth native intron of the Rg31 gene (RG31_intron13, position 16,296 to 16,373 of SEQ ID NO: 3); and (xiv) a fourteenth native intron of the Rg31 gene (RG31_intron14, position 16,441 to 16,803 of SEQ ID NO: 3), or an active variant or fragment thereof are provided. An active variant or fragment of the intron sequence will retain the ability to regulate the expression of an operably linked polynucleotide sequence. As such, active variants of the intron sequence comprise at least 80%, 85%, 90%, 91%, 92%, 93%, 94%, 95%, 96%, 97%, 98% or 99% identity to the above listed intron sequences and retain the ability to direct expression of an operably linked nucleotide sequence. Fragments of such introns sequences are also provided and can comprise at least 100, 200, 250, 300, 350, 400 or more nucleotides of the sequence set forth at any of the following positions: position 996 to 1,171; position 4,173 to 4,357; position 4,532 to 8,649; position 8,805 to 9,496; position 9,654 to 10,351; position 10,515 to 12,562; position 12,654 to 12,723; position 12,764 to 13,781; position 13,917 to 14,981; position 15,207 to 15,308; position 15,492 to 15,587; position 15,818 to 16,249; position 16,296 to 16,373; and position 16,441 to 16,803 of SEQ ID NO: 3. Fragments of such introns can be an active fragment and retain the ability to regulate expression of an operably linked nucleotide sequence.

Regulatory elements comprising intron sequences as set forth at as (i) a first native intron of the Rg35 gene (RG35_intron1, position 141 to 1,068 of SEQ ID NO: 5), (ii) a second native intron of the Rg35 gene (RG35_intron2, position 1,246 to 1,487 of SEQ ID NO: 5), (iii) a third native intron of the Rg35 gene (RG35_intron3, position 2,121 to 2,296 of SEQ ID NO: 5), (iv) a fourth native intron of the Rg35 gene (RG35_intron4, position 5,289 to 6,007 of SEQ ID NO: 5), (v) a fifth native intron of the Rg35 gene (RG35_intron5, position 6,173 to 8,273 of SEQ ID NO: 5); (vi) a sixth native intron of the Rg35 gene (RG35_intron6, position 8,426 to 9,122 of SEQ ID NO: 5); (vii) a seventh native intron of the Rg35 gene (RG35_intron7, position 9,280 to 9,973 of SEQ ID NO: 5); (viii) an eighth native intron of the Rg35 gene (RG35_intron8, position 10,139 to 12,192 of SEQ ID NO: 5); (ix) a ninth native intron of the Rg35 gene (RG35_intron9, position 12,322 to 13,324 of SEQ ID NO: 5); (x) a tenth native intron of the Rg35 gene (RG35_intron10, position 13,460 to 14,524 of SEQ ID NO: 5); (xi) an eleventh native intron of the Rg35 gene (RG35_intron11, position 14,750 to 14,851 of SEQ ID NO: 5); (xii) a twelfth native intron of the Rg35 gene (RG35_intron12, position 15,035 to 15,130 of SEQ ID NO: 5); (xiii) a thirteenth native intron of the Rg35 gene (RG35_intron13, position 15,361 to 15,780 of SEQ ID NO: 5); (xiv) a fourteenth native intron of the Rg35 gene (RG35_intron14, position 15,827 to 15,904 of SEQ ID NO: 5); and (xv) a fifteenth native intron of the Rg35 gene (RG35_intron15, position 15,972 to 16,334 of SEQ ID NO: 5), or an active variant or fragment thereof are provided. An active variant or fragment of the intron sequence will retain the ability to regulate the expression of an operably linked polynucleotide sequence. As such, active variants of the intron sequence comprise at least 80%, 85%, 90%, 91%, 92%, 93%, 94%, 95%, 96%, 97%, 98% or 99% identity to the above listed intron sequences and retain the ability to direct expression of an operably linked nucleotide sequence. Fragments of such introns sequences are also provided and can comprise at least 100, 200, 250, 300, 350, 400 or more nucleotides of the sequence set forth at any of the following positions: position 141 to 1,068, position 1,246 to 1,487, position 2,121 to 2,296, position 5,289 to 6,007, position 6,173 to 8,273, position 8,426 to 9,122, position 9,280 to 9,973, position 10,139 to 12,192, position 12,322 to 13,324, position 13,460 to 14,524, position 14,750 to 14,851, position 15,035 to 15,130, position 15,361 to 15,780, position 15,827 to 15,904 and position 15,972 to 16,334 of SEQ ID NO: 5. Fragments of such introns can be an active fragment and retain the ability to regulate expression of an operably linked nucleotide sequence.

Regulatory elements comprising intron sequences are also set forth as (i) a first native intron at position 141 to 1,068 of SEQ ID NO: 5, (ii) a second native intron at position 1,246 to 1,490 of SEQ ID NO: 5, (iii) a third native intron at position 2,121 to 2,296 of SEQ ID NO: 5, (iv) a fourth native intron at position 5,289 to 6,007 of SEQ ID NO: 5, (v) a fifth native intron at position 6,173 to 12192 of SEQ ID NO: 5; (vi) a sixth native intron at position 12,322 to 13,324 of SEQ ID NO: 5; (vii) a seventh native at position 13,460 to 14,524 of SEQ ID NO: 5; (xi) an eighth native intron at position 14,750 to 14,851 of SEQ ID NO: 5; (xii) a ninth native intron at position 15,035 to 15,130 of SEQ ID NO: 5; (xiii) a tenth native intron at position 15,361 to 15,780 of SEQ ID NO: 5; (xiv) an eleventh native intron at position 15,827 to 15,904 of SEQ ID NO: 5; and (xv) a twelfth native intron at position 15,972 to 16,334 of SEQ ID NO: 5; or an active variant or fragment thereof. An active variant or fragment of the intron sequence will retain the ability to regulate the expression of an operably linked polynucleotide sequence. As such, active variants of the intron sequence comprise at least 80%, 85%, 90%, 91%, 92%, 93%, 94%, 95%, 96%, 97%, 98% or 99% identity to the above listed intron sequences and retain the ability to direct expression of an operably linked nucleotide sequence. Fragments of such introns sequences are also provided and can comprise at least 100, 200, 250, 300, 350, 400 or more nucleotides of the sequence set forth at any of the above listed positions. Fragments of such introns can be an active fragment and retain the ability to regulate expression of an operably linked nucleotide sequence.

In some aspects, the disclosure provides an expression cassette. In some embodiments, the expression cassette comprises a nucleotide sequence comprising any one of SEQ ID NOS: 3-4 or any one of SEQ ID NOS: 5-6 or 21-22, wherein the nucleotide sequence is operably linked to a heterologous nucleotide sequence. In some embodiments, the expression cassette further comprises a selectable marker.

In some embodiments, the heterologous sequence of interest is a nucleic acid of interest that encodes an RNA or protein of interest. In some embodiments, the RNA or protein of interest is capable of conferring upon a plant a desired characteristic such as antibiotic resistance, virus resistance, insect resistance, disease resistance, resistance to other pests, herbicide tolerance, improved nutritional value, improved performance in an industrial process or altered reproductive capability. In some embodiments, the RNA or protein of interest comprises a genome editing agent, e.g., a CRISPR/Cas agent (such as a Cas protein and/or guide RNA), a TALEN, a DNA-guided nuclease, a meganuclease, a recombinase, or a zinc finger nuclease. In some embodiments, the heterologous nucleotide sequence encodes a selectable marker.

The heterologous nucleotide sequence of interest can comprise a sequence encoding a polypeptide of interest and in more specific embodiments, the heterologous nucleotide sequence of interest encodes a protein that increases disease resistance (e.g., increases fungal pathogen resistance such as ASR resistance) in a plant. Such sequences include but are not limited to polynucleotides encoding proteins that confer increased ASR resistance as described in US Patent publication Nos. US20200354739, and PCT Publications Nos. WO2019103918, WO2021000878, WO2021154632A1, WO2021022022, WO2021022026, WO2021022101, WO2022173659, WO2021260673, WO2021263249, and U.S. Provisional Applications 63/481,627, 63/426,524, 63/509586, and 63/383,609, each of which is incorporated by reference in its entirety.

In some embodiments, the expression cassette in a vector, such as a plasmid, virus, or Agrobacterium. In some embodiments, the expression cassette is in a plant cell as discussed elsewhere herein.

### 4. PLANTS, PLANT CELLS AND PLANT PARTS

Plants, plant parts, plant cells and seed are provided which comprise in their genome a nucleic acid sequence operably linked to a promoter active in the plant, wherein the nucleic acid sequence comprises a polynucleotide encoding an RG31 polypeptide as set forth in SEQ ID NO: 1, or an active variant or fragment thereof, or wherein the nucleic acid sequence comprises a polynucleotide encoding an RG35 polypeptide as set forth in SEQ ID NO: 2 or 20, or an active variant or fragment thereof. In particular embodiments, the plants, plant parts, plant cells and seed express the RG31 or RG35 polypeptide, or their active variants and fragments, in their genome. In still further embodiments, plants, plant parts, plant cells and seed are provided which comprise in their genome a heterologous nucleic acid sequence comprising a polynucleotide as set forth in any one of SEQ ID NOS: 3-6 or 21-22, or active variants and fragments thereof. Such heterologous polynucleotides can be transiently expressed or stably integrated into the genome.

Although soybean plants are used to exemplify the composition and methods throughout the application, a polynucleotide as provided herein may be introduced to any plant species, including, but not limited to, monocots and dicots. Examples of plants of interest include, but are not limited to, corn (maize), sorghum, wheat, sunflower, tomato, crucifers, peppers, potato, cotton, rice, soybean, sugarbeet, sugarcane, tobacco, barley, and oilseed rape, Brassica sp., alfalfa, rye, millet, safflower, peanuts, sweet potato, cassava, coffee, coconut, pineapple, citrus trees, cocoa, tea, banana, avocado, fig, guava, mango, olive, papaya, cashew, macadamia, almond, oats, vegetables, ornamentals, and conifers.

In specific embodiments, the plant is a legume. Examples of legumes include, but are not limited to, the genus Phaseolus (e.g., French bean, dwarf bean, climbing bean (Phaseolus vulgaris), Lima bean (Phaseolus lunatus), Tepary bean (Phaseolus acutifolius), runner bean (Phaseolus coccineus)); the genus Glycine (e.g., Glycine soja, soybeans (Glycine max (L.))); pea (Pisum) (e.g., shelling peas (sometime called smooth or round-seeded peas; Pisum sativum); marrowfat pea (Pisum sativum), sugar pea (Pisum sativum), also called snow pea, edible-podded pea or mangetout (Pisum granda); peanut (Arachis hypogaea), clover (Trifolium spp.), medick (Medicago), kudzu vine (Pueraria lobata), common lucerne, alfalfa (Medicago sativa), chickpea (Cicer), lentils (Lens culinaris), lupins (Lupinus); vetches (Vicia), field bean, broad bean (Vicia faba), vetchling (Lathyrus) (e.g., chickling pea (Lathyrus sativus), heath pea (Lathyrus tuberosus)); genus Vigna (e.g., moth bean (Vigna aconiti folia), adzuki bean (Vigna angularis), urad bean (Vigna mungo), mung bean (Vigna radiata), bambara groundnut (Vigna subterrane), rice bean (Vigna umbellata), Vigna vexillata, Vigna unguiculata (also known as asparagus bean, cowpea); pigeon pea (Cajanus cajari; Cajanus cajan), the genus Macrotyloma (e.g., geocarpa groundnut (Macrotyloma geocarpum), horse bean (Macrotyloma uniflorum); goa bean (Psophocarpus tetragonolobus), African yam bean (Sphenostylis stenocarpa), Egyptian black bean, lablab bean (Lablab purpureus), yam bean (Pachyrhizus erosus), guar bean (Cyamopsis tetragonolobus); and/or the genus Canavalia (e.g., jack bean (Canavalia ensiformis)), sword bean (Canavalia gladiata).

In one embodiment, the legume plant is soybean, and more particularly Glycine max.

Glycine (soybean or soya bean) is a genus in the bean family Fabaceae. The Glycine plants provided herein can be Glycine arenaria, Glycine argyrea, Glycine cyrtoloba, Glycine canescens, Glycine clandestine, Glycine curvata, Glycine falcata, Glycine latifolia, Glycine microphylla, Glycine pescadrensis, Glycine stenophita, Glycine syndetica, Glycine soja Seib. Et Zucc., Glycine max (L.) Merrill., Glycine tabacina, or Glycine tomentella.

In some embodiments, the plants provided herein (legumes or soybeans plants) are elite plants, elite germplasm or are derived from an elite line or an elite germplasm. Numerous elite lines are available and known to those of skill in the art of soybean breeding and are discussed in further detail elsewhere herein.

In some embodiments, the plants provided herein can comprise one or more additional polynucleotides that encode an additional polypeptide that increases disease resistance of the plant. Such combinations are described in further detail elsewhere herein.

In specific embodiments, the plants, plant parts or seeds having the heterologous polynucleotide or polypeptide disclosed herein or active variants and fragments thereof can have an increased expression of the polynucleotide or polypeptide. In other embodiments, the plants, plant parts or seeds having the heterologous polynucleotide or polypeptide disclosed herein or active variants and fragments thereof can have an increased level of activity of the polypeptide. Methods to generate such increased levels of expression or activity include, but are not limited to, breeding, gene editing, and transgenic techniques.

In some embodiments, a plant cell, seed, or plant part or harvest product can be obtained from the plant produced as above and the plant cell, seed, or plant part can be screened using methods disclosed above for the evidence of stable incorporation of the polynucleotide. The term “stable incorporation” refers to the integration of a nucleic acid sequence into the genome of a plant and the nucleic acid sequence is capable of being inherited by the progeny thereof.

In some embodiments, plant products can be harvested from the plant disclosed above and processed to produce processed products, such as flour, soy meal, oil, starch, and the like. These processed products are also within the scope of this invention provided that they comprise a polynucleotide or polypeptide or variant or fragment thereof disclosed herein. Other soybean plant products include but are not limited to protein concentrate, protein isolate, soybean hulls, meal, flower, oil and the whole soybean itself.

Seed lots are provided comprising populations of seeds that comprise in their genome a heterologous nucleic acid sequence comprising a polynucleotide encoding a RG31 polypeptide set forth in SEQ ID NO: 1, or an active variant or fragment thereof, or a polynucleotide encoding a RG35 polypeptide as set forth in SEQ ID NO: 2 or 20, or active variant or fragment thereof; and having an increased disease resistance. In other embodiments, the seed lots comprises populations of seed, which comprise in their genome a heterologous nucleic acid sequence comprising a polynucleotide as set forth in any one of SEQ ID NOS: 3-6 or 21-22 or variants and fragments thereof.

Such seed can be from any plant, including but not limited to dicotyledonous crop plants, legumes, or soybean. Methods of making a seed lot comprise harvesting seed from a plant having the increase resistance to a plant pathogen. Such seed lots can comprise at least 50, 100, 1000, 100000 seeds or more of the invention.

Further provided is an ensemble of plants that produce seeds having an increased resistance to a plant pathogen as described herein. Such an ensemble of plants have stably integrated into their genomes a heterologous nucleic acid sequence comprising a polynucleotide encoding a RG31 polypeptide as set forth in SEQ ID NO: 1 or an active variant or fragment thereof, or a heterologous nucleic acid sequence comprising a polynucleotide encoding a RG35 polypeptide as set forth in SEQ ID NO: 2 or 20 or an active variant or fragment thereof; and wherein the ensemble of plants have increased disease resistance. In other embodiments, the seed lots comprises populations of seed, which comprise in their genome a heterologous nucleic acid sequence comprising a polynucleotide as set forth in any one of SEQ ID NOS: 3-6 or 21-22 or variants and fragments thereof. The term ensemble encompasses any collection of plants linked together by proximity, such as plants in a field, a greenhouse, or a tray. The ensemble of plants comprises at least 50, 100, 1000, 10000, 100000 or more plants of the invention.

### 5. METHODS FOR PRODUCING A PLANT THAT HAS INCREASED DISEASE RESISTANCE

Provided herein are methods of producing a plant, plant part or a seed that has increased disease resistance by introducing into the plant, plant part, or plant cell, a nucleic acid sequence comprising a polynucleotide encoding a RG31 polypeptide as set forth in SEQ ID NO: 1 or an active variant or fragment thereof, or by introducing a nucleic acid sequence comprising a polynucleotide encoding a RG35 polypeptide as set forth in SEQ ID NO: 2 or 20 or an active variant or fragment thereof, wherein expression of the RG31 polypeptide or the RG35 polypeptide increases disease resistance of the plant. In other embodiments, the method comprises introducing into the plant, plant cell or plant part a nucleic acid sequence comprising a polynucleotide as set forth in any of SEQ ID NOS: 3-4 or SEQ ID NOS: 5-6 or 21-22, or variants and fragments thereof, wherein expression of the polynucleotide increases disease resistance of the plant.

A nucleic acid sequence may be introduced into a plant cell by various ways, for example, by transformation, by genome modification techniques (such as by genome editing or targeted integration), or by breeding. In one aspect, the plant can be produced by transforming the nucleic acid sequence encoding the RG31 or RG35 polypeptide, or an active variant or fragment thereof, into a recipient plant. In one aspect, the method can comprise editing the genome of the recipient plant so that the resulting plant comprises a polynucleotide encoding the RG31 or RG35 polypeptide, or an active variant or fragment thereof. In another aspect, the method can comprise breeding a donor plant comprising a polynucleotide encoding the RG31 or the RG35 polypeptide provided herein or an active variant or fragment thereof with a recipient plant and selecting for incorporation of the polynucleotide into the recipient plant genome.

A. Methods and Compositions to Increase Expression and/or Activity of a Polypeptide of Interest

Methods and compositions are provided that increase disease resistance in a plant by increasing expression and/or activity of the RG31 polypeptide, or active variant or fragment thereof, or increasing expression and/or activity of the RG35 polypeptide, or active variant or fragment thereof.

In particular embodiments, the methods and compositions provided herein increase expression of the RG31 or RG35 polypeptide, or their fragments and variants. As used herein, “increasing the expression” or “increased expression” of a RG31 or a RG35 polypeptide, or an active variant or fragment of the RG31 or RG35 polypeptide, means the level of the RG31 or RG35 polypeptide, or active variant or fragment thereof, produced by the given plant, plant cell, plant part, or seed is statistically higher than the expression level compared against an appropriate control plant, plant part, plant cell or seed. In specific embodiments, the increase in expression can comprise any statistically significant increase in the concentration of the polypeptide (e.g., increase in the concentration of the RG31 polypeptide or increase in the concentration of the RG35 polypeptide) by at least 1%, 2%, 3%, 4%, 5%, 6%, 7%, 8%, 9%, 10%, 15%, 20%, 30%, 40%, 50%, 60%, 70%, 80%, 90%, or 100% when compared to an appropriate control. In other embodiments, the increase in expression can comprise an increase in the level of the target protein concentration by at least 1-fold, 10-fold, 20 fold, 30 fold, 40 fold, 50 fold, 60 fold, 70 fold, 80 fold, 90 fold, 100 fold, 150 fold, 175 fold, 200 fold or more when compared to an appropriate control plant. Methods to assay for an increase in the level or expression of a target polypeptide are known and include, for example, the detection of the protein via antibodies or detection of an increase in the level of expression of the mRNA encoding the protein.

In other embodiments, methods and compositions are provided that increase the activity of the RG31 polypeptide, or active variant or fragment thereof, or increase the activity of the RG35 polypeptide or active variant or fragment thereof, in a plant, plant cell or plant part. As used herein, “increasing the activity” or “increased activity” of the RG31 or RG35 polypeptide, or active variant or fragment thereof, means the level of the enzyme activity or protein functionality of the RG31 or RG35 polypeptide is statistically higher when compared to an appropriate control. In particular embodiments, the increased activity of the RG31 polypeptide or active variant or fragment thereof means one or more of: (i) the ability of the RG31 polypeptide (or active variant or fragment thereof) to mount an immune response in a plant cell is increased and/or (ii) the ability of the RG31 polypeptide (or active variant or fragment thereof) to confer disease resistance to a plant when expressed in a plant, plant part, or seed, is increased. In other particular embodiments, the increased activity of the RG35 polypeptide or active variant or fragments thereof means one or more of: (i) the ability of the RG35 polypeptide (or active variant or fragment thereof) to mount an immune response in a plant cell is increased, and/or (ii) the ability of the RG35 polypeptide (or active variant or fragment thereof) to confer disease resistance to a plant when expressed in a plant, plant part, or seed, is increased.

In specific embodiments, the increase in activity can comprise an increase in the level of enzyme activity or protein functionality of the RG31 or RG35 polypeptide or active variant or fragment thereof by at least 1%, 2%, 3%, 4%, 5%, 6%, 7%, 8%, 9%, 10%, 15%, 20%, 30%, 40%, 50%, 60%, 70%, 80%, 90%, 100% when compared to an appropriate control plant, plant part, plant cell or seed. In other embodiments, the increase in the activity of the RG31 or RG35 polypeptide or active variant or fragment thereof can comprise an increase in the level of enzymatic activity or protein functionality by at least 1-fold, 10 fold, 20 fold, 30 fold, 40 fold, 50 fold, 60 fold, 70 fold, 80 fold, 90 fold, 100 fold, 150 fold, 175 fold, 200 fold or more when compared to an appropriate control plant, plant part, plant cell or seed. Methods to assay for an increase in the enzyme activity or protein functionality include direct assays for the activity of the specific protein and as well as indirect assays.

In some embodiments, increasing the activity and/or expression level of a RG31 or a RG35 polypeptide includes the introduction of a nucleic acid construct into a plant that results in the increased expression and/or activity of the RG31 or RG35 polypeptide, or active variants or fragments thereof. The nucleic acid construct can be stably integrated in the genome or be provided transiently. For example, the nucleic acid construct can comprise the nucleic acid sequence encoding the RG31 polypeptide of SEQ ID NO: 1 (such as the nucleic acid sequence of SEQ ID NOS: 3 or 4) or encoding the RG35 polypeptide of SEQ ID NO: 2 or 20 (such as the nucleic acid sequence of SEQ ID NOS: 5 or 6), or active variants or fragments thereof.

As such, plants, plant parts, seeds and plant cells are provided having stably incorporated into their genome a polynucleotide operably linked to a promoter active in the plant, wherein the polynucleotide encodes a polypeptide having an amino acid sequence having at least 80%, 85%, 90%, 91%, 92%, 93%, 94%, 95%, 96%, 97%, 98%, 99% or 100% sequence identity to SEQ ID NO: 1, or an active variant or active fragment thereof, or wherein the polynucleotide encodes a polypeptide having an amino acid sequence having at least 80%, 85%, 90%, 91%, 92%, 93%, 94%, 95%, 96%, 97%, 98%, 99% or 100% sequence identity to any one of SEQ ID NO: 2 or 20, or an active variant or active fragment thereof, wherein the increase in expression or activity of the polypeptide of SEQ ID NO: 1 or 2 or 20, or active variants or fragments thereof, increases the disease resistance and/or the pathogen resistance of the plant. Upon transformation, the target polypeptides are integrated into the genome and expressed in a manner that increases the activity or expression level of the RG31 or RG35 polypeptide in the plant.

Methods are therefore provided whereby the pathogen resistance of a plant is increased by introducing into the genome of the plant a nucleic acid construct that results in the increased expression and/or activity of a polypeptide having an amino acid sequence having at least 80%, 85%, 90%, 91%, 92%, 93%, 94%, 95%, 96%, 97%, 98%, 99% or 100% sequence identity to any one of SEQ ID NO: 1, or an active variant or active fragment thereof, or that results in the increased expression and/or activity of a polypeptide having an amino acid sequence having at least 80%, 85%, 90%, 91%, 92%, 93%, 94%, 95%, 96%, 97%, 98%, 99% or 100% sequence identity to any one of SEQ ID NO: 2 or 20, or an active variant or active fragment thereof, wherein the introduction of the nucleic acid construct into the genome increases the pathogen resistance and/or the disease resistance of the plant. In one embodiment, the method comprises introducing into the genome of a plant a nucleic acid sequence encoding a polypeptide having an amino acid sequence having at least 80%, 85%, 90%, 91%, 92%, 93%, 94%, 95%, 96%, 97%, 98%, 99% or 100% sequence identity to any one of SEQ ID NO: 1, or an active variant or active fragment thereof, or encoding a polypeptide having an amino acid sequence having at least 80%, 85%, 90%, 91%, 92%, 93%, 94%, 95%, 96%, 97%, 98%, 99% or 100% sequence identity to any one of SEQ ID NO: 2 or 20, or an active variant or active fragment thereof, where the increased activity or expression of the polypeptides increases the pathogen resistance and/or the disease resistance of the plant. In some embodiments, an expression cassette comprising a promoter active in the plant is operably linked to the polynucleotide of interest encoding the RG31 or RG35 polypeptide, or their active variants or fragments thereof, is introduced into the genome of the plant. In other embodiments, the polynucleotide of interest can be introduced into the genome of the plant and integrated at a genomic location (via for example, targeted integration) that allows for the expression of the polypeptide.

In other embodiments, the methods comprise introducing a nucleic acid construct that produces a modification in the genome of the plant that results in an increased expression or an increased activity of a RG31 polypeptide having an amino acid sequence with at least 80%, 85%, 90%, 91%, 92%, 93%, 94%, 95%, 96%, 97%, 98%, 99%, or 100% identity to any one of SEQ ID NO: 1 or an active variant or fragment thereof, or that results in an increased expression or an increased activity of a RG35 polypeptide having an amino acid sequence with at least 80%, 85%, 90%, 91%, 92%, 93%, 94%, 95%, 96%, 97%, 98%, 99%, or 100% identity to any one of SEQ ID NO: 2 or 20, or an active variant or fragment thereof, where the modification increases the pathogen resistance of the plant.

### B. Transformation Methods

In some embodiments, methods of introducing a polynucleotide into a plant comprises transforming a polynucleotide disclosed herein or an active variant or fragment thereof into a recipient plant to obtain a transgenic plant and said transgenic plant has increased pathogen resistance and/or disease resistance. Expression cassettes comprising polynucleotides encoding the polypeptides as described above can be used to transform plants of interest.

Transformation results in the introduction of a heterologous nucleic acid into a plant, including whole plants, as well as plant organs (e.g., leaves, stems, roots, etc.), seeds, plant cells, propagules, embryos and progeny of the same. Plant cells can be differentiated or undifferentiated (e.g., callus, suspension culture cells, protoplasts, leaf cells, root cells, phloem cells, pollen). Transformation may result in stable or transient incorporation of the nucleic acid into the cell. “Stable transformation” is intended to mean that the nucleotide construct introduced into a host cell integrates into the genome of the host cell and is capable of being inherited by the progeny thereof. In some embodiments, the stable transformation occurs via a random integration event. In other embodiments, the stable transformation occurs via a targeted integration into the genome of the sequence of interest employing genome modification machinery such as, for example, CRISPRs or TALENs. “Transient transformation” is intended to mean that a polynucleotide is introduced into the host cell and does not integrate into the genome of the host cell.

Methods for transformation typically involve introducing a nucleotide construct into a plant. In some embodiments, the transformation method is an Agrobacterium-mediated transformation. In some embodiments, the transformation method is a biolistic-mediated transformation. Transformation may also be performed by infection, transfection, microinjection, electroporation, microprojection, biolistics or particle bombardment, electroporation, silica/carbon fibers, ultrasound mediated, PEG mediated, calcium phosphate co-precipitation, poly cation DMSO technique, DEAE dextran procedure, Agrobacterium and viral mediated (e.g., Caulimoriviruses, Geminiviruses, RNA plant viruses), liposome mediated and the like.

Transformation protocols as well as protocols for introducing polypeptides or polynucleotide sequences into plants may vary depending on the type of plant or plant cell, i.e., monocot or dicot, targeted for transformation. Methods for transformation are known in the art and include those set forth in U.S. Pat. Nos. 8,575,425; 7,692,068; 8,802,934; and 7,541,517; each of which is herein incorporated by reference. See, also, Rakoczy-Trojanowska, M. (2002) Cell Mol Biol Lett. 7:849-858; Jones et al. (2005) Plant Methods, Vol. 1, Article 5; Rivera et al. (2012) Physics of Life Reviews 9:308-345; Bartlett et al. (2008) Plant Methods 4:1-12; Bates, G. W. (1999) Methods in Molecular Biology 111:359-366; Binns and Thomashow (1988) Annual Reviews in Microbiology 42:57 Sup′/Sup5-606; Christou, P. (1992) The Plant Journal 2:275-281; Christou, P. (1995) Euphytica 85:13-27; Tzfira et al. (2004) TRENDS in Genetics 20:375-383; Yao et al. (2006) Journal of Experimental Botany 57:3737-3746; Zupan and Zambryski (1995) Plant Physiology 107:1041-1047.

Methods of transformation of plant cells or tissues include but are not limited to Agrobacterium mediated transformation method and the Biolistics or particle-gun mediated transformation method. Suitable plant transformation vectors for the purpose of Agrobacterium mediated transformation include-those elements derived from a tumor inducing (Ti) plasmid of Agrobacterium tumefaciens, for example, right border (RB) regions and left border (LB) regions, and others disclosed by Herrera-Estrella et al., Nature 303:209 (1983); Bevan, Nucleic Acids Res. 12:8711-8721 (1984); Klee et al., Bio-Technology 3 (7): 637-642 (1985). In addition to plant transformation vectors derived from the Ti or root-inducing (Ri) plasmids of Agrobacterium, alternative methods can be used to insert the DNA constructs of this invention into plant cells. Such methods may involve, but are not limited to, for example, the use of liposomes, electroporation, chemicals that increase free DNA uptake, free DNA delivery via microprojectile bombardment, and transformation using viruses or pollen.

Methods for transformation of chloroplasts are known in the art. See, for example, Svab et al. (1990) Proc. Natl. Acad. Sci. USA 87 (21): 8526-8530; Svab and Maliga (1993) Proc. Natl. Acad. Sci. USA 90 (3): 913-917; Staub and Maliga (1993) EMBO J. 12 (2): 601-606. The method relies on particle gun delivery of DNA containing a selectable marker and targeting of the DNA to the plastid genome through homologous recombination. Additionally, plastid transformation can be accomplished by transactivation of a silent plastid-borne transgene by tissue-preferred expression of a nuclear-encoded and plastid-directed RNA polymerase. Such a system has been reported in McBride et al. (1994) Proc. Natl. Acad. Sci. USA 91 (15): 7301-7305.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a nucleotide construct of the invention, for example, an expression cassette of the invention, stably incorporated into their genome.

“Regeneration” refers to the process of growing a plant from a plant cell (for example, plant protoplast or explant). Such regeneration techniques rely on manipulation of certain phytohormones in a tissue culture growth medium, typically relying on a biocide and/or herbicide marker that has been introduced together with the desired nucleotide sequences. Choice of methodology for the regeneration step is not critical See, for example, Ammirato et al., Handbook of Plant Cell Culture Crop Species. Macmillan Publ. Co. (1984); Shimamoto et al., Nature 338:274-276 (1989); Fromm, UCLA Symposium on Molecular Strategies for Crop Improvement, Apr. 16-22, 1990. Keystone, Colo. (1990); Vasil et al., Bio/Technology 8:429-434 (1990); Vasil et al., Bio/Technology 10:667-674 (1992); Hayashimoto, Plant Physiol. 93:857-863 (1990); and Datta et al., Bio-technology 8:736-740 (1990). Such regeneration techniques are described generally in Klee et al., Ann. Rev. Plant Phys. 38:467-486 (1987).

### C. Crossing

In some embodiments, the method comprises crossing a donor plant comprising a polynucleotide encoding a RG31 polypeptide or active variant or fragment thereof, or encoding a RG35 polypeptide or active variant or fragment thereof, with a recipient plant, and the polypeptide is able to confer increased pathogen resistance in the recipient plant. As used herein, the terms “crossing” and “breeding” refer to the fusion of gametes to produce progeny (e.g., by fertilization, such as to produce seed by pollination in plants). In some embodiments, a “cross,” “breeding,” or “cross-fertilization” is fertilization of one individual by another (e.g., cross-pollination in plants). The plant disclosed herein may be a whole plant, or may be a plant cell, seed, or tissue, or a plant part such as leaf, stem, pollen, or cell that can be cultivated into a whole plant.

In some embodiments, a progeny plant created by the crossing or breeding process is repeatedly crossed back to one of its parents through a process referred to herein as “backcrossing”. In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. For example, see Ragot, M. et al. Marker-assisted Backcrossing: A Practical Example, in Techniques et Utilisations des Marqueurs Moleculaires Les Colloques, Vol. 72, pp. 45-56 (1995); and Openshaw et al., Marker-assisted Selection in Backcross Breeding, in Proceedings of the Symposium “Analysis of Molecular Marker Data,” pp. 41-43 (1994). The initial cross gives rise to the F1 generation. The term “BC1” refers to the second use of the recurrent parent, “BC2” refers to the third use of the recurrent parent, and so on.

In some embodiments, the donor soybean plant is a Glycine max plant. In some embodiments, the donor soybean plant is a Glycine soja plant. In some embodiments, the recipient soybean plant is an elite Glycine max plant or an elite Glycine soja plant.

### D. Gene Editing

Further provided are plants, plant cells and seeds having a genomic modification created through gene editing. Such methods include, but are not limited to, meganucleases designed against the plant genomic sequence of interest, CRISPR-Cas9, TALENs, and other technologies for precise editing of genomes (Feng, et al. Cell Research 23:1229-1232, 2013, WO 2013/026740); Cre-lox site-specific recombination; FLP-FRT recombination (Li et al. (2009) Plant Physiol 151:1087-1095); Bxbl-mediated integration (Yau et al. Plant J (2011) 701:147-166); zinc-finger mediated integration (Wright et al. (2005) Plant J 44:693-705); Cai et al. (2009) Plant Mol Biol 69:699-709); and homologous recombination (Lieberman-Lazarovich and Levy (2011) Methods Mol Biol: 51-65).

Various embodiments of the methods described herein use gene editing. In some embodiments, gene editing is used to mutagenize the genome of a plant to produce plants having one or more of the polypeptides that are able to increase disease resistance in a plant. In other instances, the gene editing is used to allow for targeted insertion into the genome of a nucleotide sequence encoding the RG31 or RG35 polypeptide or active variants or fragments thereof.

“Target site,” “target sequence,” “target DNA,” “target locus,” “genomic target site,” “genomic target sequence,” and “genomic target locus” are used interchangeably herein and refer to a polynucleotide sequence, for example in the genome (including chloroplastic and mitochondrial DNA) of a cell, to which an endonuclease is recruited, and optionally nicks or cleaves the DNA of the target site. The target site can be an endogenous site in the plant genome, or alternatively, the target site can be heterologous to the plant and thereby not be naturally occurring in the genome, or the target site can be found in a heterologous genomic location compared to where it occurs in nature.

In some embodiments, provided herein are plants transformed with and expressing gene-editing machinery as described above, which, when crossed with a target plant, result in gene editing in the target plant.

The term “polynucleotide modification template” includes a polynucleotide that comprises at least one nucleotide modification when compared to the nucleotide sequence to be edited. A nucleotide modification can be at least one nucleotide substitution, addition, or deletion. The polynucleotide modification template can further comprise homologous nucleotide sequences flanking at least one nucleotide modification, wherein flanking homologous nucleotide sequences provides sufficient homology to the desired nucleotide sequence to be edited.

Gene editing generally refers to the use of a site-directed nuclease (including but not limited to CRISPR/Cas, zinc fingers, meganucleases, and the like) to cut a nucleotide sequence at a desired location. This may be to cause an insertion/deletion (“indel”) mutation, (i.e., “SDN1”), a base edit (i.e., “SDN2”), or allele insertion or replacement (i.e., “SDN3”). SDN2 or SDN3 gene editing may comprise the provision of one or more recombination templates (e.g., in a vector) comprising a gene sequence of interest that can be used for homology directed repair (HDR) within the plant (i.e., to be introduced into the plant genome). In some embodiments, the gene or allele of interest is one that is able to confer to the plant an improved trait, e.g., increased protein content and/or altered seed composition. The recombination template can be introduced into the plant either through transformation or through breeding with a donor plant comprising the recombination template. Breaks in the plant genome may be introduced within, upstream, and/or downstream of a target sequence. In some embodiments, a double strand DNA break is made within or near the target sequence locus. In some embodiments, breaks are made upstream and downstream of the target sequence locus, which may lead to its excision from the genome. In some embodiments, one or more single strand DNA breaks (nicks) are made within, upstream, and/or downstream of the target sequence (e.g., using a nickase Cas9 variant). Any of these DNA breaks, as well as those introduced via other methods known to one of skill in the art, may induce HDR. Through HDR, the target sequence is replaced by the sequence of the provided recombination template comprising a polynucleotide of interest, e.g., any one of SEQ ID NOS: 3-4 or 5-6 or variant or fragment thereof may be provided on/as a template. By designing the system such that one or more single strand or double strand breaks are introduced within, upstream, and/or downstream of the corresponding region in the genome of a plant not comprising the gene sequence of interest, this region can be replaced with the template.

In some embodiments, mutations in the genes of interest described herein may be generated without the use of a recombination template via targeted introduction of DNA double strand breaks. Such breaks may be repaired through the process of non-homologous end joining (NHEJ), which can result in the generation of small insertions or deletions (indels) at the repair site. Such indels may lead to frameshift mutations causing premature stop codons or other types of loss-of-function mutations in the targeted genes.

In some embodiments, gene editing may involve transient, inducible, or constitutive expression of the gene editing components or systems in the target plant. Gene editing may also involve genomic integration or episomal presence of the gene editing components or systems in the target plant.

In certain embodiments, the nucleic acid modification or mutation is affected by a (modified) zinc-finger nuclease (ZFN) system. The ZFN system uses artificial restriction enzymes generated by fusing a zinc finger DNA-binding domain to a DNA-cleavage domain that can be engineered to target desired DNA sequences. Exemplary methods of genome editing using ZFNs can be found for example in U.S. Pat. Nos. 6,534,261; 6,607,882; 6,746,838; 6,794,136; 6,824,978; 6,866,997; 6,933,113; and 6,979,539.

In certain embodiments, the nucleic acid modification is affected by a (modified) meganuclease, which are endodeoxyribonucleases characterized by a large recognition site (double-stranded DNA sequences of 12 to 40 base pairs). Exemplary methods for using meganucleases can be found in U.S. Pat. Nos. 8,163,514; 8,133,697; 8,021,867; 8,119,361; 8,119,381; 8,124,369; and 8,129,134, which are specifically incorporated by reference.

In certain embodiments, the nucleic acid modification is affected by a (modified) CRISPR/Cas complex or system. In certain embodiments, the CRISPR/Cas system or complex is a class 2 CRISPR/Cas system. In certain embodiments, said CRISPR/Cas system or complex is a type II, type V, or type VI CRISPR/Cas system or complex. The CRISPR/Cas system does not require the generation of customized proteins to target specific sequences but rather a single Cas protein can be programmed by an RNA guide (gRNA) to recognize a specific nucleic acid target, in other words the Cas enzyme protein can be recruited to a specific nucleic acid target locus (which may comprise or consist of RNA and/or DNA) of interest using said short RNA guide.

In general, the CRISPR/Cas or CRISPR system is as used herein refers collectively to transcripts and other elements involved in the expression of or directing the activity of CRISPR-associated (“Cas”) genes, including sequences encoding a Cas gene and one or more of, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and, where applicable, transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise any polynucleotide, such as DNA or RNA polynucleotides.

In certain embodiments, the gRNA is a chimeric guide RNA or single guide RNA (sgRNA). In certain embodiments, the gRNA comprises a guide sequence and a tracr mate sequence (or direct repeat). In certain embodiments, the gRNA comprises a guide sequence, a tracr mate sequence (or direct repeat), and a tracr sequence. In certain embodiments, the CRISPR/Cas system or complex as described herein does not comprise and/or does not rely on the presence of a tracr sequence (e.g. if the Cas protein is Cas12a).

The Cas protein as referred to herein, such as but not limited to Cas9, Cas12a (formerly referred to as Cpf1), Cas12b (formerly referred to as C2c1), Cas13a (formerly referred to as C2c2), C2c3, Cas13b protein, may originate from any suitable source, and hence may include different orthologues, originating from a variety of (prokaryotic) organisms, as is well documented in the art. In certain embodiments, the Cas protein is (modified) Cas9, preferably (modified) Staphylococcus aureus Cas9 (SaCas9) or (modified) Streptococcus pyogenes Cas9 (SpCas9). In certain embodiments, the Cas protein is Cas12a, optionally from Acidaminococcus sp., such as Acidaminococcus sp. BV3L6 Cpf1 (AsCas12a) or Lachnospiraceae bacterium Cas12a, such as Lachnospiraceae bacterium MA2020 or Lachnospiraceae bacterium MD2006 (LBCas12a). See U.S. Pat. No. 10,669,540, incorporated herein by reference in its entirety. Alternatively, the Cas12a protein may be from Moraxella bovoculi AAX08_00205 [Mb2Cas12a] or Moraxella bovoculi AAX11_00205 [Mb3Cas12a]. See WO 2017/189308, incorporated herein by reference in its entirety. In certain embodiments, the Cas protein is (modified) C2c2, preferably Leptotrichia wadei C2c2 (LwC2c2) or Listeria newyorkensis FSL M6-0635 C2c2 (LbFSLC2c2). In certain embodiments, the (modified) Cas protein is C2c1. In certain embodiments, the (modified) Cas protein is C2c3. In certain embodiments, the (modified) Cas protein is Cas13b. Other Cas enzymes are available to a person skilled in the art.

Gene editing methods and compositions are also disclosed in U.S. Pat. Nos. 10,519,456 and 10,285,348 82, the entire content of which is herein incorporated by reference.

The gene-editing machinery (e.g., the DNA modifying enzyme) introduced into the plants can be controlled by any promoter that can drive recombinant gene expression in plants. In some embodiments, the promoter is a constitutive promoter. In some embodiments, the promoter is a tissue-specific promoter, e.g., a pollen-specific promoter or a sperm cell specific promoter, a zygote specific promoter, or a promoter that is highly expressed in sperm, eggs and zygotes (e.g., prOsActin1). Suitable promoters are disclosed in U.S. Pat. No. 10,519,456, the entire content of which is herein incorporated by reference.

In another aspect, provided herein is a method of editing plant genomic DNA. In some embodiments, the method comprises using a first soybean plant expressing a DNA modification enzyme and at least one optional guide nucleic acid as described above to pollinate a target plant comprising genomic DNA to be edited.

### 6. STACKING

The polynucleotides encoding the RG31 polypeptide and active variants and fragments thereof, or encoding the RG35 polypeptide and active variants and fragments thereof, as provided herein, can be stacked with one or more polynucleotides encoding a desirable trait such as a polynucleotide that confers, for example, insect, disease or herbicide resistance or other desirable agronomic traits of interest including, but not limited to, traits associated with high oil content; traits associated with increased protein content, increased digestibility; balanced amino acid content; improved drought resistance, modified maturity and/or flowering time, and high energy content. Such traits may refer to properties of both seed and non-seed plant tissues, or to food or feed prepared from plants or seeds having such traits.

As used herein, gene or trait “stacking” comprises combining desired genes or traits into one transgenic plant line. The additional polynucleotide can be introduced by a variety of approaches including by transgenic means, by breeding, or by genome editing. As one approach, plant breeders stack transgenic traits by making crosses between parents that each have a desired trait and then identifying offspring that have both of these desired traits (so-called “breeding stacks”). Another way to stack genes is by transferring two or more genes into the cell nucleus of a plant at the same time during transformation. In embodiments, the two or more genes may be transferred via distinct expression cassettes or via a common expression cassette. Another way to stack genes is by re-transforming a transgenic plant comprising a desired trait with another gene of interest conferring another desired trait to thereby provide a progeny transgenic plant comprising the combination of traits. Such methods can include, for example, random integration techniques or targeted integration via a gene editing system such as Crispr or meganucleases. For example, gene stacking can be used to combine two different insect resistance traits, two different herbicide resistance traits, two different agronomic performance traits, an insect resistance trait with a disease resistance trait a herbicide resistance trait (such as, for example, Bt11), or an agronomic performance trait, etc. The use of a selectable marker in addition to a gene of interest would also be considered gene stacking. In embodiments, the offspring or progeny plant having the desired combination of traits is identified through the use of genetic markers or molecular markers including but not limited to SNPs, QTLs, primers or probes directed to desired trait-associated genes or transgenes, promoters, microRNAs, siRNAs, mRNAs, ds RNAs, transcriptional profiles, and methylation patterns.

In some embodiments, a nucleic acid molecule or vector of the disclosure can include an additional coding sequence for one or more polypeptides or double stranded RNA molecules (dsRNA) of interest for agronomic traits that primarily are of benefit to a seed company, grower or grain processor. A polypeptide of interest can be any polypeptide encoded by a nucleotide sequence of interest. Non-limiting examples of polypeptides of interest that are suitable for production in plants include those resulting in agronomically important traits such as herbicide resistance (also sometimes referred to as “herbicide tolerance”), disease resistance, virus resistance, bacterial pathogen resistance, insect resistance, nematode resistance, or fungal resistance. See, e.g., U.S. Pat. Nos. 5,569,823; 5,304,730; 5,495,071; 6,329,504; and 6,337,431. The polypeptide also can be one that increases plant vigor or yield (including traits that allow a plant to grow at different temperatures, soil conditions and levels of sunlight and precipitation), or one that allows identification of a plant exhibiting a trait of interest (e.g., a selectable marker, seed coat color, relative maturity group, etc.). Various polypeptides of interest, as well as methods for introducing these polypeptides into a plant, are described, for example, in U.S. Pat. Nos. 4,761,373; 4,769,061; 4,810,648; 4,940,835; 4,975,374; 5,013,659; 5,162,602; 5,276,268; 5,304,730; 5,495,071; 5,554,798; 5,561,236; 5,569,823; 5,767,366; 5,879,903, 5,928,937; 6,084,155; 6,329,504 and 6,337,431; as well as US Patent Publication No. 2001/0016956.

In a particular embodiment, polynucleotides may be stacked (or, alternatively, multiple expression cassettes may be stacked on a single polynucleotide) so as to express more than one polypeptide that increase disease resistance within a plant. This is a particular advantage where, for example, one polypeptide is particularly suitable for providing resistance to one class of plant pathogens (e.g., a first rust isolate) while the other provides resistance to a different class of plant pathogens (or a different result isolate). For example, a first polypeptide encoding an RG31 or an RG35 protein, is stacked with a second polypeptide encoding another disease resistance protein. In alternate embodiments, a first polypeptide is provided that provides resistance via a first mode of action against a plant pathogen (e.g., against ASR) while the other provides resistance to the same plant pathogen via a second, different mode of action. Stacking polypeptides encoded by different polypeptides is also an advantage where one polypeptide expresses inherent pathogen-resistance but is somewhat labile.

Such additional disease resistance proteins include, but are not limited to, one or more of the various resistance proteins and/or polypeptides encoded by the various resistance genes as set forth in: WO2019103918 (for example, but not limited to, RG1 as disclosed therein as SEQ ID NO: 47 and active variants or fragments thereof, or any one of SEQ ID NO: 42, 43, 44, 45, 46, 49, or 50 of WO2019103918); WO2021000878 (for example Rpp6907 as set forth therein as SEQ ID NO: 1 and active variants or fragments thereof); WO2021022022 (for example, TIRA as set forth therein as SEQ ID NO: 11 or TIRB as set forth therein as SEQ ID NO: 16, or active variants or fragments thereof); WO2021260673 and/or WO2021263249 (for example, but not limited to, RG21 as set forth therein as SEQ ID NO: 1 or RG22 as set forth therein as SEQ ID NO: 2, or active variants or fragments thereof); WO2022173659 (for example, but not limited to, RG30 as set forth therein as SEQ ID NO: 5, or active variants or fragments thereof); WO2022159341 (for example but not limited to SEQ ID NOS: 1 and 148 of WO2022159341 or active variants or fragments thereof); U.S. Pat. No. 11,492,637 (for example, but not limited to RG1 as set forth therein as SEQ ID NO: 47); U.S. Pat. No. 11,530,419 (for example, but not limited to, FIT1 as set forth therein as SEQ ID NO: 2, or an active variant or fragment thereof, or any of the FIT1 paralogs or orthologs disclosed therein as SEQ ID NOS: 4, 6, 8, 10, 12, 15, 16, 18 or 20); U.S. Pat. No. 10,842,097 (for example, but not limited to, CcRPP1 as set forth therein as SEQ ID NO: 2 or active variants or fragments thereof, or any of the resistance proteins disclosed therein as SEQ ID NOS: 4, 6, or 8); and/or WO2022140257 (for example, CcRpp2-R1Aa as set forth therein as SEQ ID NO: 2, or CcRpp2-R3Aa as set forth therein as SEQ ID NO: 4, or an active variant or fragment thereof, or any of the CcRpp2-R1 or CcRpp2-R3 homologs, orthologs or paralogs disclosed therein as SEQ ID NOS: 21-36 and 48-58); and/or or the genes encoding the resistance proteins disclosed in U.S. Provisional Application Nos. 63/426,524 and 63/509,586 as RG32 (SEQ ID NO: 1) or RG34 (SEQ ID NO: 2, 17); each of which is incorporated by reference in their entirety.

In other embodiments, the nucleic acid sequence encoding the RG31 polypeptide or the RG35 polypeptide, or an active variant or fragment of any of these polypeptides, is stacked with two or more polynucleotides encoding resistance polypeptides that require co-expression to increase disease resistance within a plant. In a particular embodiment, the RG31 polypeptide or the RG35 polypeptide is stacked with each of an RG32 polypeptide (as set forth in U.S. Provisional App. No. 63/426,524 as SEQ ID NO: 1) or an active variant or fragment thereof, and an RG34 polypeptide (as set forth in U.S. Provisional App. No. 63/426,524 as SEQ ID NO: 2), or an active variant or fragment thereof, wherein co-expression of the RG32 and RG34 polypeptide is required to increase resistance in a plant to ASR via a first mode of action different from the mode of action of the RG31 or RG35 polypeptide. In another particular embodiment, the RG31 polypeptide or the RG35 polypeptide is stacked with each of a TIRA polypeptide (as set forth in WO2021022022 as SEQ ID NO: 11) or an active variant or fragment thereof, and a TIRB polypeptide (as set forth in WO2021022022 as SEQ ID NO: 16), or an active variant or fragment thereof, wherein co-expression of the TIRA and TIRB polypeptide is required to increase resistance in a plant to ASR via a first mode of action different from the mode of action of the RG31 or RG35 polypeptide. In still another particular embodiment, the RG31 polypeptide or the RG35 polypeptide is stacked with each of a CcRpp2-R1 polypeptide (as set forth in WO2022140257 as SEQ ID NO: 2) or an active variant or fragment or homolog or ortholog thereof, and a CcRpp2-R3 polypeptide (as set forth in WO2022140257 as SEQ ID NO: 4), or an active variant or fragment or homolog or ortholog thereof, wherein co-expression of the CcRpp2-R1 and CcRpp2-R3 polypeptide is required to increase resistance in a plant to ASR via a first mode of action different from the mode of action of the RG31 or RG35 polypeptide. Each of these references is incorporated by reference herein in its entirety.

In still other embodiments, the nucleic acid sequence encoding the RG31 polypeptide or the RG35 polypeptide, or an active variant or fragment of any of these polypeptides, is stacked with a fusion protein comprising two or more resistance polypeptides, or an active variant or fragment of the fusion protein, wherein co-expression of the two or more resistance polypeptides is required to increase disease resistance within a plant. In a particular embodiment, the RG31 polypeptide or the RG35 polypeptide is stacked with a fusion protein comprising the TIRA and TIRB polypeptides of WO2021022022, or an active variant or fragment thereof, such as the fusion proteins set forth in U.S. Provisional App. No. 63/383,609 as SEQ ID NO: 9, 10, 11, 12, 17, 18, or 21), or an active variant or fragment thereof, wherein the fusion protein comprising the TIRA and TIRB polypeptides is required to increase resistance in a plant to ASR via a first mode of action different from the mode of action of the RG31 or RG35 polypeptide. In still another particular embodiment, the RG31 polypeptide or the RG35 polypeptide is stacked with a fusion protein comprising the RG32 polypeptide and RG34 polypeptides of U.S. Provisional App. No. 63/426,524, or an active variant or fragment thereof, wherein the fusion protein comprising the Rg32 and RG34 polypeptides is required to increase resistance in a plant to ASR via a first mode of action different from the mode of action of the RG31 or RG35 polypeptide. In still another particular embodiment, the RG31 polypeptide or the RG35 polypeptide is stacked with a fusion protein comprising the CcRpp2-R1 polypeptide and CcRpp2-R3 polypeptides of WO2022140257, or an active variant or fragment thereof, wherein the fusion protein comprising the CcRpp2-R1 and CcRpp2-R3 polypeptides is required to increase resistance in a plant to ASR via a first mode of action different from the mode of action of the RG31 or RG35 polypeptide. Each of these references is incorporated by reference herein in its entirety.

In other embodiments, the nucleic acid sequence encoding the RG31 polypeptide or the RG35 polypeptide, or an active variant or fragment of any of these polypeptides, is stacked with a native trait that confers disease resistance. In specific embodiments, the native trait that confers disease resistance is a protein that confers increased resistance to ASR or to pathogens from the genus Phakopsora, including the species Phakopsora pachyrhizi and Phakopsora meibomiae. For example, the various intervals, locus or resistance genes as set forth in WO2009079729, U.S. Pat. No. 9,091,681, WO2010009404, WO2017222827, WO2021000878, WO2021022026, WO2021022101, WO2021154632, WO2022173659, and PCT App. No. PCT/US23/60373 (each of which is incorporated by reference in their entirety) can be bred into a glycine max plant comprising the RG31 polypeptide or RG35 polypeptide, or active variant or fragment of any thereof.

As such, in some embodiments, the RG31 protein or the RG35 protein, can be deployed as a “native construct” or as a “native stack.” As used herein, a native construct comprises the native gene (that is, the native regulatory region and the native coding region) as found in nature, unmodified by man. Such native constructs are integrated into the genome in a heterologous location (that is, a location different from the native location in the genome). In such instances, the native constructs or native stacks of RG31 (e.g., SEQ ID NO: 3) or RG35 (e.g., SEQ ID NO: 5) are stably integrated into the genome via any method, including for example, TIN or random integration. In some embodiments, a native construct employed comprises at least the native gene of RG31 or native gene of RG35 or native variants of fragments thereof with at least one additional native gene of interest. The additional native gene of interest can include for example, the native gene, or native variant thereof, of any of the R-genes disclosed herein or any other native gene of interest including native genes related to any other pathogen resistance or agronomic performance.

The native construct or the native stack when integrated via targeted insertion can be deployed to neighbor either other native traits of interest and/or other heterologous traits of interest, such as herbicide tolerance traits or insect control traits as disclosed herein. See for example, the stacks of WO2022040134, US20220056470, and WO2023164453, the contents of which are incorporated by reference herein in their entirety.

Polynucleotides conferring resistance/tolerance to a herbicide that inhibits the growing point or meristem, such as an imidazalinone or a sulfonylurea can also be suitable in some embodiments. Exemplary polynucleotides in this category code for mutant ALS and AHAS enzymes as described, e.g., in U.S. Pat. Nos. 5,767,366 and 5,928,937. U.S. Pat. Nos. 4,761,373 and 5,013,659 are directed to plants resistant to various imidazalinone or sulfonamide herbicides. U.S. Pat. No. 4,975,374 relates to plant cells and plants containing a nucleic acid encoding a mutant glutamine synthetase (GS) resistant to inhibition by herbicides that are known to inhibit GS, e.g., phosphinothricin and methionine sulfoximine. U.S. Pat. No. 5,162,602 discloses plants resistant to inhibition by cyclohexanedione and aryloxyphenoxypropanoic acid herbicides. The resistance is conferred by an altered acetyl coenzyme A carboxylase (ACCase).

Polypeptides encoded by nucleotides sequences conferring resistance to glyphosate are also suitable for the disclosure. See, e.g., U.S. Pat. Nos. 4,940,835 and 4,769,061. U.S. Pat. No. 5,554,798 discloses transgenic glyphosate resistant maize plants, which resistance is conferred by an altered 5-enolpyruvyl-3-phosphoshikimate (EPSP) synthase gene.

Polynucleotides coding for resistance to phosphono compounds such as glufosinate ammonium or phosphinothricin, and pyridinoxy or phenoxy propionic acids and cyclohexones are also suitable. See, European Patent Application No. 0 242 246. See also, U.S. Pat. Nos. 5,879,903, 5,276,268 and 5,561,236.

Other suitable polynucleotides include those coding for resistance to herbicides that inhibit photosynthesis, such as a triazine and a benzonitrile (nitrilase) See, U.S. Pat. No. 4,810,648. Additional suitable polynucleotides coding for herbicide resistance include those coding for resistance to 2,2-dichloropropionic acid, sethoxydim, haloxyfop, imidazolinone herbicides, sulfonylurea herbicides, triazolopyrimidine herbicides, s-triazine herbicides and bromoxynil. Also suitable are polynucleotides conferring resistance to a protox enzyme, or that provide enhanced resistance to plant diseases; enhanced tolerance of adverse environmental conditions (abiotic stresses) including but not limited to drought, excessive cold, excessive heat, or excessive soil salinity or extreme acidity or alkalinity; and alterations in plant architecture or development, including changes in developmental timing. See, e.g., U.S. Patent Publication No. 2001/0016956 and U.S. Pat. No. 6,084,155.

Additional herbicide tolerant traits include, PPO tolerant traits including, for example, one or more PPO trait set forth in US20190062777, U.S. Pat. Nos. 10,370,677, 11,124,803, WO2017217793, WO2020251313, U.S. Pat. Nos. 10,392,630, 10,378,023, WO2016099153, WO2019117579, WO2019117578, and U.S. Pat. No. 10,100,329, each of which is herein incorporated by reference in their entirety. HPPD tolerant traits include: WO2009144079, U.S. Pat. No. 8,642,748, EP2453012, WO2013026740, U.S. Pat. Nos. 9,078,446, 10,793,872, 10,508,089, 10,400,249, 10,597,674, WO2018119364, WO2018119361, U.S. Pat. No. 11,180,770, US20200157086, US20210147866, U.S. Pat. No. 11,279,944, US202000331866, WO2019227036, WO2019227028, WO2022115296, and WO2011068567, each of which is herein incorporated by reference in their entirety. ACCase tolerant traits include: US20120284812, US20120284853, US20160108423, US20160244780, US20160264990, US20170275645, US20210153448, U.S. Pat. No. 10,696,975B2, U.S. Pat. No. 10,370,678, CN109082416, U.S. Pat. No. 10,694,694, US20170265469, US20170231225, each of which is herein incorporated by reference. Dicamba tolerant traits include, for example, RE45048 or U.S. Pat. No. 7,884,262. Various traits the confer tolerance to AOPP herbicides, phenoxy acid herbicides and/or pyridinyloxy acid herbicides include, for example, U.S. Pat. Nos. 10,174,337, 8,278,505, WO05107437, WO11022469, U.S. Pat. No. 10,023,874, and US2019241903 (and other traits therein), each of which is herein incorporated by reference. Additional herbicides tolerant traits of interest for stacking include glucosyl transferase polypeptides as set forth in 2018213022 or Solanesyl Diphosphate Synthase polypeptides as set forth in WO2020236790, each of which is herein incorporated by reference in their entirety.

Additional suitable polynucleotides include those coding for insecticidal polypeptides. These polypeptides may be produced in amounts sufficient to control, for example, insect pests (i.e., insect controlling amounts). It is recognized that the amount of production of an insecticidal polypeptide in a plant necessary to control insects or other pests may vary depending upon the cultivar, type of pest, environmental factors and the like. Polynucleotides useful for additional insect or pest resistance include, for example, those that encode toxins identified in Bacillus organisms. Polynucleotides comprising nucleotide sequences encoding Bacillus thuringiensis (Bt) Cry proteins from several subspecies have been cloned and recombinant clones have been found to be toxic to lepidopteran, dipteran and/or coleopteran insect larvae. Examples of such Bt insecticidal proteins include the Cry proteins such as Cry1Aa, Cry1Ab, Cry1Ac, Cry1B, Cry1C, Cry1D, Cry1Ea, Cry1Fa, Cry3A, Cry9A, Cry9B, Cry9C, and the like, as well as vegetative insecticidal proteins such as Vip1, Vip2, Vip3, and the like. A full list of Bt-derived proteins can be found on the worldwide web at Bacillus thuringiensis Toxin Nomenclature Database maintained by the University of Sussex (see also, Crickmore et al. (1998) Microbiol. Mol. Biol. Rev. 62:807-813).

In embodiments, an additional polypeptide is an insecticidal polypeptide derived from a non-Bt source, including without limitation, an alpha-amylase, a peroxidase, a cholesterol oxidase, a patatin, a protease, a protease inhibitor, a urease, an alpha-amylase inhibitor, a pore-forming protein, a chitinase, a lectin, an engineered antibody or antibody fragment, a Bacillus cereus insecticidal protein, a Xenorhabdus spp. (such as X. nematophila or X. bovienii) insecticidal protein, a Photorhabdus spp. (such as P. luminescens or P. asymobiotica) insecticidal protein, a Brevibacillus spp. (such as B. laterosporous) insecticidal protein, a Lysinibacillus spp. (such as L. sphearicus) insecticidal protein, a Chromobacterium spp. (such as C. subtsugae or C. piscinae) insecticidal protein, a Yersinia spp. (such as Y. entomophaga) insecticidal protein, a Paenibacillus spp. (such as P. propylaea) insecticidal protein, a Clostridium spp. (such as C. bifermentans) insecticidal protein, a Pseudomonas spp. (such as P. fluorescens) and a lignin.

In certain embodiments, the additional polypeptide is a resistance protein conferring enhanced pathogen resistance, such as enhanced resistance to any one of the following pathogens: soy cyst nematode, bacterial pustule, root knot nematode, frog eye leaf spot, phytopthora, brown stem rot, nematode, Asian Soybean Rust, smut, Golovinomyces cichoracearum, Erysiphe cichoracearum, Blumeria graminis, Podosphaera xanthii, Sphaerotheca fuliginea, Pythium ultimum, Uncinula necator, Mycosphaerella pinodes, Magnaporthe grisea, Bipolaris oryzae, Magnaporthe grisea, Rhizoctonia solani, Phytophthora sojae, Schizaphis graminum, Bemisia tabaci, Rhopalosiphum maidis, Deroceras reticulatum, Diatraea saccharalis, Schizaphis graminum, Myzus persicae, Sclerotinia sclerotiorum, Macrophomina phaseolina, Fusarium virguliforme, sucking and piercing pests such as aphid, stinkbug, and whitefly, or bacterial pathogens including Pseudomonas syringae. Exemplary polynucleotides encoding proteins that confer increased pathogen resistance that may be stacked with polynucleotides of the invention include polynucleotides encoding proteins that confer increased ASR resistance as described in US Patent publication Nos. US20200354739 and PCT Publications Nos. WO2019103918, WO2021154632A1, WO2021022022, WO2021022026, WO2021022101, WO2021260673, and WO2021263249, each of which is incorporated by reference in its entirety.

Disease resistance proteins that increase resistance to various plant disease including rust, include, but are not limited to, one or more of the various resistance genes set forth in: WO2019103918; WO202100878; WO2021022022; WO2021260673; WO2022173659; WO2022159341; WO2021154632A1, WO2021022026, WO2021022101, US20220135997; U.S. Pat. No. 10,842,097; or WO2022140257; each of which is incorporated by reference in their entirety. In other embodiments, the nucleic acid sequence encoding the RG32 and/or RG34 polypeptide or active variant or fragments thereof is stacked with a native trait that confers disease resistance. For example, the various intervals, loci, or resistance genes as set forth in WO2009079729, U.S. Pat. No. 9,091,681, WO2010009404, WO2017222827, WO2021000878, WO2021022026, WO2021022101, WO2021154632, WO2022173659, (each of which is incorporated by reference in their entirety) can used to introduce the trait of interest into Glycine max. Disease resistance proteins and/or native traits that increase resistance to various plant diseases including Northern Corn Leaf Blight (NCLB) include, for example, U.S. Pat. No. 8,921,646, US2021000059, U.S. Pat. No. 10,858,668, US20200199610, WO2022/013268, WO2022/013268, U.S. Pat. Nos. 9,040,772, 10,897,862, EP3839073, each of which is herein incorporated by reference.

Polypeptides that are suitable for production in plants further include those that improve or otherwise facilitate the conversion of harvested plants or plant parts into a commercially useful product, including, for example, increased or altered carbohydrate content or distribution, improved fermentation properties, increased oil content, increased protein content, improved digestibility, and increased nutraceutical content, e.g., increased phytosterol content, increased tocopherol content, increased stanol content or increased vitamin content. Polypeptides of interest also include, for example, those resulting in or contributing to a reduced content of an unwanted component in a harvested crop, e.g., phytic acid, or sugar degrading enzymes. By “resulting in” or “contributing to” is intended that the polypeptide of interest can directly or indirectly contribute to the existence of a trait of interest (e.g., increasing cellulose degradation by the use of a heterologous cellulase enzyme).

In some embodiments, the polypeptide contributes to improved digestibility for food or feed. Xylanases are hemicellulolytic enzymes that improve the breakdown of plant cell walls, which leads to better utilization of the plant nutrients by an animal. This leads to improved growth rate and feed conversion. Also, the viscosity of the feeds containing xylan can be reduced. Heterologous production of xylanases in plant cells also can facilitate lignocellulosic conversion to fermentable sugars in industrial processing.

Numerous xylanases from fungal and bacterial microorganisms have been identified and characterized (see, e.g., U.S. Pat. No. 5,437,992; Coughlin et al. (1993) “Proceedings of the Second TRICEL Symposium on Trichoderma reesei Cellulases and Other Hydrolases” Espoo; Souminen and Reinikainen, eds. (1993) Foundation for Biotechnical and Industrial Fermentation Research 8:125-135; U.S. Patent Publication No. 2005/0208178; and PCT Publication No. WO 03/16654). In particular, three specific xylanases (XYL-I, XYL-II, and XYL-III) have been identified in T. reesei (Tenkanen et al. (1992) Enzyme Microb. Technol. 14:566; Torronen et al. (1992) Bio/Technology 10:1461; and Xu et al. (1998) Appl. Microbiol. Biotechnol. 49:718).

In other embodiments, a polypeptide useful for the disclosure can be a polysaccharide degrading enzyme. Plants of this disclosure producing such an enzyme may be useful for generating, for example, fermentation feedstocks for bioprocessing. In some embodiments, enzymes useful for a fermentation process include alpha amylases, proteases, pullulanases, isoamylases, cellulases, hemicellulases, xylanases, cyclodextrin glycotransferases, lipases, phytases, laccases, oxidases, esterases, cutinases, granular starch hydrolyzing enzyme and other glucoamylases.

Polysaccharide-degrading enzymes include: starch degrading enzymes such as α-amylases (EC 3.2.1.1), glucuronidases (E.C. 3.2.1.131); exo-1,4-α-D glucanases such as amyloglucosidases and glucoamylase (EC 3.2.1.3), β-amylases (EC 3.2.1.2), α-glucosidases (EC 3.2.1.20), and other exo-amylases; starch debranching enzymes, such as a) isoamylase (EC 3.2.1.68), pullulanase (EC 3.2.1.41), and the like; b) cellulases such as exo-1,4-3-cellobiohydrolase (EC 3.2.1.91), exo-1,3-β-D-glucanase (EC 3.2.1.39), β-glucosidase (EC 3.2.1.21); c) L-arabinases, such as endo-1,5-α-L-arabinase (EC 3.2.1.99), α-arabinosidases (EC 3.2.1.55) and the like; d) galactanases such as endo-1,4-β-D-galactanase (EC 3.2.1.89), endo-1,3-β-D-galactanase (EC 3.2.1.90), α-galactosidase (EC 3.2.1.22), β-galactosidase (EC 3.2.1.23) and the like; e) mannanases, such as endo-1,4-β-D-mannanase (EC 3.2.1.78), β-mannosidase (EC 3.2.1.25), α-mannosidase (EC 3.2.1.24) and the like; f) xylanases, such as endo-1,4-β-xylanase (EC 3.2.1.8), β-D-xylosidase (EC 3.2.1.37), 1,3-β-D-xylanase, and the like; and g) other enzymes such as α-L-fucosidase (EC 3.2.1.51), α-L-rhamnosidase (EC 3.2.1.40), levanase (EC 3.2.1.65), inulanase (EC 3.2.1.7), and the like. In one embodiment, the α-amylase is the synthetic α-amylase, Amy797E, described is U.S. Pat. No. 8,093,453, herein incorporated by reference in its entirety.

Further enzymes which may be used with the disclosure include proteases, such as fungal and bacterial proteases. Fungal proteases include, but are not limited to, those obtained from Aspergillus, Trichoderma, Mucor and Rhizopus, such as A. niger, A. awamori, A. oryzae and M. miehei. In some embodiments, the polypeptides of this disclosure can be cellobiohydrolase (CBH) enzymes (EC 3.2.1.91). In one embodiment, the cellobiohydrolase enzyme can be CBH1 or CBH2.

Other enzymes useful with the disclosure include, but are not limited to, hemicellulases, such as mannases and arabinofuranosidases (EC 3.2.1.55); ligninases; lipases (e.g., E.C. 3.1.1.3), glucose oxidases, pectinases, xylanases, transglucosidases, alpha 1,6 glucosidases (e.g., E.C. 3.2.1.20); esterases such as ferulic acid esterase (EC 3.1.1.73) and acetyl xylan esterases (EC 3.1.1.72); and cutinases (e.g. E.C. 3.1.1.74).

In other embodiments, the polynucleotides provided herein are stacked with polynucleotides that increase protein content, and/or alter seed composition and/or fatty acid content. Such sequences include, but are not limited to, sequences disclosed in PCT Appl. No. PCT/CN2022/075977 and PCT Appl. No. PCT/CN2022/075982 both filed on Feb. 11, 2022, WO2021/044027 (which discloses various amino acid permease (AAP) polypeptides from soybean and a variety of other plants including, for example KJN37208 (a soybean AAP8 polypeptide), XP XP_003526513 (a soybean AAP8 polypeptide), NP_001242816 LOC100777963 (a soybean AAP polypeptide) and AA XP 028228300 (a soybean AAP6-like polypeptide)); US2020/0131524 which discloses various UPL3 polypeptides from a variety of plants including, for example, SEQ ID NO: 28 and a variety of UPL3 polypeptide homologs listed in Table 1; and US2021/0403933 which discloses various HECT E3 ligases, including HEL1 and HEL2, and other polypeptides and gene edits of interest, each of which is incorporated by reference in its entirety.

Double stranded RNA molecules useful with the disclosure include but are not limited to those that suppress target genes. As used herein the words “gene suppression”, when taken together, are intended to refer to any of the well-known methods for reducing the levels of protein produced as a result of gene transcription to mRNA and subsequent translation of the mRNA. Gene suppression is also intended to mean the reduction of protein expression from a gene or a coding sequence including posttranscriptional gene suppression and transcriptional suppression. Posttranscriptional gene suppression is mediated by the homology between of all or a part of a mRNA transcribed from a gene or coding sequence targeted for suppression and the corresponding double stranded RNA used for suppression and refers to the substantial and measurable reduction of the amount of available mRNA available in the cell for binding by ribosomes. The transcribed RNA can be in the sense orientation to effect what is called co-suppression, in the anti-sense orientation to effect what is called anti-sense suppression, or in both orientations producing a dsRNA to effect what is called RNA interference (RNAi). Transcriptional suppression is mediated by the presence in the cell of a dsRNA, a gene suppression agent, exhibiting substantial sequence identity to a promoter DNA sequence or the complement thereof to effect what is referred to as promoter trans suppression. Gene suppression may be effective against a native plant gene associated with a trait, e.g., to provide plants with reduced levels of a protein encoded by the native gene or with enhanced or reduced levels of an affected metabolite. Gene suppression can also be effective against target genes in plant pests that may ingest or contact plant material containing gene suppression agents, specifically designed to inhibit or suppress the expression of one or more homologous or complementary sequences in the cells of the pest. Such genes targeted for suppression can encode an essential protein, the predicted function of which is selected from the group consisting of muscle formation, juvenile hormone formation, juvenile hormone regulation, ion regulation and transport, digestive enzyme synthesis, maintenance of cell membrane potential, amino acid biosynthesis, amino acid degradation, sperm formation, pheromone synthesis, pheromone sensing, antennae formation, wing formation, leg formation, development and differentiation, egg formation, larval maturation, digestive enzyme formation, hemolymph synthesis, hemolymph maintenance, neurotransmission, cell division, energy metabolism, respiration, and apoptosis.

As used herein, “selectable marker” means a nucleotide sequence that when expressed imparts a distinct phenotype to the plant, plant part and/or plant cell expressing the marker and thus allows such transformed plants, plant parts and/or plant cells to be distinguished from those that do not have the marker. Such a nucleotide sequence may encode either a selectable or screenable marker, depending on whether the marker confers a trait that can be selected for by chemical means, such as by using a selective agent (e.g., an antibiotic, herbicide, or the like), or on whether the marker is simply a trait that one can identify through observation or testing, such as by screening. In some examples, the trait can be identified through visual observation, such as by comparing the plant height, plant vigor, or plant flowering time of a plant with the desired combination of traits with a control plant (e.g., a plant not comprising the combination of traits or a parent plant from which the selected plant was derived).

### 7. ASSAY, KITS AND PRIMERS

Also provided are the kits, probes, primers and antibodies that can be used to introduce a polynucleotide sequence as described in this disclosure into a recipient plant or to detect a polynucleotide or polypeptide sequence as described in this disclosure in a plant. The polypeptide and the polynucleotide or variant and fragments thereof provided herein can be packaged as components of a kit with instructions for completing the assay described herein. A DNA detection kit is provided for use in detecting the nucleotides sequences encoding the RG31 or RG35 polypeptide or variants and fragments thereof or for detecting a transgenic event or a gene edit comprising a RG31 or RG35 polypeptide in a plant are provided.

In some embodiments, the kit may comprise one or more probes having a sequence corresponding to or complementary to a sequence having 80%, 85%, 86%, 87%, 88%, 89%, 90%, 91%, 92%, 93%, 94%, 95%, 96%, 97%, 98%, 99% or 100% sequence identity with a specific region of the nucleotides sequence set forth in any of SEQ ID NOS: 3-6 or 21-22, which allows for the detection of the sequence. In some embodiments, the kit may comprise any reagent and material required to perform the assay or detection method. In specific embodiments, the probes can be used to specifically hybridize to target polynucleotide and thereby detect the nucleotide sequence set forth in any of SEQ ID NOS: 3-6 or 21-22 or variants or fragments thereof.

Further provided are antibodies to the polypeptides of the present invention, or to variants or fragments thereof, are also encompassed. Methods for producing antibodies are well known in the art (see, for example, Harlow and Lane (1988) Antibodies: A Laboratory Manual, Cold Spring Harbor Laboratory, Cold Spring Harbor, N.Y.; and U.S. Pat. No. 4,196,265). These antibodies can be used in kits for the detection and isolation of toxin polypeptides. Thus, this disclosure provides kits comprising antibodies that specifically bind to the polypeptides described herein, including, for example, polypeptides having the sequence of SEQ ID NOs: 1 or 2 or variants or fragments thereof.

### 8. METHODS OF USE OF PLANTS AND SEEDS

Methods of preventing pathogen damage or disease damage or ASR damage to a plant are provided. Methods of controlling disease, or ASR, in an area of cultivation are also provided. Such methods comprise planting in an area of cultivation a plant or seed having stably integrated into its genome a nucleotide sequence encoding the RG31 or RG35 polypeptide or an active variant or fragment thereof and growing said plant or said seed, wherein expression of said RG31 or RG35 polypeptide or an active variant or fragment thereof increases the resistance of the plant to a plant disease and/or plant pathogen.

The various compositions and methods disclosed herein can provide an increased or enhanced resistance to various plant pathogens and/or plant diseases. Exemplary plant pathogens and/or plant diseases include, but are not limited to, soy cyst nematode, bacterial pustule, root knot nematode, frog eye leaf spot, phytopthora, brown stem rot, nematode, Asian Soybean Rust, smut, Golovinomyces cichoracearum, Erysiphe cichoracearum, Blumeria graminis, Podosphaera xanthii, Sphaerotheca fuliginea, Pythium ultimum, Uncinula necator, Mycosphaerella pinodes, Magnaporthe grisea, Bipolaris oryzae, Magnaporthe grisea, Rhizoctonia solani, Phytophthora sojae, Schizaphis graminum, Bemisia tabaci, Rhopalosiphum maidis, Deroceras reticulatum, Diatraea saccharalis, Schizaphis graminum, Myzus persicae, Sclerotinia sclerotiorum, Macrophomina phaseolina, or Fusarium virguliforme.

In specific embodiments the plant disease is Asian Soybean rust. In other embodiments, the plant pathogen is from the genus Phakopsora, including the species Phakopsora pachyrhizi and Phakopsora meibomiae, known to cause ASR in plants.

In other embodiments, the plants expressing the RG31 or RG35 polypeptide, or an active variant or fragment thereof, are contacted with one or more fungicides to further prevent ASR associated damage or any other plant disease of interest to the plant (i.e., the legume or the soybean). Such fungicides can be applied to any part of the plant, including for example, the seed or the leaf or to the area of cultivation. Such fungicidal compounds can be formulated or tank mixed with other fungicides or applied sequentially with other fungicides. Such fungicides may include a fungicide from one or more of the following chemical classes: Benzimidazoles, Dicarboximides, Azoles, Pyrimidines, Phenylamides, Morpholines, Carboxamides, Anilinopyrimidine, Strobilurins, Carboxylic acid amides, Inorganics, Dithiocarbamates, or Phthalimides. See, Morton, V. and Staub, T. 2008 A Short History of Fungicides. Online, APSnet Features. doi: 10.1094/APSnetFeature-2008-0308, herein incorporated by reference.

Methods are provided for screening or assaying a plant (a legume plant or a soybean plant) for resistance, to determine immunity or susceptibility of the plant to a plant disease. Such methods include, but are not limited to, screening a plant (a legume plant or a soybean plant), assaying a plant for immunity, resistance or susceptibility to a plant disease by contacting a plant cell, tissue or organ to a pathogen (e.g., Phakopsora pachyrhizi, Phakopsora meibomiae) and measuring the resistance, immunity or susceptibility of the plant or plant part to a plant disease (e.g., ASR) caused by the pathogen. Any observable phenotype of the plant pathogen and/or disease can be measured. These include, but are not limited to, number, size, and/or density of disease related lesions, color of lesions (e.g., colors ranging anywhere from a tan coloration to a reddish-brown coloration), number and density of pustule formation, sporulation, defoliation, yield loss, or any combination thereof. Further embodiments include change in any of the aforementioned phenotypes. Still further embodiments include measured delays or expediting of proliferation of a pathogen (e.g., fungus).

### 9. METHODS AND COMPOSITIONS TO GENERATE A RESISTANCE AND/OR VIRULENCE PROFILE FOR A PLANT PATHOGEN IN AN AREA OF CULTIVATION

Resistance management of fungi in crops currently involves applying a combination of fungicides with a different mode of action at specific time points throughout the crop growing season based on product availability for the control of a specific pathogen and/or employing ASR genetic resistance traits within the crop. Approximate knowledge of the presence of resistance of a pathogen in a region or country and general recommendations are today provided, however, this only offers general information on the presence of resistance and recommendations on how to deal with it. Examples of the present disclosure provide methods of how resistance management can be improved and tailored to a specific situation. This may be achieved by determining a resistance and/or virulence profile for a plant pathogen through qualitative and quantitative pesticide (e.g., fungicide) sensitivity of the plant pathogen to a specific pesticide and/or qualitative and quantitative virulence of a plant pathogen to a crop in a specific location. This can be determined rapidly, for example close to real-time or within 48 hours as is explained in more detail below. By knowing the genetic variances of a plant pathogen population and their respective quantity or frequency at a specific location and at a given time, a more tailored treatment program or disease control measure can be provided within season that also considers resistance development of the plant pathogen. It may also be combined with a recommendation of an optimal or particular resistant crop. This will allow for a more sustainable use of the tools available for pathogen management and avoid non-effective solutions for that particular location. In addition, it can help to improve agronomic practice in order to deliver the best control of the pathogen and a sounder resistance management strategy.

Methods and compositions are provided to generate a resistance and/or virulence profile for a plant pathogen in an area of cultivation. The resistance and/or virulence profile of the area of cultivation can be employed to determine a recommended pesticide application protocol for controlling the development, reproduction, and/or viability of the ASR plant pathogen population either within the area of cultivation and/or within neighboring areas of cultivation.

a. Resistance and/or Virulence Profile

Methods and compositions are provided to generate a resistance and/or virulence profile for a plant pathogen, such as an ASR plant pathogen, in an area of cultivation.

As used herein, “sensitivity” refers to the susceptibility of a ASR plant pathogen to an ASR pesticide such as an ASR fungicide or to an ASR genetic resistance trait. Variations of sensitivity to ASR pesticides and/or ASR genetic resistance traits can result from a range of different mechanisms.

“Resistance” refers to the ability of an ASR plant pathogen to survive the exposure to ASR pesticides designed to control it, including fungicides or through exposure to an ASR genetic resistance trait. Several mechanisms can shape the resistance or adaptation level. In contrast to sensitivity, resistance of an ASR plant pathogen means that the ASR plant pathogen or the ASR plant pathogen population becomes less sensitive to such an extent that a pesticide such as a fungicide or an ASR genetic resistance trait is no longer effective in controlling the development, reproduction and viability of the ASR plant pathogen and/or the ASR plant pathogen population.

Resistance development refers to an ASR plant pathogen and/or ASR plant pathogen population which develops a lower sensitivity to an ASR pesticide and/or an ASR genetic resistance trait. Lower sensitivity can result in complete resistance or gradual resistance to a fungicide or an ASR genetic resistance trait. A single mechanism or a combination of mechanisms can shape resistance development. The resistance development depends on how fast these mechanisms are selected in a population of time and space.

A resistance management program used herein should be understood as measures that can be adopted to minimize development of resistance of an ASR pathogen and/or ASR pathogen population to a plant protection product, such as a pesticide or an ASR genetic resistance trait.

As used herein, resistance profile refers to the ability of an ASR plant pathogen or a population of ASR plant pathogens to overcome and survive the exposure to an ASR genetic resistance trait, a pesticide, a fungicide, a pesticide class or fungicide class as defined by FRAC, or other chemical or biological agent used for controlling development, viability and reproduction of the ASR plant pathogen.

As used herein, virulence profile refers to the ability of an ASR plant pathogen or a population of an ASR plant pathogen to infect and cause damage to a host, wherein the host may have a type of built-in resistance to the pathogen (i.e, ASR genetic resistance trait).

b. Methods of Generating a Resistance and/or Virulence Profile for an ASR Plant Pathogen

A variety of methods can be used to obtain a resistance and/or virulence profile for a population of ASR plant pathogens within an area of cultivation. In one non-limiting embodiment, the frequency of the presence of one or more ASR plant pathogen effectors from an ASR plant pathogen sample is obtained from a location within the area of cultivation. In other embodiments, the frequency of the presence of one or more ASR plant pathogen effectors is obtained from a plurality of locations within the area of cultivation and a map of the frequencies throughout the area of cultivation is generated. In other cases, an average frequency of the presence of one or more of the ASR plant pathogen effectors is determined for the area of cultivation as a whole.

i. Obtaining Samples and Information Indicating ASR Plant Pathogen Susceptibility of the Plants in the Area of Cultivation

The frequency of an ASR effector of interest with the ASR plant pathogen population can be obtained from an ASR plant pathogen sample taken from a single location within the area of cultivation, or the information can be obtained from a plurality of locations within the area of cultivation, such that least 1, 2, 3, 4, 5, 6, 7, 8, 9, 10, 20, 30, 50 or more samples within the area of cultivation. The geographical location, the size of the area of cultivation, and the disease pressure in the area can impact the exact number of samples take and the exact location from which the sample are obtained within the area of cultivation. In addition, the selection of locations within the area of cultivation where ASR plant pathogens are collected can be influenced by previously acquired disease pressure data. In specific embodiments, the resistance profile and/or the virulence profile is generated from an ASR plant pathogen sample obtained from a single location within an area of cultivation. In other embodiments, the resistance profile and/or the virulence profile is generated from information obtained from multiple ASR plant pathogen samples taken from more than one location within the area of cultivation.

The area of cultivation can be any area location where a plant is grown. The area of cultivation includes agricultural fields, cultivated fields, and protected areas such as greenhouses.

A “sample” can be obtained by collecting at least some plant material or at least one environmental sample (aerial or soil sample) from the area of cultivation. Therefore, a sample may be a field sample such as a leaf, several leaves and/or other part of the crop that is collected from a crop or a field or alternative host plants (e.g., crop wilt type forms or volunteer crops). Additionally, or alternatively, a sample may be an environmental sample comprising soil and/or aerial material collected from where a single crop grows or collected from several points across an area of cultivation. In one example, a “sample” is to be understood as a sample of a plurality of plants, a spore trap or soil. The sample comprises at least one ASR plant pathogen and should be understood as a representation of a local population of the at least one plant pathogen.

In some embodiments, the area of cultivation can comprise a plant and/or the sample collected is from a plant or any part of a plant. The plants within the area of cultivation comprise any plant type including a legume plant. Examples are listed elsewhere herein. The frequency of the presence of an ASR plant pathogen effector within an ASR plant pathogen sample can be obtained via a variety of methods, including through an analysis of proteomic information (protein expression/amino acid sequence), transcriptional information, or genomic DNA information. Such information can be obtained through various biological materials and methods. As used herein, “biological material” means any material from an ASR plant pathogen that allows for the determination of the frequency of the one or more ASR plant pathogen effector of interest. For example, the biological material can comprise a polynucleotide. The polynucleotide can comprise DNA (genomic DNA, regulatory regions or variants or fragments thereof that influence the expression of the ASR effector of interest, introns or coding regions or variants or fragments thereof of the ASR effector(s) of interest, or a SNP or a haplotype associated with the ASR effector of interest) or RNA encoding the ASR effector of interest. Polynucleotides within the biological material can be amplified and detected using a variety of techniques employed in the art. In other embodiments, the biological material comprises a polypeptide encoding the ASR effector of interest or an active variant or fragment thereof.

For example, the amino acid sequence of given ASR effector or an active variant or fragment thereof can be detected through an antibody assay which specifically detects the ASR effector polypeptide of interest. The use of proteomics or expression of genes to produce a particular protein can employ detection of a single protein of interest or multiple differentially expressed proteins to determine the frequency of a given protein within the ASR pathogen population.

Genotypic information can be obtained by isolating, amplifying, and/or sequencing the genomic DNA of the ASR plant pathogen population with the sample, followed by the detection of the genomic DNA (including, for example, a haplotype or SNP) corresponding to the ASR effector sequence of interest. Alternatively, the transcriptome can be assayed to determine the frequency of the ASR effector within the ASR plant pathogen sample.

Test kits are provided that provide for detection of the specific set of ASR effector proteins of interest via a variety of different diagnostic methods, including DNA detection, RNA detection and protein detection. Such kits may comprise a set of nucleic acid probes and/or primers, each comprising a nucleotides sequence that specifically hybridizes to a nucleotide sequence encoding the ASR effector protein of interest. In other embodiments, the kit comprises an antibody that specifically recognizes an ASR effector polypeptide of interest. In some embodiments, these kits are employed in a field setting. See, for example, Shin et al. (2015) Vector Ecology 41:1, 63-71 detecting particular markers for resistance and Tian et al. (2018) Scientific Rep 8, no. 12587, identifying pest resistance related to increase expression of detoxifying genes.

Examples of nucleotide sequences and antibodies that can detect the various ASR effector proteins or DNA or RNA sequences encoding the ASR effector proteins can be found, for example in Krasileva et al. (2010) Plant Cell 22:2444-2458, Activation of an Arabidopsis resistance protein is specified by the in planta association of its leucine-rich repeat domain with the cognate oomycete effector, and Gupta et al. (2023) Nature Communications 14, Article 1835, Major proliferation of transposable elements shaped the genome of the soybean rust pathogen Phakopsora pachyrhizi.

ii. ASR Genetic Resistance Traits; ASR Plant Pathogen Effectors; and Methods of Detection

### 1. ASR Plant Pathogen Effectors

An ASR plant pathogen effector comprises a polypeptide produced by the ASR pathogen that can suppress the plant host defence mechanisms and facilitate infection of the plant by the ASR pathogen. The frequency of any ASR plant pathogen effector can be detected in the methods provided herein. In some embodiments, the frequency within the ASR plant pathogen population of one or more of the following ASR effector proteins is determined: SEQ ID NO: 16-17 of effector protein SPE35, or active variants or fragments thereof; and SEQ ID NO: 23-24 of effector protein SPE38, or active variants or fragments thereof. The SEQ ID NOS of each of the nucleotide (NT) sequence and the protein (AA) sequence of each of these ASR effector proteins is set forth in Table B.

TABLE B
Effector sequences

| Effector | SEQ ID |
|---|---|
| SPE-35 NT | 17 |
| SPE-35 AA | 16 |
| SPE-38 NT | 24 |
| SPE-38 AA | 23 |

### 2. ASR Genetic Resistance Traits

ASR genetic resistance traits comprise any trait (native trait, gene edit, or transgenic trait) stably integrated into the genome of a plant, in particular a legume plant or a soybean plant, whereby expression of the trait increases plant disease resistance against the ASR plant pathogen. ASR genetic resistance traits include R genes or disease resistance polypeptides which encode for proteins that recognize (either directly or indirectly) ASR effector proteins. A given protein encoded by an R-gene or a disease resistance polypeptide will recognize (either directly or indirectly) a specific ASR effector protein or a specific combination of ASR effector proteins.

In specific embodiments of the methods provided herein, the frequency of the ASR pathogen effector determined will vary depending on the plant planted in the area of cultivation. For example, the frequency of the presence of a given ASR effector within the ASR pathogen population can be determined based the ASR genetic resistance trait within the legume, such a soybean plant, within the area of cultivation. Table C provides a summary of the ASR genetic resistance trait and the trait's corresponding ASR effector(s). As used herein, a “corresponding” ASR effector protein or active variant or fragment thereof comprises an ASR effector protein which interacts (directly or indirectly) with the ASR genetic resistance trait, whereby the interaction (direct or indirect) increases the tolerance of the plant (soybean plant or legume plant) to the ASR pathogen.

TABLE C
Summary ASR genetic resistance trait
and corresponding ASR effector

| ASR Resistance Gene | Corresponding Effector Protein |
|---|---|
| RG31 | SPE-35 (SEQ ID NO: 16) |
| | SPE-38 (SEQ ID NO: 23) |
| RG35 | SPE-35 (SEQ ID NO: 16) |
| | SPE-38 (SEQ ID NO: 23) |

A given legume or soybean plant or seed within the area of cultivation can comprise one or more of the ASR resistance proteins or active variants or fragments thereof set forth in Table C. As such, the frequency of one or more of the corresponding ASR effector proteins or active variants or fragments thereof to a given ASR genetic resistance trait will be determined in order to generate a resistance and/or virulence profile of the area of cultivation.

In one embodiment, where an area of cultivation comprising a plant comprising a polynucleotide encoding a polypeptide comprising the amino acid sequence set forth in SEQ ID NO: 1 (RG31) and SEQ ID NO: 2 or 20 (RG35), or an active variant or active fragment of either, wherein expression of said polypeptide or active variant or fragment thereof increases tolerance of the plant to an ASR plant pathogen; the frequency of the presence of one or more of the corresponding ASR effector SPE-35 (SEQ ID NO: 16-17) and/or SPE-38 (SEQ ID NO: 23-24) or active variant or fragment thereof can be determined within the sample and/or within the area of cultivation. As discussed elsewhere herein, the frequency of the effector can be determined at the polynucleotide or polypeptide level.

In specific embodiments, the polynucleotide encoding the ASR genetic resistance trait is heterologous to the plant.

### 3. Methods of Detection

Determining the frequency of an ASR effector polypeptide or active variant or fragment thereof within a sample from an area of cultivation can be performed via detection of polypeptides or polypeptides as discussed elsewhere herein, including through the use of primers, probes, PCR, etc.

In one embodiment, a PCR reaction is employed to determine the frequency of the ASR effector polypeptide or active variant for fragment thereof within the ASR plant pathogen population in the area of cultivation. In other embodiments, when detecting a polynucleotide of interest (ie. a polynucleotide of an ASR effective protein), stringent hybridization conditions can be used. employed to determine the frequency of the ASR effector polypeptide or active variant for fragment thereof within the ASR plant pathogen population in the area of cultivation. Such methods are described elsewhere herein. Such methods are described elsewhere herein.

In other embodiments, the biological material is subjected to DNA sequencing for determining if a given ASR effector is present. As used herein, a genetic variance, gene variance or a variance of a plant pathogen is to be understood as a mutation in the DNA sequence different from the wild type sequence. For example, in a genetic variance at least one nucleotide in a DNA sequence has been permanently changed, deleted or inserted.

Genotype refers to the unique combination of multiple genetic variances, gene variances or variances of a plant pathogen. If multiple independent genetic variations can be brought together into different unique combinations, then each of these unique combinations might express a different sensitivity or virulence profile.

In embodiments where the biological material is subjected to DNA sequencing for determining the nucleic acid sequence in the sample, this operation may comprise using a sequencer capable of sequencing at least 200, 300, 400 or 500 base pairs in a single read. For example, Oxford Nanopore sequencing Technologies such as MinION, GridION or PromethION or PacBio Sequel Systems implementing single-molecule real-time sequencing as provided by PACBIO may offer the required capability. These technologies may be referred to as third generation sequencer and they provide a high throughput combined with larger sequenced genetic regions from a few hundred base pairs up to 10,000 base pairs. The technology provided by Oxford Nanopore Technologies comprises flow cells which contain an array of tiny holes referred to as nanopores that are embedded in an electro-resistant membrane. Each nanopore corresponds to its own electrode connected to a channel and sensor chip, which measures the electric current that flows through the nanopore. When a molecule passes through a nanopore, the current is disrupted to produce a characteristic ‘squiggle’ or a current intensity value. The squiggle is then decoded using base calling algorithms to determine the DNA. Base calling is a computational process of translating the squiggle into DNA sequences. Specific bioinformatics pipelines may be combined to enable quantification of one or more ASR effector polypeptides in a given ASR pathogen populations.

Before subjecting a sample to DNA sequence, DNA may be extracted from the received sample. This DNA sample may include a diversity of individuals from a plant pathogenic species representative of genetic variation of the disease. In one example, specific panels of multiplex PCR are designed to co-amplify multiple targets in a single PCR reaction. This optimized step requires the identification of non-self-cross-hybridizing primers in conserved regions to cope with natural variability between individuals composing a population. Through this optimization genetic loci comprising one or more of the ASR effector polypeptides of interest are amplified from the largest fraction of strains in a natural population. Primers may also be checked to ensure that other plant pathogens or other ASR effector proteins that could occur in the same area are not unspecific amplified. Thereafter, DNA barcoding may be performed to identify a specific sample if multiple samples are bulked before sequencing the plant pathogen. Thereafter, sequencing preparation may be performed followed by the operation of DNA sequencing using a third-generation sequencer as described above.

It should be understood that the present disclosure is not limited to the specific third generation sequencers as described herein but that any sequencing technology can be applied that generates data within a timeframe giving a current insight into a disease of a plant.

Referring now again to the operation determining the presence of genetic polymorphisms based on the DNA sequencing. This operation may comprise comparing the DNA sequence of the sample with a reference DNA sequence of a plant pathogen and identifying genetic polymorphisms that are unique to a given ASR effector of interest.

Polypeptides can be detected using antibodies. Techniques for detecting polypeptides using antibodies include enzyme linked immunosorbent assays (ELISAs), Western blots, immunoprecipitations and immunofluorescence. An antibody can be polyclonal or monoclonal. An antibody having specific binding affinity for a polypeptide can be generated using methods well known in the art. The antibody can be attached to a solid support such as a microtiter plate using methods known in the art. In the presence of a polypeptide, an antibody-polypeptide complex is formed.

Detection (e.g., of an amplification product, a hybridization complex, or a polypeptide) is usually accomplished using detectable labels. The term “label” is intended to encompass the use of direct labels as well as indirect labels. Detectable labels include enzymes, prosthetic groups, fluorescent materials, luminescent materials, bioluminescent materials, and radioactive materials.

### 4. Assay, Kits and Primers

Also provided are the kits, probes, primers and antibodies that can be used to determine the frequency of an ASR plant pathogen effector in an area of cultivation. The polypeptide and the polynucleotide or variant and fragments thereof of the ASR plant pathogen effector can be packaged as components of a kit with instructions for completing the assay described herein. A DNA or RNA detection kit is provided for use in detecting the nucleotides sequences encoding the ASR plant pathogen effector polypeptide or variants and fragments thereof.

In some embodiments, the kit may comprise one or more probes having a sequence corresponding to or complementary to a sequence having 80%, 85%, 86%, 87%, 88%, 89%, 90%, 91%, 92%, 93%, 94%, 95%, 96%, 97%, 98%, 99% or 100% sequence identity with a specific region of the nucleotides sequence set forth in any of SEQ ID NOS: 3-6 or 21-22, which allows for the detection of the sequence of interest. In some embodiments, the kit may comprise any reagent and material required to perform the assay or detection method. In specific embodiments, the probes can be used to specifically hybridize to target polynucleotide and thereby detect the nucleotide sequence set forth in any of SEQ ID NOS: 3-6 or 21-22 or variants or fragments thereof.

Further provided are antibodies to the polypeptides of the present invention, or to variants or fragments thereof, are also encompassed. Methods for producing antibodies are well known in the art (see, for example, Harlow and Lane (1988) Antibodies: A Laboratory Manual, Cold Spring Harbor Laboratory, Cold Spring Harbor, N.Y.; and U.S. Pat. No. 4,196,265). These antibodies can be used in kits for the detection and isolation of toxin polypeptides. Thus, this disclosure provides kits comprising antibodies that specifically bind to the polypeptides described herein, including, for example, polypeptides having the sequence of SEQ ID NO: 1, 2 or 20 or variants or fragments thereof.

c. Determining the Frequency of the Presence of an ASR Plant Pathogen Effector in the Biological Material and Generating Recommended Pesticide Application

Quantification of one or more of an ASR effector of interest (e.g., SPE-35, SPE-38, or an active variant thereof) can be expressed as a frequency within the ASR plant pathogen population within the area of cultivation. The frequency of occurrence can be determined as a percentage of the whole population of the ASR plant pathogen present in the sample. As an example, one or more ASR effector of interest can be determined to be present at a certain frequency within the ASR population. Such a frequency within the ASR population may be at least about 1%, 2%, 5%, 10%, 15%, 20%, 25%, 30%, 35%, 40%, 45%, 50%, 55%, 60%, 65%, 70%, 75%, 80%, 85%, 90%, 95% or 100% of the ASR plant pathogen population from the sample and/or within the area of cultivation; or alternatively between about 1% to about 10%, between about 10% to 20%, between about 20% to 30%, between about 30% to 40%, between about 40% to 50%, between about 50% to 60%, between about 60% to 70%, between about 70% to 80%, between about 80% to 90% or between about 90% to 100% of the ASR effectors within a sample and/or within the ASR plant pathogen population in the area of cultivation. The frequency of the ASR effector polypeptides can also be expressed in alternative ways, for example, heatmaps, boxplots, or pies associated with or without maps.

The legume plant or soybean plant in the area of cultivation can comprise one or more ASR genetic resistance traits, such as RG31 or RG35 or an active variant or fragment thereof. As explained elsewhere herein, depending on the ASR genetic resistance trait within the crop planted in the area of cultivation, the ASR effector(s) detected within the population will change. For example, when the area of cultivation comprises a legume or soybean plant expressing RG31 or RG35 or an active variant or fragment thereof, any one of the corresponding ASR effectors of RG31 and/or RG35 can be detected in the ASR plant pathogen population. For example, SPE-35 or SPE-38 or an active variant or fragment thereof can be detected. A resistance profile in such an area of cultivation where the frequency of SPE-35 or SPE-38 or an active variant or fragment thereof is present at a high frequency within the population indicates that a pesticide application protocol with a greater ability to control the ASR pathogen will be needed. A resistance profile in such an area of cultivation where the frequency of SPE-35 or SPE-38, or an active variant or fragment thereof, is present at a low frequency within the population indicates that a pesticide application protocol with a decreased ability to control the ASR pathogen will be needed, as the ASR genetic resistance within the plants will be able to control the ASR pathogen population. Such methods allow for improved ASR pathogen resistance management in a given area of cultivation. While plants having RG31 and RG35 were used in the example above, a similar protocol can be used for any of the ASR genetic resistance traits disclosed herein with their corresponding ASR effector proteins or any combination thereof. See, for example, Table C herein for the corresponding ASR effector protein for a given ASR genetic resistance trait.

As used herein, “high frequency within the population” of the corresponding ASR effector protein for the ASR genetic resistance trait will result in a more susceptible plant population. A high effector protein frequency within the population occurs when the one or more of the ASR effectors, either alone or in combination, are present within the ASR plant pathogen population at least a frequency of about 1%, 2%, 5%, 10%, 15%, 20%, 25%, 30%, 35%, 40%, 45%, 50%, 55%, 60%, 65%, 70%, 75%, 80%, 85%, 90%, 95% or 100% frequency; or alternatively between about 1% to about 10%, between about 10% to 20%, between about 20% to 30%, between about 30% to 40%, between about 40% to 50%, between about 50% to 60%, between about 60% to 70%, between about 70% to 80%, between about 80% to 90% or between about 90% to 100% of the ASR effectors within the ASR plant pathogen population.

As used herein, “low frequency within the population” of the corresponding ASR effector protein for the ASR genetic resistance trait will result in a less susceptible plant population. A low ASR effector protein frequency within the population occurs when the one or more of the ASR effectors, either alone or in combination, are present within the ASR plant pathogen population at least a frequency of about 1%, 2%, 5%, 10%, 15%, 20%, 25%, 30%, 35%, 40%, 45%, 50%, 55%, 60%, 65%, 70%, 75%, 80%, 85%, 90%, 95% or 100% frequency; or alternatively between about 1% to about 10%, between about 10% to 20%, between about 20% to 30%, between about 30% to 40%, between about 40% to 50%, between about 50% to 60%, between about 60% to 70%, between about 70% to 80%, between about 80% to 90% or between about 90% to 100% of the ASR effectors within the ASR plant pathogen population.

The frequency of an ASR plant pathogen effector of interest at the sampling locations can be used to generate the frequency of the various ASR plant pathogen effectors within the area of cultivation. By combining data from different sampling locations within the area of cultivation the predicted local frequency of a given ASR plant pathogen effector within the ASR plant pathogen population can be determined for the local situation. A resistance map and/or a virulence map for that given area of cultivation can then be determined.

The resistance profile map and/or virulence profile map may then be employed to identify candidate pesticides for use in a pesticide application protocol to increase the resistance of the legume plants or soybean plants to the ASR plant pathogens within the area of cultivation. A recommended pesticide application protocol for the area of cultivation comprising at least one disease control measure for controlling the development, reproduction and/or viability of the ASR plant pathogen based on the resistance profile and/or virulence profile can then be generated.

Disease control measure or treatment program is to be understood as management of the ASR plant pathogen, for example, by application of a plant protection product such as a pesticide, timing or intervals of application of a plant protection product or selection of an optimal or particular resistant crop in order to control the development, reproduction and/or viability of the pathogen whilst minimizing resistance development. As discussed elsewhere herein, the disease control measure assembled will take into consideration the resistance profile and/or virulence profile of the ASR plant pathogens detected in the area of cultivation.

A disease control measure can include the application of a fungicide that can increase the development, reproduction and/or viability of the ASR plant pathogen population within the area of cultivation. Such fungicide applications include, but are not limited to, demethylation inhibitors (DMI's-tebuconazole, cyproconazole, protioconazole (triazolintione), epoxiconazole, flutriafol and others); quinone oxidase inhibitors (QoI's-azoxystrobin, trifloxystrobin, picoxystrobin and pyraclostrobin), succinate dehydrogenase inhibitors (SDHI's-fluxpyroxade, bixafen and benzovindiflupyr), cupric (oxychloride), nitriles (e 3), strobilurins, and mancozeb. Additional fungicidal application include fungicides belonging to the Carboxamide group, which have a specific mode of action, inhibiting fungal respiration, of complex II-succinate dehydrogenase (SDHI), to control ASR. Members of the carboxamide group include bixafen, fluxopyiraxade and benzovindiflupyr. Fungicides benzovindiflupyr, bixafen, fluxapyroxad, furametpyr, isopyrazam, penflufen, penthiopyrad and silkxane; or, multi-site fungicides such as mancozeb, chlorothalonil, and/or metiram.

In other embodiments, the method further comprises determining at least one disease control measure for applying to a neighbouring location based on the resistance and/or virulence profile.

Exemplary methods for determining a resistance profile and/or virulence profile in an ASR plant pathogen population present in an area of cultivation according to the present disclosure are as follows. The method comprises collecting samples from the area of cultivation wherein the samples could be a field sample or an environmental sample, for example an aerial sampling by spore traps, plant material or soil material as discussed above. Biological material from the sample is obtained, including for example, polynucleotides or polypeptides. In some embodiments, polynucleotides from the biological material are sequenced and/or amplified and/or detected or polypeptides are detected in order to detect an ASR plant pathogen effector of interest that possibly confer a higher virulence to a resistant plant or cultivar. The method further comprises determining the qualitative presence of the ASR plant pathogen effector in the plant pathogen or pathogen population and quantifying the frequency of the one or more ASR plant pathogen effector of interest. Thereafter, the method further comprises generating a resistance profile and/or virulence profile of the ASR plant pathogen or ASR pathogen population by combining the results with information or knowledge obtained from dedicated studies assessing the impact of a given frequency of the one or more ASR plant pathogen effector on crop productivity.

Such methods enable the quantitative and qualitative description of the resistance profile and/or the virulence profile of a population of a ASR plant pathogen present in a location to be determined rapidly such as close to real-time, within 48 hours or a few days. This provides opportunities to have a clear and more detailed understanding of the possible resistance profiles present in a location and to help determine a more tailored disease control measure and/or resistance management program for the current season and/or future seasons. As such, the most effective type of pesticide(s) for controlling the pathogen and/or the best crop variety can be selected such that pesticides with high presence of resistance can be avoided.

Referring now again to the operation of collecting a sample of an ASR plant pathogen, this operation may comprise receiving a sample from a farmer who has collected the sample from a field or receiving a sample from another source, e.g., a wild population of spontaneous crops or flowers, where the profile of resistance of a plant pathogen population is to be determined. The sample may be specimens that the farmer has collected from various plants across a field. Alternatively, or additionally, a sample may be environmental (e.g., soil and/or aerial samples).

Referring now again to the operation of generating a resistance profile and/or virulence profile of the plant pathogen. This operation may comprise interpreting the data determined by polynucleotide sequencing, polynucleotide amplification and detection and/or by polypeptide detection in order to detect the ASR effectors within the biological material. By applying knowledge of the susceptibility of the legume and/or soybean plants in the area or cultivation or to be planted in the area of cultivation in combination with the frequency of the different ASR effectors present in the sample(s) from the area of cultivation can be determined. More specifically, generating a resistance profile may comprise associating the presence of one or more ASR effector and corresponding frequency in the ASR plant pathogen population of the sample as determined in the preceding operation 103 with a level of susceptibility of the legumes and/or soybean plants in the area of cultivation or to be planted in the area of cultivation. The resistance level can be considered to be an index, which condenses complex information of frequencies and resistance factors of each ASR effector or a combination of ASR effectors to a single value comparable between different samples. The resistance level of a plant pathogen population of a sample requires an understanding of the effect of each ASR effector detected to the resistance of the plant (legumes and/or soybeans) within the area of cultivation.

Further provided is a method comprising determining at least one pesticide or other disease control measure for controlling the development, reproduction and/or viability of the crop pathogen based on the resistance profile and/or virulence profile. For example, a resistance profile and/or virulence profile may be determined before any use of pesticides to control the ASR pathogen. The resistance profile of the plants in the area of cultivation to specific ASR effectors (e.g., SPE-35 or SPE-38 or an active variant thereof) of the pathogen population can be generated or measured as described above in example method by monitoring the presence of one or more of the ASR effectors in the sample and quantify their frequency. Similarly, a virulence profile can be generated as described above. Based on the resistance profile and/or virulence profile a pesticide or other disease control measure can be determined or recommended. The method may further comprise an operation of collecting or receiving a subsequent or additional sample from the same location, crop or plant as the initial sample, wherein the subsequent sample has been treated with the pesticide or other disease control measure determined in operation. The subsequent sample is subjected to the operations described above and thereafter a resistance profile and/or virulence profile is generated similar to operation. Finally, the generated resistance profile and/or virulence profile is compared with that of the initial sample. For example, if a resistance profile was generated for the initial sample then it is compared with a resistance profile generated for the subsequent sample, similarly if a virulence profile was generated for the initial sample then it is compared with a virulence profile of the subsequent sample. From the comparison of the two resistance profiles, it is possible to determine if the population resistance to a fungicide class improved or deteriorated as a consequence of the agronomic decisions taken to control the disease. For example, the ratio between a sensitivity index generated before and after a pesticide treatment provides information about the sustainability of the disease control measure applied and can be used as a tool to identify and inform about suboptimal disease control measures and resistance management. The sharing of information about the disease control measures such as date of application, plant protection product applied, rate applied and other information will enable alerts about a risk decision and propose alternatives for the following seasons.

In one example, based on the comparison, the method may further comprise determining at least one pesticide or other disease control measure for controlling the plant pathogen. This pesticide or other disease control measure may be the same or different to the one(s) applied before the subsequent sample was collected and received.

The first or initial sample described herein may be collected at the beginning of the season and the subsequent or additional sample may be collected during or at the end of the season (middle or end). The beginning and the middle/end of the season may be a period of a day, several days, a week or several weeks, a month or several months, a year or several years depending on the crop, pathogen or sampling frequency needed for the particular situation. In the event that a more frequent analysis of a resistance profile is required, other samplings could be done for example after each application or over a time series.

Although the samples have been described to be collected at different time points relative to a season, it should be understood that the samples may alternatively or additionally be collected when the relevant plant is in a particular growth stage.

Furthermore, the present disclosure is not limited to an initial sample and a single subsequent sample. Several subsequent samples may be collected at different time points and then processed so as to generate a resistance profile and/or virulence profile.

It is also envisaged that the methods described herein for determining at least one pesticide or other disease control measure (as described above) for a location can be applied to neighboring locations. This is based on the assumption that the variant(s) of the plant pathogen present in the location are also present in the neighboring locations. This may be the case for environmental aerial samples wherein the plants or crops are untreated with pesticides. It is well known that different situations can be found in neighboring locations, often due to different resistant plants or crops being cultivated and/or due to different treatment programs of pesticides, such as fungicides, having been adopted in previous years. In such cases, additional considerations need to be taken before applying disease control measures to a location that has been determined for an adjacent location.

A method for determining a resistance profile and/or virulence profile for a plant pathogen in a location is provided. The method comprises receiving a sample of a plant pathogen from the location wherein the samples could be a field sample or an environmental sample. The method further comprises subjecting the sample to polynucleotide sequencing, polynucleotide amplification, polynucleotide detection and/or polypeptide detection to determine the presence of one or more ASR effectors withing the ASR population. The presence of at least one ASR effector within the ASR plant pathogen population is determined and said at least one ASR effector is quantified. Thereafter, the method further comprises generating a resistance profile and/or virulence profile of the plant pathogen or pathogen population.

### 10. METHODS FOR IDENTIFYING VARIANT POLYPEPTIDES ENCODING AN R GENE OR OTHER PLANT PATHOGEN GENETIC RESISTANCE TRAIT

Variants of a polypeptide encoding an R gene or other plant pathogen genetic resistance trait (e.g., ASR genetic resistance trait), including sequences from other organisms, can be identified based on their sequence identity and/or functional identity with the given RG31 and/or RG35 polypeptide and genes disclosed herein. In one example, variant polypeptides and polynucleotides of the RG31 and/or RG35 polypeptides, and polynucleotide sequences encoding such polypeptides, comprise proteins that interact with the same set of effector proteins (see Table C) to generate a localized hypersensitive response via a common mode of action when expressed in a plant (see also Example 3). Variant polypeptide and polynucleotides including orthologs and allelic variants of RG31 and/or RG35 polypeptides of SEQ ID NOS: 1 or 2 or 20 and/or RG31 and/or RG35 genes of SEQ ID NOS: 3-6 or 21-22 are expected to interact with effector proteins to generate a localized hypersensitive cell death response. On the other hand, polypeptides that interact with a different set of effector proteins than the effector proteins recognized by RG31 and RG35 and/or polypeptides that do not generate a localized hypersensitive response with the effectors recognized by RG31 and/or RG35 (such as SPE-35, or an active variant or fragment thereof), as well as the polynucleotides encoding such polypeptides, are assumed to not have the same mode of action as RG31 and/or RG35, irrespective of their high or low degree of sequence identity with RG31 and/or RG35. In one particular embodiment, as elaborated at Example 3, even though RG31 and RG35 have a high degree of sequence identity with the R-protein RG1 (disclosed in WIPO Publication No. WO2019103918A1 as SEQ ID NO: 47 and encoded by the polynucleotide disclosed as SEQ ID NO: 6), they do not interact with the same set of effector proteins, indicating their distinct modes of action in conferring disease resistance.

Accordingly, in some embodiments, methods are disclosed of identifying novel disease resistance polypeptides, the method comprising: identifying a set of plant pathogen effector proteins that interact with the RG31 polypeptide of SEQ ID NO: 1 or the RG35 polypeptide of SEQ ID NO: 2 or 20; assaying for interaction of the identified set of plant pathogen effector proteins with a putative RG31 polypeptide or an RG31 polypeptide having at least 40%, at least 50%, at least 60%, at least 70%, at least 80%, at least 90% or at least 95% sequence identity to SEQ ID NO: 1, or for a putative RG35 polypeptide or an RG35 polypeptide having at least 40%, at least 50%, at least 60%, at least 70%, at least 80%, at least 90% or at least 95% sequence identity to SEQ ID NO: 2 or 20; and in response to detection of interaction between the putative RG31 or RG35 polypeptide and the identified effector(s), such as via the presence of a hypersensitive response or localized cell death, indicating that the putative RG31 or RG35 polypeptide has the same mode of action as the corresponding RG31 or RG35 polypeptide.

In some embodiments, the effector proteins are expressed in a cell, purified, and infiltrated into a plant cell for an interaction assay with the putative RG31 or RG35 polypeptide. In other embodiments, a transient expression system, such as an Agrobacterium-mediated transient expression system in a plant can be used to observe the hypersensitive response cell death phenotype (HR) triggered by co-expression of a putative RG31 or RG35 gene system comprising (a) an RG31 or RG35 variant (e.g., orthologs or homologs or allelic variants of RG31 or RG35) and their putative cognate effector (e.g., an effector protein recognized by the RG31 or RG35 polypeptide of SEQ ID NOS: 1 or 2 or 20). Presence of an interaction, e.g., presence of an HR response and localized cell death (such as shown in Example 3), indicates that the protein encoded by the putative Rg31 or Rg35 genes have functional identity and a common mode of action as the RG31 or RG35 polypeptides, respectively, of the present disclosure, even if the sequence identity is low (e.g., lower than 60% sequence identity, such as 50% identity or 40% identity or lower). Absence of an interaction, e.g., absence of an HR response and localized cell death (such as shown in Example 3), indicates that the protein encoded by polypeptide does not have a common mode of action as the RG31 or RG35 polypeptides, respectively, of the present disclosure, even if the sequence identity is high (e.g., higher than 60% sequence identity, such as 80% identity or 90% identity or higher).

### Non-Limiting Embodiments Include:

### Example Embodiments of Nucleic Acids and Polypeptides for Controlling Disease Resistance

A1. A nucleic acid molecule comprising a nucleotide sequence operably linked to a heterologous regulatory element, wherein the nucleotide sequence comprises a polynucleotide encoding an RG31 or RG35 polypeptide, or active variant thereof, comprising an amino acid sequence having at least 80%, 85%, 90%, 91%, 92%, 93%, 94%, 95%, 96%, 97%, 98% 99% or 100% identity to SEQ ID NO: 1 or 2 or 20,
   wherein increased expression of the RG31 or RG35 polypeptide, or active variant thereof, confers the plant with increased disease resistance relative to a control plant.
A2. The nucleic acid molecule of embodiment A1, wherein the polynucleotide of comprises:
   (i) a nucleotide sequence encoding a RG31 polypeptide having at least 80% sequence identity to SEQ ID NO: 1 or an RG35 polypeptide having at least 80% sequence identity to SEQ ID NO: 2 or 20;
   (ii) a nucleotide sequence encoding the RG31 polypeptide of SEQ ID NO: 1 or the RG35 polypeptide of SEQ ID NO: 2 or 20; or
   (iii) a nucleotide sequence having at least 80%, 85%, 90%, 91%, 92%, 93%, 94%, 95%, 96%, 97%, 98% 99% or 100% identity to any of SEQ ID NOS: 3, 4, 5, 6, 21 or 22.
A3. The nucleic acid molecule of embodiment A1 or A2 wherein the heterologous regulatory element is a heterologous promoter active in a plant, and wherein the heterologous promoter is a constitutive promoter, an inducible promoter, or an endogenous promoter, and wherein the inducible promoter is optionally a rust inducible promoter.
A4. The nucleic acid molecule of embodiment A3, wherein the heterologous promoter has at least 95% sequence identity to a sequence selected from the group comprising SEQ ID NOS: 7, 8, 10, 11, 12, 14 and 15.
A5. The nucleic acid molecule of embodiment A3, wherein the rust inducible promoter has a nucleotide sequence having at least 95% sequence identity to SEQ ID NO: 14 or 15, or wherein the rust inducible promoter comprises SEQ ID NO: 14 or 15.
A6. The nucleic acid molecule of embodiment A3, wherein the endogenous promoter has a nucleotide sequence having at least 95% sequence identity to any one of SEQ ID NOS: 7, 8 and 10, or wherein the rust inducible promoter comprises SEQ ID NO: 7, 8, or 10.
A7. The nucleic acid molecule of any one of embodiments A1-A3, wherein said heterologous regulatory element comprises a terminator sequence, an intron, a 5′ UTR or a 3′ UTR.
A8. The nucleic acid molecule of any one of embodiments A1-A7, wherein the polynucleotide encoding the RG31 or RG35 polypeptide, or an active variant thereof, comprises at least one native intron or at least one heterologous intron.
A9. The nucleic acid molecule of any one of embodiments A1-A8, wherein the RG31 or RG35 polypeptide, or active variant or fragment thereof, is tagged with a detectable marker.
A10. A vector comprising the nucleic acid molecule of any one of embodiments A1-A9.
A11. A vector comprising a polynucleotide encoding:
(a) an RG31 polypeptide, or an active variant thereof, comprising an amino acid sequence having at least 90%, 91%, 92%, 93%, 94%, 95%, 96%, 97%, 98% 99% or 100% identity to SEQ ID NO: 1; or
(b) an RG35 polypeptide, or an active variant thereof, comprising an amino acid sequence having at least 90%, 91%, 92%, 93%, 94%, 95%, 96%, 97%, 98% 99% or 100% identity to SEQ ID NO: 2 or 20,
wherein increased expression of the RG31 or RG35 polypeptide, or active variant thereof, in a plant confers enhanced disease resistance in the plant, relative to a control plant.
A12. The vector of embodiment A11, wherein said polynucleotide comprises a nucleotide sequence having at least 90%, 91%, 92%, 93%, 94%, 95%, 96%, 97%, 98% 99% or 100% identity to any one of SEQ ID NOS: 3, 4, 5, 6, 21 or 22.
A13. A transgenic cell comprising the nucleic acid molecule of any one of embodiments A1-A9 or the vector of any one of embodiments A10-A12.
A14. A transgenic cell comprising a heterologous polynucleotide encoding an RG31 polypeptide or an RG35 polypeptide, or an active variant of an RG31 polypeptide or an RG35 polypeptide, comprising an amino acid sequence having at least 90%, 91%, 92%, 93%, 94%, 95%, 96%, 97%, 98% 99% or 100% identity to any of SEQ ID NOS: 1, 2, or 20.
A15. The transgenic cell of embodiment A14, wherein said polynucleotide comprises a nucleotide sequence having at least 90%, 91%, 92%, 93%, 94%, 95%, 96%, 97%, 98% 99% or 100% identity to any one of SEQ ID NOS: 3-6, 21 or 22.
A16. The transgenic cell of any of embodiments A13-A15, wherein the transgenic cell is a transgenic plant cell.
A17. The transgenic plant cell of embodiment A16, wherein said polynucleotide is stably integrated into the genome of the cell.
A18. The transgenic plant cell of embodiment A16 or A17, wherein the transgenic plant cell has an increased level of expression of the RG31 polypeptide, or the RG35 polypeptide, or an active variant thereof, and wherein the transgenic plant cell has increased disease resistance relative to a control plant cell.
A19. The transgenic plant cell of embodiment A19, wherein the transgenic plant cell has increased resistance to Asian Soy rust relative to a control plant cell.
A20. The transgenic plant cell of any of embodiments A16-A19, wherein the transgenic plant cell is a transgenic plant cell of a legume plant, optionally wherein the legume plant is selected from the group comprising alfalfa, clover, pea, bean lentil, lupin, mesquite, carob, soybean, pigeon pea, peanut, and tamarind.
A21. The transgenic plant cell of embodiment A20, wherein the transgenic plant cell is a transgenic soybean plant cell.
A22. The transgenic plant cell of any of embodiments A16-A19, wherein the transgenic plant cell is a monocot cell, a dicot cell, a legume cell, a soybean cell, a barley cell, a maize cell, an oat cell, a rice cell, a sorghum cell, a sugarcane cell, a wheat cell, a sunflower cell, a tomato cell, a cotton cell, a sugar beet cell or a tobacco cell.
A23. A plant, or plant part, derived from the transgenic cell of any one of embodiments A13-A22.
A24. The plant of embodiment A23, wherein the plant is a soybean plant, and wherein the soybean plant is an elite soybean plant.
A25. The plant part of embodiment A24, wherein the plant part is a transgenic seed, wherein said transgenic seed has stably incorporated the polynucleotide into its genome.
A26. A harvested product derived from the transgenic seed of embodiment A25, and wherein the harvested product comprises the polynucleotide.
A27. A processed product derived from the harvested product of embodiment A26, wherein the processed product is a flour, a meal, an oil, a starch, or a product derived from any of the foregoing, and wherein the processed product comprises the polynucleotide.
A28. A DNA construct comprising a polynucleotide operably linked to a heterologous regulatory element, wherein the polynucleotide encodes a polypeptide comprising:
(a) an amino acid sequence having at least 90% identity to SEQ ID NO: 1 or 2 or 20, wherein increased expression of the polypeptide in a plant increases disease resistance in the plant;
(b) an amino acid sequence having at least 95% identity to SEQ ID NO: 1 or 2 or 20, wherein increased expression of the polypeptide in a plant increases disease resistance in the plant; or,
(c) an amino acid sequence comprising SEQ ID NO: 1 or 2 or 20.
A29. The DNA construct of embodiment A28, wherein the polynucleotide that encodes the polypeptide comprises:
(a) a nucleotide sequence having at least 90% sequence identity to any of SEQ ID NOs: 3, 4, 5, 6, 21 or 22;
(b) a nucleotide sequence having at least 95% identity to any of SEQ ID NOS. 3, 4, 5, 6, 21 or 22; or
(c) a nucleotide sequence comprising any of SEQ ID NOS: 3, 4, 5, 6, 21 or 22.
A30. The DNA construct of any one of embodiments A28-29, wherein said heterologous regulatory element comprises a promoter active in a plant.
A31. The DNA construct of embodiment A30, wherein said promoter is a tissue-specific promoter or a constitutive promoter or a rust-reactive promoter.
A32. The DNA construct of embodiment A28, wherein said heterologous regulatory element comprises a terminator sequence, an intron, a 5′ UTR or a 3′ UTR.
A33. The DNA construct of embodiment A32, wherein said polynucleotide encoding the polypeptide is operably linked to a promoter sequence comprising:
   (a) a nucleotide sequence having at least 95% identity to any one of SEQ ID NOS: 7, 8, 10, 12, 14 or 15, where said nucleotide sequence is capable of driving expression of the polypeptide of interest in plant cell; or
   (b) a nucleotide comprising any one of SEQ ID NOS: 7, 8, 10, 12, 14 or 15.
A34. The DNA construct of any one of embodiments A28-33, wherein the polynucleotide encoding the polypeptide comprises at least 1 native intron or at least one heterologous intron.
A35. A vector comprising the DNA construct of any of embodiments A28-34.
A36. A vector comprising a polynucleotide encoding a polypeptide comprising:
(a) an amino acid sequence having at least 90% identity to SEQ ID NO: 1 or 2 or 20, wherein increased expression of the polypeptide in a plant increases disease resistance in the plant;
(b) an amino acid sequence having at least 95% identity to SEQ ID NO: 1 or 2 or 20, wherein increased expression of the polypeptide in a plant increases disease resistance in the plant; or,
(c) an amino acid sequence comprising SEQ ID NO: 1 or 2 or 20.
A37. The vector of embodiment A37, wherein said polynucleotide comprises
(a) a nucleotide sequence having at least 90% sequence identity to any one of SEQ ID NOS: 3, 4, 5, 6, 21 or 22;
(b) a nucleotide sequence having at least 95% identity to any one of SEQ ID NOS: 3, 4, 5, 6, 21 or 22; or
(c) a nucleotide sequence comprising any one of SEQ ID NOS: 3, 4, 5, 6, 21 or 22.
A38. A cell comprising the DNA construct of any of embodiments A28-34, or the vector of any of embodiments A35-37.
A39. A cell comprising a heterologous polynucleotide comprising encoding a polypeptide comprising:
(a) an amino acid sequence having at least 90% identity to SEQ ID NO: 1 or 2 or 20, wherein increased expression of the polypeptide in a plant increases disease resistance in the plant;
(a) an amino acid sequence having at least 95% identity to SEQ ID NO: 1 or 2 or 20, wherein increased expression of the polypeptide in a plant increases disease resistance in the plant; or,
(b) an amino acid sequence comprising SEQ ID NO: 1 or 2 or 20.
A40. The cell of embodiment A39, wherein said polynucleotide comprises:
(a) a nucleotide sequence having at least 90% sequence identity to any one of SEQ ID NOS: 3, 4, 5, 6, 21 or 22;
(b) a nucleotide sequence having at least 95% identity to any one of SEQ ID NOS: 3, 4, 5, 6, 21 or 22; or
(c) a nucleotide sequence comprising any one of SEQ ID NOS: 3, 4, 5, 6, 21 or 22.
A41. The cell of any one of embodiments A38-40, wherein said cell is a plant cell.
A42. The cell of any one of embodiments A38-41, wherein said polynucleotide is stably integrated into the genome of the cell.
A43. The plant cell of embodiment A41 or A42, wherein the plant cell has an increased level of expression of the polypeptide and the plant cell has increased disease resistance relative to a control plant cell.
A44. The plant cell of any one of embodiments A41-43, wherein the plant cell is (a) a monocot cell, (b) a dicot cell, (c) a legume cell, (d) a soybean cell, (e) a barley cell, a maize cell, an oat cell, a rice cell, a sorghum cell, a sugarcane cell or a wheat cell; or (f) a sunflower cell, a tomato cell, a cotton cell, a sugar beet cell or a tobacco cell.
A45. A plant comprising the plant cell of any of embodiments A41-44.
A46. The plant of embodiment A45, wherein the transgenic plant has increased resistance to Asian Soybean Rust.
A47. The plant of embodiment A46, wherein the transgenic plant is a legume plant.
A48. The plant of embodiment A47, wherein the legume plant is a soybean plant.
A49. The plant of embodiment A48, wherein the soybean plant is an elite soybean plant.
A50. A seed of the plant of any of embodiments A45-49, wherein said seed has stably integrated into its genome the said heterologous polynucleotide.
A51. A harvested product derived from the plant of any of embodiments A45-49 or the transgenic seed of embodiment A50.
A52. A processed product derived from the harvested product of embodiment A51, wherein the processed product is a flour, a meal, an oil, a starch, or a product derived from any of the foregoing.
A53. A method of producing a plant having an increased disease resistance comprising:
(a) introducing into the genome of a plant cell a heterologous polynucleotide encoding a polypeptide comprising:
   i) an amino acid sequence a having at least 90% identity to SEQ ID NO: 1 or 2 or 20, wherein increased expression of the polypeptide in a plant increases disease resistance in the plant;
   ii) an amino acid sequence having at least 95% identity to SEQ ID NO: 1 or 2 or 20, wherein increased expression of the polypeptide in a plant increases disease resistance in the plant; or,
   iii) an amino acid sequence comprising SEQ ID NO: 1 or 2 or 20; and
(b) regenerating the soybean plant cell of (a) into a soybean plant wherein expression of said polypeptide in the plant increases the disease resistance of the plant.
A54. The method of embodiment A53, wherein said heterologous polynucleotide comprises:
(a) a nucleotide sequence having at least 90% sequence identity to any one of SEQ ID NOs: 3, 4, 5, 6, 21 or 22;
(b) a nucleotide sequence having at least 95% identity to any one of SEQ ID NOS: 3, 4, 5, 6, 21 or 22; or
(c) a nucleotide sequence comprising any one of SEQ ID NOS. 3, 4, 5, 6, 21 or 22.
A55. The method of embodiments A54, wherein said plant is a legume plant.
A56. The method of embodiment A55, wherein said legume plant is a soybean plant.
A57. The method of any one of embodiments A53-56, wherein said increase in disease resistance comprises an increased resistance to Asian Soybean Rust.
A58. The method of any one of embodiments A53-57, wherein said increase in disease resistance comprises an increased resistance to the pathogens Phakopsora pachyrhizi and/or Phakopsora meibomiae.
A59. The method of any one of embodiments A53-58, wherein introducing said heterologous polynucleotide is through gene editing.
A60. The method of any one of embodiments A53-58, wherein introducing said heterologous polynucleotide into the plant genome is through transformation of the plant cell with a DNA construct of any one of embodiments A28-34 or the vector of embodiments A35-37.
A61. The method of any one of embodiments A53-60, further comprising crossing a first soybean plant comprising the heterologous polynucleotide or vector with a second, different legume plant or soybean plant.
A62. The method of embodiment A61, wherein the method further comprises obtaining a progeny legume plant or soybean plant for one or more generations from the transgenic soybean plant, wherein the progeny soybean plant comprises the heterologous polynucleotide and has increased resistance to the soybean pathogen.
A63. A method of decreasing Asian Soybean Rust damage or controlling an ASR pathogen in an area of cultivation comprising the planting in said area of cultivation a plant of any one of embodiments A45-49 or seed of embodiment 50.
A64. A method of determining the presence of a polypeptide having at least 90%, 95% or 100% sequence identity to SEQ ID NO: 1 or 2 or 20, comprising the steps of:
(a) isolating nucleic acid molecules from a legume plant and generating an amplicon comprising at least a fragment of a polynucleotide encoding said polypeptide using a probe and/or primer; or
(b) isolating proteins from said soybean plant and detecting presence of said polypeptide; thereby determining the presence of the polypeptide in the legume plant.
A65. A method for producing an Asian Soybean Rust (ASR) resistant soybean plant comprising the steps of:
(a) selecting a soybean plant from a plurality of soybean plants by detecting the presence of a polynucleotide encoding a polypeptide comprising the amino acid sequence having at least 90%, 95% or 100% sequence identity to SEQ ID NO: 1 or 2 or 20; and
(b) generating an ASR resistant progeny soybean plant from said selected soybean plant in a breeding program.
A66. The method of embodiment A65, wherein said polynucleotide comprises a sequence having at least 90%, 95%, or 100% identity to any one of SEQ ID NOS: 3, 4, 5, 6, 21 or 22.

### Example Embodiments of Methods for Controlling Disease Resistance.

B1. A method of controlling disease resistance in an area of cultivation comprising the step of planting in the area of cultivation a legume plant, a plant part or a seed having stably incorporated into its genome:
   (a) a nucleotide sequence encoding an RG31 or an RG35 polypeptide, or an active variant thereof, having at least 80%, 85%, 90%, 91%, 92%, 93%, 94%, 95%, 96%, 97%, 98% 99% or 100% identity to SEQ ID NO: 1 or 2 or 20; and
   (b) a heterologous promoter operably coupled to the nucleotide sequence, wherein increased expression of the RG31 or RG35 polypeptide, or active variant thereof, confers increased disease resistance to the legume plant, plant or seed.
B2. The method of embodiment B1, wherein the RG31 or RG35 polypeptide, or active variant thereof, is tagged with a detectable marker.
B3. The method of embodiment B1 or B2, wherein the heterologous promoter is an endogenous promoter, a rust inducible promoter, or a constitutive promoter active in said plant.
B4. The method of embodiment B3, wherein the heterologous promoter comprises a nucleotide sequence having at least 95% sequence identity to any one of SEQ ID NOS: 7, 8, 10, 12, 14 and 15 or wherein the promoter comprises the sequence of any one of SEQ ID NOS: 7, 8, 10, 12, 14 and 15.
B5. The method of embodiment B3 or B4, wherein:
   (i) the endogenous promoter comprises a nucleotide sequence having at least 95% sequence identity to any one of SEQ ID NOS: 7, 8, and 10, or wherein the endogenous promoter comprises any one of SEQ ID NOS: 7, 8, and 10; and
   (ii) the rust inducible promoter comprises a nucleotide sequence having at least 95% sequence identity to any one of SEQ ID NOS: 14 and 15, or wherein the constitutive promoter comprises any one of SEQ ID NOS: 14 and 15.
B6. The method of any one of embodiments B1-B5, wherein said increased disease resistance comprises an increased resistance to the pathogens Phakopsora pachyrhizi and Phakopsora meibomiae.
B7. A legume plant produced by the method of any one of embodiments B1-B6.
B8. The legume plant of embodiment B7, wherein the legume plant is a legume crop plant, and optionally wherein the legume crop plant is alfalfa, clover, pea, bean, lentil, lupin, mesquite, carob, soybean, pigeon pea, peanut or tamarind.
B9. The legume plant of embodiment B8, wherein the legume crop plant is a soybean plant.
B10. The legume plant of embodiment B9, wherein the soybean plant is an elite soybean plant.
B11. The legume plant of any one of embodiments B7-B10, wherein the plant has resistance to one or more of the following: soy cyst nematode, bacterial pustule, root knot nematode, frog eye leaf spot, phytopthora, brown stem rot, nematode, Asian Soybean Rust, smut, Golovinomyces cichoracearum, Erysiphe cichoracearum, Blumeria graminis, Podosphaera xanthii, Sphaerotheca fuliginea, Pythium ultimum, Uncinula necator, Mycosphaerella pinodes, Magnaporthe grisea, Bipolaris oryzae, Magnaporthe grisea, Rhizoctonia solani, Phytophthora sojae, Schizaphis graminum, Bemisia tabaci, Rhopalosiphum maidis, Deroceras reticulatum, Diatraea saccharalis, Schizaphis graminum or Myzus persicae.
B12. The legume plant of embodiment B11, wherein the plant has resistance to Asian Soybean Rust.
B13. A method of decreasing Asian Soybean Rust damage or controlling an ASR pathogen in an area of cultivation comprising planting in said area of cultivation, a plant of any one of embodiments A23-A24 or A45-49 or B7-12 or seed of embodiment A25 or A50.

### Example Embodiments of Plants Having Increased Disease Resistance.

C1. A plant comprising in its genome a stably integrated nucleic acid molecule, the nucleic acid molecule comprising a heterologous promoter operably coupled to a polynucleotide encoding a RG31 or RG35 polypeptide, or active variant thereof, comprising an amino acid sequence having at least 80%, 85%, 90%, 91%, 92%, 93%, 94%, 95%, 96%, 97%, 98% 99% or 100% identity to SEQ ID NO: 1 or 2 or 20, wherein presence of said nucleic acid molecule in the plant confers the plant with increased disease resistance relative to a control plant not comprising the nucleic acid molecule.
C2. The plant of embodiment C1, wherein the polynucleotide encoding the RG31 or RG32 polypeptide, or active variant thereof, comprises a nucleotide sequence having at least 80%, 85%, 90%, 91%, 92%, 93%, 94%, 95%, 96%, 97%, 98% 99% or 100% identity to any one of SEQ ID NOS: 3, 4, 5, 6, 21 or 22.
C3. The plant of any one of embodiments C1-C2, wherein the plant has increased resistance to Asian Soy Rust.
C4. A method for producing an Asian Soybean Rust (ASR) resistant soybean plant comprising the steps of:
(a) selecting a soybean plant from a plurality of soybean plants by detecting the presence of a polynucleotide encoding a polypeptide comprising the amino acid sequence having at least 90%, 95% or 100% sequence identity to SEQ ID NO: 1 or 2 or 20; and
(b) generating an ASR resistant progeny soybean plant from said selected soybean plant in a breeding program.
C5. The method of embodiment C4, wherein said polynucleotide comprises a sequence having at least 90%, 95%, or 100% identity to any one of SEQ ID NOS: 3, 4, 5, 6, 21 or 22.

### Example Embodiments of Compositions for Controlling Disease Resistance.

D1. An isolated or recombinant polypeptide comprising
(a) an amino acid sequence having at least 90% identity to SEQ ID NO: 1 or 2 or 20, wherein increased expression of the polypeptide in a plant increases disease resistance in the plant;
(b) an amino acid sequence having at least 95% identity to SEQ ID NO: 1 or 2 or 20, wherein increased expression of the polypeptide in a plant increases disease resistance in the plant; or,
(c) an amino acid sequence comprising SEQ ID NO: 1 or 2 or 20.
D2. The isolated or recombinant polypeptide of embodiment D1, further comprising a heterologous amino acid sequence.
D3. A composition comprising the isolated or recombinant polypeptide of embodiment D1 or D2.

### EXAMPLES

The following examples are not intended to be a detailed catalog of all the different ways in which the present invention may be implemented or of all the features that may be added to the present invention. Persons skilled in the art will appreciate that numerous variations and additions to the various embodiments may be made without departing from the present invention. Hence, the following descriptions are intended to illustrate some particular embodiments of the invention, and not to exhaustively specify all permutations, combinations and variations thereof.

### Example 1: Identification of Novel Resistance Polypeptides, Rg31 and Rg35, Encoded by Novel Resistance Genes Rg31 and Rg35 Genes, Respectively

Two resistant Glycine tomentella lines (PI446958 and PI483224), exhibiting ASR resistance against multiple rust strains, were crossed to a susceptible Glycine tomentella line (PI441010 or 446948). Using segregation and marker analysis, the ASR resistance locus for PI446958 was mapped to a first genetic interval located on soybean chromosome 5. Using segregation and marker analysis, the ASR resistance locus for PI483224 was mapped to a second, distinct genetic interval also located on soybean chromosome 5. Both mappings were performed using Glycine max reference genome William82 V2.

Further genotyping and analysis of the genetic interval derived from G. tomentella PI483224 led to the identification of a causative ASR resistance gene Rg31 (SEQ ID NO: 3-4) encoding a resistance protein herein referred to as RG31 (SEQ ID NO: 1). Genotyping and analysis of the genetic interval derived from G. tomentella PI446958 led to the identification of a causative ASR resistance gene Rg35 (SEQ ID NO: 5-6) encoding a resistance protein herein referred to as RG35 (SEQ ID NO: 2). The genes were identified based on the presence of conserved TNLKW motifs, as shown in Table A. The RG31 (SEQ ID NO: 1) and RG35 (SEQ ID NO: 2) polypeptides share 97.7% sequence identity with each other.

### 1. RG31

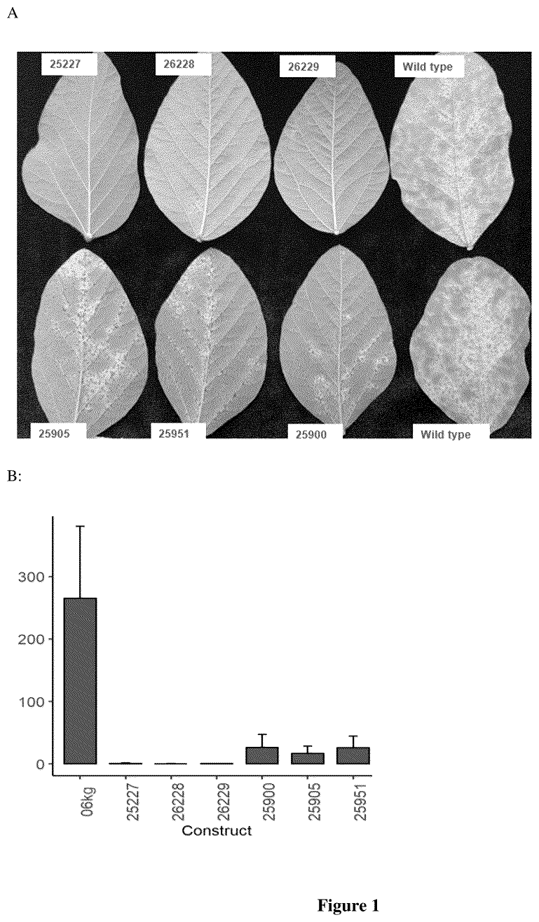

RG31 encodes a resistance gene, or R-gene, with a TNLWK motif derived from Glycine tomentella PI483224. The sequence of the polypeptide encoded by Rg31 is depicted at SEQ ID NO: 1. The genomic sequence for the R-gene is depicted in SEQ ID NO: 3 and the cDNA sequence of the gene is encoded by SEQ ID NO: 4. The native promoter for this gene is provided at SEQ ID NO: 7. A modified native promoter derived from the Rg31 genomic sequence is provided at SEQ ID NO: 8 and has one or more ORFs within the 5′-UTR of the promoter sequence removed and/or silenced. The native terminator for this gene is provided at SEQ ID NO: 9. Details regarding the validation and efficacy of the R-gene is provided at Example 2 and FIGS. 1A-B and 2A-B.

### 2. RG35

RG35 encodes a resistance gene, or R-gene, with a TNLWK motif derived from Glycine tomentella PI446958. The sequence of the polypeptide encoded by Rg35 is depicted at SEQ ID NO: 2. The genomic sequence for the R-gene is depicted in SEQ ID NO: 5 and the cDNA sequence of the gene is encoded by SEQ ID NO: 6. The native promoter for this gene is provided at SEQ ID NO: 10. The native terminator for this gene is provided at SEQ ID NO: 11. Details regarding the validation and efficacy of the R-gene is provided at Example 2 and FIGS. 1A-B and 2A-B.

It is contemplated that the R-genes of the present invention, or variants, fragments, homologs or orthologs thereof, can be employed in a transgenic, gene editing, or breeding method utilizing embryo rescue, tetraploid soy, or other introgression methods, as described above, to generate plants having increased resistance to fungal pathogens including ASR. Additionally, or optionally, nucleic acid molecules comprising the R-gene coding sequence of the present invention (e.g., any of SEQ ID NOS: 3-6), or nucleic acid molecules with a nucleotide sequence substantially identical to the R-gene coding sequence of the present invention (e.g., any nucleotide sequence having at least 85%, 90%, 91%, 92%, 93%, 94%, 95%, 96%, 97%, 98%, or 99% sequence identity to any of SEQ ID NOS: 3-6 or 21 or 22), or nucleic acid molecules comprising a nucleotide sequence encoding a polypeptide having the amino acid sequence of any of SEQ ID NOS: 1, 2, or 20, or a polypeptide having an amino acid sequence substantially identical to any of SEQ ID NOS: 1, 2, or 20 (e.g., any nucleotide sequence encoding a polypeptide having at least 85%, 90%, 91%, 92%, 93%, 94%, 95%, 96%, 97%, 98%, or 99% sequence identity to any of SEQ ID NOS: 1, 2, or 20), can be employed in a transgenic, gene editing, or breeding method utilizing embryo rescue, tetraploid soy, or other introgression methods, as described above to generate pathogen resistant (e.g., ASR resistant) plants.

Given the sequence of the R-gene and the associated trait (e.g., ASR resistance), oligonucleotide primers can be developed and said primers can be used to identify plants carrying the gene with the nucleotide sequence depicted in any of SEQ ID NOS: 3-6. A TAQMAN® assay (e.g., generally a two-step allelic discrimination assay or similar), a KASP™ assay (generally a one-step allelic discrimination assay defined below or similar), or both can be employed to assay the genes. In an exemplary two-step assay, a primer pair comprising a forward primer and a reverse primer are employed to amplify the gene, or a functional part thereof, associated with conferring ASR resistance. Further, an assay probe (or hybridization oligo) can be employed with the primer pair to detect a target sequence present in the amplified gene. The probe may be labeled with, for example, fluorophores to permit easy detection. Further, the probes may include a minor groove binder (MGB) moiety at the 3′ end that increases the melting temperature (Tₘ) of the probe and stabilizes probe-target hybrids. This allows the length of the probe to be shortened while still providing sequence discrimination and flexibility to accommodate the target. Further, the probe can include a nonfluorescent quencher (NFQ) to absorb (quench) signal from the fluorescent dye label at the other end of the probe, reducing background noise and improving sensitivity of the probe. In some embodiments, the assay primers and the reaction conditions are designed such that an assay primer will only hybridize to the reverse complement of a 100% perfectly matched sequence, thereby permitting detection of the gene based upon detection of hybridizations.

### Example 2: Expression of Rg31 or Rg35 Confers Increased Rust Resistance to Soybean

### I. Cloning of Rg31 and Rg35 Genes

DNA constructs, including expression cassettes and vectors, were generated comprising a polynucleotide encoding the identified RG31 or RG35 polypeptide operably coupled to a heterologous regulatory element. In particular embodiments, the constructs comprise the resistance gene Rg31 or Rg35 operably linked to a heterologous promoter active in a plant cell and capable of driving expression of the gene in the plant cell. Binary vectors with a gene cassette comprising the Rg31 gene or the Rg35 gene, or a polynucleotide encoding the RG31 or RG35 polypeptide, were created. A list of the binary vectors created comprising the Rg31 or Rg35 gene or a polynucleotide encoding the RG31 or RG35 polypeptide is provided at Table 1. Table 1 details the coding sequence used as well as the promoter driving the expression of the gene.

Target sequences (including available restriction enzyme sites for construction) were synthesized by GenScript. Then the target sequences and base vectors were cut by restriction enzymes and ligated by T4 ligase to generate the final vector. Positive clones were identified by RDA and Sanger sequencing. Target sequences (including overlap region in the end for HiFi ligation) were synthesized by GenScript. Then the target sequences and base vector were cut by suitable restriction enzymes and ligated by HiFi DNA Assembly kit (NEB) to generate final vector. Positive clones were identified by RDA and Sanger sequencing.

TABLE 1
List of binary vectors created for R-genes RG31 and RG35

| Construct | R protein | | | |
|---|---|---|---|---|
| ID # | encoded | Promoter | Terminator | Marker |
| 25227 | RG35 (SEQ | Native promoter | Native terminator | EPSPS |
| | ID NO: 2) | (SEQ ID NO: 10) | (SEQ ID NO: 11) | |
| 26228 | RG35 (SEQ | prMt51186 | tMt51186 | ALS |
| | ID NO: 2) | (SEQ ID NO: 12) | (SEQ ID NO: 13) | |
| 26229 | RG35 (SEQ | prMt51186 | tMt51186 | ALS |
| | ID NO: 2) | (SEQ ID NO: 12) | (SEQ ID NO: 13) | |
| 25905 | RG31 (SEQ | Native promoter | Native terminator | ALS |
| | ID NO: 1) | (SEQ ID NO: 7) | (SEQ ID NO: 9) | |
| 25951 | RG31 (SEQ | Native promoter | Native terminator | ALS |
| | ID NO: 1) | (SEQ ID NO: 7) | (SEQ ID NO: 9) | |
| 25900 | RG31 (SEQ | prMt51186 | tMt51186 | ALS |
| | ID NO: 1) | (SEQ ID NO: 12) | (SEQ ID NO: 13) | |

The base vector used for creation of construct 25227 comprised the features listed in Table 2. The base vector used for creation of constructs 26228, 26229, 259065, 25951 and 25900 comprised the features listed in Table 3.

TABLE 2
Features of Base vector used for creation of construct 25227

| | |
|---|---|
| bNLB | Left border repeat region of T-DNA of Agrobacterium tumefaciens |
| | nopaline ti-plasmid |
| bNRB | Right border region of T-DNA of Agrobacterium tumefaciens nopaline ti- |
| | plasmid. |
| cCP4EPSPSCTP2 | Arabidopsis CTP2 (Chloroplast transit peptide) and CP4EPSPS (RR2) |
| | fusion. |
| cRepA | RepA gene with A to G at nt735 |
| cSpec | Also called aadA; gene encoding the enzyme aminoglycoside |
| | 3′adenyltransferase that confers resistance to spectinomycin and |
| | streptomycin for maintenance of the vector in E. coli and Agrobacterium |
| cVirG | virG (putative) from pAD1289 with TTG start codon. pAD1289 is |
| | described in Hansen et al. 1994, PNAS 91: 7603-7607 |
| iGmEF | The first intron of the soybean elongation factor (EF) gene |
| oCOLE | The ColE1 origin of replication functional in E. coli |
| oVS1 | origin of replication and partitioning region from plasmid pVS1 of |
| | Pseudomonas (Itoh et al. 1984, Plasmid 11: 206-220) |
| prGmEF | Translation elongation factor EF-1 alpha/Tu promoter, including the first |
| | intron and neighboring UTR, from soybean (Williams 82). |
| prVirG | virG promoter (Winans J. Bact. 172: 2433-38 (1990)) composed of two |
| | promoter elements, one responsive to acetosyringone and phosphate- |
| | starvation (bp 45 to 83) and another to medium acidification (86 to 128) |
| tPsE9 | 3′-UTR of the pea (Pisum sativum) rib-1,5-bisphospate carboxylase (rbcS2) |
| | small subunit E9. |
| u5GmEF | 5′ UTR of the soybean elongation factor (EF) gene. |
| xAtCTP2 | Chloroplast transit peptide of Arabidopsis thaliana 3-phosphoshikimate 1- |
| | carboxyvinyltransferase/5-enolpyruvylshikimate-3-phosphate/EPSP |
| | synthase (AT2G45300) |
| xSTOPS | 6-frame stop to minimize unintended ORF read-through |
| xTAG | 40 bp site for plant insert intactness testing and to stop readthrough ORFs. |

TABLE 3
Features of Base vector used for creation of constructs
26228, 26229, 259065, 25951 and 25900

| | |
|---|---|
| bNLB | Left border repeat region of T-DNA of Agrobacterium tumefaciens |
| | nopaline ti-plasmid |
| bNRB | Right border region of T-DNA of Agrobacterium tumefaciens nopaline ti- |
| | plasmid |
| cNtALS | Acetolactate synthase double mutant (P191A, W568L) from Nicotiana |
| | tabacum, codon-optimized for soybean expression |
| cRepA | RepA gene with A to G at nt735 |
| cSpec | Also called aadA; gene encoding the enzyme aminoglycoside |
| | 3′adenyltransferase that confers resistance to spectinomycin and |
| | streptomycin for maintenance of the vector in E. coli and Agrobacterium |
| cVirG | virG (putative) from pAD1289 with TTG start codon. virGN54D came from |
| | pAD1289 described in Hansen et al. 1994, PNAS 91: 7603-7607 |
| iGmEF | The first intron of the soybean elongation factor (EF) gene |
| oCOLE | The ColE1 origin of replication functional in E. coli |
| oVS1 | origin of replication and partitioning region from plasmid pVS1 of |
| | Pseudomonas (Itoh et al. 1984, Plasmid 11: 206-220) |
| prGmEF | Translation elongation factor EF-1 alpha/Tu promoter, including the first |
| | intron and neighboring utr, from soybean (williams 82). |
| prVirG | virG promoter (Winans J. Bact. 172: 2433-38 (1990)) composed of two |
| | promoter elements, one responsive to acetosyringone and phosphate- |
| | starvation (bp 45 to 83) and another to medium acidification (86 to 128) |
| tGmEPSPS-04 | Modified EPSPS terminator from Glycine max |
| u5GmEF | 5′ UTR of the soybean elongation factor (EF) gene |
| xSTOPS | 6-frame stop to minimize unintended ORF read-through |
| xTAG | 40 bp site for plant insert intactness testing and to stop readthrough ORFs |

### II. Soybean Transformation

Binary vectors 25227, 26228, 26229, 259065, 25951 and 25900 were electroporated into Escherichia coli. Transformants were selected and plasmid DNA was isolated using standard miniprep methods. Transformants were characterized by diagnostic restriction enzyme digestions of the miniprep DNA. Positive clones displaying the expected pattern of digestion bands were selected and used for transformation into Agrobacterium tumefaciens. Transformants were selected and sequence verified for the presence of the appropriate binary construct before being submitted for Agrobacterium-mediated transformation.

Transgenic soybean lines were produced from immature seed cultures following the Agrobacterium-mediated transformation protocol. Briefly, immature seeds were harvested from soybean pods of ASR susceptible plant lines grown in the greenhouse under standard conditions. Seeds were surface sterilized, immature cotyledons were aseptically excised and cultures were maintained in flasks containing liquid media on rotary shakers under cool fluorescent light. Agrobacterium tumefaciens carrying plasmids with genes of interest were used to transform the immature cotyledons of the susceptible plant line. Selection of primary soybean events was achieved using an appropriate selection agent. In addition, Taqman analysis of the primary soybean events was performed to identify events harboring the Rg31 or Rg35 cassettes. Transgenic events were selected and regenerated to maturity. These plants were grown under the same conditions as the wild-type plants but in separate growth chambers.

### III. Characterization of Rust Resistance of Transgenic Events

Soybean transgenic events at T0 generations, created from constructs 25227, 26228, 26229, 259065, 25951 and 25900, were then characterized for their resistance against soybean rust. Leaves from primary events comprising the Rg31 or Rg35 gene were placed in a petri dish on a moist paper towel and then inoculated with a spore suspension of different soybean rust populations. Leaves from null events served as negative control. After 14 days, these leaves were evaluated for resistance to soybean rust. The rating and evaluation were performed using methods well known in the art. The symptom evaluation is a modified version of a rust rating scale from Burdon and Speer (Euphytica, 33:891-896, 1984; also, TAG 1984). The ratings for all the assessed transgenic events are shown in Table 4.

The transgenic events were compared to a control (which comprises the same genetic background without the transgene). The results use standard soy rust rating scales with Reddish-brown (RB) types indicative of being resistant while Tan ratings are indicative of being susceptible. Numbers after the RB ratings are based on a combination of density of lesions or size of the lesions with a 1-4 scale from high to intermediate resistance, and indication of no sporulation (NSP) or very little sporulation (SPL). Numbers after Tan ratings are based on a combination of density of pustules and level of sporulation with 1-5 scale from low to high sporulation.

As shown at Table 4, soybean transgenic T0 events expressing either the RG31 polypeptide or the RG35 polypeptide conferred resistance to soybean rust.

TABLE 4
Ratings of multiple T0 homozygous events generated from
constructs 25227, 25900, 25905, 25951, 26228, and 26229

| Material IDs | Construct | RTP1 | RTP1 | 21BRM | 21BRM | 21BR08 | 21BR08 |
|---|---|---|---|---|---|---|---|
| 21SBI220582 | 25227 | RB0-NSP | RB0-NSP | RB0-NSP | RB0-NSP | RB0-NSP | NA |
| 21SBI220595 | 25227 | RB0-NSP | RB0-NSP | RB0-NSP | RB0-NSP | RB0-NSP | NA |
| 21SBI220535 | 25227 | RB0-NSP | RB0-NSP | RB0-NSP | RB0-NSP | RB0-NSP | NA |
| 21SBI220600 | 25227 | RB0-NSP | RB0-NSP | RB0-NSP | RB0-NSP | RB0-NSP | NA |
| 21SBI220531 | 25227 | RB0-NSP | RB0-NSP | RB0-NSP | RB0-NSP | RB0-NSP | NA |
| 21SBI220770 | 25900 | RB1-NSP | RB2-NSP | RB1/2-NSP | RB3-NSP | RB3-SPL | RB3-NSP |
| 21SBI220770 | 25900 | RB1/2-NSP | RB1-NSP | RB1-NSP | RB1/2-NSP | RB3-SPL | RB4-NSP |
| 21SBI220770 | 25900 | RB1/2-NSP | RB1-NSP | RB1/2-NSP | RB2-NSP | RB4-SPL | RB3-SPL |
| 21SBI220770 | 25900 | RB1-NSP | RB1-NSP | RB1/2-NSP | RB3-NSP | RB2-NSP | RB2-SPL |
| 21SBI220813 | 25900 | RB4-SPL | RB2-SPL | RB2-NSP | NA | RB3-SPL | RB2-SPL |
| 21SBI220774 | 25905 | RB2-NSP | RB1-NSP | RB1-NSP | RB2-SPL | RB1-NSP | RB1-NSP |
| 21SBI220774 | 25905 | RB1/2-NSP | RB2-SPL | RB3-NSP | RB2-NSP | RB1/2-NSP | RB2-NSP |
| 21SBI220774 | 25905 | RB1-NSP | RB1-NSP | RB1-NSP | RB1/2-NSP | RB1-NSP | RB2-NSP |
| 21SBI220809 | 25905 | RB1/2-NSP | RB1/2-NSP | RB2-NSP | RB2-NSP | RB2-NSP | RB1/2-NSP |
| 21SBI220809 | 25905 | RB1-NSP | RB1/2-NSP | RB1/2-NSP | RB1/2-NSP | RB1-NSP | RB3-NSP |
| 21SBI220809 | 25905 | RB1-NSP | RB1-NSP | RB1-NSP | RB1/2-NSP | RB1-NSP | RB1-NSP |
| 21SBI220809 | 25905 | RB1-NSP | RB1-NSP | RB1-NSP | RB2-NSP | RB1/2-NSP | RB2-NSP |
| 21SBI220908 | 25951 | RB2-NSP | RB1/2-NSP | RB1/2-NSP | RB2-NSP | RB1-NSP | RB1/2-NSP |
| 21SBI220916 | 25951 | RB3-SPL | RB2-NSP | RB3-SPL | RB4-NSP | RB3-NSP | RB3-NSP |
| 21SBI220925 | 25951 | RB2-NSP | RB1/2-NSP | RB1/2-NSP | RB3-NSP | RB3-NSP | RB4-NSP |
| 21SBI220925 | 25951 | RB2-NSP | RB2-NSP | RB2-NSP | RB2-NSP | RB2-NSP | RB3-NSP |
| 21SBI221052 | 25951 | RB1/2-NSP | RB1/2-NSP | RB2-NSP | RB3-NSP | RB1/2-NSP | RB2-NSP |
| 21SBI221052 | 25951 | RB3-NSP | RB2-NSP | RB3-NSP | RB1/2-NSP | RB2-NSP | RB2-NSP |
| 21SBI221052 | 25951 | RB1-NSP | RB1/2-NSP | RB1-NSP | RB1-NSP | RB1/2-NSP | RB2-NSP |
| 21SBI221052 | 25951 | RB1-NSP | RB1-NSP | RB1-NSP | RB1-NSP | RB2-NSP | RB1/2-NSP |
| 21SBI221065 | 25951 | RB1/2-NSP | RB4-SPL | RB3-NSP | RB2-NSP | RB4-NSP | RB4-NSP |
| 21SBI221065 | 25951 | RB2-NSP | RB3-SPL | RB3-NSP | RB2-NSP | RB4-NSP | RB3-NSP |
| 21SBI221091 | 25951 | RB4-SPL | RB3-NSP | RB3-NSP | RB3-NSP | RB3-NSP | RB4-NSP |
| 21SBI221091 | 25951 | RB3-SPL | RB3-NSP | RB4-NSP | RB4-SPL | RB4-SPL | RB4-NSP |
| 21SBI221091 | 25951 | RB3-NSP | RB3-NSP | RB3-NSP | RB3-SPL | RB2-NSP | RB2-NSP |
| 21SBI221091 | 25951 | RB3-NSP | RB3-NSP | RB2-NSP | RB4-SPL | RB2-NSP | RB3-NSP |
| 21SBI221091 | 25951 | RB3-NSP | RB3-NSP | RB3-NSP | RB4-SPL | RB1/2-NSP | RB4-NSP |
| 21SBI221104 | 25951 | RB2-NSP | RB3-NSP | RB1/2-NSP | RB4-SPL | RB3-NSP | RB2-NSP |
| 21SBI221104 | 25951 | RB2-NSP | RB3-NSP | RB1/2-NSP | RB2-NSP | RB2-NSP | RB4-NSP |
| 21SBI221104 | 25951 | RB2-NSP | RB3-NSP | RB2-NSP | RB1-NSP | RB2-NSP | RB3-NSP |
| 21SBI221104 | 25951 | RB2-NSP | RB3-NSP | RB2-NSP | RB1/2-NSP | RB1-NSP | RB1/2-NSP |
| 21SBI221104 | 25951 | RB2-NSP | RB3-NSP | RB1/2-NSP | RB2-NSP | RB2-NSP | RB2-NSP |
| 21SBI221114 | 25951 | RB2-NSP | RB3-NSP | RB3-NSP | RB1-NSP | RB1/2-NSP | RB4-NSP |
| 21SBI221114 | 25951 | RB1/2-NSP | RB1-NSP | RB1/2-NSP | RB1/2-NSP | RB1/2-NSP | RB2-SPL |
| 21SBI221114 | 25951 | RB1/2-NSP | RB3-NSP | RB1/2-NSP | RB3-NSP | RB3-NSP | RB3-NSP |
| 21SBI221114 | 25951 | RB2-NSP | RB3-NSP | RB1/2-NSP | RB2-NSP | RB3-NSP | RB3-SPL |
| 21SBI221114 | 25951 | RB1/2-NSP | RB3-NSP | RB1/2-NSP | RB2-NSP | RB3-NSP | RB2-NSP |
| 21SBI221114 | 25951 | RB1/2-NSP | RB2-NSP | RB1/2-NSP | RB3-NSP | RB1/2-NSP | RB2-SPL |
| 21SBI225251 | 26228 | RB0-NSP | RB0-NSP | RB0-NSP | RB0-NSP | RB0-NSP | RB0-NSP |
| 21SBI225251 | 26228 | RB0-NSP | RB0-NSP | RB0-NSP | RB0-NSP | RB0-NSP | RB0-NSP |
| 21SBI225262 | 26228 | RB0-NSP | RB0-NSP | RB0-NSP | RB0-NSP | RB0/1-NSP | RB0/1-NSP |
| 21SBI225262 | 26228 | RB0-NSP | RB0-NSP | RB0-NSP | RB0-NSP | RB0/1-NSP | RB0-NSP |
| 21SBI225262 | 26228 | RB0-NSP | RB0-NSP | RB0-NSP | RB0-NSP | RB0/1-NSP | RB0-NSP |
| 21SBI225262 | 26228 | RB0-NSP | RB0-NSP | RB0-NSP | RB0-NSP | RB0-NSP | RB0-NSP |
| 21SBI225262 | 26228 | RB0-NSP | RB0-NSP | RB0-NSP | RB0-NSP | RB0/1-NSP | RB0-NSP |
| 21SBI225262 | 26228 | RB0-NSP | RB0-NSP | RB0-NSP | RB0-NSP | RB0/1-NSP | RB0-NSP |
| 21SBI225282 | 26228 | RB0-NSP | RB0-NSP | RB0-NSP | RB0-NSP | RB0-NSP | RB0-NSP |
| 21SBI225282 | 26228 | RB0-NSP | RB0-NSP | RB0-NSP | RB0-NSP | RB0-NSP | RB0-NSP |
| 21SBI225282 | 26228 | RB0-NSP | RB0-NSP | RB0-NSP | RB0-NSP | RB0-NSP | RB0-NSP |
| 21SBI225282 | 26228 | RB0-NSP | RB0-NSP | RB0-NSP | RB0-NSP | RB0-NSP | RB0-NSP |
| 21SBI225239 | 26229 | RB0-NSP | RB0-NSP | RB0-NSP | RB0-NSP | RB0-NSP | RB0-NSP |
| 21SBI225239 | 26229 | RB0-NSP | RB0-NSP | RB0-NSP | RB0-NSP | RB0-NSP | RB0-NSP |
| 21SBI225239 | 26229 | RB0-NSP | RB0-NSP | RB0-NSP | RB0-NSP | RB0-NSP | RB0-NSP |
| 21SBI225239 | 26229 | RB0-NSP | RB0-NSP | RB0-NSP | RB0-NSP | RB0-NSP | RB0-NSP |

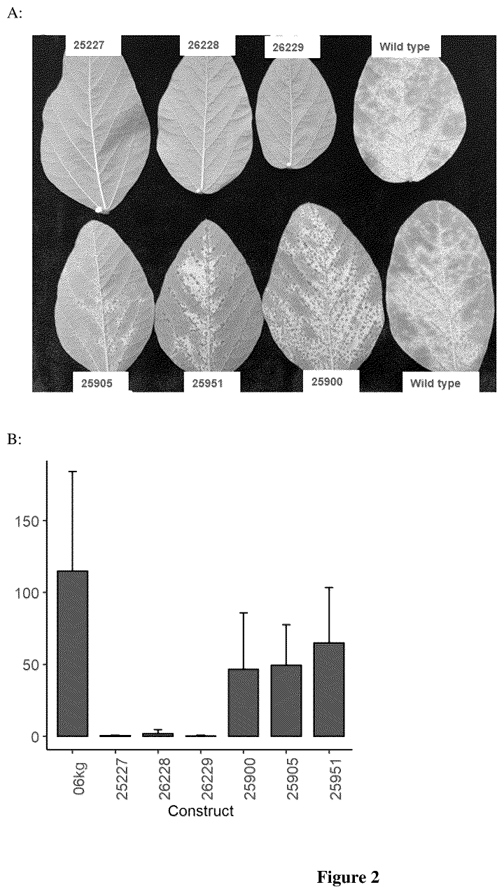

As further illustrated at FIGS. 1A and 2A, rust bioassay experiments conducted on leaves collected from TO soybean events comprising any one of constructs 25227, 26228, 26229, 259065, 25951 and 25900 showed either no lesions or small reddish-brown lesions while leaves from the control showed tan reaction and were heavy sporulating against the tested rust populations (population 21BRM shown in FIG. 2A, population 21BR08 shown in FIG. 1A). The T0 events repeatedly showed a high level of disease resistance compared to the control. Resistance evaluations of subsequent generations further confirmed that Rg31 or Rg35 are the causative genes.

Quantitative measurements taken using fungal β-tubulin transcripts were consistent with the phenotypic observations, as illustrated at FIGS. 1B and 2B. The level of resistance was measured molecularly with fungal β-tubulin via qRT-PCR on the event and the control. Each of the events comprising either the Rg31 or Rg35 gene (events from any one of constructs 25227, 26228, 26229, 259065, 25951 and 25900) showed a high level of resistance with more than 90% reduction in fungal biomass as compared to the control. In this validation, we demonstrated that the Rg31 and Rg35 genes show strong resistance (>90%) and broad spectrum against all rusts tested.

### IV. Expression of RG35 Confers Higher Resistance to ASR Relative to Expression of RG31

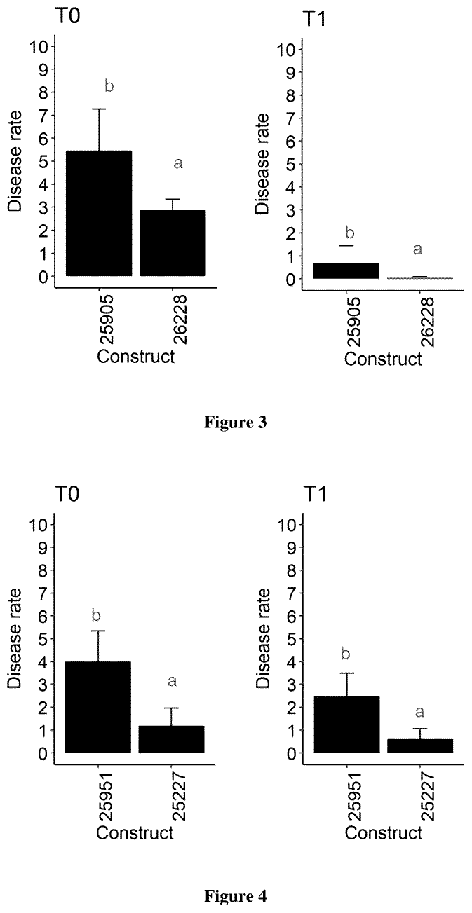

Rust bioassay experiments were conducted on single-copy T0 events and single-copy T1 events comprising either the RG35 gene (constructs 26228 and 25227) or the RG31 gene (constructs 25905 and 25951). FIG. 3 shows a comparison of the disease rates with expression of the Rg31 gene (construct 25905) and Rg35 gene (construct 26228) driven by a Medicago promoter (prMt51186, SEQ ID NO: 12). The average and standard deviation of disease rate for each construct was calculated. T-test analysis of the data revealed that events comprising the RG35 gene exhibited significantly higher resistance to soy rust as compared to events comprising the RG31 gene. The same trend was observed for T0 events (FIG. 3, left) and T1 events (FIG. 3, right). The results indicate that Medicago promoter driven RG35 expression has higher disease resistance than Medicago promoter driven RG31 expression.

FIG. 4 shows a comparison of the disease rates with expression of the Rg31 gene (construct 25951) and Rg35 gene (construct 25227) driven by their corresponding native promoter (that is, RG31 expression was driven by the native RG31 promoter of SEQ ID NO: 7 or 8 and RG35 expression was driven by the native RG35 promoter of SEQ ID NO: 10). The average and standard deviation of disease rate for each construct was calculated. T-test analysis of the data revealed that events comprising the RG35 gene exhibited significantly higher resistance to soy rust as compared to events comprising the RG31 gene. The same trend was observed for TO (FIG. 4, left) and T1 (FIG. 4, right) events. The results indicate that native promoter driven RG35 expression has higher disease resistance than native promoter driven RG31 expression.

### Example 3: Assaying Variant Rg31 and Rg35 Polypeptides and Variant Rg31 and Rg35 Genes for Mode/Site of Action

Variants of the RG31 and RG35 polypeptides and their genes, including ortholog sequences from other organisms, paralog sequences from the same organism, as well as allelic variants can be identified based on their sequence identity and/or functional identity with the RG31 and RG35 polypeptides and genes disclosed herein. In one example, variant polypeptides and polynucleotides of the RG31 and RG35 polypeptides, and polynucleotide sequences encoding such polypeptides, comprise proteins that interact with the same set of effector proteins to generate a localized hypersensitive response via a common mode of action when expressed in a plant. Variant polypeptide and polynucleotides including orthologs, paralogs, and allelic variants of the RG31 polypeptide of SEQ ID NO: 1 or the RG35 polypeptide of SEQ ID NO: 2 or 20 or the Rg31 gene of any of SEQ ID NOS: 3-4 or the Rg35 gene of any of SEQ ID NOS: 5-6 or 21-22 are expected to interact with effector proteins that interact with the RG31 and/or RG35 polypeptides of SEQ ID NOS: 1 and/or 2 and/or 20 to generate a localized hypersensitive cell death response.

Accordingly, in some embodiments, methods are disclosed of identifying novel disease resistance polypeptides having the same mode and/or site of action as the RG31 and RG35 polypeptides of SEQ ID NOS: 1 and 2 or 20, respectively, the method comprising: identifying a set of plant pathogen effector proteins that interact with the RG31 polypeptide of SEQ ID NO: 1 or the RG35 polypeptide of SEQ ID NO: 2 or 20, assaying for interaction of the identified set of plant pathogen effector proteins with a putative RG31-like or RG35-like polypeptide having at least 40%, at least 50%, at least 60%, at least 70%, at least 80%, at least 90% or at least 95% sequence identity to SEQ ID NO: 1 or SEQ ID NO: 2 or 20; and in response to detection of interaction between the putative RG31-like or RG35-like polypeptide and the identified effector(s), such as via the presence of a hypersensitive response or localized cell death, indicating that the putative RG31-like or RG35-like polypeptide has the same mode of action as the RG31 or RG35 polypeptide. In some embodiments, the effector proteins are expressed in a cell, purified, and infiltrated into a plant cell for an interaction assay with the putative RG31-like or RG35-like polypeptide. In other embodiments, a transient expression system, such as an Agrobacterium-mediated transient expression system in a plant can be used to observe the hypersensitive response cell death phenotype (HR) triggered by expression of a putative Rg31-like or Rg35-like gene comprising a Rg31 or a Rg35 variant (e.g., Rg31 or Rg35 orthologs or homologs or allelic variants) and their putative cognate effector (e.g., an effector protein recognized by the RG31 polypeptide of SEQ ID NO: 1 or the RG35 polypeptide of SEQ ID NO: 2 or 20). Presence of an interaction, e.g., presence of an HR response and localized cell death, indicates that the protein encoded by the putative R-genes have functional identity and a common mode of action as the novel R-polypeptides of the disclosure (that is, same mode of action as RG31 as set forth in SEQ ID NO: 1 or RG35 as set forth in SEQ ID NO: 2 or 20, and active variants and fragments thereof) of the present disclosure, even if the sequence identity is low (e.g., lower than 60% sequence identity, such as 50% identity or 40% identity or lower) or even if the sequence identity is high (e.g., higher than 90% sequence identity, such as 92% identity or 95% identity or higher).

I. Identification of Effector Proteins that Interact with RG31 and RG35

More than 300 curated soy rust effectors were assessed for interaction with RG31 and/or RG35. Of the more than 300 effectors, two effectors, SPE-35 (SEQ ID NO: 16 (protein sequence); SEQ ID NO: 17 (nucleotide sequence)) and SPE-35 (SEQ ID NO: 23 (protein sequence); SEQ ID NO: 24 (nucleotide sequence)) were recognized when assessed using heterologous Nicotiana spp. Agrobacterium strains containing R-gene RG31 or RG35 or a given soy rust effector were grown in Luria-Bertani agar plates, supplemented with 50 μg/mL spectinomycin and 50 μg/mL kanamycin, overnight at 22 °C in a dark chamber. The cultures were scooped with a toothpick and diluted in induction medium (5 mM MgSO₄, 2 mg/ml MES, and 10 μM acetosyringone, adjusted to pH 5.6 with HCl). Bacterial concentrations were measured and adjusted with induction medium to OD₆₀₀=0.5. Resulting cultures were pre-induced for 2 to 3 h at room temperature. For co-infiltrations, cultures carrying individual constructs were induced separately and mixed in a 1:1 ratio just before infiltration. Young tobacco leaves were inoculated with Agrobacterium cultures using a blunt syringe. The Control construct expressed GUS (Beta-glucuronidase). Cell death was observed 3-5 h after infiltration.

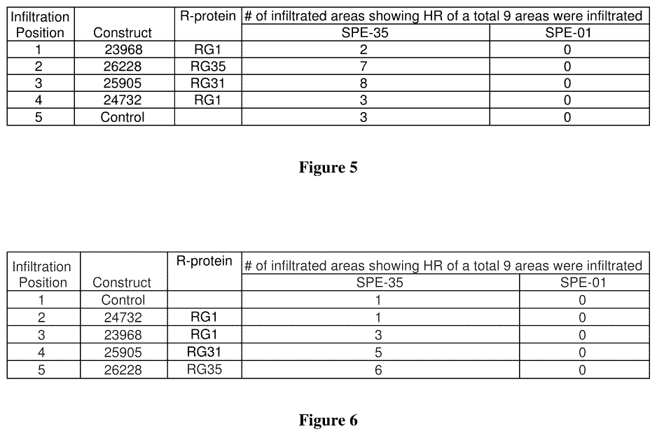

As shown at FIGS. 5-6, soy rust effector SPE-35, when expressed in N. tabacum via Agrobacterium mediated transient assay, specifically triggered a localized cell death response when co-infiltrated with construct 25905 (expressing the RG31 protein) or construct 26228 (expressing the RG35 protein). A localized cell death response was not observed when the soy rust effector SPE-35 (SEQ ID NO: 16 (protein sequence); SEQ ID NO: 17 (nucleotide sequence)) was co-infiltrated with the control construct, or when constructs 25905 or 26228 were co-infiltrated with an alternate soy effector, such as SPE-01 (SEQ ID NO: 18 (protein sequence); SEQ ID NO: 19 (nucleotide sequence)). This indicates that RG31 and RG35 specifically recognize and interact with SPE-35 over other effector proteins.

II. Mode of Action of RG31 and RG35 is Distinct from that of RG1

Putative RG31 and RG35 variants can be identified by co-expression of the putative RG31 or RG35 variant in N. tabacum via Agrobacterium mediated transient assay. Triggering of a localized cell death response when co-infiltrated with soy rust effector SPE-35 will indicate that the putative RG31 or RG35 variant has the same mode of action as RG31 and/or RG35. Similarly, an R-protein having high sequence identity with the RG31 and/or RG35 protein can be distinguished in terms of its mode of action from the RG31 and/or RG35 protein through the interaction with distinct sets of effector proteins.

An example of this is shown at FIGS. 5-6, wherein interaction between an effector protein and the RG31 or the RG35 proteins is analyzed through the presence of an infiltrated area showing a Hypersensitive Response (HR) in Plant cells. Soy rust effectors (in this example, SPE-35 and SPE-01) were expressed in different halves of N. tabacum leaves via Agrobacterium mediated transient assay with RG31, RG35, or RG1. RG31 (SEQ ID NO: 1) and RG35 (SEQ ID NO: 2 or 20) have high sequence homology to the RG1 resistance protein disclosed in WIPO Publication No. WO2019103918A1 as SEQ ID NO: 47.

With reference to FIG. 5, the effector protein SPE-35 was found to specifically trigger a hypersensitive localized cell death response at the site of inoculation when co-infiltrated with RG31 (construct 25905; infiltration position 3) or RG35 (construct 26228; infiltration position 2). Co-infiltration of the effector with RG1 (construct 23968, infiltration position 1; or construct 24732, infiltration position 4) did not trigger a similar response, indicating the specificity of SPE-35 for RG31 and RG35. Co-infiltration of the effector with the control construct expressing GUS (R-gene control, infiltration position 5) also did not trigger a hypersensitive localized cell death response at the site of inoculation, as expected. Co-infiltration of a different effector, SPE-01, with the construct expressing the RG31 or RG35 proteins also did not trigger a hypersensitive localized cell death response at the site of inoculation.

FIG. 6 shows a replicate of the same experiment wherein SPE-35 specifically triggered a hypersensitive localized cell death response at the site of inoculation when co-infiltrated with RG31 (construct 25905; infiltration position 4) or RG35 (construct 26228; infiltration position 5) but did not trigger a response when co-infiltrated with RG1 (construct 23968, infiltration position 3; or construct 24732, infiltration position 2). In both experiments (FIGS. 5 and 6), the number of infiltration areas showing an HR response with SPE-35 was significantly higher for RG31 and RG35 as compared to Rg1, or the control construct. Co-infiltration of a different effector, SPE-01, with the construct expressing the RG31 or RG35 proteins also did not trigger a hypersensitive localized cell death response at the site of inoculation.

These results suggest that the mode of action for resistance proteins RG31 and RG35 is surprisingly different from the mode of action for resistance protein RG1, despite the sequence similarity.

The above examples clearly illustrate the advantages of various embodiments of the invention. Although the present invention has been described with reference to specific details of certain embodiments thereof, it is not intended that such details should be regarded as limitations upon the scope of the invention except as and to the extent that they are included in the accompanying claims.

Throughout this application, various patents, patent publications and non-patent publications are referenced. The disclosures of these patents, patent publications and non-patent publications in their entireties are incorporated by reference herein into this application in order to more fully describe the state of the art to which this invention pertains.

1. A DNA construct comprising a polynucleotide operably linked to a heterologous regulatory element, wherein the polynucleotide encodes a polypeptide comprising:
(a) an amino acid sequence having at least 90% identity to SEQ ID NO: 1 or 2 or 20, wherein increased expression of the polypeptide in a plant increases disease resistance in the plant;
(b) an amino acid sequence having at least 95% identity to SEQ ID NO: 1 or 2 or 20, wherein increased expression of the polypeptide in a plant increases disease resistance in the plant; or
(c) an amino acid sequence comprising SEQ ID NO: 1 or 2 or 20.

2. The DNA construct of claim 1, wherein the polynucleotide that encodes the polypeptide comprises:
(a) a nucleotide sequence having at least 90% sequence identity to any of SEQ ID NOS: 3, 4, 5, 6, 21 or 22;
(b) a nucleotide sequence having at least 95% identity to any of SEQ ID NOS. 3, 4, 5, 6, 21 or 22; or
(c) a nucleotide sequence comprising any of SEQ ID NOS: 3, 4, 5, 6, 21 or 22.

3. The DNA construct of claim 1, wherein said heterologous regulatory element comprises a promoter active in a plant.

4. The DNA construct of claim 3, wherein said promoter is a tissue-specific promoter or a constitutive promoter or a rust-reactive promoter.

5. The DNA construct of claim 1, wherein said heterologous regulatory element comprises a terminator sequence, an intron, a 5′ UTR or a 3′ UTR.

6. The DNA construct of claim 5, wherein said polynucleotide encoding the polypeptide is operably linked to a promoter sequence comprising:
(a) a nucleotide sequence having at least 95% identity to any one of SEQ ID NOS: 7, 8, 10, 12, 14, or 15, wherein said nucleotide sequence is capable of driving expression of the polypeptide of interest in plant cell; or
(b) a nucleotide sequence comprising any one of SEQ ID NOS: 7, 8, 10, 12, 14, or 15.

7. The DNA construct of claim 1, wherein the polynucleotide encoding the polypeptide comprises at least 1 native intron or at least one heterologous intron.

8. A vector comprising the DNA construct of claim 1.

9. A vector comprising a polynucleotide encoding a polypeptide comprising:
(a) an amino acid sequence having at least 90% identity to SEQ ID NO: 1 or 2 or 20, wherein increased expression of the polypeptide in a plant increases disease resistance in the plant;
(b) an amino acid sequence having at least 95% identity to SEQ ID NO: 1 or 2 or 20, wherein increased expression of the polypeptide in a plant increases disease resistance in the plant;
or,
(c) an amino acid sequence comprising SEQ ID NO: 1 or 2 or 20.

10. The vector of claim 9, wherein said polynucleotide comprises (a) a nucleotide sequence having at least 90% sequence identity to any one of SEQ ID NOS: 3, 4, 5, 6, 21 or 22;
(b) a nucleotide sequence having at least 95% identity to any one of SEQ ID NOS: 3, 4, 5, 6, 21 or 22; or
(c) a nucleotide sequence comprising any one of SEQ ID NOS: 3, 4, 5, 6, 21 or 22.

11. A cell comprising the DNA construct of claim 1.

12. A cell comprising a heterologous polynucleotide comprising encoding a polypeptide comprising:
(a) an amino acid sequence having at least 90% identity to SEQ ID NO: 1 or 2 or 20, wherein increased expression of the polypeptide in a plant increases disease resistance in the plant;
(b) an amino acid sequence having at least 95% identity to SEQ ID NO: 1 or 2 or 20, wherein increased expression of the polypeptide in a plant increases disease resistance in the plant;
or,
(c) an amino acid sequence comprising SEQ ID NO: 1 or 2 or 20.

13. The cell of claim 12, wherein said polynucleotide comprises:
(a) a nucleotide sequence having at least 90% sequence identity to any one of SEQ ID NOS: 3, 4, 5, 6, 21 or 22;
(b) a nucleotide sequence having at least 95% identity to any one of SEQ ID NOS: 3, 4, 5, 6, 21 or 22; or
(c) a nucleotide sequence comprising any one of SEQ ID NOS: 3, 4, 5, 6, 21 or 22.

14. The cell of claim 12, wherein said cell is a plant cell, optionally wherein the plant cell is (a) a monocot cell, (b) a dicot cell, (c) a legume cell, (d) a soybean cell, (e) a barley cell, a maize cell, an oat cell, a rice cell, a sorghum cell, a sugarcane cell or a wheat cell; or (f) a sunflower cell, a tomato cell, a cotton cell, a sugar beet cell or a tobacco cell.

15. The plant cell of claim 14, wherein said polynucleotide is stably integrated into the genome of the cell, and wherein the plant cell has an increased level of expression of the polypeptide and the plant cell has increased disease resistance relative to a control plant cell.

16. A plant comprising the plant cell of claim 15.

17. The plant of claim 16, wherein the transgenic plant has increased resistance to Asian Soybean Rust, wherein the transgenic plant is a legume plant, optionally wherein the legume plant is a soybean plant.

18. A method of producing a plant having an increased disease resistance comprising:
(a) introducing into the genome of a plant cell, a heterologous polynucleotide encoding a polypeptide comprising:
i) an amino acid sequence having at least 90% identity to SEQ ID NO: 1 or 2 or 20;
ii) an amino acid sequence having at least 95% identity to SEQ ID NO: 1 or 2 or 20; or,
iii) an amino acid sequence comprising SEQ ID NO: 1 or 2 or 20; and
(b) regenerating the soybean plant cell of (a) into a soybean plant,
wherein expression of said polypeptide in the plant confers the plant with increased disease resistance relative to a control plant not expressing said polypeptide.

19. The method of claim 18, wherein said heterologous polynucleotide comprises:
(a) a nucleotide sequence having at least 90% sequence identity to any one of SEQ ID NOs: 3, 4, 5, 6, 21 or 22;
(b) a nucleotide sequence having at least 95% identity to any one of SEQ ID NOS: 3, 4, 5, 6, or 21; or
(c) a nucleotide sequence comprising any one of SEQ ID NOS. 3, 4, 5, 6, 21 or 22.

20. The method of claim 18, wherein said plant is a legume plant.

21. The method of claim 20, wherein said legume plant is a soybean plant.

22. The method of any claim 20, wherein said increase in disease resistance comprises an increased resistance to Asian Soybean Rust.

23. The method of claim 18, wherein introducing said heterologous polynucleotide into the plant genome is through transformation of the plant cell with a DNA construct comprising said heterologous polynucleotide.

24. A method of decreasing Asian Soybean Rust damage or controlling an ASR pathogen in an area of cultivation comprising the planting in said area of cultivation a plant of claim 17.

25. (canceled)

26. A method of controlling disease resistance in an area of cultivation comprising the step of planting in the area of cultivation a soybean plant, or a seed having stably incorporated into its genome:
(a) a nucleotide sequence encoding a RG31 polypeptide having at least 90%, 91%, 92%, 93%, 94%, 95%, 96%, 97%, 98% 99% or 100% identity to SEQ ID NO: 1; or a heterologous nucleotide sequence encoding a RG35 polypeptide having at least 90%, 91%, 92%, 93%, 94%, 95%, 96%, 97%, 98% 99% or 100% identity to SEQ ID NO: 2 or 20; and
(b) a heterologous promoter operably coupled to the nucleotide sequence,
wherein expression of the RG31 polypeptide, or RG35 polypeptide confers increased Asian Soy Rust disease resistance to the legume plant, plant or seed.

27. (canceled)

28. The method of claim 26, wherein the heterologous promoter comprises a nucleotide sequence having at least 95% sequence identity to any one of SEQ ID NOS: 7, 8, 10, 12, 14 and 15 or wherein the promoter comprises the sequence of any one of SEQ ID NOS: 7, 8, 10, 12, 14 and 15.

29-34. (canceled)

35. A composition comprising:
(a) a RG31 polypeptide, or active variant thereof, comprising an amino acid sequence having at least 90%, 91%, 92%, 93%, 94%, 95%, 96%, 97%, 98%, 99%, or 100% identity to SEQ ID NO:
1; or
(b) a RG35 polypeptide, or active variant thereof, comprising an amino acid sequence having at least 90%, 91%, 92%, 93%, 94%, 95%, 96%, 97%, 98%, 99%, or 100% identity to SEQ ID NO: 2 or 20,
wherein presence of said composition in a plant confers the plant with increased disease resistance relative to a control plant not comprising the composition.

36. (canceled)

37. A plant comprising in its genome a stably integrated nucleic acid molecule, the nucleic acid molecule comprising a heterologous promoter operably coupled to:
(a) a polynucleotide encoding a RG31 polypeptide, or active variant thereof, comprising an amino acid sequence having at least 90%, 91%, 92%, 93%, 94%, 95%, 96%, 97%, 98%, 99%, or 100% identity to SEQ ID NO: 1; or
(b) a polynucleotide encoding a RG35 polypeptide, or active variant thereof, comprising an amino acid sequence having at least 80%, 85%, 90%, 91%, 92%, 93%, 94%, 95%, 96%, 97%, 98% 99% or 100% identity to SEQ ID NO: 2 or 20,
wherein expression of said nucleic acid molecule in the plant confers the plant with increased disease resistance relative to a control plant not comprising the nucleic acid molecule.

38. The plant of claim 37, wherein the polynucleotide encoding the RG31 polypeptide, or active variant thereof, comprises a nucleotide sequence having at least 90%, 95%, 98%, 99% or 100% identity to any one of SEQ ID NOS: 3-4; and wherein the polynucleotide encoding the RG35 polypeptide, or active variant thereof, comprises a nucleotide sequence having at least 90%, 95%, 98%, 99% or 100% identity to any one of SEQ ID NOS: 5-6 or 21-22.

39. The plant of any one of claims 37-38, wherein the plant is a soybean plant or an elite soybean plant, and wherein the plant has increased resistance to Asian Soy Rust.

40-41. (canceled)


SEQUENCE LISTING (24 sequences)

SEQ ID NO: 1 - protein, 1735 aa, Glycine tomentella
MASISSVQSSSSAYDVFLSFHGEDTRNNNNNSTGFLFQALRRKGIVAFKGHTLPEKGEPELLHAIEGSRLFIVVLSKNYASSTRCLRELAHIFNCIETSPRRLLPIFYDVDPSDVRKQTGWYEEAFWKHEQRFLVDKERMEEVRTWRKALTRVANLPGWYIPNKPQYTEIEEFVQYIITILGPNISTLQNEAEGTSSDKDEVLYNELSGLMRIRDFISLVLTYPGKLEDENILRSWISAFPEWKAQLLSTGDDTVMDKPWCVLTDKALGPMKGFMPMEIISKVESGERSIFSHNDVDAIPEAILQQSVDLAVNQLLQTLFSGDYCARYIRLLTRNLAEKGSVVDTIVSAVEDKHTMFGIDKDFLRALWINASTYETDAEVRVQEQIDINMMMEVDDMGSNGQLVIVVDADSNNKLDLQKLRFPTGIVVLITTEPSTQAEKDVDFRIACTMDLNIWTQDHLLPWKVFCSYVGSCISSSMVSSSMAIQKIAEEIVKECHGHLLAIVLVAKYLTYVQDFKQWELALDKLSSLNPSYDYQDSDRIGISRVMVNAFVNIIWEDIDDAQKLCLELSLPVHNIKNGVRDDILVSDWAKIISGYTQELGEYRRQLQHYINELQDRFVLLKYESGDVYLPVETYDIIKSLHTSKPSILRHGALGLTEPPYIGRWHSLVQIELMDNKICELPQSPDCPKLKVVLLQGNVDLMDIPDSFFNHMPLLQHLDLSYTSIRDLPPSVSKLIQLQKFYLRGCDLFMELPHQIGQLKNLKELDLDGTLITHLPIEIRELINLQSLSLCFDGTSRSTIIPPGLVSNLTQLNYLSINVDPEDERWNENVISVLREIFFGLWNLQRLSIYVPKADLLEFIPTQKSLNFRLVVGQHMRRLISRVPPELETKFKHCDYSIKFVNGVNVPNGVKMHLGRFKALYLDRHMTIKSLSDFDLRNLWGLEVCILAECNEMETIVSGSNSPDRPASLMLKFLSVFYMKNLRSVCEGSSPFFLCLKSIALHTCPMLTTIFTLDSLKKLSFLEEIIIEDCPKVTTLISHATPEQKPVFFLPKLRMISLLYLPELVNIFNGIHVGPFIEEMIFYYCPSLQSLSKSELSSKSLKIIKGESIWWEALKWNVAEWGDAGRPNYFDRIFSPINEEADMMNQLAALQETRLNEHHNTTYQGISSSTHSIAKSSLEIPVLSPLAPVSSVGRGSSNSEAQKRKVVTEPKILQDASKKIIKQVREQRTVQTEVWTEPEVWTEKISVNAEGYLNDGYSWRKYGQKSPLGAKFPRGYYKCAYGSVQGCPARKQVQRLDENPATFEITYQGRHTCTKEQNLIIKSGDEGISSSTQSIAKSSLEIPVLSPSAPVSSVGRGSSNIEAQKRKVVTEPKIFQDASEKCNLIMKKGNLGLGLKLSVPVSDQSNFAKFLDGVRIVSQNDVEAPPQIKPTDNQLTLADIDVIKVVGEGNGGVVQLVQHKWTRQFFVLKVIQMNIEESMRKQIAQELKITQQAQCPYVVVCYQSFYENGVISILLEYMDGGSLADLLKKVKTIPEDYLAAICKQVLKGLVYLHHEKHIIHRDLKPSNLLINHIGEVKITDFGVSTIMESTSGQANTFIGTYNYMSPERIIGSQRGYNYKSDIWSLGLILLECALGRFPYAPPDQSETWESIFELMETIVDKPPPSPPSEQFSTEFCSFISACLQKDPKDRLSAQELMAHPFVNMYDDLEVDLSAYFSNADELKNQLLESLQGFCI

SEQ ID NO: 2 - protein, 1728 aa, Glycine tomentella
MASISGVQSSSSAYDVFLSFHGEDTRNNNNNFTGFLFQALRRKGIVAFKGHTLPEKGEPGLLHAIEGSRLFIVVLSKNYASSTRCLRELAHIFNCIETSPRRLLPIFYDVDPSDVRKQTGRYEEAFWKHEQRFLVDKERMEEVRTWRKALSRVANLPGWYIPNKPQYTEIEEFVQYIITILGPNISTLQNEAEGTSSDKDEVSYNELSGLMRIRDFISLVLTYPGKLEDENILRSWISAFPEWKAQLLSTGDDTVMDKPWCVLTDKALGPMKGFMPMEIISKVESGERSIFSHNDVDAIPEAILQQSVDLAVNQLLQTLFSGDYCARYIRLLTRNLAEKGSVVDTIVSAVEDKHTMFGIDKDFLRALWINASTYETDAEVRVQEEIDINMMMEVDDMGSNGQLVIVVDADSNNKLDLQKLRFPTGIVVLITTEPSTQAEKDVDFRIACTMDLNIWTQDHLLPWKVFCSYVGSCISSSMVSSSMAIQKIAVEIVKESHGHLLAIVLVAKYLTYVQDFKQWELALDKLSSLNPSYDYQDSDRIGISRVMVNAFVNIIWEDIDDAQKLCLELSLPVHNIKNGVRDDILVSDWAKIISGYTQELGEYRRQLQHYINELQDRFVLLKYESGDVYLPVETYDIIKSLHTSKPSILRHGALGLTEPPYIGRWHSLIQIELMDNKICELPQSPDCPKLKVLLLQGNVDLMDIPDSFFNHMPLLQHLDLSYTSIRDLPPSVSKLIQLQKFYLRGCDLFMELPHQIGQLKNLKELDLDGTLITHLPIEIRELINLQSLSLCFDGTSRSTIIPPGLVSNLTQLNYLSINVDPEDERWNENVISVLREIFFGLWNLQRLSIYVPKADLLEFIPLLPAQKSLNFRLVVGQHMRRLISRVPPELETKFKHCDYSIKFVNGVNVPNGVKMHLERFKALYLDRHMTIKSLSDFDLRNLWGLEVCILAECNEMETIVRGSNSLMLKFLSVFYMKNLRSICEGSSPFFLCLKSIALHTCPMLTTIFTLDSLEKLSFLEEIIIEDCPKVTTLISHATPEQKPVFFLPKLRMISLLYLPELVNIFNGMHVGPFIEEMIFYYCPSLQSLSKSELSSKSLKIIKGESIWWEALKWNVAEWGDAGRPNYFERIFSPINEEADMMNRLAALQETQLNEYHNTTYQGISSSTQSIAKSSLEIPVLSPLAPVSSVGRGSSNSEAQKRKVVTEPKILQDASKKVREQRTVQTEVWTEPEVWTEKISVNADGSLDDGYSWRKYGLKKILGAKFPRGYYKCTYGSERGCPARKQVQRLDENPATFEITYQGRHTCTKEQNLIIKSGDEGISSSTQSIAKSSLEIPVLSLSAPVSSVGRGSSNIEAQKRKVVTEPKIFQDASKKCNLIVKKGNLGRDLSVMCGTFKDGDLLVNRDGVRIVSQNDVEAPPQIKPTDNQLTLADIDVIKVVGEGNGGVVQLVQHKWTRQFFVLKVIQMNIEESMRKQIAQELKITQQAQCPYVVVCYQSFYENGVISILLEYMDGGSLADLLKKVKTIPEDYLAAICKQVLKGLVYLHHEKHIIHRDLKPSNLLINHIGEVKITDFGVSTIMESTSGQANTFIGTYNYMSPERIIGSQRGYNYKSDIWSLGLILLECALGRFPYAPPDQSETWESIFELMETIVDKPPPSPPSEQFSTEFCSFISACLQKDPKDRLSAQELMAHPFVNMYDDLEVDLSAYFSNADELKNQLLESLQGFCI

SEQ ID NO: 3 - DNA, 17357 bp, Glycine tomentella
  source 1..17357: mol_type = genomic DNA
catttagtaaaatctgtgcacaagaggtgctggcagaactagacatgcatgaatctttcttaactgttgcttctgattcccccgagatggatctcaattcttaacagttagcagcagaggtacacatgaatctttcttacttaactcttgctcccgattcctcctcagatacattccaatcattttgtatatcagtttcaacatggacggattttggggggtatgggggaagcaggggccttggccctccctcaagatcctttatatatacttgagaaaaaagaaaaagaaaatattactagtataaaatgaataagaaaaatagagtattgacttgatgtttctttaatttttttttctagtagtagttcccgccacaggccacagcttttcttccattggatcccattttatcccaactccagcaatcgagaaatacttgtccttaatttacaccttctgtccttcttcacaaacaagtttacttccactttcgcaattccaatggcttctatctccagcgtccagagcagctcttctgcatatgacgtgtttctcagcttccacggtgaagacacacgcaacaacaacaacaactccacaggttttctttttcaagctctcagaagaaaaggcatcgtcgccttcaaaggtcatacacttcccgaaaagggtgaacccgagctgttacatgcaattgaaggctcccgccttttcattgtggtcctttcaaagaactatgcttcctcaactcggtgtttgcgtgaactagcacacatcttcaattgcattgaaacttcgccaagacgtcttctgcctattttttatgatgttgatccatcagatgtgcgaaaacagactggatggtacgaggaagccttttggaaacacgaacaaagattcttggtagataaagagaggatggaggaagtgcggacatggagaaaagctctgactcgagtggccaatctccctggttggtatatcccaaataagtaagtaacccgctctcatctcatgaaagatgtaacttgcaaattgggtccaaatttaatatagatgtttgatgcaaatggtatttaaaagttgaaaactactcttctattatcttctcctgttttgcttctcttgggtgatgatttcgaattcttcattttctttaaaatttcaggccacaatatacagagattgaagaatttgttcaatatataataactatattgggtcctaatatttcaactcttcaaaatgaagcagagggaacatcttcagataaagatgaagttttatacaatgagctgtcaggcttgatgaggataagagattttatcagcctcgtgttgacttatcctgggaaattagaagatgaaaatattttaagatcttggattagtgcatttcctgaatggaaagctcagttattgtccactggtgatgacactgtgatggacaaaccatggtgtgtactaactgataaggcactgggacctatgaagggatttatgccaatggaaataataagtaaagtagaaagtggtgagagatccatcttcagccataatgatgttgatgcaataccagaagccattctccaacaatctgttgatctcgcagttaaccaactacttcaaactttgttttctggggattactgtgcacgctatatcagactgttaaccagaaacttggctgaaaaaggatctgtagtggacacaatagtttcagccgttgaagataagcacaccatgtttggaattgacaaagattttcttagagccctatggatcaatgcctcaacatatgagactgatgcagaggttcgagttcaagaacaaatcgatatcaatatgatgatggaagttgatgacatgggaagcaatgggcagttggtaattgtagtggatgcagatagcaacaacaagctggacctccaaaagttgcgttttccaactggaattgtggtgttgataacaactgagccgtcaacacaagcagagaaggatgttgactttagaattgcatgcacaatggatttgaatatttggacccaggatcatctattgccttggaaagttttctgcagctatgtcggcagttgtatcagcagtagcatggtaagttcatcaatggccattcagaaaattgcagaggaaatagttaaggaatgccatggtcatcttcttgccattgtccttgtggcaaagtatcttacgtatgtgcaagattttaaacagtgggaacttgcacttgacaaattaagcagtctgaatccttcctatgattatcaagatagtgatcgaattggcattagtagagtcatggtcaatgcattcgtaaatattatttgggaagacattgatgatgcacaaaagctttgccttgaactcagtttgcctgttcacaacattaaaaatggggtgagagatgatatcttggtgtctgattgggctaagataatatcaggatacacacaagaacttggtgaatacagaaggcaattacaacattacatcaatgagcttcaagaccgttttgtcttgttgaaatatgaaagtggagatgtctatttgccagtagaaacctatgatataataaaatcattgcacacctcaaagccttccatcctaaggcatggtgcattaggattgacagagccaccctacattggacgatggcatagtcttgttcagatagaattgatggacaataaaatatgtgaattaccacaatctccagattgccctaaactcaaagtagtgttgttgcagggcaatgttgatttgatggatatccctgactcatttttcaaccacatgcccctgcttcaacacttggacttatcctacactagtataagggatttacccccttctgtctctaaattaatacaacttcagaaattttatcttagaggctgtgatctcttcatggaactaccacatcagattggacagctaaagaacctcaaagagcttgatcttgatgggactttgatcacccatctgccaatagagatccgagaattgatcaacctgcaaagcttaagcctctgctttgatggaacttccaggtcaacaattattcctccaggattggtatcaaatcttactcaattgaattatttaagcatcaacgttgaccctgaagatgagcgatggaatgagaatgttattagcgtcttgagggaaattttttttggattatggaatttgcagaggttgagtatatatgtcccaaaggctgacctcttggaattcataccaacccaaaaatctctcaatttcagattggttgttggtcaacatatgcgacgtttgatatcgcgtgtaccacctgaactggagacaaaatttaaacactgtgactatagcataaagtttgttaatggtgtgaatgttccaaatggagtgaaaatgcatcttggacgcttcaaagccttgtacctagaccgacatatgaccatcaagagtctctctgatttcgatttgaggaatttatgggggttggaagtatgcatattggcagagtgtaatgaaatggaaacaattgtcagtggaagcaattcacctgataggccagcttctctaatgttgaagtttttgagtgtattctatatgaagaatttaagaagcgtctgcgaagggagtagtcccttctttctctgcttaaagtctatagcattacacacttgccccatgttaaccaccatcttcaccttagattctcttaaaaaactttcctttttggaggagattattattgaagactgtccaaaagtaaccaccctgataagtcatgccacaccagaacaaaaaccagtgtttttcctccccaagttgagaatgatatcgcttctctatcttcctgaattggtcaacattttcaatggcatacatgttggaccttttatagaagaaatgatattttattattgcccaagtctccaaagtctttccaaatcagagttgtcaagcaaatctttgaagatcatcaaaggagaaagcatctggtgggaagcattgaaatggaatgtagcagagtggggagatgctggtcgacccaattattttgaccgaattttttcaccaattaacgaggaggctgacatgatgaaccaactagctgcacttcaagagactcgactaaatgaacatcacaatacgacgtatcaggtaatttttagttatcctcttttcaaattgcatggtgttattggcatgttggtattttccaatgactaatgagttgattcttatttttactattttgtatctctgtatgaattgcttttgctaatatgatcataccctcttagcttcatttttcgtactgtattacttgaatgttttcctttcagggaatttcaagttctacacattcgatagcaaagtcatctttggaaatacctgttttgtcacctttagccccagtttcatctgtgggtaggggtagttcaaacagtgaagcccaaaagcggaaagtcgttactgagccaaaaatattacaagatgcgtcaaaaaagataataaagcaggtttgagatgccttgtcaccgttcgtgattgattgcttttggttttcttatatgcacgagttggtttacttacattaaaggcatgtttgataaacctgtaaaattatacgtcgaatgtgttttgcttgaaaagattataattttaagtgctgaaaaactcatttaattaagaaaacttaaaacatttagatgctaaacatggtgacaccaaaggaaattatgtcatttaattattcagatagggaagtagttgctgctttattcattcagtatcagcaatgtgctgaatgtatttaacatatttcacgtaaagaattattttcagtttttatatgactagttgccaatagcaacaaatagtggtgctagcgcagaattgcattgcagagcggctagagcatgctatgaaaattgaaaagtgtatcagtagcgaccatcacagctgtcatatctagcaaaatagcagaaattgctcctggagcaggctttattggagtgggtcacaaaaaaacctattttaatttaaccattaaacaactttatttagggtgttttagtgggcaatgattttgcccccttctttgaataacaactgaaactctactttctcaattacttacacacactgttatgacatattgttatcctttttccaccaagacacccttttctggcaacatttttggcaattccctgcattttcgggccttatgtgagttttttgccatccccaacctctgcagcttctattctccatagtttccagtcattcctgacctttatggccattttctggcgaacctgtcactgtttctggccaaccgcgacctttgctgccgtgctgttcactgttctgtccttgcattgttcatctctctaatacagatccttcatctctctgttttgtgctctcttctatatttttctttaacatttaatttaatatttgtcatattttattttattttattatcaattacgaaattattgatcaaagttttataagaaagtctaaagtatttctagcattttactgtcaatatataaaatgacaaaatcattcaaattatttcataacatagttaatatcaaataagattttagtatgtaacataaaaaagatttgagtaattttagtttgaaaagcaactttcaagaataataataataattactaggtttattaataatattagaaacaatgcatgtatttgaaagaaaaatacaatatttttaactaaacgatatttttaatgaaatatgaattttttttctatacttcttgcataaatgtcctaatgaggagaatttgcaaaaatttagagtagcgagaaaagatgcaaagaaagcagtgcgagaagctagaggaaaagtttttgatgatttgtataaaaatcttgggagcaaagatggggaaaaaggtatttataaacttgcaaagatgagagataagaagagtaaagatctcacccaagttagatgtatcaaggatgaaaatcaaaaacttttggtaagggatgaagatgtaagtgaaagatggttacactactttaacaatctttttaatggaaatggtgacaagagtgaggtggaattggaggacgtttctgatgatctgaatagaagatttgtgcgcagaattaatacatatgaggttaaagagactttaaagaggatgaagaataataaagcgttaggacctgatgatgtccctattgaggtgtggaggggattgggagatgtggcaataaaatggttaaccaatctttttaataggattcttcaaacaaacaagatgcctgatgcttggaggaaaagtatgcttgtgcctatttacaaaaacaagggagatattcaaaattgttcaaactatagaggcatcaaacttatgagccatactatgaagttgtgggagagagttatcgaacatcgtctaaggagggagaccaagatttcagtaaatcaatttggatttatgtcaggaagatgtaccatggaagctattttcctgatgagacaattgatggaaaggtatagagaaaggaaaaaagacttacatatggtgttcatagatctcgagaaagcatatgatagagtcccacggaaagtcttatggtggactcttgagaagaaaaaggtagcattaaagtatattaatgcaatcaaagatatgtatgatggggctaccacatgtgtgaaagcttgtaatggaacatcaaggtatttccctatcacggtcggtgtctaccaaggttcagctcttagtccttatctctttgccttagtgatggatgaactaacgaaagatattcaagaagaagtgccgtggtgcatgctctttgcagatgatgtagtacttattgatgagaccaaagatggcgttaatagaaaattggaattgtggagaagcactttggagtctaaaggattcaaggtaagtaggagcaagacggagtatatgaagtgtgagttttcgaggattaaagacggtgagcaagacacagtgcatctgcttgggcaagaggtacccaagaaagacgtttttagatatttgggatcaatgttgcaaaaaggtggggagatcgatgaggatgtaagtcatagaattaaagcagggtggatgaaatggcggagtgcatcggggtttttgtgtgaccgcaagattcctattaagctaaaaggaaaattctatagaacagctgttagaccggcaatgctttatggagcggaatgctggccagcacataggcaacatgagcataaaatagaggtagcagaaatgaggatgttaaggtggatgtgcggatacacgaagatggataagataagaaatgaggatattcgacgaaaggtatcagtggtaccagtatgtgaaaagatgagagagcatagattgagatggtttgggcatgttatgagacgaccattagatgctcctgttagaacaagagtatgttaccaagagctagatacaaagagaggtagaggtcgaccaaaacttacctggggaaaggtagttaaacaagacctaatggagtgggatttagattggcggacggtatgtcttaggacggagtggagaaaagctataagtctgatggagggttaaaccctttagtagttctatgttccagttgttccctgttttaattcttttcttcctcttttttcttctcctctcaccattcttttatatttaactctgtttctctcttttgctttattttcgatttatcattgggttcatctaccaccgaccccaattaatttgggttaaaggtgctgttgatgttgatattgatgttgacttcttgcaatatctttattttaaaaataaagaaaatattttcagtattataattcttagtagaacttaaaagtattttcaattcatatcttcatctaaagttttcaattgttcaattaaaatacaatattaaattacagtcttaataataaaaatagtaatatatatcgttataaaatttatcacctaataattcaaattaataataaatatttttcaaaaagtataaattttttaggcttaaggtaactacttaaatggccatggccttgtggttgccacagccctagttagaagtgaatcttataggggtctgaaggtgctaacagacggattctctccatttttagtggaatttgcttactctttatcagcaacttattttgttcattgctgtcacgaaaaagaaaaaaaggaaaggaaaacatcactttggaaatgtgtcaatcctatgaacctatgatgcatggtcatataagtagggagttatacgcctgcaacttgaaattgtttgttctctactttgactatgcatcaattataccacaaaagattcagaaggtagatattctctatattgcaaatcaatctttagcttcaacacaatgtaagctacttccccaactgaaaaatgatgatgatggttttattcatgaatcgagatcaaatactgttgctgagttcctctatgaagatgtgtccgtaggaggcgtctctatagtctttagttaaacaaaaataaacttcacgaacctaacatgttgaataaaagactattgtggtttttatattttggcatggcaattaacaggttcgtgaacaaagaacagttcaaactgaagtttggacagaacctgaagtttggacagaaaagataagtgtcaatgcggaagggtatttaaatgatggatatagctggagaaaatatgggcaaaagagtcctcttggagctaagtttccaaggttagttaaacggtcttttctcgtttaactttttcttgttataaaattaaacaagtttaattagcaatttttttcctagtataaatttatataattaacatttgatttgaatgtgacagaaaggttcacccctctaggttattgacgtacaagtttcacccctctcgtttagttaataccagaggccttggacctggtaactggaaaatcagtagccaattagctaataagttctctttcattgttttaatggttctatgcccccagtgtttttaaaatgacctgcaatttgaaaaattatctaaaggaccatgtcagctggtttcaaatgggcccaaattagtgctttctgttggaaacttgtttttgacattctcaacaaaaacttgttcatttgacaactacctttgcaaaaatttaaaaccgagaacaaccacaaactccagataacatctgattctttatttttggacatagactatactataacattcacagcaactaaaaagagaatgtaggtttcttctccaacttccttatcaaggcttaaaacaagaggggtttctattaatatttttatttgctgttcgtaccatactaactttgccagtctaatgtttatattaagaacataaaaagaagttattttctagttttgccgaaaaattgacaactagtcgtttgcattgcagaggatattacaaatgcgcatatggatctgtacaagggtgcccagcaagaaaacaagttcaaaggttagacgaaaacccagcaacatttgagataacctaccaaggaagacatacatgcaccaaagaacagaatttgatcattaagagtggggatgaggcaagtttgaacaaaaagactagtggtcaaatcttgcaagttataaaaaaacttgtcttgcaaatttgcattactaaaaataaaaatagacaaatttaaaatatattgctgatgttatctacatttcagtaaaaaaaatatcagtaaataacattatttaatatgatcataaatacatttcatccaaaaaatgttactggtaatccaccaaacttggaacatacaaaacatcaagctagtatccaatgaatgctaatcaaagattgttacaatgcatgtatctctggttgaatcatcttcatcatcaaaggttgcctacataacaatacaaaatacctcccattacaaggcaaaaaagtggggaaaaaatgggttaaatgattttatgtagtgaaactaaattgatttttggatgtttgaagaggggggtgggggatcaaaagtattcatgataaattaacataaaattataagaacgagaaattattagttgacttaaattatgttttttgtctatactaaatgctcaaagttcatatttaatctgtcattctaatcaaaacctatgagacatttgcttgttccagaaaacttgacagctgtaactttatatctctgtatgaattgcttttgctaatatgatcataccctcttagcttcattttttgtattttattacttgaatgttttcctttcagggaatttcaagttctacacagtcgatagcaaagtcatctttggaaatacctgttttgtcaccttcagcccctgtttcatctgtgggtaggggtagttcaaatattgaagcccaaaagcggaaagtcgttactgagccaaaaatatttcaagatgcatcagaaaaggtaataaagcaggtttgagatgccttgtcaccgttcgtgattcattgcttttggttttcttatatgcacgagttggtttacttacattaaaggcatgtttgataaacctgtaaaattatacgtcgaatgtgttttgcttgaaaattataattttaagtgctgaaaaactcatttaattaagaaaacttaaaacatttagatgctaaacatggtgacaccaaaggaaattattgtcatttaattattcagatagggaagtagttactgctttattcattcagtatcatcaatgtgctgaatgtattcaacatatttcacgtaaagaattattttcagtttttatatgactagttgccaatagcaacaaatagtggtgctagcgcagaattgcattccagagctgctagagcatgctatgaaaattgaaaagtgtatcagtttgaagtagcgaccatcacagctgccatatctagcaaaatagctgaaattgctcctggagcaggctttattggagtgggtcacaaaaaaacctattttaatttaaccattaaacaactttatttggggtgttttagtgggcaatgattttgcccccttctttgaatcacaaccgaaactctgctttctcaatcacttgcacacactgttatgacattgttatccttttcccacctgcacacccttttctggcaacatttttggcatttccctgcattttgggggcttctgtgagttttttgccatccccaacctctgcagcctctattctccatagtttccggtcattcctgacctttatggccattttctggcgaacctgtcactgtttctggccaaccgcgacctttgctgccgtgctctgttcactgttctgtccttggattgttcatctctctaataaagatccttcatctctctgttttgtgctctcttctataattttctttaacatttaatttaatatttgtcaaatttttatttatttattatcaattacgaaatcattgatcaaagttttataagaaagtataaagtatttctagcattttattgtcaatatataaaatgacaaagtcattcaaattatttcgtaacatagttaatatcaaataagattttagtatgtaacataaaaaagatttgagtaattttagttttaaaaactactttcaataataataagaattactagtttattaataatattagaaataatgcatgtatttgaaagaaaaatacaatatttttagtgaaatatgaatttctttttctatacttcttgcaatatctttattttaaaaataaatcaagattttcaatattataatttgcttactcttctgtatttttctttaatatttgtcatactttattttatttttttatcaattacgaaattattgatcaaagttttatagaaagtgtaaagtatttctagcattttattgtcaatatataaaatgacaaaatcattcaaattatttcaatatcaaataagattttagtatatcaaataagatttttagtatgttaaaaaagatttgagtatttttagtttaaaaaactacttttaataataataataataatatttacaagtattattaataatattagaaacaatgcatgtatttgaaagaaaaatacaattttttaaataaataatgtttttagtgaaatatgaatttttttgtctatccttcttgcaatatctttattttgaaaataaatcaagattttcaaaattataattcttagtagaacttaaaagtattttcaatccatatcttcatctaaagttttcaattgttcaattaatacaatcttaaaattacagtcttaataataaaaatagtaatatatgtcattataaaatttatcacctaataattcaaattaataataaatatttttcgaaaagtatacattttttaaggctaaaggtaactacttaactggccatggccttttggttgccacagccctagttagaagtgaatcttagaggggtctgaaagtgctaacagacggattctctccatttttagtgcaatttaatcatgaagaaaggaaacttggggcttggtctcaaactctcagttcctgtgtccgatcaatctaattttgccaagttcctgtgagtctctctctctctctctctttgtgatgtgtgggacttttaaggatggggatctgcttgtcaacagggatggagttcgaattgtctctcagaatgatgtcgaagcggtaacatatttctctcacttttttatgcattctttgcctttgtaattgtttgtttgttaacaatttgtttatttcattatgtgtccagtccgattgctccattgtttgtttttttgcatttgatcttttggatctgatgaaatgcctgatagataagataacaagggattcatgatttttctgtagattccttattctcaggttaccatacccaagtggagtattatatgatttaaattgaataaaaaaattatgagatggattatttgctgtttctccttggccattatttttcttgtgtgtgtgttttcggcggtattgaccctactttcacttttcctagcctttgctattggattttaaatgaacctgatattgttaacgaggttatgcatttcttatcttatttacattgtctttaatcaaatggacaacaagttgcaagtttatgaaatgaaattgtgctaagcatacgtcatccacttcatgagccactaaacaaattgagtctttcatttctactcttaaacgaagtttgttgcataaaattgtttgcattctgagttaatgttggatcctcttcttccttcctacgtctttttttttttttttttttggggggggggggtggggggactcagggaggctttcacactgtcaatcatgaaaaaaaataaaaaataatctgactaattatattggaaaacttcctatgcatactcttatatggtcaatacattttagaagacaaaatgagttgatagtagatacatagccatgttaaatgaggacccaaatttataagtgactaagtttaaatttattcctgttaaactccccatatctatagttgtgcattccaaccagtggcttatggtataaaaagagcaggacaagggggagttgaatgttgaaattcatgcatattttttttatctagtcacaatacacaatacattgctggctgaattctgacaatatgttgtatatgattctaacattcatgttattttttcagccacctcaaatcaagccgacagacaaccagttaactttggcagatatagatgtaatcaaagttgttggagagggaaatggaggggtagttcaattggtgcaacacaaatggactcgtcagttttttgtgttgaaggtacacttgctgcatgttgcataccatgctttgagtttgcatggtcttttctctcatttttagttagtttatccttttgtattggcattgtctacccatattacataatttttgataatgtcattcccctctccattacatccatttcattgagcaaggatgtttgaaacttgttttctcttggaatttcagtgtcacccaaagtcattattagtaagtttcataaaatgttatttctttcaatcaacatttaatgatttaatttcattaggagtggttcattttcattcaaatagtaatttagctcattactattatgctgctggtgctttctttctttgttatgtcagaacatgatatgaaccaagaattaggaatgggaaaggaaagaaagattttattgaataggatgaaaagaaccaggaataggaaagaaatctctcctccaagggtctccactccaaggatttccccttccaaaagaactaatgctctatttacaaatgacaacttattccccgaatcctgctatctgacctcccctatttatatctaattatcccctctaactaactgattaatattctaaacttcccatctccttataccctaacagaacatcattttgttcacccaacttcgaacatttgaattgtgtttaacatactgattgaaaatggttctcttccataggaacttgtatatattaatcaatcattgaacaagacaaatttaaacttgtgttaacatctaaggtcttcaaccccccctcccccttcattattgaaatgttcagtcttttgtcaattcattttctattaattttcctactagtttttgaaaatagagcagaaatgattgggtaacatgtcccatattggtttccctcctgcttttaacaattttaaatttccctcttgcttggttaacagtttagtatttgtatttgaactaataaactaattactttccttttgttctgatatattcccaaccttaatgttatcatttaaatctattcctcttttttcatttgtcactatgtatgcattcttcacaattcaggtaattcagatgaatattgaggagtctatgcgcaagcagattgcacaagagctgaaaattactcaacaagcacaatgtccttatgttgttgtctgctaccaatcattctatgaaaacggtgtcatatcaatcctcttagagtacatggatggaggttccttagctgatctgctgaagaaagttaaaacaattccggaggattatcttgctgccatctgtaagcaggtatggagtaaggaatttcccttcagtctagtacccataattcttttcactgaatcgcaaacaagatctgatctctttcttttttcttttttaatgatgtaggtgctgaagggtttagtttatctccaccacgaaaagcatattatccacagagacttaaaaccttctaatttgttaataaatcatattggtgaagtaaagattactgactttggtgtgagtacaattatggaaagtacatctggtcaagcaaatactttcattggaacctacaactatatgtctgtgagtaatatgtgcaagtatatttatcattttaccaccaaaatttaccagtgttgcctagaatatacagtaacattcactctgccaattttatagccagagagaatcattggaagccagcgaggatacaactacaaaagtgatatatggagtttgggactaatattactcgagtgtgctttgggtcggtttccatatgcaccgcctgatcaaagtgaaacatgggaaagcatttttgagcttatggaaactattgtcgataaacctcctcctagtcctccatctgaacaattttctacagaattttgctcatttatctcagcatggtaattgttatatgtgatactagttctgtgcagtcaagagtcttaatccaccataagaaaatgaaaacacaaccacacacacacacacacacacacacacagagaacactaacataacggtagcagtttcttaaaggtcatcctgaaattagtgagtggatgcttgaggagctttgatttgctgattgtttatgttgacaaaacaataatagaaatgtaatgcacttgccctacctacatccacaaaccattattttcattttttattcttatttttaaggtatctttcataatttcatcgaagaaaccatttaaacatgtataaatattagcaccttccgtcactattctgctgcctgttttgccaaaaactctatttcactgtgtgctttttatgtgtctactgatcttccttttttcaatcttgtttagtctacagaaagacccaaaggatcgactgtcagctcaggaattaatggtatgtgtaaatcttgcttatgtacacataccatgtaaaattgtctttctgatgctatatatttgattgaaactccaggcacatccttttgtcaacatgtatgatgacttggaggtggatctttcagcttacttctcgaatgcaggttctcctcttgcaaccttataaaagtttgtatgttccaatcttggactctattattaatttaagtaaaaggttattcgtttgttctttacttgcgaatctatcaggtgttcttgaatgtagatccctatataatgtattgattttatcataagtagaagtattttttcttctatattaagaaattgtttttggttctgtaaatgatactaagggtattgttttctaaattacgatgataggatgtgctctcatgcaaccatccagttttagatattaatatttctggataatctgagtaccttctttctaaacaatcggagttatttgtaactgtagttttccatttctgttgcgtttgcagatgagttgaagaaccagctcctagagtcacttcaaggattctgtatttagttgttgagtggttgagttcaatttgttgataaactcagatgcaagggtcaagaaattgcaatagttgacatatgcaagaactttaatgctgaagttaagtatcaggggctgcttttgcaacaaaatgtttgaaatttatgattgcaacaaaacgtatcacaaggatttgcctgagttgctgataatatgatctgatgtacaaattttcatgaaataaaagttttgtcaagctgtcccaaatgaacattttgagtcacgctaactgaacagttcctcaagttatatttgagactttttatttggttaaagcttgtcaaatttcctgattgatgatgggttttcattgcttgcaattactaatcaaatttaagacttgggaaccattttgttacgtgatgccctagggaatgccatatccgcatctgtagtcttcagttgctgcgttgtttcgttttctgttttttctcattacgtaaccaaaataaaataaaatttaagagtggc

SEQ ID NO: 4 - DNA, 5208 bp, Glycine tomentella
  source 1..5208: mol_type = other DNA
atggcttctatctccagcgtccagagcagctcttctgcatatgacgtgtttctcagcttccacggtgaagacacacgcaacaacaacaacaactccacaggttttctttttcaagctctcagaagaaaaggcatcgtcgccttcaaaggtcatacacttcccgaaaagggtgaacccgagctgttacatgcaattgaaggctcccgccttttcattgtggtcctttcaaagaactatgcttcctcaactcggtgtttgcgtgaactagcacacatcttcaattgcattgaaacttcgccaagacgtcttctgcctattttttatgatgttgatccatcagatgtgcgaaaacagactggatggtacgaggaagccttttggaaacacgaacaaagattcttggtagataaagagaggatggaggaagtgcggacatggagaaaagctctgactcgagtggccaatctccctggttggtatatcccaaataagccacaatatacagagattgaagaatttgttcaatatataataactatattgggtcctaatatttcaactcttcaaaatgaagcagagggaacatcttcagataaagatgaagttttatacaatgagctgtcaggcttgatgaggataagagattttatcagcctcgtgttgacttatcctgggaaattagaagatgaaaatattttaagatcttggattagtgcatttcctgaatggaaagctcagttattgtccactggtgatgacactgtgatggacaaaccatggtgtgtactaactgataaggcactgggacctatgaagggatttatgccaatggaaataataagtaaagtagaaagtggtgagagatccatcttcagccataatgatgttgatgcaataccagaagccattctccaacaatctgttgatctcgcagttaaccaactacttcaaactttgttttctggggattactgtgcacgctatatcagactgttaaccagaaacttggctgaaaaaggatctgtagtggacacaatagtttcagccgttgaagataagcacaccatgtttggaattgacaaagattttcttagagccctatggatcaatgcctcaacatatgagactgatgcagaggttcgagttcaagaacaaatcgatatcaatatgatgatggaagttgatgacatgggaagcaatgggcagttggtaattgtagtggatgcagatagcaacaacaagctggacctccaaaagttgcgttttccaactggaattgtggtgttgataacaactgagccgtcaacacaagcagagaaggatgttgactttagaattgcatgcacaatggatttgaatatttggacccaggatcatctattgccttggaaagttttctgcagctatgtcggcagttgtatcagcagtagcatggtaagttcatcaatggccattcagaaaattgcagaggaaatagttaaggaatgccatggtcatcttcttgccattgtccttgtggcaaagtatcttacgtatgtgcaagattttaaacagtgggaacttgcacttgacaaattaagcagtctgaatccttcctatgattatcaagatagtgatcgaattggcattagtagagtcatggtcaatgcattcgtaaatattatttgggaagacattgatgatgcacaaaagctttgccttgaactcagtttgcctgttcacaacattaaaaatggggtgagagatgatatcttggtgtctgattgggctaagataatatcaggatacacacaagaacttggtgaatacagaaggcaattacaacattacatcaatgagcttcaagaccgttttgtcttgttgaaatatgaaagtggagatgtctatttgccagtagaaacctatgatataataaaatcattgcacacctcaaagccttccatcctaaggcatggtgcattaggattgacagagccaccctacattggacgatggcatagtcttgttcagatagaattgatggacaataaaatatgtgaattaccacaatctccagattgccctaaactcaaagtagtgttgttgcagggcaatgttgatttgatggatatccctgactcatttttcaaccacatgcccctgcttcaacacttggacttatcctacactagtataagggatttacccccttctgtctctaaattaatacaacttcagaaattttatcttagaggctgtgatctcttcatggaactaccacatcagattggacagctaaagaacctcaaagagcttgatcttgatgggactttgatcacccatctgccaatagagatccgagaattgatcaacctgcaaagcttaagcctctgctttgatggaacttccaggtcaacaattattcctccaggattggtatcaaatcttactcaattgaattatttaagcatcaacgttgaccctgaagatgagcgatggaatgagaatgttattagcgtcttgagggaaattttttttggattatggaatttgcagaggttgagtatatatgtcccaaaggctgacctcttggaattcataccaacccaaaaatctctcaatttcagattggttgttggtcaacatatgcgacgtttgatatcgcgtgtaccacctgaactggagacaaaatttaaacactgtgactatagcataaagtttgttaatggtgtgaatgttccaaatggagtgaaaatgcatcttggacgcttcaaagccttgtacctagaccgacatatgaccatcaagagtctctctgatttcgatttgaggaatttatgggggttggaagtatgcatattggcagagtgtaatgaaatggaaacaattgtcagtggaagcaattcacctgataggccagcttctctaatgttgaagtttttgagtgtattctatatgaagaatttaagaagcgtctgcgaagggagtagtcccttctttctctgcttaaagtctatagcattacacacttgccccatgttaaccaccatcttcaccttagattctcttaaaaaactttcctttttggaggagattattattgaagactgtccaaaagtaaccaccctgataagtcatgccacaccagaacaaaaaccagtgtttttcctccccaagttgagaatgatatcgcttctctatcttcctgaattggtcaacattttcaatggcatacatgttggaccttttatagaagaaatgatattttattattgcccaagtctccaaagtctttccaaatcagagttgtcaagcaaatctttgaagatcatcaaaggagaaagcatctggtgggaagcattgaaatggaatgtagcagagtggggagatgctggtcgacccaattattttgaccgaattttttcaccaattaacgaggaggctgacatgatgaaccaactagctgcacttcaagagactcgactaaatgaacatcacaatacgacgtatcagggaatttcaagttctacacattcgatagcaaagtcatctttggaaatacctgttttgtcacctttagccccagtttcatctgtgggtaggggtagttcaaacagtgaagcccaaaagcggaaagtcgttactgagccaaaaatattacaagatgcgtcaaaaaagataataaagcaggttcgtgaacaaagaacagttcaaactgaagtttggacagaacctgaagtttggacagaaaagataagtgtcaatgcggaagggtatttaaatgatggatatagctggagaaaatatgggcaaaagagtcctcttggagctaagtttccaagaggatattacaaatgcgcatatggatctgtacaagggtgcccagcaagaaaacaagttcaaaggttagacgaaaacccagcaacatttgagataacctaccaaggaagacatacatgcaccaaagaacagaatttgatcattaagagtggggatgagggaatttcaagttctacacagtcgatagcaaagtcatctttggaaatacctgttttgtcaccttcagcccctgtttcatctgtgggtaggggtagttcaaatattgaagcccaaaagcggaaagtcgttactgagccaaaaatatttcaagatgcatcagaaaagtgcaatttaatcatgaagaaaggaaacttggggcttggtctcaaactctcagttcctgtgtccgatcaatctaattttgccaagttcctggatggagttcgaattgtctctcagaatgatgtcgaagcgccacctcaaatcaagccgacagacaaccagttaactttggcagatatagatgtaatcaaagttgttggagagggaaatggaggggtagttcaattggtgcaacacaaatggactcgtcagttttttgtgttgaaggtaattcagatgaatattgaggagtctatgcgcaagcagattgcacaagagctgaaaattactcaacaagcacaatgtccttatgttgttgtctgctaccaatcattctatgaaaacggtgtcatatcaatcctcttagagtacatggatggaggttccttagctgatctgctgaagaaagttaaaacaattccggaggattatcttgctgccatctgtaagcaggtgctgaagggtttagtttatctccaccacgaaaagcatattatccacagagacttaaaaccttctaatttgttaataaatcatattggtgaagtaaagattactgactttggtgtgagtacaattatggaaagtacatctggtcaagcaaatactttcattggaacctacaactatatgtctccagagagaatcattggaagccagcgaggatacaactacaaaagtgatatatggagtttgggactaatattactcgagtgtgctttgggtcggtttccatatgcaccgcctgatcaaagtgaaacatgggaaagcatttttgagcttatggaaactattgtcgataaacctcctcctagtcctccatctgaacaattttctacagaattttgctcatttatctcagcatgtctacagaaagacccaaaggatcgactgtcagctcaggaattaatggcacatccttttgtcaacatgtatgatgacttggaggtggatctttcagcttacttctcgaatgcagatgagttgaagaaccagctcctagagtcacttcaaggattctgtatttag

SEQ ID NO: 5 - DNA, 16798 bp, Glycine tomentella
  source 1..16798: mol_type = genomic DNA
acacactcaagtgataaacgcgttgacttgagtccattgtgaaacaaataaagcaagcaataatcaaacacttgatcgatcattttacaaaatctgggtgtcttaatatctgtcggctacggctgctcgcactcgcagaggtacacagtcatcatcataaatctttgttaactgttgtttatgattcctcactcacccttcagatggatctcaaatctaacctcttaagtcttaagtagtttgattctgtatatcactttcaactaattaaggaataactaatttattgtcttctatttttcttttcatctttctctaaaatacaataatataacttataacacaaaagttttatgattgttgttttttttttttattttgtcagctaatttccaaaaatgatattattttactccttcctaattaataaaattcgatcgtattttcaacaaattatatttgtatatttaattatttcacttttattttttttataaatatgttatacctaataatagtaaataatccatatctatctaaatctattacatttttaattatacacacgcagactaacacatatatgggataattagtttattagataatttttactaaatatttcatattcaatagctttcaaactatatctgacaaaaataattctaagttggtgcaatgcttaaaagtgatttttttaactaaatgaagtcattaaggtagaacaaacttataaattaaataagttatcgattatttaaaatcacttatcaaacacattcttgggtatattatatttagtaagactcataaataaataaaatggcttaaagttgaaaaattatttgaagttaaagttgttaaactcaatcatttaattagaagtgttcaataaaaataatcattgaagtaactaataaatataaaatgatatgaaatattctttttattttgtagtcaaaagtatatatttttaaaatgtagatgttatagattaaacagagatagaataatcaaatgtatcttaatcagtagtatttattttggttggtgcctgtcctccactagtattgaacaaggagaccagagactcaatttggtgctgctcatgcgttcactaacccatcatttagtaaaatctgtgcacaagaggtgctggcagagctagacatgcatgaatctttcttaactgttgcttctgattcccccgagatggatctcaattcttaacagttagcagcagaggtacacatgaatctttctcacttaactcttgctcccgattcctcctcagatacattccaatcattttgtatatcagtttcaacatggacggattttgggggggatgggggaagcaggggccttggccctccctcaagatcctttatatatacttgaggaaaaagaaaaagaaaatattactagtataaaatgaataataaaaatagagtattgacttaatgtttctttaattttttttctagtagtagttcccgccacaggccacagcttttcttccattggattccattttatcccaactccagcaatcgagaaatacttgtccttgatttacaccttctgtccttcttgacaaacaagtttacttccactttcgcaattccaatggcttctatctccggcgtccagagcagctcttctgcatatgacgtgtttctcagcttccacggtgaagacacacgcaacaacaacaacaacttcacaggttttctttttcaagctctcagaagaaaaggcatcgtcgccttcaaaggtcatacacttcccgaaaagggtgaacccgggctgttacatgcaattgaaggctcccgccttttcattgtggtcctttcaaagaactatgcttcctcaactcggtgtttgcgtgaactagcacacatcttcaattgcattgaaacttcgccaagacgtcttctgcctattttttatgacgttgatccatcagatgtgcgaaaacagactggacggtacgaggaagccttttggaaacacgaacaaagattcttggtagataaagagaggatggaggaagtgcggacatggagaaaagctctgagtcgagtggccaatctccctggttggtatatcccaaataagtaagtaacccgctctcatctcatgaaagatgtaacttgcaaattgggtccaaatttaatatagatgtttgatgcaaatggtatttaaaagttgaaaactactcttctattatcttctcctgttttgcttctcttgggtgatgatttcaatttcttcattttctttaaaatttcaggccacaatatacagagattgaagaatttgttcaatatataataactatattgggtcctaatatttcaactcttcaaaatgaagcagagggaacatcttcagataaagatgaagtttcatacaatgagctgtcaggcttgatgaggataagagattttatcagcctcgtgttgacttatcctgggaaattagaagatgaaaatattttaagatcttggattagtgcatttcctgaatggaaagctcagttattgtccactggtgatgacactgtgatggacaaaccatggtgtgtactaactgataaggcactgggacctatgaagggatttatgccaatggaaataataagtaaagtagaaagtggtgagagatccatcttcagccataatgatgttgatgcaataccagaagccattctccaacaatctgttgatctcgcagttaaccaactacttcaaactttgttttctggggattactgtgcacgctatatcagactgttaaccagaaacttggctgaaaaaggatctgtagtggacacaatagtttcagccgttgaagataagcacaccatgtttggaattgacaaagattttcttagagccctatggataaatgcctcaacatatgagactgatgcagaggttcgagttcaagaagaaatcgatatcaatatgatgatggaagttgatgacatgggaagcaatgggcagttggtaattgtagtggatgcagatagcaacaacaagctggacctccaaaagttgcgttttccaactggaattgtggtgttgataacaactgagccgtcaacacaagcagagaaggatgttgactttagaattgcatgcacaatggatttgaatatttggacccaggatcatctattgccttggaaagtcttctgcagctatgtcggcagttgtatcagcagtagcatggtaagttcatcaatggccattcagaaaattgcagtggaaatagttaaggaaagccatggtcatcttcttgccattgtccttgtggcaaagtatcttacgtatgtgcaagattttaaacagtgggaacttgcacttgacaaattaagcagtctgaatccttcctatgattatcaagatagtgatcgaattggcattagtagagtcatggtcaatgcattcgtaaatattatttgggaagacattgatgatgcacaaaagctttgccttgaactcagtttgcctgttcacaacattaaaaatggggtgagagatgatatcttggtgtctgattgggctaagataatatcaggatacacacaagaacttggtgaatacagaaggcaattacaacattacatcaatgagcttcaagaccgttttgtcttgttgaaatatgaaagtggagatgtctatttgccagtagaaacctatgatataataaaatcattgcacacctcaaagccttccatcctaaggcatggtgcattaggattgacagagccaccctacattggacgatggcatagtcttattcagatagaattgatggacaataaaatatgtgaattaccacaatcaccagattgccctaaactcaaagtgttgttgttgcagggcaatgttgatttgatggatatccctgactcatttttcaaccacatgcccctgcttcaacacttggacttatcctacactagtataagggatttacccccttctgtctctaaattaatacaacttcagaaattttatcttagaggctgtgatctcttcatggaactaccacatcagattggacagctaaagaacctcaaagagcttgatcttgatgggactttgatcacccatctgccaatagagatccgagaattgatcaacctgcaaagcttaagcctctgctttgatggaacttccaggtcaacaattattcctccaggattggtatcaaatcttactcaattgaattatttaagcatcaacgttgaccctgaagatgagcgatggaatgagaatgttattagtgtcttgagggaaattttttttggattatggaatttgcagaggttgagtatatatgtcccaaaggctgacctcttggaattcataccactgctaccagctcaaaaatctctcaatttcagattggttgttggtcaacatatgcgacgtttgatatcgcgtgtaccacctgaactggagacaaaatttaaacactgtgactatagcataaagtttgttaatggtgtgaatgttccaaatggagtgaaaatgcatcttgaacgcttcaaagccttgtacctagaccgacatatgaccatcaagagtctctctgatttcgatttgaggaatttatgggggttggaagtatgcatattggcagagtgtaatgaaatggaaacaattgtcagaggaagcaattctctaatgttgaagtttttgagcgtattctatatgaagaatttaagaagcatctgcgaagggagtagtcccttctttctctgcttaaagtctatagcattacacacttgccccatgttaaccaccatcttcaccttagattctcttgaaaaactttcctttttggaggagattattattgaagactgtccaaaagtaaccaccctgataagtcatgccacaccagaacaaaaaccagtgtttttcctccccaagttgagaatgatatcgcttctctatcttcctgaattggtcaacattttcaatggcatgcatgttggaccttttatagaagaaatgatattttattattgcccaagtctccaaagtctttccaaatcagagttgtcaagcaaatctttgaagatcatcaaaggagaaagcatctggtgggaagcattgaaatggaatgtagcagagtggggagatgctggtcgacccaattattttgagcgaattttttcaccaattaacgaggaggctgacatgatgaaccggctagctgcacttcaagagactcaactaaatgaatatcacaatacgacttatcaggtaatttttagttatcctcttttcaaatttcatggtgttatcggcatgttggtattttcaaatttttgctaatttttatataactcgttatgttctttttctattaatttcactatcatctttcatttcttttacttcattactattactatgctttttttgttctcagattgatagtgactctgtgcataaaatgcagactatttatcatgaatttctttaattttggattttttttctttcttgattttaatatatatgtagtataaactatttagcttgcattataatatttttaaaaatgaccatcctgcttcctgctatagtgttttaagctatgtacttctatgtgagattaataactatgattagttttcatgtgtaggatatcttcctatttttgtatatctctttcttaatgaacatttttttatgtcaaaacataattaacttcacaacaattcttcttggtgctttaaaaattgtggcaaattttttttccagcactttatgatcatacttaattatgtttggagatttaaataaacaagtatgttcatatttatctccctactcatcaaactccaaccatgggtcatactgagctagtgttataattataataattttgtatctctgtatgaattgcttttgctaatatgatcataccctcttagcttcatttttcgtactgtattacttgaatgttttcctttcagggaatttcaagttctacacagtcgatagcaaagtcatctttggaaatacctgttttgtcacctttagccccagtttcatctgtgggtaggggtagttcaaacagtgaagcccaaaagcggaaagtcgttactgagccaaaaatattacaagatgcatcaaaaaaggtaataaagcaggtttgagatgccttgtcaccgttcgtgattgattgcttttggtttttttatatgcacgagttggtttacttacattaaaggcatgtttgataaacctgtaaaattatacgtcgaatgtgttttgcttgaaaagattataattttaagtgatgaaaaactcattcaattaagaaaacttaaagcatttagatgctaaacatggtgacaccaaaggaaattatgtcatttaattattcagatagggaagtagttgctgctttattcattcagtatcagcaatgtgctgaatgtatttaacatatttcacgtaaagaattattttcagtttttatatgactagttgccaatagcaacaaatagtggtgctagcgcagaattgcattgcagagcggctagagcatgctatgaaaattgaaaagtgtatcagtagcgaccatcacagctgtcatatctagcaaaatagcagaaattgctcctggagcaggctttattggagtgggtcacaaaaaaacctattttaatttaaccattaaacaactttatttagggtgttttagtgggcaatgattttgcccccttctttgaatcacaactgaaactctactttctcaattacttacacacactgttatgacatattgctatcctttttccaccaagacacccttttctggcaacatttttggcatttccctgcattttgggggcttctgtgagttttttgccatccccaacctctgcagcctctattctccatagtttccggtcattcctgacctttatggccattttctggcgatcctgtcactgtttctggccaaccgcgacctttgctgccgtgctctgttcactgttctgtccttgcattgttcatctctctaatacagatccttcatctctgttttgtgctctcttctatatttttctttaacatttaatttaatatttgtcatattttattttattttattatcaattacgaaattattgatcaaagttttataagaaagtataaagtatttctagcattttattgtcaatatataaaatgacaaaatcattcaaattatttcataacatagttaatatcaaataagattttagtatgtaacataaaaaagatttgagtaattttagtttgaaaagcaactttcaacaataataataataattactaggtttattaataatattagaaacaatgcatgtatttgaaagaaaaatactatatttttaaataaacgatatttttaatgaaatatgaaattttttcctatacttcttgcaatatctttattttaaaaataaagaaaatattttcagtattataattcttagtagaacttaaaagtattttcaatccatatcttcatctaaagttttcaattgttcaattaaaatacaatattaaattacagtcttaataataaaaatagtaatatatatcgttatcaaatttatcacctaataattcaaattaataataaatatttttcaaaaagtataaattttttaggcttaaggtaactacttaaatggccatggccttgtggttgccacagccctagttagaagtgaatcttataggggtctgaaggttctaacagacggattctctccatttttagtggaatttgcttactctttatcagcaacttattttgttcattgctgtcacgaaaaagaaaaaaagaaaaggaaaatatcactttggaaatgtgtcaatcctatgaacctatgatgcatggtcatataagtagggagttatacgcctgcaacttgaaattgtttgttctctactttgactatgcatcaattataccacaaaagattgagaagttagatattctctatattgcaaatcaatctttagcttcaacacaatgtaacctacttccccaactgaaaaatgatgatgatggttttattcatgaatcgagatcaaatactgttgctgagttcctctatgaagatgtgtccgtaggaggtgtctctatagtctttagttaaacaaaaataaatttcatgaacctaacatgttgaataaaagactattgtggtttttatattttggcatggcaattaacaggttcgtgaacaaagaacagttcaaactgaagtttggacagaacctgaagtttggacggaaaagataagtgtcaatgcggacggttctttagatgatggatatagctggagaaaatatgggctaaagaaaatccttggagctaagtttccaaggttagttaaacggtcttttctcgtttaactttttcttgttataaaattaaacaagtttaattagcaattttttttctagtataaatttatataattgacatttgatttgaatgtgacagaaaggttcacccctctccctctaggttattgacgtacaagtttcacccctctcgtttagttaataccagaggccttggacctggtaactggaaaatcagtagccaattagctaatgagttctctttcattgttttaatggttctatgcccccgtgtttttaaaatgacctgcaatttgaaaaattatctaaaggaccatgtcaactggtttcaaatgggcccaaattagtgctttctgttggaaacttgtttttgacattctcaacaaaaacttgttcatttgacaactacctttgcaaaaatttaaaaccgagaacaaccacaaactccagataacttctgattctttgtttttggacatagactatactataacattcacagcaactaaaaagagaatgtaggtttcttctccaacttccttatcaaggcttaaaacaagaggggtttctattaatatttttatttgctgttcgtaccatactaactttgccagtctaatgtttatattaagaacataaaaagaagttattttctagttttgccgaaaaattgacaactagtcgtttgcattgcagaggatattacaaatgcacatatggatctgaacgagggtgcccagcaagaaaacaagttcaaaggttagacgaaaacccagcaacatttgagataacctaccaaggaagacatacatgcaccaaagaacagaatttgatcattaagagtggggatgaggcaagtttgaacaaaaagactagtaatcaatcttgcaagttataaaaaaacttttcttgcaaatttgcattactaaaaataaaaatagacaaatttaaaatatattgctgatgttatctacatttcagtaaagaaatataaataacattatttaatatgatcataaatacatttcatccaaaaaatgttactggtaatccaccaaacttggaacatacaaaacatcaagctagtatccaatgaatgctaatcaaagattgttacaatgcatgtaatctctggttgaatcatcttcatcatcaaaggttgcctacataacaatacaaaatacctcccattacaaggcaaaaaaagtggggaaaaaatgggttaaatgattttatgtagtgaaactaaattgatttttggatgtttgaagaggggggtgggggatcaaaagtattcatgataaattaacataaaattataagaacgagaaattattagttgacttaaattatgttttttgtctatacaaaatgctcaaagttcatatttaatctgtcattctaatcaaaacctatgtgacatttgcttgttccagaaaacttgacagctgtaactttatatctctgtatgaattgcttttgctaatatgatcataccctcttagcttcattttttgtattttattagttgaatgttttcctttcagggaatttcaagttctacacagtcgatagcaaagtcatctttggaaatacctgttttgtcactttcagcccctgtttcatctgtgggtaggggtagttcaaatattgaagcccaaaagcggaaagtcgttactgagccaaaaatatttcaagatgcatcaaaaaaggtaataaagcaggtttgagatgccttgtcaccgttcgtgattgattgcttttggttttcttatatgcacgagttggtttacttacattaaaggcatgtttgataaacctgtaaaattatacgtcgaatgtgttttgcttgaaaagattataattttaagtgctgaaaaactcatttaattaagaaaacttaaaacagttagatgctaaacatggtgacaccaaatgaaattattgtcatttaattattcagatagggaagtagttgctgctttattcattcagtatcatcaatgtgctgaatgtattcaacatatttcacgtaaagaattattttcagtttttatatgactagttgccaatagcaacaaatagtggtgctagcgcagaattgcattgcagagcggctagagcatgctatgaaaattgaaaagtgtatcagtttgaagtagcgaccatcacagctgccatatctagcaaaatagctgaaattgctcctggagcaggctttattggagtggatcacaaaaaaacctattttaatctaaccattaaacaactttatttagggtgttttagtgggcaatgattttgcccccttctttgaatcacaactgaaactctgctttctcaatcacttgtacacactgttatgacattgttatccttttcccaccaatacacacccttttctggcaacatttttggcatttccctgcattttcgggccttctgtgagttttttgccatccccaacctctgcagcctctattctccatagtttccggtcattcctgacctttatggccattttctggcgaacctgtcactgtttctggccaaccgcgacctttgctgccgtgctctgttcactgttctgtccttggattgttcatctctctaataaagatccttcatctctctgttttgtgctctcttctataattttctttaacatttaatttaatatttgtcaaatttttatttatttattatcaattacgaaatcattgatcaaagttttataagaaagtataaagtatttctagcattttattgtcaatatataaaatgacaaagtcattcaaattatttcgtaacatagttaatatcaaataagattttagtatgtaacataaaaagatttgagtaattttagttttaaaaactactttcaataataacaagaattactagtttattaataatattagaaataatgcatgtatttgaaagaaaaatacaatatttttagtgaaatatgaatttctttttctatacttcttgcaatgtctttattataaaaataaatcaagattttcaatattataatttgcttactcttctgtatttttctttaatatttgtcatactttattttatttcttttatcaattacgaaattattgatcaaagttttatagaaagtgtaaagtatttctagcattttattgtcaatatataatatgacaaaatcattcaaattatttcaatatcaaataagattttagtatatcaaataagatttttagtatgttaaaaaagatttgagtatttttagtttaaaaactacttttaataataataataataataatatttacaagtattattaataatattagaaacaatgcatgtatttgaaagaaaaatacaattttttaaataaataatgtttttagtgaaatatgaatttttttgtctatccttcttgcaatatctttattttgaaaataaatcaagattttcaatattataattcttagtagaacttaaaagtattttcaatccatatcttcatctaaagttttcaattgttcaattaatacaatcttaaaattacagtcttaataataaaaatagtaatatatgtcattataaaatttatcacctaataattcaaattaataataaatatttttcgaaaagtatacattttttaaggctaaaggtaactacttaactggccatggccttttggttgccacagccctagttagaagtgaatcttagaggggtctgaaagtgctaacagacggattctctccatttttagtgcaatttaatcgtgaagaaaggaaacttggggcgtgatctctctgtgatgtgtgggacttttaaggatggggatctgcttgtcaacagggatggagttcgaattgtctctcagaatgatgtcgaagcggtaacatatttctctcacttttttatgcattctttgcctttgtaattgtttgtttgttaacaatttgtttatttcattatgtgtccagtccgattgctccattgtttgtttttttgcatttgatcttttggatctgatgaaatgcctgatagataagataacaagggattcatgatttttctgtagattccttattctcaggttaccatacccaagtggagtattatatgatttaaattgaataaaaaaaattatgagatggattatttgctgtttctccttggccattatttttcttgtgtgtgtgttttcggcggtggggattgaccctactttcacttttcctagcctttgctattggattttaaatgaacctgatattgttaacgaggttatccatttcttatcttatttacgttgtctttaatcaaatggacaacaagttgcaagtttatgaaatgaaattgtgctaagcatacgtcatccacttcatgagccactaaacaaattgagtctttcatttctgctcttaaacgaagtttgttgcataaaattgtttgcattctgagttaatgttggatcctcttcttccttcctacgtcttttttttttttggtgggactcagggaggctttcacactgtcaatcatgaaaaaaaataaaaaataatctgactaattatattggaaaacttcctatgcatactcttatatggtcaatacattttagaagacaaaatgagttgatagtagatacatagccatgttaaatgaggacccaaatttataagtgactaagtttaaatttattcctgttaaactccccataactatagttgtgcattccaaccagtggcttatggtataaaaagagcaggacaagggggagttgaatgttgaaattcatgcatattttttttttgtagtcacaatacacaatacattgctggctgaattctgacaatatgttgtatatgattctaacattcatgttattttttcagccacctcaaatcaagccgacagacaaccagttaactttggcagatatagatgtaatcaaagttgttggagagggaaatggaggggtagttcaattggtgcaacacaaatggactcgtcagttttttgtgttgaaggtacacttgctgcatgttgcataccatgctttgagtttgcatggtcttttctctcatttttagttagtttatccttttgtattggcattgtctacccatattacataatttttgataatgtcattcccctctccattgcatccatttcattgagcaaggatgtttgaaacttgttttctcttggaatttcagtgtcacccaaagtcattattagtaagtttcataaaatgttatttctttcaatcaacatttaatgatttaatttcattaggagtggttcattttcattcaaatagtaatttagctcattactattatgctgctggtgctttctttctttgttatatcagaacatgatatgaaccaagaattaggaatggtaaagaaaagaaagattttattgaataggatgaaaagaaccaggaataggaaagaaatctctcctccaagggtctccactccaaggatttccccttccaaaagaactaatgctctatttacaaatgacaacttattccccgaatcctgctatccgacctcccctatttatatctaattatcccctctaactaactgattaatattctaaacttcccatctccttataccctaacagaacatcattttgttcacccaacttcgaacatttgaattgtgtttaacatactgattgaaaatggttctcttccataggaacttgtatatattaatcaatcattgaacaagacaaatttaaacttgtgttaacatctaaggtcttcaaccccccctcccccttcattattgaaatgttcagtcttttgtcaattcattttctattaattttcctactagtttttgaaaatagagcagaaatgattgggtaacatgtcccatattggtttccctcctgcttttaacaattttacatttccctcttgcttggttaacaatttagtatttgtatttgaactaataaactaattactttccttttgttctgatatattcacaaccttaatgttatcatttaaatctattcctcttttttcatttgtcactatgtatgcattcttcacaattcaggtaattcagatgaatattgaggagtctatgcgcaagcagattgcacaagagctgaaaattactcaacaagcacaatgtccttatgttgttgtctgctaccaatcattctatgaaaacggtgtcatatcaatcctcttagagtacatggatggaggttccttagctgatctgctgaagaaagttaaaacaattccggaggattatcttgctgccatctgtaagcaggtatggagtaaggaatttcccttcagtctagtacccataattcttttcaccgaatcgcaaacaagatctgatctctttcttttttcttttttaatgatgtaggtgctgaagggtttagtttatctccaccacgaaaagcatattatccacagagacttaaaaccttctaatttgttaataaatcatattggtgaagtaaagattactgactttggtgtgagtacaattatggaaagtacatctggtcaagcaaatactttcattggaacctacaactatatgtctgtgagtaatatgtgcaagtatatttatcattttaccaccaaaatttaccagtgttgcctagaatatacagtaacattcactctgccaattttatagccagagagaatcattggaagccagcgaggatacaactacaaaagtgatatatggagtttgggactaatattactcgagtgtgctttgggtcggtttccatatgcaccgcctgatcaaagtgaaacatgggaaagcatttttgagcttatggaaactattgtcgataaacctcctcctagtcctccatctgaacaattttctacagaattttgctcatttatctcagcatggtaattgttatatgtgatactagttctgtgcagtcaagagtcttaatccaccataagaaaatgaaaacacagccacacacacacacacagagaacactaacataacggtgggagtttcttaaaggtcatcctgaaattagtgagtggatgctcgaggagctttgatttgctgattgtttatgttgacaaaacaataatagaaatgtaatgcacttgccctacctacatgcacaaaccattattttcattttttattcttatttttaaggtatctttcataatttcattgaagaaaccatttaaacatgtataaatattagcaccttccgtcactattctgctgcctgttttgccaaaaactctatttcactgtgtgctttttatgtgtctacttatcttccttttttcaatcttgtttagtctacagaaagacccaaaggatcgactgtcagctcaggaattaatggtatgtgtaaatcttgcttatgtacacataccatgtaaaattgtctttctgatgctatatatttgattgaaactccaggcacatccttttgtcaacatgtatgatgacttggaggtggatctttcagcttacttctcgaatgcaggttctcctcttgcaaccttataaaagtttgtatgttccaatcttggactctattattaatttaagtaaaaggttattcttttgttctttacttgcgaatctatcaggtgctcttgaatgtagatccctatataatgtattgattttatcataagtagaagtattttttcttctatattaagaaattgtttttggttctgtaaatgatactaagggtattgttttctaaattacgatgataggatgtgctctcatgcaaccatccagttttagatattaatatttctggataatctgagtaccttctttctaaacaatcggagttatttgtaactgtagttttccatttctgttgcgtttgcagatgagttgaagaaccagctcctagagtcacttcaaggattctgtatttagttgttgagtggttgagttcaatttgttgataaactcagatgcaagggtcaagaaattgcaatagttgacatatgcaagaactttaatgctgaagttaagtatcaggggctgcttttgcaacaaaatgtttgaaatttatgattgcaacaaaacgtatcacaaggatttgcctgagttgctgataatatgatctgatgtacaaattttcatgaaataaaagttttgtcaagctgtcccaaatgaacattttgagtcacgctaactgaacagttcctcaagttatatttgagactttttatttggttaaagcttgtcaaatttcctgattgatgatgggttttcattgcttgcaattactaatcaaatttaagacttgggaaccattttgttacgtgatgccctagggaatgccat

SEQ ID NO: 6 - DNA, 5187 bp, Glycine tomentella
  source 1..5187: mol_type = other DNA
atggcttctatctccggcgtccagagcagctcttctgcatatgacgtgtttctcagcttccacggtgaagacacacgcaacaacaacaacaacttcacaggttttctttttcaagctctcagaagaaaaggcatcgtcgccttcaaaggtcatacacttcccgaaaagggtgaacccgggctgttacatgcaattgaaggctcccgccttttcattgtggtcctttcaaagaactatgcttcctcaactcggtgtttgcgtgaactagcacacatcttcaattgcattgaaacttcgccaagacgtcttctgcctattttttatgacgttgatccatcagatgtgcgaaaacagactggacggtacgaggaagccttttggaaacacgaacaaagattcttggtagataaagagaggatggaggaagtgcggacatggagaaaagctctgagtcgagtggccaatctccctggttggtatatcccaaataagccacaatatacagagattgaagaatttgttcaatatataataactatattgggtcctaatatttcaactcttcaaaatgaagcagagggaacatcttcagataaagatgaagtttcatacaatgagctgtcaggcttgatgaggataagagattttatcagcctcgtgttgacttatcctgggaaattagaagatgaaaatattttaagatcttggattagtgcatttcctgaatggaaagctcagttattgtccactggtgatgacactgtgatggacaaaccatggtgtgtactaactgataaggcactgggacctatgaagggatttatgccaatggaaataataagtaaagtagaaagtggtgagagatccatcttcagccataatgatgttgatgcaataccagaagccattctccaacaatctgttgatctcgcagttaaccaactacttcaaactttgttttctggggattactgtgcacgctatatcagactgttaaccagaaacttggctgaaaaaggatctgtagtggacacaatagtttcagccgttgaagataagcacaccatgtttggaattgacaaagattttcttagagccctatggataaatgcctcaacatatgagactgatgcagaggttcgagttcaagaagaaatcgatatcaatatgatgatggaagttgatgacatgggaagcaatgggcagttggtaattgtagtggatgcagatagcaacaacaagctggacctccaaaagttgcgttttccaactggaattgtggtgttgataacaactgagccgtcaacacaagcagagaaggatgttgactttagaattgcatgcacaatggatttgaatatttggacccaggatcatctattgccttggaaagtcttctgcagctatgtcggcagttgtatcagcagtagcatggtaagttcatcaatggccattcagaaaattgcagtggaaatagttaaggaaagccatggtcatcttcttgccattgtccttgtggcaaagtatcttacgtatgtgcaagattttaaacagtgggaacttgcacttgacaaattaagcagtctgaatccttcctatgattatcaagatagtgatcgaattggcattagtagagtcatggtcaatgcattcgtaaatattatttgggaagacattgatgatgcacaaaagctttgccttgaactcagtttgcctgttcacaacattaaaaatggggtgagagatgatatcttggtgtctgattgggctaagataatatcaggatacacacaagaacttggtgaatacagaaggcaattacaacattacatcaatgagcttcaagaccgttttgtcttgttgaaatatgaaagtggagatgtctatttgccagtagaaacctatgatataataaaatcattgcacacctcaaagccttccatcctaaggcatggtgcattaggattgacagagccaccctacattggacgatggcatagtcttattcagatagaattgatggacaataaaatatgtgaattaccacaatcaccagattgccctaaactcaaagtgttgttgttgcagggcaatgttgatttgatggatatccctgactcatttttcaaccacatgcccctgcttcaacacttggacttatcctacactagtataagggatttacccccttctgtctctaaattaatacaacttcagaaattttatcttagaggctgtgatctcttcatggaactaccacatcagattggacagctaaagaacctcaaagagcttgatcttgatgggactttgatcacccatctgccaatagagatccgagaattgatcaacctgcaaagcttaagcctctgctttgatggaacttccaggtcaacaattattcctccaggattggtatcaaatcttactcaattgaattatttaagcatcaacgttgaccctgaagatgagcgatggaatgagaatgttattagtgtcttgagggaaattttttttggattatggaatttgcagaggttgagtatatatgtcccaaaggctgacctcttggaattcataccactgctaccagctcaaaaatctctcaatttcagattggttgttggtcaacatatgcgacgtttgatatcgcgtgtaccacctgaactggagacaaaatttaaacactgtgactatagcataaagtttgttaatggtgtgaatgttccaaatggagtgaaaatgcatcttgaacgcttcaaagccttgtacctagaccgacatatgaccatcaagagtctctctgatttcgatttgaggaatttatgggggttggaagtatgcatattggcagagtgtaatgaaatggaaacaattgtcagaggaagcaattctctaatgttgaagtttttgagcgtattctatatgaagaatttaagaagcatctgcgaagggagtagtcccttctttctctgcttaaagtctatagcattacacacttgccccatgttaaccaccatcttcaccttagattctcttgaaaaactttcctttttggaggagattattattgaagactgtccaaaagtaaccaccctgataagtcatgccacaccagaacaaaaaccagtgtttttcctccccaagttgagaatgatatcgcttctctatcttcctgaattggtcaacattttcaatggcatgcatgttggaccttttatagaagaaatgatattttattattgcccaagtctccaaagtctttccaaatcagagttgtcaagcaaatctttgaagatcatcaaaggagaaagcatctggtgggaagcattgaaatggaatgtagcagagtggggagatgctggtcgacccaattattttgagcgaattttttcaccaattaacgaggaggctgacatgatgaaccggctagctgcacttcaagagactcaactaaatgaatatcacaatacgacttatcagggaatttcaagttctacacagtcgatagcaaagtcatctttggaaatacctgttttgtcacctttagccccagtttcatctgtgggtaggggtagttcaaacagtgaagcccaaaagcggaaagtcgttactgagccaaaaatattacaagatgcatcaaaaaaggttcgtgaacaaagaacagttcaaactgaagtttggacagaacctgaagtttggacggaaaagataagtgtcaatgcggacggttctttagatgatggatatagctggagaaaatatgggctaaagaaaatccttggagctaagtttccaagaggatattacaaatgcacatatggatctgaacgagggtgcccagcaagaaaacaagttcaaaggttagacgaaaacccagcaacatttgagataacctaccaaggaagacatacatgcaccaaagaacagaatttgatcattaagagtggggatgagggaatttcaagttctacacagtcgatagcaaagtcatctttggaaatacctgttttgtcactttcagcccctgtttcatctgtgggtaggggtagttcaaatattgaagcccaaaagcggaaagtcgttactgagccaaaaatatttcaagatgcatcaaaaaagtgcaatttaatcgtgaagaaaggaaacttggggcgtgatctctctgtgatgtgtgggacttttaaggatggggatctgcttgtcaacagggatggagttcgaattgtctctcagaatgatgtcgaagcgccacctcaaatcaagccgacagacaaccagttaactttggcagatatagatgtaatcaaagttgttggagagggaaatggaggggtagttcaattggtgcaacacaaatggactcgtcagttttttgtgttgaaggtaattcagatgaatattgaggagtctatgcgcaagcagattgcacaagagctgaaaattactcaacaagcacaatgtccttatgttgttgtctgctaccaatcattctatgaaaacggtgtcatatcaatcctcttagagtacatggatggaggttccttagctgatctgctgaagaaagttaaaacaattccggaggattatcttgctgccatctgtaagcaggtgctgaagggtttagtttatctccaccacgaaaagcatattatccacagagacttaaaaccttctaatttgttaataaatcatattggtgaagtaaagattactgactttggtgtgagtacaattatggaaagtacatctggtcaagcaaatactttcattggaacctacaactatatgtctccagagagaatcattggaagccagcgaggatacaactacaaaagtgatatatggagtttgggactaatattactcgagtgtgctttgggtcggtttccatatgcaccgcctgatcaaagtgaaacatgggaaagcatttttgagcttatggaaactattgtcgataaacctcctcctagtcctccatctgaacaattttctacagaattttgctcatttatctcagcatgtctacagaaagacccaaaggatcgactgtcagctcaggaattaatggcacatccttttgtcaacatgtatgatgacttggaggtggatctttcagcttacttctcgaatgcagatgagttgaagaaccagctcctagagtcacttcaaggattctgtatttag

SEQ ID NO: 7 - DNA, 2104 bp, Glycine tomentella
  source 1..2104: mol_type = other DNA
agctttttcagttataatggaaatagatcgacccttggtgaaattcgttattaactgaccgataaattaaaatataaaaagatgtatttatgtacagatcaatttattgaaaaataaagttatagcttttatttaaatacattataaacaaaagatcaattttatttaaacacattaaataataaaataaattcagatttttaaatatatttattttaatcatatatttcatgtatataatcaaatgtattggattaagtctctttgatcaaagtggtaaataagttatcgattttgaatttttggaggattatattaaatgtgattaattagattttttaatagtcaataacatgataaaaaaacgtatttgattaaaagtaaaagtaattgtagatatatttttaatttaaaatcataatcaataacattatctttactttttgtcctaaattggtatcgagtcgacaccacacactcaagtgataaacgcgttgacttgagtccattgtgaaacaaataaagcaagcaataatcaaacacttgatcgatcattttacaaaatctgggtgaagaatatctgtcggctacggctgctcgcactcgcagaggtacacagtcatcatcataaatctttgttaactgttgtttatgattcctcactcacccttcagatggatctcaaatctaacctcttaagtcttaagtagtttgattctgtatatcactttcaactaattaaggaataactaatttattgtcttctatttttcttttcatctttctctaaaacacaataatataacttataacacaaaagttttatgattgttgtttttttttttttttattttgtcagctaatttccaaaaatgatattattttactccttcctaattaataaaattcgatcgtattttcaacaaattatatttgtatatttaattatttcacttttattttttttataaatatgttatacctaataatagtaaataatccatatctatctaaatctattacatttttaattatacacacgcagactaacacatatatgggataattagtttattagataatttttactaaatatttcatattcaatagctttcaaactatatctgacaaaaataattctaagttggtgcaatgcttaaaagtgatttttttaactaaatgaagtcattaaggtagaacaaacttataaattaaataagttatcgattatttaaaatcacttatcaaacacattcttgggtatattatatttagtaagactcataaataaataaaatggcttaaagttgaaaaattatttgaagttaaagttgttaaactcaatcatttaattagaagtgttcaataaaaataatcattgaagtaactaataaatataaaatgatatgaaatattctttttattttgtagtcaaaagtatatatttttaaaatgtagatgttatagattaaacagagatagaataatcaaatgtatcttaatcagtagtatttattttggttggtgcctgtcctccattagtattgaacaaggagaccagagactcaatttggtgctgctcatgcgttcacttacccatcatttagtaaaatctgtgcacaagaggtgctggcagaactagacatgcatgaatctttcttaactgttgcttctgattcccccgagatggatctcaattcttaacagttagcagcagaggtacacatgaatctttcttacttaactcttgctcccgattcctcctcagatacattccaatcattttgtatatcagtttcaacatggacggattttggggggtatgggggaagcaggggccttggccctccctcaagatcctttatatatacttgagaaaaaagaaaaagaaaatattactagtataaaatgaataagaaaaatagagtattgacttgatgtttctttaatttttttttctagtagtagttcccgccacaggccacagcttttcttccattggatcccattttatcccaactccagcaatcgagaaatacttgtccttaatttacaccttctgtccttcttcacaaacaagtttacttccactttcgcaattcca

SEQ ID NO: 8 - DNA, 2104 bp, Glycine tomentella
  source 1..2104: mol_type = other DNA
agctttttcagttataatggaaatagatcgacccttggtgaaattcgttattaactgaccgataaattaaaatataaaaagatgtatttatgtacagatcaatttattgaaaaataaagttatagcttttatttaaatacattataaacaaaagatcaattttatttaaacacattaaataataaaataaattcagatttttaaatatatttattttaatcatatatttcatgtatataatcaaatgtattggattaagtctctttgatcaaagtggtaaataagttatcgattttgaatttttggaggattatattaaatgtgattaattagattttttaatagtcaataacatgataaaaaaacgtatttgattaaaagtaaaagtaattgtagatatatttttaatttaaaatcataatcaataacattatctttactttttgtcctaaattggtatcgagtcgacaccacacactcaagtgataaacgcgttgacttgagtccattgtgaaacaaataaagcaagcaataatcaaacacttgatcgatcattttacaaaatctgggtgaagaatatctgtcggctacggctgctcgcactcgcagaggtacacagtcatcatcataaatctttgttaactgttgtttatgattcctcactcacccttcagatggatctcaaatctaacctcttaagtcttaagtagtttgattctgtatatcactttcaactaattaaggaataactaatttattgtcttctatttttcttttcatctttctctaaaacacaataatataacttataacacaaaagttttatgattgttgtttttttttttttttattttgtcagctaatttccaaaaatgatattattttactccttcctaattaataaaattcgatcgtattttcaacaaattatatttgtatatttaattatttcacttttattttttttataaatatgttatacctaataatagtaaataatccatatctatctaaatctattacatttttaattatacacacgcagactaacacatatatgggataattagtttattagataatttttactaaatatttcatattcaatagctttcaaactatatctgacaaaaataattctaagttggtgcaatgcttaaaagtgatttttttaactaaatgaagtcattaaggtagaacaaacttataaattaaataagttatcgattatttaaaatcacttatcaaacacattcttgggtatattatatttagtaagactcataaataaataaaatggcttaaagttgaaaaattatttgaagttaaagttgttaaactcaatcatttaattagaagtgttcaataaaaataatcattgaagtaactaataaatataaaatgatatgaaatattctttttattttgtagtcaaaagtatatatttttaaaatgtagatgttatagattaaacagagatagaataatcaaatgtatcttaatcagtagtatttattttggttggtgcctgtcctccattagtattgaacaaggagaccagagactcaatttggtgctgctcatgcgttcacttacccatcatttagtaaaatctgtgcacaagaggtgctggcagaactagacatccatgaatctttcttaactgttgcttctgattcccccgagatggatctcaattcttaacagttagcagcagaggtacacatcaatctttcttacttaactcttgctcccgattcctcctcagatacattccaatcattttgtatatcagtttcaacatcgacggattttggggggtatcggggaagcaggggccttggccctccctcaagatcctttatatatacttgagaaaaaagaaaaagaaaatattactagtataaaatcaataagaaaaatagagtattgacttgatgtttctttaatttttttttctagtagtagttcccgccacaggccacagcttttcttccattggatcccattttatcccaactccagcaatcgagaaatacttgtccttaatttacaccttctgtccttcttcacaaacaagtttacttccactttcgcaattcca

SEQ ID NO: 9 - DNA, 1033 bp, Glycine tomentella
  source 1..1033: mol_type = other DNA
ttgttgagtggttgagttcaatttgttgataaactcagatgcaagggtcaagaaattgcaatagttgacatatgcaagaactttaatgctgaagttaagtatcaggggctgcttttgcaacaaaatgtttgaaatttatgattgcaacaaaacgtatcacaaggatttgcctgagttgctgataatatgatctgatgtacaaattttcatgaaataaaagttttgtcaagctgtcccaaatgaacattttgagtcacgctaactgaacagttcctcaagttatatttgagactttttatttggttaaagcttgtcaaatttcctgattgatgatgggttttcattgcttgcaattactaatcaaatttaagacttgggaaccattttgttacgtgatgccctagggaatgccatatccgcatctgtagtcttcagttgctgcgttgtttcgttttctgttttttctcattacgtaaccaaaataaaataaaatttaagagtggctccaaggtgcgtatgtaagagacaatgagcttatcaattgattagctaacacaacatattggctcttaacattttttttataaaaaaaaaaaaaaaaaaaaaacctttgttatgcttggaaagttttcagagacagtgataactcatccaagctgaaggtatccttgtacagtgcaaatattagcatatataacttagcttagccctggtgaaaaactgacgggctaaaaagtgccaatgactctaaaatacaataaatcaatagcttcaaatgaagatatgccagatataaaacacatcactttatggctttctcttaatttttctttgagcctttaagtctatcacaattacagagatgttatcttggcttcccctgtggatggcaagcttagttagatactctgcagcggactgagctgcaggatcagctccttcactgcgtcctgttgtgtcgttgtcaccatacttcttgtgccaaagaaggattcgcttctttgcaacctcgcatgcttcttcatttgtcatt

SEQ ID NO: 10 - DNA, 3000 bp, Glycine tomentella
  source 1..3000: mol_type = other DNA
aatttcaaattgtttggtaagaagaagaaacagcccaaacagttatctcaagtggtcacagagactcacttaagttcacaattgtctctcaaatctcaaaatgtaccatctttgtttcttgattttacaaccattttctttctttctttcttgagagagaattatgagactagtgtgagtctttgagatttactgtttgtcctgcctcttcttcttgtcaaacaatttgcatgcgggcgtgcatatttcctaactcatcaaatcatgtaacacgagagtgagattaagcttgagaattaatgaaagaaagcatgatgaagtgcgaactagtttagaaggataatcgaagataggaaagaaggctaataaggataaatccactatggccatggtaccagaaagaagatgccaaaataatgtatacaatatataatctcaggtactcaagtttgtaatcaatgtgatcataaaacaaataatatttattattataaggagttgatcaaataagaacgaagagctagaaagaaaagaaaatacctataatgtataagattaatatttggtttgaaggacaaaatagataagaaaacagagataattaacgatctttttctctaattggattggagaaaaatataaagaaaagaggggaaaagttaatttttaaaaaacaaaaattcaaatttcttttgacttttcttcatattcaaattttaggttttcttttcttctgtctttctctcgttacaaaaacaaaatttttgaaggatagaaggagtcaaatgaaaataaataaataaaaacttaaagaacaattaataaattttaagagataaatattaagcaaactgttaatattttttaaaaatattaagcaaacacatataaaaaaagttagctttttcagttataatggaaatagatcgacccttggtgaaattcgttattaactgaccgataaattaaaatataaaaagatgtatttatgtacagatcaatttattgaaaaataaagttatagcttttatttaaatacattataaacaaaagatcaattttatttaaacacattaaataataaaataaattcagatttttaaatatatttattttaatcatatatttcatgtatataatcaaatgtattggattaagtctctctgatcaaagtggtaaataagttatcgattttgaatttttggaggattatattaaatgtgattaattagattttttaatagtcaataacatgataaaaaaacgtatttgattaaaagtaaaagtaattgtagatatatttttaatttaaaatcataatcaataacattatctttactttttgtcctaaattggtatcgagtcgacaccacacactcaagtgataaacgcgttgacttgagtccattgtgaaacaaataaagcaagcaataatcaaacacttgatcgatcattttacaaaatctgggtgtcttaatatctgtcggctacggctgctcgcactcgcagaggtacacagtcatcatcataaatctttgttaactgttgtttatgattcctcactcacccttcagatggatctcaaatctaacctcttaagtcttaagtagtttgattctgtatatcactttcaactaattaaggaataactaatttattgtcttctatttttcttttcatctttctctaaaatacaataatataacttataacacaaaagttttatgattgttgttttttttttttattttgtcagctaatttccaaaaatgatattattttactccttcctaattaataaaattcgatcgtattttcaacaaattatatttgtatatttaattatttcacttttattttttttataaatatgttatacctaataatagtaaataatccatatctatctaaatctattacatttttaattatacacacgcagactaacacatatatgggataattagtttattagataatttttactaaatatttcatattcaatagctttcaaactatatctgacaaaaataattctaagttggtgcaatgcttaaaagtgatttttttaactaaatgaagtcattaaggtagaacaaacttataaattaaataagttatcgattatttaaaatcacttatcaaacacattcttgggtatattatatttagtaagactcataaataaataaaatggcttaaagttgaaaaattatttgaagttaaagttgttaaactcaatcatttaattagaagtgttcaataaaaataatcattgaagtaactaataaatataaaatgatatgaaatattctttttattttgtagtcaaaagtatatatttttaaaatgtagatgttatagattaaacagagatagaataatcaaatgtatcttaatcagtagtatttattttggttggtgcctgtcctccactagtattgaacaaggagaccagagactcaatttggtgctgctcatgcgttcactaacccatcatttagtaaaatctgtgcacaagaggtgctggcagagctagacatgcatgaatctttcttaactgttgcttctgattcccccgagatggatctcaattcttaacagttagcagcagaggtacacatgaatctttctcacttaactcttgctcccgattcctcctcagatacattccaatcattttgtatatcagtttcaacatggacggattttgggggggatgggggaagcaggggccttggccctccctcaagatcctttatatatacttgaggaaaaagaaaaagaaaatattactagtataaaatgaataataaaaatagagtattgacttaatgtttctttaattttttttctagtagtagttcccgccacaggccacagcttttcttccattggattccattttatcccaactccagcaatcgagaaatacttgtccttgatttacaccttctgtccttcttgacaaacaagtttacttccactttcgcaattcca

SEQ ID NO: 11 - DNA, 2000 bp, Glycine tomentella
  source 1..2000: mol_type = other DNA
ttgttgagtggttgagttcaatttgttgataaactcagatgcaagggtcaagaaattgcaatagttgacatatgcaagaactttaatgctgaagttaagtatcaggggctgcttttgcaacaaaatgtttgaaatttatgattgcaacaaaacgtatcacaaggatttgcctgagttgctgataatatgatctgatgtacaaattttcatgaaataaaagttttgtcaagctgtcccaaatgaacattttgagtcacgctaactgaacagttcctcaagttatatttgagactttttatttggttaaagcttgtcaaatttcctgattgatgatgggttttcattgcttgcaattactaatcaaatttaagacttgggaaccattttgttacgtgatgccctagggaatgccatatccgcatctgtagtcttcagttgctgcgttgtttcgttttctgttttttctcattacgtaaccaaaataaaataaaatttaagagtggctccaaggtgcgtatgtaagagacaatgagcttatcaattgattagctaacacaacatattggctcttaacattttttttataaaacaaaacaaaaaaaaaaaaacctttgttatgcttggaaagttttcagagacagtgataactcatccaagctgaaggtatccttgtacagtgcaaatattagcatatgtaacttagcttagccctggtgaaaaactgacgggctaaaaagtgccaatgactctaaaatacaataaatcaatagcttcaaatgaagatatgccagatataaaacacatcactttatggctttctcttcatttttctttgagcctttaagtctatcacaattacagagatgttatcttggcttcccctgtggatggcaagcttagttagatactctgcagcggactgagctgcaggatcagctccttcactgcgtcctgttgtgtcgttgtcaccatacttcttgtgccaaagaaggattcgcttctttgcaacctcgcatgcttcttcatttgtcattacatcccataaaccatcacttgctaaaataagacactcatcatttttctctcgacgcacaatattgacttcaggttctggaattatccatggtttcaggtatctatcacctgatgggcaagaagaaaaattagacaacaaagattattggaataagaaatttatgtagaaaaaagttgtagagtgaaatcacattgaaggaagcttacttaagtcacattttgctccaaacgttattgaaggaggtggaataaaagaaacttactacgatgtaccaataaaagaagagtttatgctctatgtactagcactgtcaaaatttctacaatcatgataggtaagacaaaactttaaaatggctattgttaagttcataaacttattatacatgtcattttgtgattggatggcaatgtaaaaattgcatacattgttgtttacttgtaaaagaacacttgtatagaagaataatgtatgtcctcacattcaacttcagcacaaactcgtgattaataatattgtattgtagtagagatgtagaactgaaaaaataacggctaaaccatttgattatagtttcatataaccacataatattgcaagaaaaccaatcatattctagagatcatgcaacaaacacatacctatagaccttgacatcgccaagacaccaagaactcaatatcctttccaatgtatgactcttcctcccgcagcttctatccgagcccgctcatcctctctatttggatgcaaaacaggaagtcaagtttcctctatcagacatcttcagcttctaatataaattcaccaaaaatactataaaattagaatgaaatgccttactttgtggtcagaagacaatggcatggcttctttaccgcggtataagacagtccttgaatctccacaatttgcaattatgatgtgggtttggcttaaaatggcaaccactgcagtggagcca

SEQ ID NO: 12 - DNA, 2505 bp, Medicago truncatula
  source 1..2505: mol_type = other DNA
gttgccatcgtttcatcctagggttttattgttactgttattggcaattagatatctgaaatttggtggcatgcttcagagttagaggatggagttttcaagtttttttggtttttgtttgatgatgttctaagttttttggtttagcttttgctggtctattaatggcatgatgcttcactgatgacaaataacaatgtaatgaagtgtcagttttgattttaagccatattcgtgtattgttaaagcatccaattttgtcaatcaattgatgcttcaagtatttcttttattaatgcaatgaattttcgttttaccttataatccatatgagaactttctttaattttccagttatgtgtatattgattttgtattttgttgtaaagaattaatcatagtgcactgaacctagcaaaacttgggaatttgttgatattgaagctaatttaatcaatagtcttgcttaaatggaataagaatggcgtggtactcttcaaaatatcatgattgcacatttattttccctttaccgcttcacaacatttaattcagcactactatcaattattctctatcaatgcatattcccatgcaccagcaaaagtatgtcatgtaactataaatataggtgcggtgctatcgaaaggcttgccattctcattctaaactcacttagcatcacatgtttgtatcaaaaaatggctttacgtgagtttgagagcttacttgctttgatttgcttgaaaggtcccataatctttgtttattatgtttatggtttatttttatgtgaatatgaaaaccatccagcatcaaaatctaaacgaaggaccgcatattatttaccatcatttttaccaattttcattctcaacaacaccattatttacccaagaaaataattaaacaacaacatattattttccgccaaccgttgctttttctacggacacccaaatgtaaaacacccttaattccaaagatttccaagatataaataaatacaccataaacattcttcttctaacaaagaagacctaaccattgttctaacagtaacacctaaaattcagttacctaacatgtcaaagtgaaaactgagcacaaaaactaaaatcaaaacaccagttaagaaaataattaaacaacaacacaaaattttgcattttgatgtatgaaaagctttaaaaaaagtgactatttctaaaatcacttttaaaaaaagaaacatccaaagtcatgagtgagtcaaaggggttataaacttctgcattaccctaatctttctattcttgaaattccatgagaaaataaacaacagcacataaatgggttaagctttaaaaaaagggataatttgtaaaatcactttttcagacatttttgtttatgcatgtggtaaaagtcatctaaagaacatccaatggaagaatcgtacaaaaaatcattgatctaacagaatggaccaaaacgagattaaaaaaaaacttaaatactcaaacaatcctttttttagttaagaaattaaaacaataccaattaaatatttaaggaaccaaaataacatttaagcctaaattattttgactaacataaatacataattaaaaactattttaaaatttcaatacaacgacacaccttgagagactaaaatgattattattcatattatccattaaaaaggaaaaggagtgcgtgtgaaaagcaggccacacgtatatgggccttgaactcgaattcgaattcgaatcttgtggccacctaaccacttcactaaactcacgaataattggtgaacaattctttacaacgaaactaacgaatatcctaataccagatcctaatgacatttttacccctaaactcttaccctcaacactttcaaaagaatcatcactaaattgtcattttaacacactctagaacctactttgataacgaaaaagtagaaagaaacccgtgctaggtcccacccaatagacaacgcaaaccacaattaccagcgcagtatcacgggtcatgcaccggtcacgtagcgtagataaaacccggaaccaaacttgacctacccccacaaacaagtcattattttattatatttatccttaaaaccctatttctcatttcatttcttctacaaccgtctactctagctcgttagttccgctatcactctactcttctactacttcacttcatcctctttgtttttcagcctagggtggtggtaattattcataactgtttcattatttttccttaatttctcattaattttccgttaatttttcaattagtgtaagctgatctgatttttttcctttgtttttgtttcgaattttgatggatcagccaagatcaagatcgtaatcaacggtaaatattatagttccttcgatctctgttcatttatgcgtcgatcggatttatattttttaatttagtttattttttgttaattttgtttgtaactgataggatttggaagaatcggaaacc

SEQ ID NO: 13 - DNA, 1000 bp, Medicago truncatula
  source 1..1000: mol_type = other DNA
gctgtttcaatactgtggtagtgctataaattggcctactttttgtttcttacagtctattagcctagcagccgttaggcctcttttttaaatgtaataagttgaattcctgaactcgtgaagcttgccccttattgaaccgtagtgtttttggttgcttaataccattaagcattgagttgttttggatttatttgcatttaccgaattttatttggaagatagtaagcgttttttccatgttcacaaatatgccatgaaaaaagcagtaaaatttcaataaaccaacatcatgataaaacaaaagtatgcatcgtatgaaaaaaagtgggtgatgaaatgataagtctaattttggtcgaatttggtcgttgttatttttgcatgaacatctcttttgtacaacctaacaactaatttgctatttttaatatttttgtactgcttttgatttgcttgctcttcacaatattatagtgttgcattagttgttcaattttggttgtacaaataatacaacttttaagcagttatcgtgaatgttagaaaaatcatgtgacttatctctgttattgggtgattgcatttgtttcgtgacttgtctttaatcggttacttattttcaataataaataacaatatagcattattttgtgtcactttcttacttttgattattgaaaagccgtgctcctttgcctgtctcttgagccatgtgattcgtcgtgaattgtgaggacgagatcatatttgagaaagaaaatgtgagctagacgtttctatagctttattacgtagcccttgcaattcaattcagcagggaacatatcaaaatgatttgtattgaaaaaattgttctacctacaaaaatctactttattatttgtactgaacgagaaagtgccacaatacttaattagataagtagacattggagctaatctcaaaattcttgcctgctttttctatcatatatctccatatcactaattaattcatt

SEQ ID NO: 14 - DNA, 2393 bp, Glycine max
  source 1..2393: mol_type = other DNA
agatttagggctcaattaacgttaatgaccaaatagaaaaactgctttgttggtttgaaatttgtgatggagaaaatgtcattaagataagagatcactctaattaagatgataaatagaccttcaataacacataatatgataacttgacattctaattgccatgtagttttttggcactagaaaagcaaaatgaccaaattacatagactaaaaaaattagcctaaataaaataatataatattttttaacgataaaaattacaataattaaaatgtcattttacaggtttaatttttaacgttcaaagtataaataaaacaaacaaatcatatttcttaagtttaaattttaataaaatgaaataatttccattaataataaaaaatttacatctaaaataaataatataatgacaaattaaaacaatataaaatcttaggaaattaagatataaaaattataaattcatataaataagtaaattatatgttttataaaaagaaacaaataaatattttaaaaataatttatatctatttgtatttttatcaaatatgtaattttgtttgtctttaattaatttttaatttttaaaaataataaaaattgaaaatgcatctgtatcgattcttgaccttttttccccaccaagagaccagtttaatcaatttttgacagattctcaccagttttgaaatctttcccatttttttttatcaaattcaattttaaaaattatgatttccacggtcacaacaattaataacgtacaaggtacgcaaacccgtggcatgcagcctcccattatccagtttttttttttacaatattcaatttacatgttggacttaacacacttaataatttttttgtgctaagaacaaaaaaacatgataaagatgaggaggactcaagtgaccgcgatggccaagatatattctgaccaaaaccaacgtttgtgttttattaaatatgcaattattaaaacttggaaatgtagaacagaaatgtcctagctatcagttagacgccacaatttttttttgcctcctattgctcaagtggtcaaattaatggaaaagaaaaaagacaactaattggatgaaaaagaatttgtccagatataattttcaattaatggcgtccaattactaaatgtgtgtttgaaaactaagaaatcatataaaaaaaaatttacattaaaatgcagaaagttaattataaccttcttaaatatgaaaaattaatcaaatctgaaatataaaatcaaataagctaataaaattgttgaaacgcagcttagaacctgagactccaataatatgaattatttcttcgtgcttttgacttgtaagctattggttctaataaattccaggattgtcttacttattcaattaatggcatccaattattaaatgtgagttgggaaacaccttagaaattaagaaatcgttttaaaaactataacgataaacgtcttagtataaatgtgaaaaaatcacattgaaatataaaaccgaatacgctatatatgcaaaattgttgaaagacaattaaatggtagaacctcagccccaaacaatatgaatttgttctcgtgcttgtaagttgtaggttatagtaattagatggaggtaaattttgagggtataaaaacttcgtaccccatcgcatatgcaaaaaggctgtttccggattcgaacccatgaccaacaggcacaactttaccgctgcaccagggctcgcccaaattttgagggtatcaaatacataataataccatggcagatctatggttgttagacgtctgaataagtcgttcacaagcttgcataataaatacaataataaattttagaacttcatttgcaagtgccacctactgaagaaaccacgtgcttagtactacaatttaagggaaattgcaaggaagtttggtttcgcctccagttgagtccaacgttgcccaaagtagacaattcaacgacgcaaatagattcgtaaggaacacaggagaatttttaatttgtctagttgtcttcacttaactctttgcattcctataataggcacacaattagcatcaacaaacacatcaagatttgtcataagaaaaaaagaaagaaaagaagtgcgagagtaggagaacttccccgtgatcaacttagacaatctcaacggtgaggagaggaaagctacactagaccagattgaagacgcttgccagaactggggattttttgaggtatcgcacaaaccaaatcacaaaaagctttgttatatatatctgttcatttgcaagtgtacatcccaaaattttcttcatgttattcatttgaactatattaatttagctgctatatatccgaattgtagttggtgaatc

SEQ ID NO: 15 - DNA, 2309 bp, Glycine max
  source 1..2309: mol_type = other DNA
tttttttttaatttgaatttttttaaaataagtgttttttttaaataataactagatagaatctgtttcttaaataagcataaaactgaaataatttaaacaaatctactaatttttttattaagataaacgtttattttaataaataagtttaaacaaataaaaaattactttttataaaaactatttttgaaattaattttttaagtttaaaaaaacacactcaacaaaatttaaaaattttattttaaccacttatttttcttctgatgttaaaattgaaaaccctaattctttacaattatcgtctaacatcttcattttcataactgtgtcacagtatcattgcaagatcgttcttgaattttcctaatcatattaaaggcatcattaattaataattagtcgcctaacatattcatcattaacaatatagtgtaagtagtgagtccaaatatagatacttagatattttcacgtagaggataatgaacattcaagtaattggtgtggataagttcgggagataggtatttttggcttttacacacatatatgggaacagacattaattctaattccctatccaaatctgagttctaatatataatgactttcatgtgtgcttattacactagcataataatgaactttagtagaaaggatattgtaggatgtactaacatgtttttgctgcaggtgatttaggtggatttggttccataatgcttcactttctttgttaatttttttgatttgcaggcagggccccatttttttgtatcaggaggtttgaattccaccaatttcagcacaccttagttggtcattaataaaaaatttatttataaaaaaaaagcaaaatgtactttattctaaaaggtaaaaaataaataaccagtcctgtaactattttagtggtgttcacaatgaatgtgcaagtcttgattgtgatatatactttgaattctagttgaggcattctatgctagttatgctctagggaaagaggtttgttgttgcagggaataatgcattacgaatgtcatagcatggcacactttcatggaggtaatccaagtcgcaattcacacttttataaattttctttttgctaatcaaaaggaaaaatatgtcgacagttatatatgtcagttccaagatgcgatgcaaaaatctcttgtgacatatattttaaaccacacattcatattcgaatttgaagagtcgattacaataagatattataaatatcaatgttttgagccagtcaattttgagaagaaaaaatatatgaatatacaattgctacccggccccaaatatatataagattaattgaattttttggagaacagcacacaatacagatgttctcacgaatatttgttcagaactagacaatagatctagctaataaaaaatatagtgatgttctcaaccatgtaactgatgcggctgtaatctttattttcttcttcttttgaataaggtttatgtagctacttaccacgttgaatactgaaatacttttaggactaaaggacagtcagacaaagtaggagtacaaatgatcaacggcgagaagcttcaaatttctaagtaaaagcattttaattaatgtcatactgttgtacagccaaatatattaggcaaaccccccacgtttttatttgactaaataataatcgtgtgaaagagacgcacacctacacacaggtcgacgaggagagcgacattttagaaagaagaaaacaatgatggacccaataacattatttattattattcatataatttggactttggagttttgtactgttatgtacaactcagttccccattcgtgctaatataaaagaacaactatccctcgtgtgagatttgaatcaactttggtgtatatatataaacaagagagagctagcagtgcaagctttcaacatccaaaacaacagagagtttttggctttagaagctgaaggaatatagttatcacataggggagagctccatgctgtgagaaataggggttgaagagagggcctaggactccagaagaagatcaaattctcatcaattacatcaacactttaggcctagctaattggcgtgcactgcccaaactagctggtacgtgtgaaggaatataattaattaacccaatattcttattgatcaaatgcctgaaacaagttatttgaagaaaataacataggaattcaattgtggtacgtttcatgaatatagttaaccaaagtttctatatatctctttttgatcaacagggctattaaggtgtggaaaga

SEQ ID NO: 16 - protein, 135 aa, Phakopsora pachyrhizi
MMAFNRFAYLLLVSIMVASFTTGQLLPSAYKCNSVDSSRFPINREDCRDSLNLFLSPRNVLTFANGNTQSCGTCRVTIIKSGTSAPPRTAQKSWAVTALHEGYDNCNGNPVTATIGDSAMIDVSLSYGNGTPCPP

SEQ ID NO: 17 - DNA, 408 bp, Phakopsora pachyrhizi
  source 1..408: mol_type = other DNA
atgatggcgtttaaccgatttgcatatctgcttttggtgagcattatggttgcgtcctttacaactggacagctccttccaagcgcctacaaatgtaacagtgtcgattcatctcgatttccaatcaatcgagaagactgtcgagattctttaaacctgtttttgtctcccaggaacgtacttacttttgcaaatggaaatactcagagctgtggaacctgcagggtaacaataattaagagcggaacttctgcaccacctagaactgctcaaaaaagttgggcagttacagcattacatgaagggtatgacaactgcaacggaaaccctgtgactgcaaccattggagacagtgccatgatcgacgtgtctctaagctatggaaacggaactccctgtccaccgtaa

SEQ ID NO: 18 - protein, 188 aa, Phakopsora pachyrhizi
MGSHNMIKLLLLLLLPLQAISKFTFSSKIGAIDSTANINSLDNIGSTKKWAEGADVVNLSRGKNPQQASYYRSNAFKKPKTASLTRSKAMRLPDGVEFVNVPQLKQKSGFFTSIYQKFKNLLSRIVPRKSPKNKATVHAEESVFIQPNTLDASTTKALKKPRKKVTFLVDEKTGNPIVEILGESEKAV

SEQ ID NO: 19 - DNA, 567 bp, Phakopsora pachyrhizi
  source 1..567: mol_type = other DNA
atgggttctcataacatgatcaaattgctgttactgctgcttctacctctccaggctatttccaagtttactttttcttcaaaaatcggtgcaatcgattctactgcgaatataaattcgctggataatataggtagcactaaaaaatgggccgaaggcgctgatgtggtaaatttgagtagaggcaaaaaccctcaacaagcatcttattatagatcaaatgcgtttaaaaaaccaaaaacagcatcgttgactagatcgaaagccatgcgccttcccgatggcgttgaatttgtgaatgtgcctcaattgaaacaaaaaagtggattctttacttcaatatatcaaaaatttaaaaatttattaagcagaattgttccaagaaaatcccccaaaaacaaagcaacagttcacgctgaagaaagtgtgtttattcaacctaatacacttgatgcctcaaccaccaaagctctcaaaaaaccacgcaaaaaagttacgttcttagttgatgagaaaactgggaatccaattgtagagattttgggggaatcagagaaagctgtttga

SEQ ID NO: 20 - protein, 1570 aa, Glycine tomentella
MASISGVQSSSSAYDVFLSFHGEDTRNNNNNFTGFLFQALRRKGIVAFKGHTLPEKGEPGLLHAIEGSRLFIVVLSKNYASSTRCLRELAHIFNCIETSPRRLLPIFYDVDPSDVRKQTGRYEEAFWKHEQRFLVDKERMEEVRTWRKALSRVANLPGWYIPNKPQYTEIEEFVQYIITILGPNISTLQNEAEGTSSDKDEVSYNELSGLMRIRDFISLVLTYPGKLEDENILRSWISAFPEWKAQLLSTGDDTVMDKPWCVLTDKALGPMKGFMPMEIISKVESGERSIFSHNDVDAIPEAILQQSVDLAVNQLLQTLFSGDYCARYIRLLTRNLAEKGSVVDTIVSAVEDKHTMFGIDKDFLRALWINASTYETDAEVRVQEEIDINMMMEVDDMGSNGQLVIVVDADSNNKLDLQKLRFPTGIVVLITTEPSTQAEKDVDFRIACTMDLNIWTQDHLLPWKVFCSYVGSCISSSMVSSSMAIQKIAVEIVKESHGHLLAIVLVAKYLTYVQDFKQWELALDKLSSLNPSYDYQDSDRIGISRVMVNAFVNIIWEDIDDAQKLCLELSLPVHNIKNGVRDDILVSDWAKIISGYTQELGEYRRQLQHYINELQDRFVLLKYESGDVYLPVETYDIIKSLHTSKPSILRHGALGLTEPPYIGRWHSLIQIELMDNKICELPQSPDCPKLKVLLLQGNVDLMDIPDSFFNHMPLLQHLDLSYTSIRDLPPSVSKLIQLQKFYLRGCDLFMELPHQIGQLKNLKELDLDGTLITHLPIEIRELINLQSLSLCFDGTSRSTIIPPGLVSNLTQLNYLSINVDPEDERWNENVISVLREIFFGLWNLQRLSIYVPKADLLEFIPLLPAQKSLNFRLVVGQHMRRLISRVPPELETKFKHCDYSIKFVNGVNVPNGVKMHLERFKALYLDRHMTIKSLSDFDLRNLWGLEVCILAECNEMETIVRGSNSLMLKFLSVFYMKNLRSICEGSSPFFLCLKSIALHTCPMLTTIFTLDSLEKLSFLEEIIIEDCPKVTTLISHATPEQKPVFFLPKLRMISLLYLPELVNIFNGMHVGPFIEEMIFYYCPSLQSLSKSELSSKSLKIIKGESIWWEALKWNVAEWGDAGRPNYFERIFSPINEEADMMNRLAALQETQLNEYHNTTYQGISSSTQSIAKSSLEIPVLSPLAPVSSVGRGSSNSEAQKRKVVTEPKILQDASKKCNLIVKKGNLGRDLSVMCGTFKDGDLLVNRDGVRIVSQNDVEAPPQIKPTDNQLTLADIDVIKVVGEGNGGVVQLVQHKWTRQFFVLKVIQMNIEESMRKQIAQELKITQQAQCPYVVVCYQSFYENGVISILLEYMDGGSLADLLKKVKTIPEDYLAAICKQVLKGLVYLHHEKHIIHRDLKPSNLLINHIGEVKITDFGVSTIMESTSGQANTFIGTYNYMSPERIIGSQRGYNYKSDIWSLGLILLECALGRFPYAPPDQSETWESIFELMETIVDKPPPSPPSEQFSTEFCSFISACLQKDPKDRLSAQELMAHPFVNMYDDLEVDLSAYFSNADELKNQLLESLQGFCI

SEQ ID NO: 21 - DNA, 5171 bp, Glycine tomentella
  source 1..5171: mol_type = other DNA
gctgctcgcactcgcagagtattgaacaaggagaccagagactcaatttggtgctgctcatgcgttcactaacccatcatttagtaaaatctgtgcacaagaggtgctggcagagctagacatgcatgaatctttcttaactgttgcttctgattcccccgagatggatctcaattcttaacagttagcagcagagtagttcccgccacaggccacagcttttcttccattggattccattttatcccaactccagcaatcgagaaatacttgtccttgatttacaccttctgtccttcttgacaaacaagtttacttccactttcgcaattccaatggcttctatctccggcgtccagagcagctcttctgcatatgacgtgtttctcagcttccacggtgaagacacacgcaacaacaacaacaacttcacaggttttctttttcaagctctcagaagaaaaggcatcgtcgccttcaaaggtcatacacttcccgaaaagggtgaacccgggctgttacatgcaattgaaggctcccgccttttcattgtggtcctttcaaagaactatgcttcctcaactcggtgtttgcgtgaactagcacacatcttcaattgcattgaaacttcgccaagacgtcttctgcctattttttatgacgttgatccatcagatgtgcgaaaacagactggacggtacgaggaagccttttggaaacacgaacaaagattcttggtagataaagagaggatggaggaagtgcggacatggagaaaagctctgagtcgagtggccaatctccctggttggtatatcccaaataagccacaatatacagagattgaagaatttgttcaatatataataactatattgggtcctaatatttcaactcttcaaaatgaagcagagggaacatcttcagataaagatgaagtttcatacaatgagctgtcaggcttgatgaggataagagattttatcagcctcgtgttgacttatcctgggaaattagaagatgaaaatattttaagatcttggattagtgcatttcctgaatggaaagctcagttattgtccactggtgatgacactgtgatggacaaaccatggtgtgtactaactgataaggcactgggacctatgaagggatttatgccaatggaaataataagtaaagtagaaagtggtgagagatccatcttcagccataatgatgttgatgcaataccagaagccattctccaacaatctgttgatctcgcagttaaccaactacttcaaactttgttttctggggattactgtgcacgctatatcagactgttaaccagaaacttggctgaaaaaggatctgtagtggacacaatagtttcagccgttgaagataagcacaccatgtttggaattgacaaagattttcttagagccctatggataaatgcctcaacatatgagactgatgcagaggttcgagttcaagaagaaatcgatatcaatatgatgatggaagttgatgacatgggaagcaatgggcagttggtaattgtagtggatgcagatagcaacaacaagctggacctccaaaagttgcgttttccaactggaattgtggtgttgataacaactgagccgtcaacacaagcagagaaggatgttgactttagaattgcatgcacaatggatttgaatatttggacccaggatcatctattgccttggaaagtcttctgcagctatgtcggcagttgtatcagcagtagcatggtaagttcatcaatggccattcagaaaattgcagtggaaatagttaaggaaagccatggtcatcttcttgccattgtccttgtggcaaagtatcttacgtatgtgcaagattttaaacagtgggaacttgcacttgacaaattaagcagtctgaatccttcctatgattatcaagatagtgatcgaattggcattagtagagtcatggtcaatgcattcgtaaatattatttgggaagacattgatgatgcacaaaagctttgccttgaactcagtttgcctgttcacaacattaaaaatggggtgagagatgatatcttggtgtctgattgggctaagataatatcaggatacacacaagaacttggtgaatacagaaggcaattacaacattacatcaatgagcttcaagaccgttttgtcttgttgaaatatgaaagtggagatgtctatttgccagtagaaacctatgatataataaaatcattgcacacctcaaagccttccatcctaaggcatggtgcattaggattgacagagccaccctacattggacgatggcatagtcttattcagatagaattgatggacaataaaatatgtgaattaccacaatcaccagattgccctaaactcaaagtgttgttgttgcagggcaatgttgatttgatggatatccctgactcatttttcaaccacatgcccctgcttcaacacttggacttatcctacactagtataagggatttacccccttctgtctctaaattaatacaacttcagaaattttatcttagaggctgtgatctcttcatggaactaccacatcagattggacagctaaagaacctcaaagagcttgatcttgatgggactttgatcacccatctgccaatagagatccgagaattgatcaacctgcaaagcttaagcctctgctttgatggaacttccaggtcaacaattattcctccaggattggtatcaaatcttactcaattgaattatttaagcatcaacgttgaccctgaagatgagcgatggaatgagaatgttattagtgtcttgagggaaattttttttggattatggaatttgcagaggttgagtatatatgtcccaaaggctgacctcttggaattcataccactgctaccagctcaaaaatctctcaatttcagattggttgttggtcaacatatgcgacgtttgatatcgcgtgtaccacctgaactggagacaaaatttaaacactgtgactatagcataaagtttgttaatggtgtgaatgttccaaatggagtgaaaatgcatcttgaacgcttcaaagccttgtacctagaccgacatatgaccatcaagagtctctctgatttcgatttgaggaatttatgggggttggaagtatgcatattggcagagtgtaatgaaatggaaacaattgtcagaggaagcaattctctaatgttgaagtttttgagcgtattctatatgaagaatttaagaagcatctgcgaagggagtagtcccttctttctctgcttaaagtctatagcattacacacttgccccatgttaaccaccatcttcaccttagattctcttgaaaaactttcctttttggaggagattattattgaagactgtccaaaagtaaccaccctgataagtcatgccacaccagaacaaaaaccagtgtttttcctccccaagttgagaatgatatcgcttctctatcttcctgaattggtcaacattttcaatggcatgcatgttggaccttttatagaagaaatgatattttattattgcccaagtctccaaagtctttccaaatcagagttgtcaagcaaatctttgaagatcatcaaaggagaaagcatctggtgggaagcattgaaatggaatgtagcagagtggggagatgctggtcgacccaattattttgagcgaattttttcaccaattaacgaggaggctgacatgatgaaccggctagctgcacttcaagagactcaactaaatgaatatcacaatacgacttatcagggaatttcaagttctacacagtcgatagcaaagtcatctttggaaatacctgttttgtcacctttagccccagtttcatctgtgggtaggggtagttcaaacagtgaagcccaaaagcggaaagtcgttactgagccaaaaatattacaagatgcatcaaaaaagtgcaatttaatcgtgaagaaaggaaacttggggcgtgatctctctgtgatgtgtgggacttttaaggatggggatctgcttgtcaacagggatggagttcgaattgtctctcagaatgatgtcgaagcgccacctcaaatcaagccgacagacaaccagttaactttggcagatatagatgtaatcaaagttgttggagagggaaatggaggggtagttcaattggtgcaacacaaatggactcgtcagttttttgtgttgaaggtaattcagatgaatattgaggagtctatgcgcaagcagattgcacaagagctgaaaattactcaacaagcacaatgtccttatgttgttgtctgctaccaatcattctatgaaaacggtgtcatatcaatcctcttagagtacatggatggaggttccttagctgatctgctgaagaaagttaaaacaattccggaggattatcttgctgccatctgtaagcaggtgctgaagggtttagtttatctccaccacgaaaagcatattatccacagagacttaaaaccttctaatttgttaataaatcatattggtgaagtaaagattactgactttggtgtgagtacaattatggaaagtacatctggtcaagcaaatactttcattggaacctacaactatatgtctccagagagaatcattggaagccagcgaggatacaactacaaaagtgatatatggagtttgggactaatattactcgagtgtgctttgggtcggtttccatatgcaccgcctgatcaaagtgaaacatgggaaagcatttttgagcttatggaaactattgtcgataaacctcctcctagtcctccatctgaacaattttctacagaattttgctcatttatctcagcatgtctacagaaagacccaaaggatcgactgtcagctcaggaattaatggcacatccttttgtcaacatgtatgatgacttggaggtggatctttcagcttacttctcgaatgcagatgagttgaagaaccagctcctagagtcacttcaaggattctgtatttagttgttgagtggttgagttcaatttgttgataaactcagatgcaagggtcaagaaattgcaatagttgacatatgcaagaactttaatgctgaagttaagtatcaggggctgcttttgcaacaa

SEQ ID NO: 22 - DNA, 4713 bp, Glycine tomentella
  source 1..4713: mol_type = other DNA
atggcttctatctccggcgtccagagcagctcttctgcatatgacgtgtttctcagcttccacggtgaagacacacgcaacaacaacaacaacttcacaggttttctttttcaagctctcagaagaaaaggcatcgtcgccttcaaaggtcatacacttcccgaaaagggtgaacccgggctgttacatgcaattgaaggctcccgccttttcattgtggtcctttcaaagaactatgcttcctcaactcggtgtttgcgtgaactagcacacatcttcaattgcattgaaacttcgccaagacgtcttctgcctattttttatgacgttgatccatcagatgtgcgaaaacagactggacggtacgaggaagccttttggaaacacgaacaaagattcttggtagataaagagaggatggaggaagtgcggacatggagaaaagctctgagtcgagtggccaatctccctggttggtatatcccaaataagccacaatatacagagattgaagaatttgttcaatatataataactatattgggtcctaatatttcaactcttcaaaatgaagcagagggaacatcttcagataaagatgaagtttcatacaatgagctgtcaggcttgatgaggataagagattttatcagcctcgtgttgacttatcctgggaaattagaagatgaaaatattttaagatcttggattagtgcatttcctgaatggaaagctcagttattgtccactggtgatgacactgtgatggacaaaccatggtgtgtactaactgataaggcactgggacctatgaagggatttatgccaatggaaataataagtaaagtagaaagtggtgagagatccatcttcagccataatgatgttgatgcaataccagaagccattctccaacaatctgttgatctcgcagttaaccaactacttcaaactttgttttctggggattactgtgcacgctatatcagactgttaaccagaaacttggctgaaaaaggatctgtagtggacacaatagtttcagccgttgaagataagcacaccatgtttggaattgacaaagattttcttagagccctatggataaatgcctcaacatatgagactgatgcagaggttcgagttcaagaagaaatcgatatcaatatgatgatggaagttgatgacatgggaagcaatgggcagttggtaattgtagtggatgcagatagcaacaacaagctggacctccaaaagttgcgttttccaactggaattgtggtgttgataacaactgagccgtcaacacaagcagagaaggatgttgactttagaattgcatgcacaatggatttgaatatttggacccaggatcatctattgccttggaaagtcttctgcagctatgtcggcagttgtatcagcagtagcatggtaagttcatcaatggccattcagaaaattgcagtggaaatagttaaggaaagccatggtcatcttcttgccattgtccttgtggcaaagtatcttacgtatgtgcaagattttaaacagtgggaacttgcacttgacaaattaagcagtctgaatccttcctatgattatcaagatagtgatcgaattggcattagtagagtcatggtcaatgcattcgtaaatattatttgggaagacattgatgatgcacaaaagctttgccttgaactcagtttgcctgttcacaacattaaaaatggggtgagagatgatatcttggtgtctgattgggctaagataatatcaggatacacacaagaacttggtgaatacagaaggcaattacaacattacatcaatgagcttcaagaccgttttgtcttgttgaaatatgaaagtggagatgtctatttgccagtagaaacctatgatataataaaatcattgcacacctcaaagccttccatcctaaggcatggtgcattaggattgacagagccaccctacattggacgatggcatagtcttattcagatagaattgatggacaataaaatatgtgaattaccacaatcaccagattgccctaaactcaaagtgttgttgttgcagggcaatgttgatttgatggatatccctgactcatttttcaaccacatgcccctgcttcaacacttggacttatcctacactagtataagggatttacccccttctgtctctaaattaatacaacttcagaaattttatcttagaggctgtgatctcttcatggaactaccacatcagattggacagctaaagaacctcaaagagcttgatcttgatgggactttgatcacccatctgccaatagagatccgagaattgatcaacctgcaaagcttaagcctctgctttgatggaacttccaggtcaacaattattcctccaggattggtatcaaatcttactcaattgaattatttaagcatcaacgttgaccctgaagatgagcgatggaatgagaatgttattagtgtcttgagggaaattttttttggattatggaatttgcagaggttgagtatatatgtcccaaaggctgacctcttggaattcataccactgctaccagctcaaaaatctctcaatttcagattggttgttggtcaacatatgcgacgtttgatatcgcgtgtaccacctgaactggagacaaaatttaaacactgtgactatagcataaagtttgttaatggtgtgaatgttccaaatggagtgaaaatgcatcttgaacgcttcaaagccttgtacctagaccgacatatgaccatcaagagtctctctgatttcgatttgaggaatttatgggggttggaagtatgcatattggcagagtgtaatgaaatggaaacaattgtcagaggaagcaattctctaatgttgaagtttttgagcgtattctatatgaagaatttaagaagcatctgcgaagggagtagtcccttctttctctgcttaaagtctatagcattacacacttgccccatgttaaccaccatcttcaccttagattctcttgaaaaactttcctttttggaggagattattattgaagactgtccaaaagtaaccaccctgataagtcatgccacaccagaacaaaaaccagtgtttttcctccccaagttgagaatgatatcgcttctctatcttcctgaattggtcaacattttcaatggcatgcatgttggaccttttatagaagaaatgatattttattattgcccaagtctccaaagtctttccaaatcagagttgtcaagcaaatctttgaagatcatcaaaggagaaagcatctggtgggaagcattgaaatggaatgtagcagagtggggagatgctggtcgacccaattattttgagcgaattttttcaccaattaacgaggaggctgacatgatgaaccggctagctgcacttcaagagactcaactaaatgaatatcacaatacgacttatcagggaatttcaagttctacacagtcgatagcaaagtcatctttggaaatacctgttttgtcacctttagccccagtttcatctgtgggtaggggtagttcaaacagtgaagcccaaaagcggaaagtcgttactgagccaaaaatattacaagatgcatcaaaaaagtgcaatttaatcgtgaagaaaggaaacttggggcgtgatctctctgtgatgtgtgggacttttaaggatggggatctgcttgtcaacagggatggagttcgaattgtctctcagaatgatgtcgaagcgccacctcaaatcaagccgacagacaaccagttaactttggcagatatagatgtaatcaaagttgttggagagggaaatggaggggtagttcaattggtgcaacacaaatggactcgtcagttttttgtgttgaaggtaattcagatgaatattgaggagtctatgcgcaagcagattgcacaagagctgaaaattactcaacaagcacaatgtccttatgttgttgtctgctaccaatcattctatgaaaacggtgtcatatcaatcctcttagagtacatggatggaggttccttagctgatctgctgaagaaagttaaaacaattccggaggattatcttgctgccatctgtaagcaggtgctgaagggtttagtttatctccaccacgaaaagcatattatccacagagacttaaaaccttctaatttgttaataaatcatattggtgaagtaaagattactgactttggtgtgagtacaattatggaaagtacatctggtcaagcaaatactttcattggaacctacaactatatgtctccagagagaatcattggaagccagcgaggatacaactacaaaagtgatatatggagtttgggactaatattactcgagtgtgctttgggtcggtttccatatgcaccgcctgatcaaagtgaaacatgggaaagcatttttgagcttatggaaactattgtcgataaacctcctcctagtcctccatctgaacaattttctacagaattttgctcatttatctcagcatgtctacagaaagacccaaaggatcgactgtcagctcaggaattaatggcacatccttttgtcaacatgtatgatgacttggaggtggatctttcagcttacttctcgaatgcagatgagttgaagaaccagctcctagagtcacttcaaggattctgtatttag

SEQ ID NO: 23 - protein, 337 aa, Phakopsora pachyrhizi
MSKLFLIAAIAAFAPSNSYGSFNIPAAPQGSFGNISPFKDNVLSLSCGLNLLVGGRVAPLAQSGYANLCLQCKIDLFGVRVSFAYTNALSESINTHGLSVSDTSRLQGFVQGRLLDSAAQSSTSNECSRRCSDDQFCQSSIFSDGRCTFTAKNLASNNKNGALLAQAFTQVFNQGGSQYCGFCSGDSSCVNPSARARSFKRHAEIARRSQLQAKCPTGLTACPIVQMNQTAAEVWSPSPAVSYECLKVDEELSSCGGCATLGKGTDCSAVEGVLSAGCSAVSCQDGYKLSKMTNKCTLINSKPNLYQKYPVVQIIYSSYLTKHNSINLTAYFNVLQT

SEQ ID NO: 24 - DNA, 1014 bp, Phakopsora pachyrhizi
  source 1..1014: mol_type = other DNA
atgtctaaactttttctcattgctgctattgctgcattcgctccctcaaactcttatggaagctttaatatacctgctgcaccacaaggtagtttcggaaacatatctccattcaaggacaacgtcctcagcttgagctgtggactgaatcttttagttggtggaagagttgccccattagcacaatctggctacgcaaatttatgccttcagtgtaaaattgacctctttggtgttcgcgtgagctttgcctacaccaacgctctttcagaatctatcaacactcatggtttatcggttagcgacacctcccgcctccagggcttcgttcagggacggcttttagactctgctgctcaatcttcaaccagcaatgagtgctcgcggagatgctcagacgatcaattctgtcaatccagtattttcagtgatgggcgctgcacttttacagctaaaaatctagccagcaacaacaaaaacggcgccctcctagcccaagcatttacacaagtcttcaaccaaggaggaagtcagtattgcggattttgcagtggcgattcatcatgtgtcaaccctagcgctcgtgcacgaagcttcaagcgccatgcggaaattgcacggagaagtcaattgcaggcaaaatgtcccactggcctcactgcctgtccaattgttcaaatgaatcagacggcagctgaagtctggagtccatcaccagcagtgtcctatgagtgcttgaaggtagatgaggaattaagctcctgcggtggttgcgcaacgctaggcaaagggacagactgttcagctgttgagggtgttttgtctgctggctgctcagccgtctcttgtcaagacggatacaaactttcaaagatgacgaacaaatgcacccttataaattctaaacctaatctgtaccaaaagtaccccgtagttcaaataatatactcatcatatcttacgaaacataatagtataaatttgactgcatattttaatgtattgcaaacttag